(19) United States
(12) Patent Application Publication
(10) Pub. No.: US 2019/0218583 A1
(43) Pub. Date: Jul. 18, 2019

(54) POLYSACCHARIDE-OXIDIZING COMPOSITION AND USES THEREOF

(71) Applicants: INSTITUT NATIONAL DE LA RECHERCHE AGRONOMIQUE (INRA), Paris (FR); UNIVERSITE AIX MARSEILLE, Marseile Cedex 07 (FR); CENTRE NATIONAL DE LA RECHERCHE SCIENTIFIQUE (CNRS), Paris (FR)

(72) Inventors: Jean-Guy BERRIN, Roquevaire (FR); Marie COUTURIER, Marseille (FR); Bernard HENRISSAT, Marseille (FR); Marie-Noelle ROSSO, Marseille (FR); Gerlind SULZENBACHER, Marseille (FR); Simon LADEVEZE, Mauvezin (FR)

(21) Appl. No.: 16/330,928

(22) PCT Filed: Jun. 13, 2017
(86) PCT No.: PCT/EP2017/064414

§ 371 Date: Mar. 6, 2019

(30) Foreign Application Priority Data
Sep. 13, 2016 (EP) EP16306162

(51) Int. Cl. C12P 19/14; C12P 19/04; C12N 9/24; C12N 9/04

(52) U.S. Cl. CPC C12P 19/14; C12P 19/04; C12N 15/81; C12N 9/0006; C12N 9/24

(57) ABSTRACT

The invention relates to a polysaccharide-oxidizing composition comprising a polysaccharide-oxidizing enzyme wherein, when the said polysaccharide-oxidizing enzyme is compared to the reference polypeptide of SEQ ID NO. 1 as described in the specification by using the BLAST-P comparison method, (i) the said polysaccharide-oxidizing enzyme possesses an amino acid identity of 20% or more with the said reference polypeptide and (ii) the BLAST-P comparison method results in an E-value of 10 e−3 or less.

### FIELD OF THE INVENTION

The present invention relates to field of production of sugar products by enzyme oxidation and hydrolysis of polysaccharide-containing material, and especially ligno-cellulosic biomass.

### BACKGROUND OF THE INVENTION

Lignocellulosic biomass is a renewable source for the production of biofuels and platform molecules for the industry. Its conversion into valuable products requires the combined action of a variety of enzymes, most of which are obtained from fungal sources. In particular, the efficient conversion of cellulose to small molecules is carried out by the synergistic action of cellulases, i.e. endoglucanases (EG), cellobiohydrolases (CBH) and β-glucosidases. EG cleave β-1,4 linkages randomly within cellulose chains, thereby releasing new ends for the action of cellobiohydrolases (CBH) which in turn release cellobiose units. β-glucosidases produce glucose molecules from cellobiose, thereby alleviating the inhibiting effect of cellobiose on CBH. These enzymes are classified in various glycoside hydrolase (GH) families of the carbohydrate-active enzyme database (also termed CAZy; described notably at the internet address www.cazy.org, and by Lombard et al, 2014, Nucleic Acids Res, Vol. 42: 490-495). More recently, the contribution of lytic polysaccharide monooxygenase (LPMOs) to cellulose degradation has been demonstrated (Vaaje-Kolstad et al. 2010, Science, Vol. 330: 219-222; Harris et al. 2010, Biochemistry, Vol. 49: 3305-3316; Quinlan et al. 2011, Proc Acad Natl Sci USA, Vol. 108: 15079-15084). In industry, addition of LPMOs to cellulolytic cocktails leads to the reduction of enzyme loading required for efficient saccharification of cellulosic biomass (Harris et al. 2010, Biochemistry, Vol. 49: 3305-3316; Johansen et al., 2016, Biochem Soc Trans, Vol. 44: 143-149).

In spite of much research effort, there remains a need for improved cellulose enzyme mixtures for the hydrolysis of cellulose in a pretreated lignocellulosic feedstock. The absence of such an enzyme mixture represents a large hurdle in the commercialization of cellulose conversion to soluble sugars including glucose for the production of ethanol and other products.

### SUMMARY OF THE INVENTION

This invention provides for novel compositions comprising one or more specific polysaccharide-oxidizing enzymes.

The present invention relates to a polysaccharide-oxidizing composition comprising a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less.

In some embodiments of the composition, the said polysaccharide-oxidizing enzyme has at least 30% amino acid identity with a polypeptide selected in a group comprising the polypeptides of SEQ ID NO. 1, SEQ ID NO. 2 and SEQ ID NO. 3 3.

In some embodiments of the composition, the said polysaccharide-oxidizing enzyme is encoded by a nucleic acid having at least 90% nucleotide identity with a nucleic acid selected in a group comprising the nucleic acids of SEQ ID NO. 4, SEQ ID NO. 5 and SEQ ID NO. 6.

In some embodiments, the said composition further comprises one or more lytic polysaccharide monooxygenases.

In some embodiments, the said composition further comprises one or more polysaccharide-degrading enzymes, selected in a group comprising cellulases, hemicellulases, ligninases, and carbohydrate oxidases. In some embodiments, the cellulases are selected in a group comprising exoglucanases, endoglucanases, cellobiohydrolases, cellulose phosphorylases, pectinases, pectate lyases, polygalacturonase, pectin esterases, cellobiose dehydrogenases, beta mannanases, arabinofuranosidases, feruoyl esterases, arabinofuranosidases, fructofuranosidases, alpha galactosidases, beta galactosidases, alpha amylases, acetylxylan esterases, chitin deacetylases, chitinases, and beta glucosidases. In some of these embodiments, the lytic polysaccharide monooxygenase is selected in a group comprising AA9, AA10, AA11 and AA13.

In some embodiments, the said one or more other polysaccharide degrading enzymes are comprised in an enzyme preparation containing the said one or more other polysaccharide degrading enzymes. In some embodiments, the said enzyme preparation comprises one or more enzymes originating from one or more fungus organisms or one or more bacterial organisms.

In some embodiments of the polysaccharide-oxidizing composition, one or more of the said other polysaccharide degrading enzymes are recombinant proteins.

This invention also pertains to a yeast cell recombinantly expressing a polysaccharide-oxidizing enzyme as defined in the present specification.

This invention also concerns a method for oxidizing a polysaccharide comprising a step of contacting one or more polysaccharides with a polysaccharide-oxidizing enzyme as defined in the present specification, or with a composition comprising the said polysaccharide-oxidizing enzyme. In some embodiments, the said one or more polysaccharides are contained in a lignocellulosic-containing material.

The present invention further relates to a method for the preparation of a sugar product from a polysaccharide-containing material comprising a step of treating the said polysaccharide-containing material in the presence of a polysaccharide degrading composition as described in the present specification. In some embodiments, the said method which comprises the steps of:
a) providing a polysaccharide-containing material,
b) subjecting the said polysaccharide-containing material to hydrolysis in the presence of a polysaccharide-degrading composition as defined herein, and
c) collecting the sugar product obtained at the end of step b).

This invention also provides for a method for the preparation of a fermentation product from a polysaccharide-containing material comprising the steps of:
a) providing a polysaccharide-containing material,
b) subjecting the said polysaccharide-containing material to hydrolysis in the presence of a polysaccharide-degrading composition as defined herein, whereby a sugar product is obtained, and
c) fermenting the sugar product collected at step c), whereby a fermentation product is obtained.

### DESCRIPTION OF THE FIGURES

FIG. 1 illustrates a photograph of the gel electrophoresis on SDS-PAGE of the recombinant proteins of SEQ ID NO. 1, SEQ ID NO. 2 and SEQ ID NO. 3 produced in Pichia pastoris. Lane 1: 10 μg of the purified protein of SEQ ID NO. 3. Lane 2:10 μg of protein of SEQ ID NO. 1. Lane 3: 10 μg of protein of SEQ ID NO. 2. Lane 4: molecular weight protein markers.

FIG. 2 illustrates the effect of enzyme concentration on H₂O₂ production. (i) Upper curve with symbol □: protein of SEQ ID NO. 1, (ii) Lower curve with symbol Δ: protein of SEQ ID NO.2. (iii) Medium curve with symbol ×: protein of SEQ ID NO.3. (iv) symbol ⋄: Control containing the reaction mixture without proteins of SEQ ID NO. 1, SEQ ID NO. 2 or SEQ ID NO. 3, and shows the background level of H₂O₂ production (without enzyme). Abscissa: Concentration of protein, as expressed in micromolar. Ordinate: slope of hydrogen peroxide production, as expressed in Arbitrary Units.

FIG. 3 illustrates the results of a saccharification process in the presence of SEQ ID NO. 1 or SEQ ID NO.2. Supplementation of T. reesei enzymatic cocktail with SEQ ID NO. 1 (FIG. 3A) or SEQ ID NO. 2 (FIG. 3B) for the saccharification of pretreated poplar (filled bars), birchwood fibres (dashed bars), cellulose (dotted bars). Quantification of glucose release was monitored by ionic chromatography, the measured values being expressed as Arbitrary Units of glucose peak area. The synergistic effect of SEQ ID NO.1 or SEQ ID NO.2 with T. reesei cellulases is compared with the control (T. reesei cellulases only) FIG. 4 illustrates the results of a saccharification process in the presence of T. reesei cellulase cocktail supplemented with increasing amounts of either SEQ ID NO. 1 or SEQ ID NO.2 by measuring the release of glucose by ionic chromatography. FIG. 4A: Graph of the measured glucose peak area for increasing amounts of SEQ ID NO. 1.

FIG. 4B: Graph of the measured glucose peak area for increasing amounts of the protein of SEQ ID NO. 2. Ordinate: chromatography signal as expressed in Arbitrary Units. Abscissa of FIG. 4A: bars from the left side to the right side of the FIG. 1. absence of protein of SEQ ID NO. 1 or SEQ ID NO.2; 2. Final concentration in the assay of 0.22 μM of protein mixture of SEQ ID NO. 1 and SEQ ID NO.2; 3. Final concentration in the assay of 1.1 μM of a protein mixture of SEQ ID NO. 1 and SEQ ID NO.2; 4. Final concentration in the assay of 2.2 μM of a protein mixture of SEQ ID NO. 1 and SEQ ID NO.2

Abscissa of FIG. 4B: bars from the left side to the right side of the figure: 1. absence of protein of SEQ ID NO. 1 or SEQ ID NO. 2, 2. Final concentration in the assay of 0.22 μM of protein mixture of SEQ ID NO. 2; 3. Final concentration in the assay of 1.1 μM of a protein mixture of SEQ ID NO. 2; 4. Final concentration in the assay of 2.2 μM of a protein mixture of SEQ ID NO. 2

FIG. 5 illustrates the results of a saccharification process in the presence of T. reesei enzymatic cocktail supplemented by a AA9 lytic polysaccharide monooxygenase and protein of SEQ ID NO.1, by measuring the release of glucose by ionic chromatography. Abscissa: bars from the left side to the right side of the figure: 1. absence of protein of SEQ ID NO. 1 or AA9, 2. 2.2 μM of protein of SEQ ID NO. 1, 3. 2.2 μM of AA9, 4. 1.1 μM of a protein of SEQ ID NO. 1 and 1.1 g M of AA9.

FIG. 6: Phylogenetic tree of the AAxx family showing that the AAxx family members strongly cluster together and are very distant from AA9, AA10, AA11 and AA13 sequences respectively.

FIG. 7: Line diagram of PcAAxx LPMO active site.

FIG. 8A-8B: Consensus sequence logo based on alignment of 283 sequences belonging to the catalytic module of AAxx family revealing the first Histidine as a conserved residue amongst the family.

FIG. 9: Contribution of PcAAxx enzymes to the saccharification of woody biomass. Glucose release upon saccharification of pretreated pine and poplar by the CL847 Trichoderma reesei enzyme cocktail (18) in the presence of 1 μM of PcAAxxA or PcAAxxB and 1 mM ascorbic acid. Glucose was quantified using ionic chromatography. Error bars indicate standard deviations from triplicate independent experiments.

FIG. 10: Contribution of PcAAxx enzymes to the saccharification of woody biomass in the presence and in the absence of Ascorbate. Saccharification assays in the presence and absence of ascorbate on pine (A), and poplar (B). PcAAxxA (of SEQ ID No. 1) and PcAAxxB (of SEQ ID No. 2) were added to a concentration of 1 μM. Error bars indicate standard deviations from triplicate independent experiments.

### DETAILED DESCRIPTION OF THE INVENTION

Any citation mentioned herein is incorporated by reference.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e. to one or at least one) of the grammatical object of the article. By way of example, “an element” may mean one element or more than one element.

The inventors have identified a family of proteins endowed with a polysaccharide-degrading activity that may be used in processes requiring the production of sugar products from starting polysaccharide materials, in particular polysaccharide biomass, and especially in processes requiring the production of sugar products from starting lignocellulosic materials, such as highly refractory xylan-coated cellulose fibers.

Further, the inventors have shown herein that the said protein family members have the ability to substantially increase the rate or the level of polysaccharide hydrolysis, in an amount-dependent manner. The members of the said family of proteins that has been identified herein has the property of producing hydrogen peroxide in the presence of oxygen and of an electron donor such as ascorbic acid. This is why these proteins which are described in the present application may also be encompassed by the term “polysaccharide-oxidizing enzyme” herein.

This novel family of polysaccharide-oxidizing enzymes is also referred herein as the “AAxx” family of proteins. The inventors have further characterized structurally the reference protein PcAAxxB (Genbank #KY769370) from P. coccineus, by solving the crystallographic structure of its catalytic module at a resolution of 3 Å, thus providing a structural template for identifying all the relevant members of this AAxx family of enzymes, in complement to a sequence alignment of more than 300 proteins with significant similarity to PcAAxxB.

In particular, the polysaccharide-oxidizing enzymes of the invention are characterized by the presence of a conserved copper-binding active site, also referred herein as a copper-binding “histidine brace active site”, formed by two Histidine residues and a Tyrosine, one of those two Histidine residues being the N-terminal histidine after cleavage of the signal peptide.

Even more particularly, the inventors have shown herein that a plurality of proteins belonging to this novel AAxx family can be distinguished from other lytic polysaccharide monooxygenases in that they do not harbor a carbohydrate-binding module (CBM).

Illustratively, the examples herein show that the said protein family members substantially increase the level of polysaccharide degradation caused by a polysaccharide-degrading enzyme mixture. Notably, the examples herein show that protein members of the family of polysaccharide-oxidizing enzymes identified by the inventors substantially increase the level of glucose release caused by the action of an enzyme mixture originating from T. reesei on a cellulose-containing material, which encompasses a lignocellulosic material.

Still further, the inventors have shown that a polysaccharide-oxidizing protein that has been newly identified herein may act in synergy with known other polysaccharide-oxidizing enzymes such as lytic polysaccharide monooxygenases (which are also commonly termed LPMOs) for enhancing the polysaccharide hydrolysis caused by a polysaccharide-degrading enzyme mixture, and especially for enhancing the hydrolysis of a lignocellulosic material caused by an enzyme mixture comprising cellulases.

These further experimental evidences described herein show that the polysaccharide-oxidizing enzymes identified by the inventors may target distinct sugar units constitutive of a polysaccharide (e.g. cellulose, hemicellulose or lignocellulose), or alternatively distinct chemical groups of same sugar units, as compared to the sugar units, or the chemical groups, which are targeted by the known LPMOs, such as AA9 (also termed GH61), AA10, AA11 and AA13.

Thus, it has been unexpectedly found by the inventors that the polysaccharide-oxidizing enzymes identified herein may synergize with cellulases for degrading polysaccharide-containing material, such as cellulose-containing material like lignocellulose.

It has also been unexpectedly found herein that the polysaccharide-oxidizing enzymes identified herein may synergize with LPMOs for degrading polysaccharide-containing material, such as cellulose-containing material like lignocellulose.

Without wishing to be bound by the theory, there inventors are of the opinion that AAxx enzymes of the invention may act preferably on the xylans that are bound to cellulose, especially xylans that have a rigidity and a conformation similar to that of the underlying cellulose chains.

Accordingly, the polysaccharide-oxidizing enzymes of the invention can be considered either alone, or in combination with other polysaccharide-oxidizing and/or polysaccharide-degrading enzymes, and mixtures thereof.

Thus, the inventors have identified a novel class of polysaccharide-oxidizing enzymes that may be used in a large variety of processes for degrading polysaccharide-containing material, and especially in a large variety of processes for degrading lignocellulosic material.

The present invention provides for a novel class of polysaccharide-oxidizing enzymes which, when a polysaccharide-oxidizing enzyme thereof is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison result for the said polysaccharide-oxidizing enzyme comprises an E-value of 10 e⁻³ or less.

In particular, the inventors have identified a crystallographic structure of a polypeptide of sequence SEQ ID NO. 2. Thus the present invention also relates to polysaccharide-oxidizing enzymes which, when a polysaccharide-oxidizing enzyme thereof is compared to the reference polypeptide of SEQ ID NO. 2 by using the BLAST-P comparison method, (i) the said polysaccharide-oxidizing enzyme possesses an amino acid identity of 20% or more with the said reference polypeptide and (ii) the BLAST-P comparison result for the said polysaccharide-oxidizing enzyme comprises an E-value of 10 e⁻³ or less.

BLAST-P method (also termed Protein Basic Local Alignment Search Tool method) is well known from the one skilled in the art. BLAST-P method is notably described by Altschul et al. (1990, J Mol Biol, Vol. 215 (No. 3):403-410), Altschul et al. (1997, Nucleic Acids Res. Vol. 25:3389-3402) and Altschul et al. (2005, FEBS J. Vol. 272:5101-5109). BLAST-P method may be performed by using the NCBI tool that is available on-line (internet address: http://blast.ncbi.nlm.nih.gov/Blast.cgi?PAGE=Proteins).

When used herein, the BLAST-P method shall preferably be used with the following parameters: (i) Expected threshold: 10; (ii) Word Size: 6; (iii) Max Matches in a Query range: 0; (iv) Matrix: BLOSSUM62; (v) Gap costs: Existence 11, Extension 1; (vi) Compositional Adjustments: Conditional compositional score matrix adjustment, (vii) No filter; (viii) No mask.

As it is well known, the score of an alignment, S, is calculated as the sum of substitution and gap scores. Substitution scores are given by a look-up table (see PAM, BLOSUM hereunder). Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15) and a low value for L (1-2).

An optimal alignment means an alignment of two sequences with the highest possible score.

The amino acid identity means the extent to which two amino acid sequences have the same residues at the same positions in an alignment, often expressed as a percentage.

A Blocks Substitution Matrix (BLOSSUM) is a substitution scoring matrix in which scores for each position are derived from observations of the frequencies of substitutions in blocks of local alignments in related proteins. Each matrix is tailored to a particular evolutionary distance. In the BLOSUM62 matrix, for example, the alignment from which scores were derived was created using sequences sharing no more than 62% identity. Sequences more identical than 62% are represented by a single sequence in the alignment so as to avoid over-weighting closely related family members.

As used herein, an “E-value” (also termed Expect Value”) is a parameter calculated when using the BLAST-P method, the said parameter representing the number of different alignments with scores equivalent to or better than S that is expected to occur in a database search by chance. The lower the E value, the more significant the score and the alignment.

The inventors have identified more than 300 polypeptides that display significant comparison scores over their entire sequence length, when these polypeptides are compared to the polypeptide of SEQ ID NO. 1 by using the BLAST-P method.

Accordingly, the inventors have identified more than 300 polypeptides wherein, when any one of these polypeptides is compared to the reference polypeptide of SEQ ID NO. 1 by using the BLAST-P comparison method, (i) the said member possesses an amino acid identity of 20% or more with the said reference polypeptide and (ii) the BLAST-P comparison method results in an E-value of 10 e⁻³ or less.

Illustratively, there is described herein the polysaccharide-oxidizing enzyme of SEQ ID NO. 2, which possesses an amino acid identity of 66% with the reference polypeptide of SEQ ID NO. 1 and has an E-value of 4 e¹³³, when using the BLAST-P comparison method.

As shown in the examples herein, the polysaccharide-oxidizing enzyme of SEQ ID NO. 2, like the polysaccharide-oxidizing enzyme of SEQ ID NO. 1, has the ability to produce H₂O₂ in the presence of oxygen and an electron donor compound. Further, the polysaccharide-oxidizing enzyme of SEQ ID NO. 2, possesses polysaccharide degrading activity, as shown herein in a sequential lignocellulose degradation assay.

Still illustratively, there is described herein the polysaccharide-oxidizing enzyme of SEQ ID NO. 3, which possesses an amino acid identity of 34% with the reference polypeptide of SEQ ID NO. 1 and has an E-value of 2 e⁻⁴⁰, when using the BLAST-P comparison method.

As shown in the examples herein, the polysaccharide-oxidizing enzyme of SEQ ID NO. 2, like the polysaccharide-oxidizing enzyme of SEQ ID NO. 1, has the ability to produce H₂O₂ in the presence of oxygen and an electron donor compound, like the polysaccharide-oxidizing enzyme of SEQ ID NO. 2 and SEQ ID NO. 3.

Analyses of the sequence alignment by the inventors revealed that a conserved histidine residue is always present at the N-terminal position of each of these polypeptide sequences.

In particular, the polysaccharide-oxidizing enzymes of the invention are characterized by the presence of a conserved copper-binding active site, also referred herein as a copper-binding “histidine brace active site”, formed by two Histidine residues and a Tyrosine, one of those two Histidine residues being the N-terminal histidine.

According to another (non-mutually exclusive) embodiment, the polysaccharide-oxidizing enzymes of the invention may be N- and or O-glycosylated.

For reference, a polysaccharide-oxidizing enzyme of SEQ ID NO.2 may be N-glycosylated on at least one Asparagine (Asn) residue, selected from Asn 13, Asn76, Asn133, Asn183 and Asn217.

As used herein, a N-glycosylation on at least one Asparagine residue, may include one residue, two, three, four or five of said Asn residues; or if applicable all of Asn residues.

As used herein, a “polysaccharide-oxidizing enzyme” encompasses a polypeptide having the following properties:
the said polypeptide produces hydrogen peroxide in the presence of oxygen and an electron donor compound, such as ascorbate,
the said polypeptide increases in a dose-dependent manner, in the presence or in the absence of an electron donor, the degradation of a polysaccharide-containing material, such as lignocellulose, caused by cellulases and/or xylanases.
the said polypeptide increases in a dose-dependent manner, in the presence or in the absence of an electron donor, the degradation of a polysaccharide-containing material, such as lignocellulose, caused by cellulases, in the presence of one or more LPMOs, such as LPMOs selected in a group comprising AA9, AA10, AA11 and AA13.

In particular, a “polysaccharide-oxidizing enzyme” of the invention has been shown to be particularly efficient in oxidizing xylans, especially xylans that are absorbed onto cellulose.

The term “electron donor” is used herein in its usual meaning for the one skilled in the art. Thus, an electron donor compound is a chemical entity that donates electrons to another compound. An electron donor compound is a reducing agent by virtue of its donating electrons and is itself oxidized when donating electrons to another chemical entity. An electron donor, as specified above for the polysaccharide-oxidizing properties, encompasses, in a non-exhaustive manner, ascorbate and cellobiose dehydrogenase (CDH).

In the absence of a reductant, such as ascorbate, the reducing agent may advantageously be provided by the biomass (e.g. lignin), which could act as an electron donor.

The present invention also provides for uses of the said class of polysaccharide-oxidizing enzymes in various polysaccharide degradation processes, including in processes for degrading lignocellulosic material.

The present invention also provides for compositions comprising one or more of the said polysaccharide-oxidizing enzymes, and optionally polysaccharide degrading enzymes such as cellulases or lytic polysaccharide monooxygenases (LPMOs).

The present invention provides for a polysaccharide-oxidizing composition comprising a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less.

In some embodiments, a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less consists of the polypeptide of SEQ ID NO. 2. As already specified elsewhere herein, the polypeptide of SEQ ID NO. 2 has 66% amino acid identity with SEQ ID NO. 1 and an E value of 4 e⁻¹³³.

As specified herein a polypeptide comprising SEQ ID No. 2 may comprise, or consist of, a polypeptide of SEQ ID No. 7.

In some other embodiments, a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less consists of the polypeptide of SEQ ID NO. 3. As already specified elsewhere herein, the polypeptide of SEQ ID NO. 3 has 34% amino acid identity with SEQ ID NO. 1 and an E value of 2 e⁻⁴⁰.

Further, the polypeptide of SEQ ID NO. 3 has 37% amino acid identity with SEQ ID NO. 2 and an E value of 5 e⁻⁴⁴.

This invention also pertains to such compositions, wherein the said polysaccharide-oxidizing enzyme is encoded by a nucleic acid having at least 20% nucleotide identity with a nucleic acid selected in a group comprising the nucleic acids of SEQ ID NO. 4, SEQ ID NO. 5 and SEQ ID NO. 6.

This invention also pertains to such compositions, wherein the said polysaccharide-oxidizing enzyme is encoded by a nucleic acid having at least 20% nucleotide identity with a nucleic acid selected in a group comprising nucleic acids encoding a polypeptide of SEQ ID NO. 7.

Within the scope of the present invention, the “percentage identity” between two polypeptides means the percentage of identical amino acids residues between the two polypeptide sequences to be compared, obtained after optimal alignment, this percentage being purely statistical and the differences between the two polypeptide sequences being distributed randomly along their length. The comparison of two polypeptide sequences is traditionally carried out by comparing the sequences after having optimally aligned them, said comparison being able to be conducted by segment or by using an “alignment window”. Optimal alignment of the sequences for comparison is carried out, by using the comparison software BLAST-P).

In its principle, the percentage identity between two amino acid sequences is determined by comparing the two optimally-aligned sequences in which the nucleic acid sequence to compare can have additions or deletions compared to the reference sequence for optimal alignment between the two polypeptide sequences. Percentage identity is calculated by determining the number of positions at which the amino acid residue is identical between the two sequences, preferably between the two complete sequences, dividing the number of identical positions by the total number of positions in the alignment window and multiplying the result by 100 to obtain the percentage identity between the two sequences.

As intended herein, polypeptide sequences having at least 20% amino acid identity with a reference sequence encompass those having at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference sequence.

In some embodiments of a composition described herein, the said polysaccharide-oxidizing enzyme has at least 30% amino acid identity with a polypeptide selected in a group comprising the polypeptides of SEQ ID NO. 1, SEQ ID NO. 2 and SEQ ID NO. 3. Particularly, the polysaccharide-oxidizing enzymes encompass those having at least 30% amino acid identity with the polypeptide of SEQ ID NO. 1

In some embodiments of a composition described herein, the said polysaccharide-oxidizing enzyme has at least 60% amino acid identity with a polypeptide selected in a group comprising the polypeptides of SEQ ID NO. 1, SEQ ID NO. 2 and SEQ ID NO. 3. Particularly, the polysaccharide-oxidizing enzymes encompass those having at least 60% amino acid identity with the polypeptide of SEQ ID NO. 1

In some embodiments of a composition described herein, the said polysaccharide-oxidizing enzyme has at least 90% amino acid identity with a polypeptide selected in a group comprising the polypeptides of SEQ ID NO. 1, SEQ ID NO. 2 and SEQ ID NO. 3. Particularly, the polysaccharide-oxidizing enzymes encompass those having at least 90% amino acid identity with the polypeptide of SEQ ID NO. 1.

In some embodiments of a composition described herein, the said polysaccharide-oxidizing enzyme has at least 30% amino acid identity with a polypeptide of SEQ ID NO. 3.

In some embodiments of a composition described herein, the said polysaccharide-oxidizing enzyme has at least 60% amino acid identity with a polypeptide of SEQ ID NO. 3.

In some embodiments of a composition described herein, the said polysaccharide-oxidizing enzyme has at least 90% amino acid identity with a polypeptide of SEQ ID NO. 3.

In some embodiments of a composition described herein, the said polysaccharide-oxidizing enzyme is encoded by a nucleic acid having at least 90% nucleotide identity with a nucleic acid selected in a group comprising the nucleic acids of SEQ ID NO. 4, SEQ ID NO. 5 and SEQ ID NO. 6.

In some embodiments of a composition described herein, the said polysaccharide-oxidizing enzyme has at least 20% (or even 30%) amino acid identity with a polypeptide of sequence SEQ ID No. 7. Particularly, the polysaccharide-oxidizing enzymes encompass those having at least 60% or even 90% amino acid identity with a polypeptide of SEQ ID NO. 7.

Similarly, the “percentage identity” between two sequences of nucleic acids means the percentage of identical nucleotide residues between the two nucleic acid sequences to be compared, obtained after optimal alignment, this percentage being purely statistical and the differences between the two sequences being distributed randomly along their length. The comparison of two nucleic acid sequences is traditionally carried out by comparing the sequences after having optimally aligned them, said comparison being able to be conducted by segment or by using an “alignment window”. Optimal alignment of the sequences for comparison is carried out, by using the comparison software BLAST-N).

In its principle, the percentage identity between two nucleic acid sequences is determined by comparing the two optimally-aligned sequences in which the nucleic acid sequence to compare can have additions or deletions compared to the reference sequence for optimal alignment between the two sequences. Percentage identity is calculated by determining the number of positions at which the nucleotide residue is identical between the two sequences, preferably between the two complete sequences, dividing the number of identical positions by the total number of positions in the alignment window and multiplying the result by 100 to obtain the percentage identity between the two sequences.

As intended herein, nucleotide sequences having at least 20% nucleotide identity with a reference sequence encompass those having at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference sequence.

As used herein, an E-value of 10 e⁻³ or less encompasses E-values of 1 e⁻³ or less, 1 e⁻⁴ or less, 1 e⁻⁵ or less, 1 e⁻⁶ or less, 1 e⁻⁷ or less, 1 e⁻⁵ or less, 1 e⁻⁹ or less, 1 e⁻¹⁰ or less, 1 e⁻²⁰ or less, 1 e⁻³⁰ or less, 1 e⁻⁴⁰ or less, 1 e⁻⁵⁰ or less, 1 e⁻⁶⁰ or less, 1 e⁻⁷⁰ or less, 1 e⁻⁸⁰ or less, 1 e⁻⁹⁰ or less and 1 e⁻¹⁰⁰ or less.

Illustratively, as already specified elsewhere herein, when the polysaccharide-oxidizing enzyme of SEQ ID NO. 2 is compared to the reference polypeptide of SEQ ID NO. 1 by using the comparison method BLAST-P, (i) the said polysaccharide-oxidizing enzyme of SEQ ID NO. 2 possesses an amino acid identity of 66% with the reference polypeptide of SEQ ID NO. 1 and (ii) the BLAST-P comparison method results in an E-value of 4 e⁻¹³³.

Still illustratively, as already specified elsewhere herein, when the polysaccharide-oxidizing enzyme of SEQ ID NO. 3 is compared to the reference polypeptide of SEQ ID NO. 1 by using the comparison method BLAST-P, (i) the said polysaccharide-oxidizing enzyme of SEQ ID NO. 3 possesses an amino acid identity of 34% with the reference polypeptide of SEQ ID NO. 1 and (ii) the BLAST-P comparison method results in an E-value of 2 e⁻⁴⁰.

In preferred embodiments, the said polysaccharide-oxidizing enzyme is selected in a group comprising the polypeptides having the following GenBank reference numbers: ALO60293.1; CCA68158.1; CCA68159.1; CCA68161.1; CCA71530.1; CCA72554.1; CCA72555.1; CCO30796.1; CCT73728.1; CDM26384.1; CDO76981.1; CDO76983.1; CDO76990.1; CDR41535.1; CDZ98469.1; CDZ98532.1; CDZ98792.1; CDZ98793.1; CEJ62913.1; CEJ80690.1; CEL55274.1; CEL55761.1; CEN61973.1; CEQ41736.1; CRL20539.1; CUA74138.1; CUA75968.1; EEB87294.1; EEB88604.1; EEB93106.1; EGU12035.1; EGU79270.1; EJU02917.1; EJU04796.1; EJU04797.1; EKC98083.1; EKD01731.1; EKD04876.1; EKG10038.1; ELU37011.1; ELU44209.1; EMD31282.1; EMD34047.1; EMT65805.1; ENH74989.1; EPT05587.1; EPT05590.1; EPT05591.1; EUC56978.1; EUC64931.1; EWG51104.1; EWY85510.1; EXK29887.1; EXU99300.1; GAO89447.1; GAQ10202.1; GAT49547.1; GAT49548.1; GAT52486.1; GAT61130.1; GAT61131.1; KDE05902.1; KDE09071.1; KDN48575.1; KDN50638.1; KDQ07356.1; KDQ08649.1; KDQ08700.1; KDQ08703.1; KDQ11515.1; KDQ12702.1; KDQ15932.1; KDQ19064.1; KDQ25667.1; KDQ34148.1; KDQ59091.1; KDQ59092.1; KDR69809.1; KDR78641.1; KDR82083.1; KEP48245.1; KEY82804.1; KFG85718.1; KFH41721.1; KFY94807.1; KFZ00858.1; KFZ20368.1; KGB74552.1; KID86720.1; KII89650.1; KII89670.1; KIJ14235.1; KIJ14422.1; KIJ36788.1; KIJ36789.1; KIJ36910.1; KIJ36911.1; KIJ59037.1; KIJ62866.1; KIJ66712.1; KIJ93961.1; KIK01335.1; KIK01364.1; KIK03019.1; KIK24220.1; KIK24223.1; KIK45012.1; KIK47453.1; KIK58046.1; KIK60325.1; KIK64405.1; KIK64418.1; KIK64426.1; KIK64427.1; K1K64461.1; KIK94802.1; KIL59842.1; KIL67972.1; KIL68458.1; KIL88744.1; KIM29500.1; KIM34148.1; KIM35038.1; KIM39331.1; KIM49751.1; KIM57407.1; KIM60439.1; KIM60441.1; KIM60443.1; KIM60444.1; KIM84967.1; KIM93034.1; KIM95301.1; KIM95307.1; KIN08100.1; KIN97734.1; KIN97736.1; KIN97737.1; KIO31600.1; KIP08019.1; KIP08026.1; KIP10435.1; KIR25380.1; KIR50229.1; KIR55806.1; KIR67208.1; KIW62805.1; KIY36322.1; KIY46248.1; KIY46262.1; KIY46497.1; KIY46927.1; KIY47293.1; KIY51548.1; KIY64670.1; KIY68736.1; KIY71843.1; KJA14486.1; KJA19114.1; KJA20550.1; KJA20613.1; KJK82496.1; KK098459.1; KKP01653.1; KLT38889.1; KLT39034.1; KLT43002.1; KLT43602.1; KLT43893.1; KLT46239.1; KMK54965.1; KNZ77897.1; KNZ78922.1; KPA38710.1; KPI34779.1; KPM37038.1; KPV71930.1; KPV77521.1; KPV77742.1; KTB29212.1; KTB33212.1; KUE98996.1; KUE99426.1; KWU44348.1; KWU44477.1; KXH42132.1; KXH43636.1; KXH51881.1; KXN82218.1; KXN84873.1; KXN89494.1; KXN90938.1; KXN93349.1; KYQ38716.1; KYQ40395.1; KYQ41811.1; KZL64940.1; KZ090689.1; KZ090691.1; KZP14545.1; KZP23879.1; KZS91941.1; KZT07581.1; KZT07590.1; KZT20429.1; KZT29895.1; KZT40257.1; KZT57664.1; KZT57666.1; KZT73200.1; KZT73202.1; KZV68182.1; KZV68185.1; KZV69208.1; KZV72373.1; KZV79310.1; KZV79844.1; KZV82398.1; KZV83782.1; KZV85461.1; KZV85472.1; KZV86197.1; KZV88440.1; KZV88442.1; KZV88448.1; KZV96582.1; KZV97371.1; KZV97738.1; KZV97742.1; KZV98356.1; KZV99282.1; KZW00468.1; KZW00469.1; OAA59408.1; OAA71978.1; OAG11613.1; OAG40496.1; OAL02191.1; OAL28870.1; OAL45637.1; OAP54840.1; OAQ60454.1; OAQ77899.1; OAQ86421.1; OAQ94383.1; OAQ97907.1; OAX34821.1; XP_001263997.1; XP_001796117.1; XP_001829371.2; XP_001835502.2; XP_001835509.1; XP_001836582.1; XP_001840021.2; XP_001877230.1; XP_001878077.1; XP_001885228.1; XP_001905249.1; XP_003035108.1; XP_003035505.1; XP_003036605.1; XP_003042172.1; XP_003191958.1; XP_006458724.1; XP_006459911.1; XP_006963793.1; XP_007001773.1; XP_007003269.1; XP_007262604.1; XP_007299807.1; XP_007301417.1; XP_007306950.1; XP_007318869.1; XP_007318871.1; XP_007319142.1; XP_007327029.1; XP_007329615.1; XP_007337360.1; XP_007343208.1; XP_007346002.1; XP_007349200.1; XP_007349275.1; XP_007351346.1; XP_007351348.1; XP_007351349.1; XP_007351518.1; XP_007352398.1; XP_007353707.1; XP_007359130.1; XP_007362490.1; XP_007362492.1; XP_007362499.1; XP_007362779.1; XP_007388801.1; XP_007388810.1; XP_007393138.1; XP_007393767.1; XP_007581903.1; XP_007600909.1; XP_007746185.1; XP_007765609.1; XP_007768205.1; XP_007792087.1; XP_007792157.1; XP_007826256.1; XP_007849383.1; XP_007867180.1; XP_007867564.1; XP_008039133.1; XP_008039347.1; XP_008039803.1; XP_008039807.1; XP_008718658.1; XP_008731148.1; XP_009256644.1; XP_009545121.1; XP_009545122.1; XP_011321625.1; XP_012046198.1; XP_012181508.1; XP_012183613.1; XP_013257070.1; XP_013271081.1; XP_013277879.1; XP_013332110.1; XP_013943298.1; XP_013944931.1; XP_013954691.1; XP_013960458.1; XP_014176455.1; XP_014180074.1; XP_014180075.1; XP_014181917.1; XP_014543483.1; XP_014573268.1; XP_016242373.1; XP_016271225.1; XP_016275235.1; XP_016275300.1; XP_016610141.1; XP_016620042.1; XP_016630521.1; XP_567250.1; XP_753127.1 and XP_778151.1.

In preferred embodiments, the said polysaccharide-oxidizing enzyme consists of a recombinant protein. In some of these preferred embodiments, the said recombinant protein is produced by a yeast cell that has been genetically transformed so as to express the said recombinant polysaccharide-oxidizing enzyme.

In some embodiments, the said polysaccharide-oxidizing composition comprises only one polysaccharide-oxidizing enzyme described herein, and especially only one polysaccharide-oxidizing enzyme having an amino acid sequence selected in a group comprising SEQ ID NO. 1, SEQ ID NO.2, SEQ ID NO. 3, and the polysaccharide-oxidizing enzymes identified by their GenBank reference number herein.

In some embodiments, the said polysaccharide-oxidizing composition comprises only one polysaccharide-oxidizing enzyme encoded by a nucleic acid having a nucleic acid sequence selected in a group comprising SEQ ID NO. 4, SEQ ID NO. 5 and SEQ ID NO. 6.

In some other embodiments, the polysaccharide-oxidizing composition according to the invention comprises more than one polysaccharide-oxidizing enzyme described herein, and especially more than one polysaccharide-oxidizing enzyme having an amino acid sequence selected in a group comprising SEQ ID NO. 1, SEQ ID NO.2, SEQ ID NO. 3 and the polysaccharide-oxidizing enzymes identified by their respective GenBank reference number herein. In some of these embodiments, the polysaccharide-oxidizing composition according to the invention comprises the polysaccharide-oxidizing enzymes of SEQ ID NO; 1, of SEQ ID NO. 2 and of SEQ ID NO. 3.

In some other embodiments, the polysaccharide-oxidizing composition according to the invention comprises more than one polysaccharide-oxidizing enzyme encoded by a nucleic acid having a nucleic acid sequence selected in a group comprising SEQ ID NO. 4, SEQ ID NO. 5, SEQ ID NO. 6 and the nucleic acid sequences encoding the polysaccharide-oxidizing enzymes identified by their respective GenBank reference number herein. In some of these other embodiments, the polysaccharide-oxidizing composition according to the invention comprises the polysaccharide-oxidizing enzymes encoded by nucleic acids having the nucleic acid sequences of SEQ ID NO. 4, of SEQ ID NO. 5 and of SEQ ID NO. 6.

According to some of these other embodiments, the polysaccharide-oxidizing composition comprises from 2 to 10 distinct polysaccharide-oxidizing enzymes having an amino acid sequence selected in a group comprising SEQ ID NO. 1, SEQ ID NO.2, SEQ ID NO. 3 and the polysaccharide-oxidizing enzymes identified by their respective GenBank reference number herein.

According to some of these other embodiments, the polysaccharide-oxidizing composition comprises from 2 to 10 distinct polysaccharide-oxidizing enzymes having an amino acid sequence selected in a group comprising SEQ ID NO. 1, SEQ ID NO.2 and SEQ ID NO. 3 and the polysaccharide-oxidizing enzymes identified by their respective GenBank reference number herein.

According to some of these other embodiments, the polysaccharide-oxidizing composition comprises from 2 to 10 distinct polysaccharide-oxidizing enzymes encoded by a nucleic acid having a nucleic acid sequence selected in a group comprising SEQ ID NO. 4, SEQ ID NO. 5, SEQ ID NO. 6 and the nucleic acids encoding the polysaccharide-oxidizing enzymes identified by their respective GenBank reference number herein.

According to these other embodiments, the polysaccharide-oxidizing composition comprises 2, 3, 4, 5, 6, 7, 8, 9 or 10 distinct polysaccharide-oxidizing enzymes as described herein.

In some further embodiments, a polysaccharide-oxidizing composition according to the invention further comprises one or more other polysaccharide-oxidizing enzymes, which other polysaccharide-oxidizing enzymes are preferably selected among lytic polysaccharide monooxygenases, which encompasses other polysaccharide-oxidizing enzymes selected in a group comprising AA9, AA10, AA11 and AA13 LPMOs.

In some embodiments, a polysaccharide-oxidizing composition described herein may be used as a ready-to-use composition for degrading a polysaccharide-containing material, which encompasses a ready-to-use composition for degrading a lignocellulosic material.

In other embodiments, a polysaccharide-oxidizing composition as described herein may consist of an auxiliary composition that may be used in combination with one or more distinct polysaccharide degrading enzymes in a process for degrading a polysaccharide-containing material. According to these other embodiments, the said one or more other polysaccharide degrading enzymes may be contained as an enzyme mixture in a polysaccharide-degrading composition. As it will be further described in the present specification, a variety of such polysaccharide-degrading enzyme mixtures are known in the art, which encompasses enzyme mixtures comprising cellulases, such as enzyme mixtures comprising fungus-derived cellulases.

As shown herein, a polysaccharide-oxidizing composition as described in the present specification, when used in combination with known polysaccharide-degrading enzymes, and especially when used in combination with a mixture of polysaccharide-degrading enzymes, allows a more easier and a more complete sugar product release from a polysaccharide-containing starting material. In particular, a polysaccharide-oxidizing composition as described in the present specification, when used in combination with known polysaccharide-degrading enzymes, and especially when used in combination with a mixture of polysaccharide-degrading enzymes, allows a more easier and a more complete glucose release from a cellulose-containing starting material, which includes a more easier and a more complete release of glucose from a lignocellulosic starting material.

As already specified herein, there is generally provided compositions comprising a polysaccharide-oxidizing enzyme selected in a group comprising SEQ ID NO. 1, SEQ ID NO.2, SEQ ID NO. 3 and the polysaccharide-oxidizing enzymes identified by their respective GenBank reference number herein, as well as polysaccharide-oxidizing enzymes encoded by a nucleic acid having a nucleic acid sequence selected in a group comprising SEQ ID NO. 4, SEQ ID NO. 5, SEQ ID NO. 6 and the nucleic acids encoding the polysaccharide-oxidizing enzymes identified by their respective GenBank reference number herein.

According to one embodiment, the invention further provides a method for preparing a polysaccharide-oxidizing enzyme comprising the steps of:

a) providing a composition comprising an enzyme polypeptide wherein, when the said polypeptide is compared to a reference polypeptide selected in a group comprising SEQ ID NO. 1, SEQ ID NO.2 and SEQ ID NO. 3, by using the BLAST-P comparison method, wherein (i) the said enzyme polypeptide possesses an amino acid identity of 20% or more with the said reference polypeptide and (ii) the BLAST-P comparison method results in an E-value of 10 e⁻³ or less;

b) incubating said enzyme polypeptide with a copper-containing composition, thereby preparing a polysaccharide-oxidizing enzyme.

The said copper-containing composition may be selected from a composition containing one or more copper salts, such as sulphate or acetate copper salts.

Optionally, the said method may further comprise a step c) of removing an excess amount of copper (or salts thereof) from said composition comprising said enzyme polypeptide.

Methods for Identifying Polypeptides Belonging to the AAxx Protein Family

The inventors disclose herein a crystallographic structure of the PcAAxxB (JGI ID 1372210; GenBank ID #KY769370) catalytic module. They also disclose herein an extensive set of polysaccharide-oxidizing enzymes belonging to this novel enzyme family, along with reference protein sequences SEQ ID No. 1, SEQ ID No. 2 and SEQ ID No. 3

For reference, SEQ ID No. 3 corresponds to SEQ ID No. 9 after peptide signal cleavage; and SEQ ID No. 8 corresponds to the cleaved peptide signal.

Accordingly, by combining the available structural information and the general consensus sequence derivable from said set of polysaccharide-oxidizing enzymes, it is thus also possible to determine any variant thereof having the desired polysaccharide-oxidizing activity.

In a non-limitative manner, program-implemented methods for determining a given set of engineered or native polypeptides belonging to a given family of polypeptides can be undergone in silico by homology or comparative modeling of protein three-dimensional structures based on an alignment of sequences and a given set of known related structures. Examples of programs suitable for homology and comparative modeling include:
MODELLER© V.9.18 (see B. Webb, A. Sali. Comparative Protein Structure Modeling Using Modeller. Current Protocols in Bioinformatics, John Wiley & Sons, Inc., 5.6.1-5.6.32, 2014);
I-TASSER V.5.1 (See J Yang, R Yan, A Roy, D Xu, J Poisson, Y Zhang. The I-TASSER Suite: Protein structure and function prediction. Nature Methods, 12: 7-8 (2015)) and
the SWISS-MODEL homology-modelling server (see Biasini M, Bienert S, Waterhouse A, Arnold K, Studer G, Schmidt T, Kiefer F, Cassarino T G, Bertoni M, Bordoli L, Schwede T (2014). SWISS-MODEL: modelling protein tertiary and quaternary structure using evolutionary information (Nucleic Acids Research 2014 (1 Jul. 2014) 42 (W1): W252-W258).

Thus, according to one embodiment, the invention relates to a method for identifying polysaccharide-oxidizing enzymes, which comprises the steps of:

a1) providing one or more candidate polypeptides which may possess a polysaccharide-oxidizing activity;

a2) providing experimental coordinates of a reference polypeptide backbone and/or side chain, wherein said reference polypeptide backbone and/or side chain is a polypeptide comprising SEQ ID NO. 2 or SEQ ID NO 7, or a fragment thereof having a polysaccharide-oxidizing activity;

a3) providing a sequence alignment of the one or more candidate polypeptide with a reference polypeptide sequence possessing an amino acid identity of 20% or more with SEQ ID NO. 1, SEQ ID NO. 2, SEQ ID NO 3 or SEQ ID NO 7 by using the BLAST-P comparison method, characterized in that the said one or more candidate polypeptides possesses an E-value of 10 e⁻³ or less;

b) determining from a1), a2) and a3) the theorical coordinates of the one or more candidate polypeptide;

c) determining the root-mean-square deviation (RMSD) of the said theorical coordinates with the experimental coordinates of the reference polypeptide;

wherein an increased RMSD of the theorical coordinates, compared to the experimental coordinates, above a reference threshold indicates a lower probability of having a polysaccharide-oxidizing activity;

wherein a decreased RMSD of the theorical coordinates, compared to the experimental coordinates, below a reference threshold indicates a higher probability of having a polysaccharide-oxidizing activity.

d) optionally selecting the one or more candidate polypeptides of which the theorical coordinates define a copper-binding “histidine brace active site”, formed by two Histidine residues and a Tyrosine, one of those two Histidine residues being a N-terminal Histidine.

This invention also pertains to more complete compositions wherein the said compositions further comprise other enzymes contributing to the degradation of a polysaccharide-containing material, notably enzymes contributing to the degradation of a cellulose-containing material, and especially enzymes contributing to the degradation of lignocellulose-containing material.

In some embodiments of a polysaccharide-degrading composition as described herein, the said composition further comprises one or more polysaccharide-degrading enzymes selected in a group comprising cellulases, hemicellulases, ligninases, and carbohydrate oxidases.

Cellulases encompass endoglucanases and cellobiohydrolases and beta-glucosidases.

Hemicellulases encompass xylanases, mannanases, xylosidases, mannosidases, arabinofuranosidaes and esterases.

Ligninases encompass peroxidases, copper radical oxidases (e.g. laccases).

Carbohydrate oxidases encompass lytic polysaccharide monooxygenases and GMC oxidoreductases (e.g. glucose dehydrogenases, cellobiose dehydrogenases, . . . ).

According to some of these embodiments, the one or more cellulases comprised in a polysaccharide-degrading composition are selected in a group comprising exo-glucanases, endo-glucanases, cellobiohydrolases, cellulose phosphorylases, pectinases, pectate lyases, polygalacturonase, pectin esterases, cellobiose dehydrogenases, mannanases, arabino furanosidases, feruoyl esterases, arabino furanosidases, fructofuranosidases, galactosidases, galactosidases, amylases, acetylxylan esterases, chitin deacetylases, chitinases, and glucosidases.

According to some of these embodiments, the one or more lytic polysaccharide monooxygenases comprised in a polysaccharide-degrading composition are selected in a group comprising AA9, AA10, AA11 and AA13.

According to some of these embodiments, wherein the said one or more other polysaccharide degrading enzymes are comprised in an enzyme preparation containing the said one or more other polysaccharide degrading enzymes.

According to some of these embodiments, the said enzyme preparation comprises one or more enzymes originating from one or more fungus organisms or one or more bacterial organisms.

According to some of these other embodiments, the said one or more fungus organism is selected in a group comprising fungi of the genus, but not limited to Achlya, Acremonium, Aspergillus, Cephalosporium, Chrysosporium, Cochliobolus, Endothia, Fusarium, Gliocladium, Humicola, Hypocrea, Myceliophthora, Mucor, Neurospora, Penicillium, Pyricularia, Thielavia, Tolypocladium, Trichoderma, Podospora, Pycnoporus, Fusarium, Thermonospora, Hypocrea, Humicola, Penicillium, Myceliophthora and Aspergillus.

According to some of these other embodiments, the said enzyme preparation comprises an enzyme extract from one or more fungus organisms or from one or more bacterial organisms.

According to some of these other embodiments, one or more of the said other polysaccharide-degrading enzymes are recombinant proteins.

The polysaccharide-oxidizing enzymes of the invention are further considered in the form of a kit, especially a kit for preparing a polysaccharide-oxidizing composition or a polysaccharide-degrading composition.

According to one embodiment, the invention relates to a kit for:

preparing a polysaccharide-oxidizing or polysaccharide-degrading composition;

oxidizing a polysaccharide;

preparing a sugar product from a polysaccharide-containing material

preparing a fermentation product from a polysaccharide-containing material; characterized in that it contains at least

(i) a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less; and

(ii) at least another distinct enzyme selected from the group consisting of a polysaccharide-oxidizing or polysaccharide-degrading enzyme, as previously defined, which may thus include cellulases, hemicellulases, ligninases, and carbohydrate oxidases, which may thus include one or more lytic polysaccharide monooxygenases (LPMOs).

Said enzymes may also be in the form of polysaccharide-oxidizing or polysaccharide-degrading composition, as previously defined.

Alternatively, a kit of the invention may also comprise one or more yeast cells expressing a polysaccharide-oxidizing or polysaccharide-degrading enzyme, as previously defined.

### Polysaccharide-Containing Material

As used herein, a polysaccharide containing material encompasses a substance or a composition comprising polysaccharide molecules.

The term “polysaccharide” is used in its conventional meaning, and designates polymeric carbohydrate molecules composed of long chains of mnonosaccharide units bound together by glycosidic linkages. On hydrolysis, polysaccharides release the constitutive monosaccharides or oligosaccharides.

Preferred polysaccharides according to the invention are plant-derived polysaccharides, and especially cellulose, such as cellulose contained in lignocellulose.

Other plant-derived polysaccharides which are particularly considered including xylans. Xylans belong to the group of hemicelluloses, and are polysaccharides made from units of xylose.

According to one embodiment, the polysaccharide-containing material is a material that comprises at least one (or a plurality of) polysaccharides selected from the group of cellulose, hemicellulose and lignin; which includes for instance any polysaccharide-containing material which contains at least 30 wt. % of cellulose and hemicellulose. For example, the polysaccharide-containing material may be a material such as birchwood cellulosic fibers, consisting of about 79% cellulose and about 21% xylan, as substrate.

The term “lignocellulose-containing material” used herein refers to material that primarily consists of cellulose, hemicellulose, and lignin. The term is synonymous with “lignocellulosic material”. Such material is often referred to as “biomass”.

Any lignocellulose-containing material is contemplated according to the present invention. The lignocellulose-containing material may be any material containing lignocellulose. In a preferred embodiment the lignocellulose-containing material contains at least 30 wt. %, preferably at least 50 wt. %, more preferably at least 70 wt. %, even more preferably at least 90 wt. % lignocellulose. It is to be understood that the lignocellulose-containing material may also comprise other constituents such as proteinaceous material, starch, sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemi-cellulose in a mixed matrix.

In some particular embodiments, the lignocellulosic-containing material is a lignocellulosic biomass selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, Agave, and combinations thereof. In a preferred embodiment the lignocellulose-containing material comprises one or more of corn stover, corn fiber, rice straw, pine wood, wood chips, poplar, bagasse, paper and pulp processing waste.

Preferably, the lignocellulosic-containing material is a lignocellulosic woody biomass. Other examples of lignocellulose-containing material include hardwood, such as poplar and birch, softwood, cereal straw, such as wheat straw, switchgrass, municipal solid waste, industrial organic waste, office paper, or mixtures thereof.

According to exemplary embodiments, the lignocellulosic-containing material is selected from pine, poplar and wheat straw.

### Other Enzymes

As already specified herein, a polysaccharide-oxidizing composition according to the invention may comprise, in addition to one or more polysaccharide-oxidizing enzyme belonging to the enzyme family specifically identified by the inventors, also one or more other polysaccharide-oxidizing enzyme, such as one or more lytic polysaccharide monooxygenases (LPMOs).

As also already specified herein, a polysaccharide-degrading composition according to the invention may comprise, in addition to one or more polysaccharide-oxidizing enzyme belonging to the enzyme family specifically identified by the inventors, also one or more polysaccharide degrading enzyme, notably cellulolytic enzymes, which are also commonly termed cellulases.

The other enzymes may be simply combined or alternatively they may be contained in an enzyme mixture, such as a fungus-derived or a bacteria-derived enzyme mixture, for example a commercial fungus-derived enzyme mixture.

Cellulases that may be used in a polysaccharide degrading composition as described herein encompass exoglucanases, endoglucanases, cellobiohydrolases, cellulose phosphorylases, pectinases, pectate lyases, polygalacturonase, pectin esterases, ceelobiose dehydrogenases, beta mannanases, arabonisidases, feruoyl esterases, arabinofuranosidases, fructofuranosidases, alpha galactosidases, beta galactosidases, alpha amylases, acetylxylan esterases, chitin deacetylases, chitinases, and beta glucosidases.

In order to be efficient, degradation of cellulose may require several types of enzymes acting cooperatively. At least three categories of enzymes are often needed to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase I1 activity” is identical, except that cellobiohydrolase II attacks from the reducing ends of the chains.

Cellulases preparation may further comprise a beta-glucosidase, such as a beta-glucosidase derived from a strain of the genus, but not limited to Humicola, Trichoderma, Podospora, Pycnoporus, Fusarium, Thermonospora, Hypocrea, Chrysosporium and Aspergillus.

Cellulases may be comprised in an enzyme mixture, which encompasses an enzyme mixture derived from Trichoderma reesei.

Thus, cellulases which may be used in a polysaccharide-degrading composition as described herein may be derived from a fungal source, such as a strain of the genus Trichoderma, preferably a strain of Trichoderma reesei; or a strain of the genus Humicola, such as a strain of Humicola insolens; or a strain of Chrysosporium

Other useful enzymes encompass alpha-amylases; glucoamylases or another carbohydrate-source generating enzymes, such as beta-amylases, maltogenic amylases and/or alpha-glucosidases; proteases; or mixtures of two of more thereof.

Other useful enzymes are the lytic polysaccharide monoxygenases (LPMOs), and especially those LPMOs selected in a group comprising AA9, AA10, AA11 and AA13, which are described notably by Busk et al. (2015, BMC Genomics, Vol. 16: 368) and by Hemsworth et al. (2015, Trends in Biotechnology, Vol. 33 (12): 747-761).

### Recombinant Yeast Expressing a Polysaccharide-Oxidizing Enzyme

In some embodiments of a polysaccharide-oxidizing composition or of a polysaccharide degrading composition as described herein, a polysaccharide-oxidizing enzyme belonging to the enzyme family specifically identified by the inventors may consist of a recombinant polypeptide, which encompasses a recombinant polypeptide produced in a yeast organism, as it is shown in the examples herein.

Thus, this invention also relates to a recombinant yeast cell expressing a polysaccharide-oxidizing enzyme as described in the present specification.

This invention concerns a recombinant yeast cell expressing a polysaccharide-oxidizing enzyme wherein, when the said polysaccharide-oxidizing enzyme is compared to the reference polypeptide of SEQ ID NO. 1 by using the BLAST-P comparison method, (i) the said polysaccharide-oxidizing enzyme possesses an amino acid identity of 20% or more with the said reference polypeptide and (ii) the BLAST-P comparison method results in an E-value of 10 e⁻³ or less. These polysaccharide-oxidizing enzymes are those which are described in the present specification.

In some embodiments, the yeast organism is selected in a group of yeast organisms comprising Saccharomyces, Kluyveromyces, Candida, Pichia, Schizosaccharomyces, Hansenula, Kloeckera, Schwanniomyces, and Yarrowia. Yeast species as host cells may include, for example, S. cerevisiae, S. bulderi, S. barnetti, S. exiguus, S. uvarum, S. diastaticus, K. lactis, K. marxianus, or K. fragilis. In some embodiments, the yeast is selected from the group consisting of Saccharomyces cerevisiae, Schizzosaccharomyces pombe, Issatchenkia orientalis, Candida albicans, Candida mexicana, Pichia pastoris, Pichia mississippiensis, Pichia mexicana, Pichia stipitis, Pichia farinosa, Clavispora opuntiae, Clavispora lusitaniae, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe, Hansenula polymorpha, and Schwanniomyces occ dentalis.

A nucleic acid allowing the expression of a polysaccharide-oxidizing enzyme of interest is introduced in the genome of the selected yeast organism or is introduced as a non-integrated vector according to genetic engineering methods that are well known from the one skilled in the art.

A “vector,” e.g., a “plasmid” or “YAC” (yeast artificial chromosome) refers to an extrachromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids or vectors of the present invention are stable and self-replicating.

An “expression vector” is a vector that is capable of directing the expression of genes to which it is operably associated.

Promoter” refers to a DNA fragment capable of controlling the expression of a coding sequence or functional RNA. In general, a coding region is located 3′ to a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions.

A coding region is “under the control” of transcriptional and translational control elements in a cell when RNA polymerase transcribes the coding region into mRNA, which is then trans-RNA spliced (if the coding region contains introns) and translated into the protein encoded by the coding region.

The present invention also relates to vectors which include a nucleic acid encoding a polysaccharide-oxidizing enzyme belonging to the family of enzymes specifically identified by the inventors, host cells, most preferably yeast host cells, which are genetically engineered with vectors of the invention and the production of the polysaccharide-oxidizing enzymes described herein by recombinant techniques.

Host cells, most preferably yeast host cells, are genetically engineered (transduced or transformed or transfected) with the vectors described above which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The appropriate nucleic acid may be inserted into the vector by a variety of procedures. In general, the nucleic acid is inserted into an appropriate restriction endonuclease site(s) by procedures known in the art. Such procedures and others are deemed to be within the scope of those skilled in the art.

The nucleic acid is inserted in the expression vector is operatively associated with an appropriate expression control sequence(s) (promoter) to direct mRNA synthesis. Representative examples of such promoters are as follows:

The expression vector may also contain a ribosome binding site for translation initiation and/or a transcription terminator. The vector may also include appropriate sequences for amplifying expression, or may include additional regulatory regions.

The vector containing the appropriate nucleic acid, as well as an appropriate promoter or control sequence, may be employed to transform an appropriate host to permit the host to express the protein.

According to one embodiment, a yeast expressing a polysaccharide-oxidizing enzyme as previously defined may further express at least one additional enzyme selected from the group consisting of a polysaccharide-oxidizing or polysaccharide-degrading enzyme.

Methods of Producing Industrial Substances from Polysaccharide-Containing Material

The present invention also relates to a method for oxidizing a polysaccharide comprising a step of contacting one or more polysaccharides with a polysaccharide-oxidizing enzyme as described herein, or with a composition comprising the said polysaccharide-oxidizing enzyme.

In some embodiments of this method, the said one or more polysaccharides are comprised in a polysaccharide-containing biomass.

In some embodiments of this method, the said one or more polysaccharides are contained in a lignocellulosic-containing material.

This invention also pertains to methods for obtaining a sugar product from a polysaccharide-containing material, wherein the said methods comprise a step of hydrolyzing a polysaccharide-containing material by using a polysaccharide-oxidizing enzyme composition according to the invention, which includes by using an polysaccharide degrading composition as described in the present specification.

Thus, the present invention also concerns a method for oxidizing a polysaccharide comprising a step of contacting one or more polysaccharides with a polysaccharide-oxidizing enzyme as described in the present specification, or with a composition comprising the said polysaccharide-oxidizing enzyme.

This invention also relates to a method for the preparation of a sugar product from a polysaccharide-containing material comprising a step of treating the said polysaccharide-containing material in the presence of a polysaccharide degrading composition described in the present specification.

In preferred embodiments of these methods, the polysaccharide-containing materiel consists of a cellulose-containing material, such as preferably a lignocellulosic material, which encompasses lignocellulose.

This invention also provides a method for the preparation of a sugar product from a polysaccharide-containing material comprising the steps of:
a) providing a polysaccharide-containing material,
b) subjecting the said polysaccharide-containing material to hydrolysis in the presence of a polysaccharide-degrading composition as described in the present specification, and
c) collecting the sugar product obtained at the end of step b).

General methods for the preparation of a sugar product from a polysaccharide-containing material which comprise a step of hydrolysing the said polysaccharide-containing material are already known in the art. More specifically, methods for the preparation of a sugar product, including glucose, from a lignocellulosic-containing material are already known in the art.

In some embodiments of such methods, a lignocellulosic material is pretreated before the step of hydrolysis so as to increase the efficiency of the hydrolysis step.

### Pre-Treatment of Lignocellulose

The structure of lignocellulose is not directly accessible to enzymatic hydrolysis.

Therefore, the lignocellulose-containing material has preferably to be pre-treated, e.g., by acid hydrolysis under adequate conditions of pressure and temperature, in order to break the lignin seal and disrupt the crystalline structure of cellulose. This causes solubilization of the hemicellulose and cellulose fractions. The cellulose and hemicellulose can then be hydrolyzed enzymatically such as described in the present specification, to convert the carbohydrate polymers into fermentable sugars

A pre-treatment step enhances the digestibility of lignocellulose and thus increases the efficiency of the hydrolysis step.

Methods for pretreating lignocellulose are well known in the art, which includes steps of chemical pretreatment, mechanical pretreatment and biological pretreatment.

When lignocellulose-containing material is pre-treated, degradation products that may inhibit enzymes and/or may be toxic to fermenting organisms are produced. These degradation products severely decrease both the hydrolysis and fermentation rate. Methods for pre-treating lignocellulose-containing material are well known in the art. The pre-treated lignocellulose degradation products include lignin degradation products, cellulose degradation products and hemicellulose degradation products. The pre-treated lignin degradation products may be phenolics in nature.

The lignocellulose-containing material may be pre-treated in any suitable way. Pre-treatment may be carried out before and/or during hydrolysis and/or fermentation. In a preferred embodiment the pre-treated material is hydrolyzed, preferably enzymatically, before and/or during fermentation. The goal of pre-treatment is to separate and/or release cellulose; hemicellulose and/or lignin and this way improve the rate of hydrolysis. Pre-treatment methods such as wet-oxidation and alkaline pre-treatment targets lignin, while dilute acid and auto-hydrolysis targets hemicellulose. Steam explosion is an example of a pre-treatment that targets cellulose.

According to the invention the pre-treatment applied in step (a) may be a conventional pre-treatment step using techniques well known in the art.

The lignocellulose-containing material may according to the invention be chemically, mechanically and/or biologically pre-treated before hydrolysis and/or fermentation. Mechanical treatment (often referred to as physical treatment) may be used alone or in combination with subsequent or simultaneous hydrolysis, especially enzymatic hydrolysis.

Preferably, chemical, mechanical and/or biological pre-treatment is carried out prior to the hydrolysis and/or fermentation. Alternatively, the chemical, mechanical and/or biological pre-treatment may be carried out simultaneously with hydrolysis, such as simultaneously with addition of one or more cellulase enzymes (cellulolytic enzymes), or other enzyme activities mentioned below, to release, e.g., fermentable sugars, such as glucose and/or maltose.

In an embodiment of the invention the pre-treated lignocellulose-containing material may be washed. However, washing is not mandatory and is in a preferred embodiment eliminated.

The term “chemical treatment” refers to any chemical pre-treatment which promotes the separation and/or release of cellulose, hemicellulose and/or lignin. Examples of suitable chemical pre-treatments include treatment with; for example, dilute acid, lime, alkaline, organic solvent, ammonia, sulfur dioxide, carbon dioxide. Further, wet oxidation and pH-controlled hydrothermolysis are also considered chemical pre-treatment. Pre-treatment methods using ammonia are notably described in the PCT applications WO 2006/110891, WO 2006/110899, WO 2006/110900, and WO 2006/110901.

Other examples of suitable pre-treatment methods are described by Schell et al., 2003, Appl. Biochem and Biotechn. Vol. 105-108: 69-85, and Mosier et al., 2005, Bioresource Technology 96: 673-686, and U.S. Publication No. 2002/0164730.

The term “mechanical pre-treatment” refers to any mechanical (or physical) treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion).

In some embodiments of a pretreatment step, the said step may combine chemical and mechanical pretreatment.

As used in the present invention the term “biological pre-treatment” refers to any biological pre-treatment which promotes the separation and/or release of cellulose, hemicellulose, and/or lignin from the lignocellulose-containing material. Biological pre-treatment techniques can involve applying lignin-solubilizing microorganisms (see, for example, Hsu, 1996, Pretreatment of biomass, in Handbook on Bioethanol: Production and Utilization, Wyman, ed., Taylor & Francis, Washington, D.C., 179-212; Ghosh and Singh, 1993, Physicochemical and biological treatments for enzymatic/microbial conversion of lignocellulosic biomass, Adv. Appl. Microbiol. 39: 295-333; McMillan, 1994, Pretreating lignocellulosic biomass: a review, in Enzymatic Conversion of Biomass for Fuels Production, Himmel, Baker, and Overend, eds., ACS Symposium Series 566, American Chemical Society, Washington, D.C., chapter 15; Gong, Cao, Du, and Tsao, 1999, Ethanol production from renewable resources, in Advances in Biochemical Engineering/Biotechnology, Scheper, ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng J Biotechnol. 42: 63-95).

### Enzyme Hydrolysis

General methods of enzyme hydrolysis of a polysaccharide-containing material, including a cellulose-containing material, such as a lignocellulosic material, are well known for the one skilled in the art. Some of these methods are notably disclosed in the PCT applications WO 2015/165954, WO 2010/080407, WO 2009/026722, WO 2008/025165 and WO 2009/135898, as well as by Mohanram et al. (2013, Sustainable Chemical Processes, Vol. 1: 15-26), Van Dyk et al. (2012, Biotechnology Advances, Vol. 30: 1458-1480), Debeire et al. (2014, FEMS Microbiol Lett, Vol. 355: 116-123), Liao et al., 2011, Biotechnol J, Vol. 6: 1-9), Kumar et al. (2012, Final Technical Report, DSM Innovation, for the US Department of Energy under Award Number DE-FG36-08GO18079), Tucer et al., 2009, Turk J Biol, Vol. 33: 291-300).

Methods described herein comprise an enzymatic hydrolysis step. The enzymatic hydrolysis includes, but is not limited to, hydrolysis for the purpose of liquefaction of the feedstock and hydrolysis for the purpose of releasing sugar from the feedstock or both. In this step optionally pretreated and optionally washed lignocellulosic material is brought into contact with the enzyme composition according to the invention. Depending on the lignocellulosic material and the pretreatment, the different reaction conditions, e.g. temperature, enzyme dosage, hydrolysis reaction time and dry matter concentration, may be adapted by the skilled person in order to achieve a desired conversion of lignocellulose to sugar. Some indications are given hereafter.

In an embodiment the enzymatic hydrolysis comprises at least a liquefaction step wherein the lignocellulosic material is hydrolyzed in at least a first container, and a saccharification step wherein the liquefied lignocellulosic material is hydrolyzed in the at least first container and/or in at least a second container. Saccharification can be done in the same container as the liquefaction (i.e. the at least first container), it can also be done in a separate container (i.e. the at least second container). So, in the enzymatic hydrolysis of the processes according to the present invention liquefaction and saccharification may be combined. Alternatively, the liquefaction and saccharification may be separate steps. Liquefaction and saccharification may be performed at different temperatures, but may also be performed at a single temperature. In an embodiment the temperature of the liquefaction is higher than the temperature of the saccharification.

In some embodiments, liquefaction may be carried out at a temperature of 60-75° C. and saccharification may be carried out at a temperature of 50-65° C.

The enzymes used in the enzymatic hydrolysis may be added before and/or during the enzymatic hydrolysis. In case the enzymatic hydrolysis comprises a liquefaction step and saccharification step, additional enzymes may be added during and/or after the liquefaction step. The additional enzymes may be added before and/or during the saccharification step. Additional enzymes may also be added after the saccharification step.

In some embodiments the hydrolysis is conducted at a temperature of 45° C. or more, 50° C. or more, 55° C. or more, 60° C. or more, 65° C. or more, or 70° C. or more. The high temperature during hydrolysis has many advantages, which include working at the optimum temperature of the enzyme composition, the reduction of risk of (bacterial) contamination, reduced viscosity, smaller amount of cooling water required, use of cooling water with a higher temperature, re-use of the enzymes and more.

For performing the step of hydrolysis, the total amount of enzymes added (herein also called enzyme dosage or enzyme load) is low. In an embodiment the amount of enzyme is 30 mg protein/g dry matter weight or lower, 20 mg protein/g dry matter or lower, 15 mg protein/g dry matter or lower, 10 mg protein/g dry matter or lower, or 5 mg protein/g dry matter or lower (expressed as protein in mg protein/g dry matter).

In an embodiment, the amount of polysaccharide-oxidizing enzyme added is 15 mg polysaccharide-oxidizing enzyme/g dry matter weight or lower, 10 mg polysaccharide-oxidizing enzyme/g dry matter weight or lower, 5 mg polysaccharide-oxidizing enzyme/g dry matter weight or lower or 1 mg enzyme/g dry matter weight or lower (expressed as total of polysaccharide-oxidizing enzymes in mg enzyme/g dry matter). Low enzyme dosage is possible, since because of the activity and stability of the enzymes, it is possible to increase the hydrolysis reaction time.

In a further aspect of the invention, the hydrolysis reaction time is 5 hours or more, 10 hours or more, 20 hours or more, 40 hours or more, 50 hours or more, 60 hours or more, 70 hours or more, 80 hours or more, 90 hours or more, 100 hours or more, 120 hours or more, 130 h or more. In another aspect, the hydrolysis reaction time is 5 to 150 hours, 40 to 130 hours, 50 to 120 hours, 60 to 120 hours, 60 to 1 10 hours, 60 to 100 hours, 70 to 100 hours, 70 to 90 hours or 70 to 80 hours. Due to the stability of the enzyme composition longer hydrolysis reaction times are possible with corresponding higher sugar yields.

The pH during hydrolysis may be chosen by the skilled person. In a further aspect of the invention, the pH during the hydrolysis may be 3.0 to 6.4. The stable enzymes of the invention may have a broad pH range of up to 2 pH units, up to 3 pH units, up to 5 pH units. The optimum pH may lie within the limits of pH 2.0 to 8.0, 3.0 to 8.0, 3.5 to 7.0, 3.5 to 6.0, 3.5 to 5.0, 3.5 to 4.5, 4.0 to 4.5 or is about 4.2.

In a further aspect of the invention the hydrolysis step is conducted until 70% or more, 80% or more, 85% or more, 90% or more, 92% or more, 95% or more of available sugar in the lignocellulosic material is released.

In preferred embodiments, the hydrolysis step is performed in the presence of an electron donor compound and of oxygen.

An electron donor is a chemical entity, compound or composition that donates directly or indirectly electrons to another compound. It is a reducing agent that, by virtue of its donating electrons capacity, is itself oxidized in the process. Examples of electron donors are vitamin C (ascorbate), gallic acid, quinones, reduced glutathione, cysteine, low molecular weight lignin, high molecular weight lignin, ferulic acid, 3-hydroxyanthranilic acid, plant photosystem, cellobiose dehydrogenase, GMC oxidoreductase

So, electron donors are chemical entities which are involved in an oxidation reaction to generate or donate electrons. For the electron donor vitamin C, 1 molecule vitamin C donates 2 electrons. Some electron donors deliver only 1 electron per molecule. Therefore, an electron donor used in the process of the invention is quantified in vitamin C (Vit C) equivalents on basis of the electrons that will be delivered.

### Production of Industrially Useful Compounds

As it is readily understood by the one skilled in the art, a sugar product which is obtained by hydrolysis of a polysaccharide-containing material as described herein may be further processed for producing a variety of industrially useful compounds through well-known methods. Industrially useful compounds encompass ethanol and methanol.

Thus, this invention also pertains to a method for the preparation of a sugar product from a polysaccharide-containing material comprising a step of treating the said polysaccharide-containing material in the presence of a polysaccharide-oxidizing composition described in the present specification.

The use of a sugar product obtained by a method comprising a step of hydrolyzing a polysaccharide-containing material, such as a cellulose-containing material like lignocellulose are well known in the art. These methods most frequently comprises a step of fermenting the said sugar product, including fermenting glucose, so as to convert it into one or more industrially useful compounds, like ethanol or methanol.

Classically, a fermentation step is performed by using a fermenting organism, e.g. a yeast, may be fermented into a desired fermentation product, such as ethanol. Optionally the fermentation product may be recovered, e.g., by distillation.

Thus, in some embodiments, a lignocellulose-containing material is fermented by at least one fermenting organism capable of fermenting fermentable sugars, such as glucose, xylose, mannose, and galactose directly or indirectly into a desired fermentation product, according to any fermentation method which is well known from the one skilled in the art.

Subsequent to fermentation the fermentation product may be separated from the fermentation medium/broth. The medium/broth may be distilled to extract the fermentation product or the fermentation product may be extracted from the fermentation medium/broth by micro or membrane filtration techniques. Alternatively the fermentation product may be recovered by stripping. Recovery methods are well known in the art.

Such methods comprising a step of hydrolysing a lignocellulosic material and a step of fermenting the sugar product issued from hydrolysis may be used for producing any fermentation product.

Fermentation products encompass alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H2 and CO2); antibiotics (e.g., penicillin and tetracycline); enzymes; vitamins (e.g., riboflavin, B12, beta-carotene); and hormones.

Thus, the present invention also concerns a method for the preparation of a fermentation product from a polysaccharide-containing material comprising the steps of:
a) providing a polysaccharide-containing material,
b) subjecting the said polysaccharide-containing material to hydrolysis in the presence of a polysaccharide-degrading composition as described in the present specification, whereby a sugar product is obtained, and
c) fermenting the sugar product collected at step c), whereby a fermentation product is obtained,

In preferred embodiments of the method, the polysaccharide-containing material is a lignocellulosic material.

Fermentation methods are notably described by Gupta et al. (2016, Trends in Biochemical Sciences, Vol. 41 (7): 633-645).

This invention is further illustrated, without in any way being limited to, the examples below.

### EXAMPLES

A. Materials and Methods

### A.1. Cloning and Production of Genes

The nucleotide sequence was synthesized with codon optimization for P. pastoris (GenScript, Piscataway, USA) and further inserted with the native signal sequence into a pPICZaA vector (Invitrogen, Cergy-Pontoise, France) using BstBI and XbaI restriction sites, in frame with the (His)₆ tag sequence at the C-terminus. P. pastoris strain X33 and the pPICZaA vector are components of the P. pastoris Easy Select Expression System (Invitrogen), all media and protocols are described in the manufacturer's manual (Invitrogen).

Transformation of competent P. pastoris X33 was performed by electroporation with PmeI-linearized pPICZaA recombinant plasmids as described in Bennati-Granier et al Biotechnol. Biofuels 8, 90 (2015)). Zeocin-resistant P. pastoris transformants were then screened for protein production. The best-producing transformant was grown in 2 l of BMGY containing 1 ml.l⁻¹ Pichia trace minerals 4 (PTM4) salts (2 g.l⁻¹ CuSO₄.5H₂O, 3 g.l⁻¹ MnSO₄.H₂O, 0.2 g.l⁻¹ Na₂MoO₄.2H₂O, 0.02 g.l⁻¹ H₃BO₃, 0.5 g.l⁻¹ CaSO₄.2H₂O, 0.5 g.l⁻¹ CaCl₂, 12.5 g.l⁻¹ ZnSO₄.7H₂O, 22 g.l⁻¹ FeSO₄.7H₂O, biotin 0.2 g.l⁻¹, H₂SO₄ 1 ml.l⁻¹) in shaken flasks at 30° C. in an orbital shaker (200 rpm) to an OD₆₀₀ of 2 to 6. Cells were then transferred to 400 ml of BMMY containing 1 ml.l⁻¹ of PTM4 salts at 20° C. in an orbital shaker (200 rpm) for 3 days, with supplementation with 3% (v/v) methanol every day. Purification was carried out as described in Bennati-Granier et al (2015) and concentrated protein was dialyzed against sodium acetate buffer, pH 5.2 and stored at 4° C.

### A.2. Biochemical Analysis

10 μg of recombinant protein samples were loaded onto 10% SDS-PAGE gels (Thermo Fisher Scientific) and were detected by staining the gel with Coomassie Blue. The molecular mass under denaturating conditions was determined with reference standard proteins (PageRuler Prestained Protein Ladder, Thermo Fisher Scientific). Protein concentration was determined by using the Bradford assay (Bio-Rad, Mames-la-Coquette, France).

### A.3. Amplex Red Method

A fluorimetric assay to assess the reactivity of the copper-containing proteins based on Amplex Red and horseradish peroxidase was used as described previously (Isaksen et al, Kittl et al, Bennati-Granier et al). Briefly, 10 μM to 40 μM of protein were incubated in 50 mM sodium acetate buffer pH 6.0 containing 50 μM Amplex Red (Sigma-Aldrich, Saint-Quentin Fallavier, France), 7.1 U.ml⁻¹ horseradish peroxidase and 50 μM ascorbate as reductant in a final volume of 100 μl. The reaction was carried out at 30° C. for 30 minutes and fluorescence was detected using an excitation wavelength of 560 nm and an emission wavelength of 595 nm in a Tecan Infinite M200 plate reader (Tecan, Mannedorf, Switzerland).

### A.4. Saccharification Assays

Cleavage assays were performed using pretreated poplar (steam explosion under acidic conditions), Avicel and birchwood cellulose fibres. Assays were carried out in 1 ml final volume containing 5 mg poplar or 0.5% cellulose, 2.2 μM protein with 1 mM ascorbate in 50 mM sodium acetate buffer pH 5.2. The enzyme reactions were performed in 2-ml tubes and incubated in a thermomixer (Eppendorf, Montesson, France) at 45° C. and 800 rpm for 48 hours. At that time, 1 to 10 μg of T. reesei cocktail TR3012 was added to the mixture and samples were further incubated at 45° C. and 800 rpm. Samples were recovered at various time points, boiled at 100° C. for 10 min to stop the enzymatic reaction and then centrifuged at 14,000 rpm for 15 min at 4° C. to separate the soluble fraction from the remaining insoluble fraction before carbohydrate determination. The AA9 LPMO used in saccharification assays originated from Podospora anserina (PaLPMO9E). It was recombinantly expressed in P. pastoris as described in Bennati-Granier et al.

### A.5. Analysis of Oxidized and Non-Oxidized Oligosaccharides

After saccharification, soluble sugar profiles were analysed using high-performance anion-exchange chromatography (HPAEC) coupled with pulsed amperometric detection (PAD) (ICS 3000; Dionex, Sunnyvale, USA) equipped with a carboPac PA-1 analytical column (250×2 mm) and guard column. Samples and standards were injected into the HPAEC system and elution was carried out using a multi-step gradient following the protocol described in Westereng et al. Briefly, the eluents were 0.1 M NaOH (eluent A) and 1 M NaOAc in 0.1 M NaOH (eluent B). Elution was performed at a constant flow rate of 0.25 ml/min at room temperature, using a linear gradient of 0-10% eluent B over 10 min, 10-30% eluent B over 25 min, and an exponential gradient of 30-100% eluent B in 5 min. The initial condition (100% eluent A) was then restored in 1 min and maintained for 9 min to recondition the column.

A.6. Production of P. coccineus AAxx LPMOs

Bioreactor production of the best-producing transformant was carried out in 1.3-L New Brunswick BioFlo® 115 fermentors (Eppendorf, Hamburg, Germany) following the P. pastoris fermentation process guidelines (Invitrogen) with some modifications. First, preculture was performed in 500 mL shake flask containing 100 mL of BMGY medium inoculated with a single colony from YPD agar (20 g·L−1 peptone, 10 g·L−1 yeast extract, 20 g·L−1 glucose, 20 g·L−1 agar) plate. Cells were grown for 16-18 h at 30° C. in a rotary shaker at 200 rpm which resulted in an OD 600 between 4 and 6. A 10% (v/v) inoculum was used to inoculate the bioreactor. The first phase consisted in a batch culture using 400 mL of basal salts medium composed of 40 g·L−1 glycerol; 26.7 mL·L−1H 3 PO4; 14.9 g·L−1 MgSO4.7H2O; 0.93 g·L−1 CaSO4.2H2O; 7.7 g·L−1 KCl; 4.13 g·L−1 KOH; 4.35 mL·L−1 PTM 1 salt solution (6 g·L−1 CuSO4.5H2O, 0.08 g·L−1 NaI, 3 g·L−1MnSO4.H 2O, 0.2 g·L−1 Na2MoO4.2H2O, 0.02 g·L−1H3BO3, 0.5 g·L−1 CoCl2, 20 g·L−1 ZnCl2.7H2O, 0.2 g·L-lbiotin, 5 mL·L−1H2SO4, 65 g·L−1 FeSO4.7H2O). Batch phase was performed at 30° C., 400 rpm and pH was controlled at 5.0 with ammonium hydroxide (28% v/v). Dissolved oxygen was controlled at 20% with oxygen enrichment cascade (0-50%) using a gas flow rate at 0.5 v.v.m. As an antifoaming agent, 200 μl of Pluriol 8100 (BASF, Ludwigshafen, Germany) were added. After 20-24 h, phase 2 consisted of the simultaneous addition of 50 g of sorbitol and 0.5% of methanol (v/v) to the bioreactor until the yeast cells switched to methanol metabolism (i.e., 5 h later). During this phase, agitation was increased to 500 rpm and pH was slowly increased to pH 6 by addition of ammonium hydroxide (28% v/v). Finally, the induction phase (phase 3) was performed by adding a solution of methanol containing 12 mL·L−1 of PTM 1 salts (containing copper) by a fed-batch mode. The initial feed rate was 1.47 mL.h-1 and was increased after about 14 h of growth at a rate of 2.94 mL.h-1. The induction phase was carried out at 20° C. Dissolved oxygen was maintained to 20% via an agitation (400-800 rpm), gas flow (0.2-1 v.v.m.) and oxygen (O-50%) cascade. The induction phase was carried out for 144 h.

### A. 7. Purification of PcAAxx LPMOs

The culture supernatants were recovered by pelleting the cells by centrifugation at 2,700 g for 5 min, 4° C. and filtered on 0.45 μm filters (Millipore, Molsheim, France) to remove any remaining cells. For (His) 6-tagged enzymes, the pH was adjusted to 7.8 and the supernatants were filtered once more on 0.2 μm filters and loaded onto 5 ml His Trap HP columns (GE healthcare, Buc, France) connected to an Akta Xpress system (GE healthcare). Prior to loading, the columns were equilibrated in Tris HCl 50 mM pH 7.8; NaCl 150 mM (buffer A). The loaded columns were then washed with 5 column volumes (CV) of 10 mM imidazole in buffer A, before the elution step with 5 CV of 150 mM imidazole in buffer A. Fractions containing the protein were pooled and concentrated onto a 3-kDa vivaspin concentrator (Sartorius, Palaiseau, France) before loading onto a HiLoad 16/600 Superdex 75 Prep Grade column (GE Helthcare) and separated in acetate buffer 50 mM pH 5.2. Gel filtration analysis showed that both PcAAxx proteins are monomeric in solution even after copper loading. For enzymes without (His) 6-tag, salts contained in the culture media were diluted ten-fold in Tris-HCl 20 mM pH 8, then culture supernatants were concentrated onto a Pellicon-2 10-kDa cutoff cassette (Millipore) to a volume of approx. 200 mL and loaded onto a 20-mL High Prep DEAE column (GE Helthcare). Proteins were eluted using a linear gradient of 1 M NaCl (0 to 700 mM in 200 mL). Fractions were then analyzed by SDS PAGE and those containing the recombinant protein were pooled and concentrated. The concentrated proteins were then incubated with one-fold molar equivalent of CuSO4 overnight before separation on a HiLoad 16/600 Superdex 75 Prep Grade column in acetate buffer 50 mM pH 5.2. Protein-containing fractions were pooled and concentrated onto a 3-kDa vivaspin concentrator (Sartorius).

### A.8. Deglycosylation Assays

To remove N-linked glycans, purified enzymes were treated with peptide EndoHf (New England Biolabs, Ipswich, Mass.) under denaturing conditions according to the manufacturer's instructions. Briefly, 10 μg of protein were incubated in 0.5% SDS and 40 mM DTT and heated for 10 min at 100° C. for complete denaturation. Denaturated samples were subsequently incubated with 1,500 units of peptide EndoHf in 50 mM sodium acetate pH 6.0 for 1 h at 37° C. Deglycosylated and control samples were analyzed by SDS-PAGE.

### A.9. Crystallization of Purified PcAAxxB Protein

Purified PcAAxxB protein (JGI ID 1372210; GenBank ID #KY769370) was concentrated using 10-kDa polyethersulfone Vivaspin concentrators (Sartorius). The concentration was determined by measuring the A 280 nm using a NanoDrop ND-2000 instrument (Wilmington, Del., USA). All crystallization experiments were carried out at 20° C. by the sitting-drop vapour-diffusion method using 96-well crystallization plates (Swissci) and a Mosquito® Crystal (TTP labtech) crystallization robot. Reservoirs consisted of 40 μL of commercial screens and crystallization drops were prepared by mixing 100 nL reservoir solution with 100, 200 and 300 nL of protein solution. An initial hit was obtained after 1 week from a condition of the AmSO 4 screen (Qiagen) consisting of 2.4 M (NH4) 2 SO04 and 0.1 M citric acid pH 4.0. This condition was further optimized to obtain diffraction-grade crystals by mixing protein solution at 28 mg·mL-1 with precipitant solution consisting of 2.4 M (NH 4) 2 SO4 and 0.1 M citric acid pH 4.4 at a volume ratio of 3:1. PcAAxxB crystals grew to dimensions of 0.15×0.15×0.05 mm in one week. Crystals belong to space group P41212 with cell axes 204×204×110 Å and two molecules per asymmetric unit.

### A.10. Data Collection, Structure Determination and Refinement

Crystals of PcAAxxB were soaked for 5 min in a solution where 2.4 M (NH4)2SO4 of the mother liquor was replaced by 2.4 M Li2SO4 for the sake of cryoprotection prior to flash-cooling in liquid nitrogen. As X-ray fluorescence scans on native crystals did not reveal a significant presence of copper within the crystals, a heavy atom derivative was prepared by soaking the crystals in reservoir solution supplemented with 55 mM of the gadolinium complex gadoteridol prior to cryo-cooling. Native diffraction data were collected on beamline ID23-1, while a MAD dataset was collected on beamline ID30B at the European Synchrotron Radiation Facility (ESRF), Grenoble, France. Data were indexed and integrated in space group P41212 using XDS and subsequent processing steps were performed with the CCP4 software suite. Determination of the Gd3+ substructure and subsequent phasing combined with solvent flattening were carried out with SHELXC/D/E, using the SAD data collected at the Gd edge and leading to a pseudo-free correlation coefficient of 71.8%. Starting from experimental phases, an initial model comprising 526 residues (out of 584), was automatically built with Buccaneer and manually completed with Coot (44). This initial model was used for rigid body refinement followed by restrained refinement against native data with the program Refmac. A random set of 5% of reflections was set aside for cross-validation purposes. Model quality was assessed with internal modules of Coot (44) and using the Molprobity server. Data collection and refinement statistics are summarized herebelow:

TABLE 1
refinement statistics

| Property | Value | |
|---|---|---|
| Space group | P41212 | |
| Unit cell parameters | 203.75 203.75 110.59 | |
| a, b, c, α, β, γ | 90.00 90.00 90.00 | |
| Wavelength (Å) | 0.97625 | |
| Resolution range (Å) | 44.47 − 3.01 | |
| Completeness (%) (outer shell) | 98.5 | (99.4) |
| Multiplicity (outer shell) | 8.3 | (8.6) |
| <I/σ (I)> (outer shell) | 15.9 | (2.0) |

| | |
|---|---|
| Rwork/Rfree | 0.23/0.24 |
| Ramachandran statistics | 100% |
| (%) Favored & Allowed | |

### A.11. Bioinformatic Analysis of AAxx LPMOs

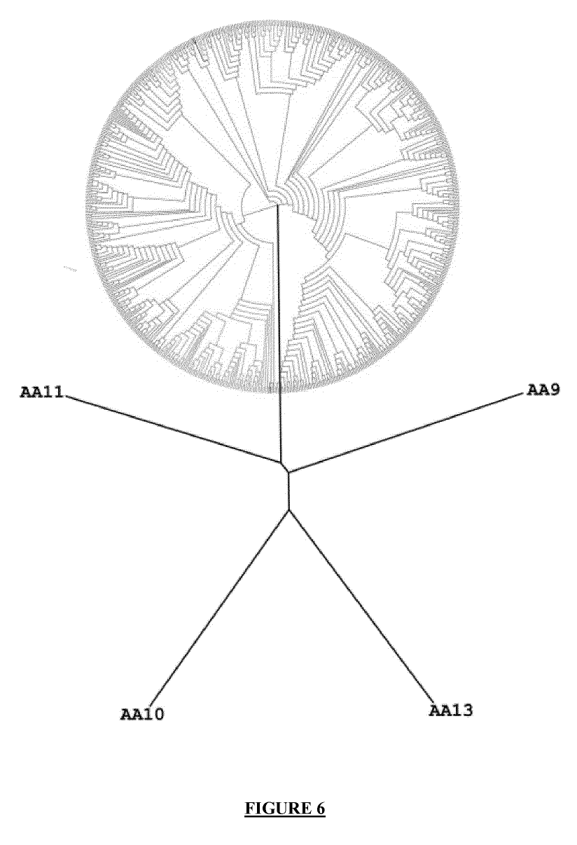

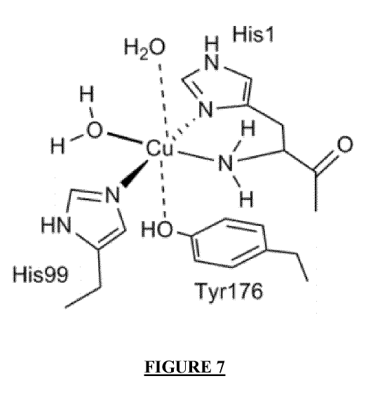

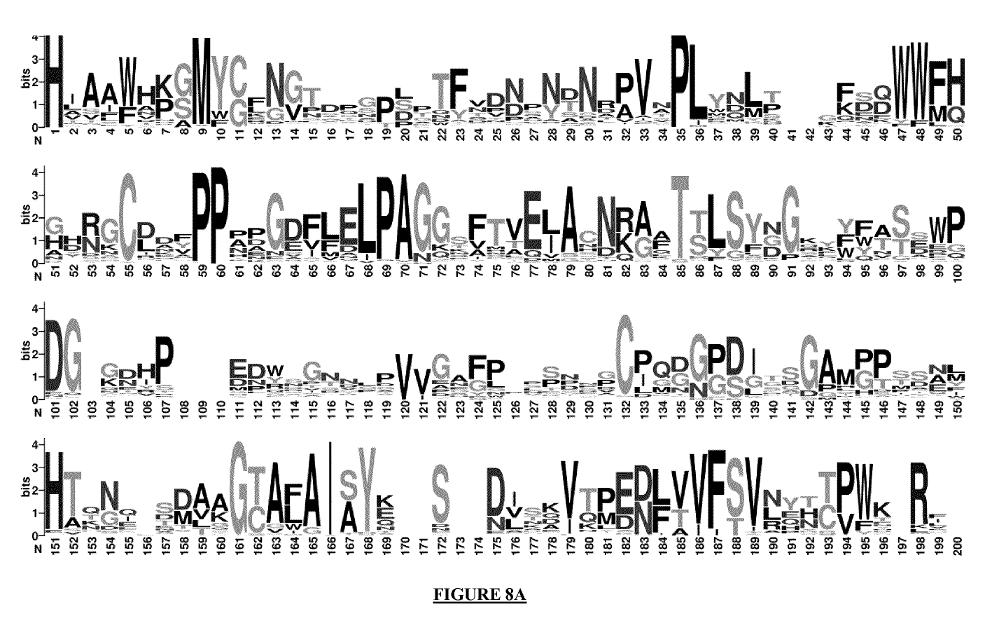

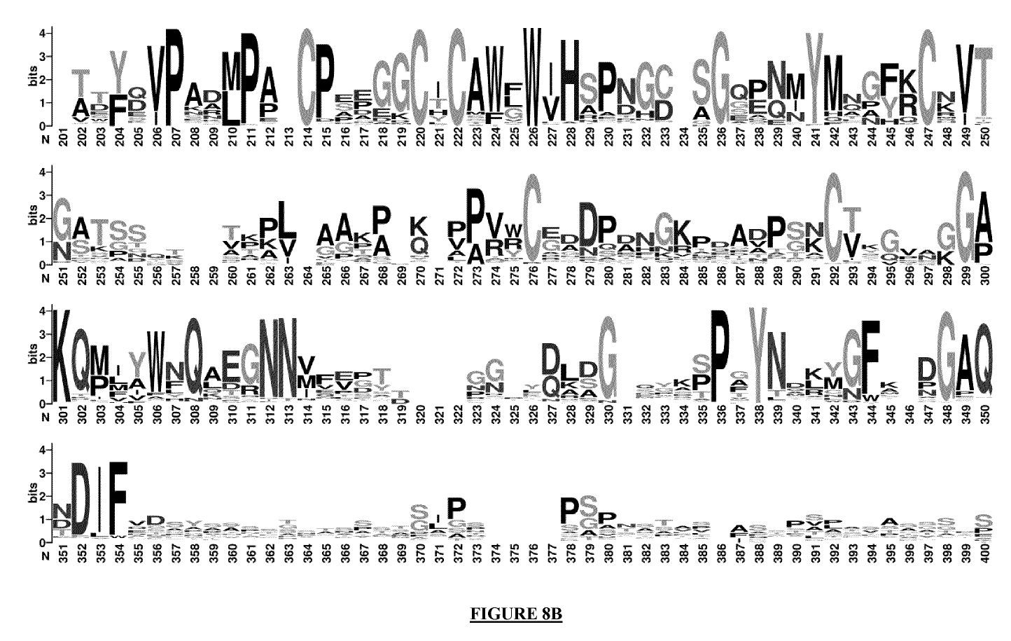

P. coccineus AAxx sequences (Genbank ID KY769369 and KY769370) were compared to the NCBI non redundant sequences database using BlastP (29) in February 2016. Blast searches conducted with AAxx did not retrieve AA9s, AA10s, AA11s or AA13s with significant scores, and vice-versa. MUSCLE was used to perform multiple alignments. To avoid interference from the presence or absence of additional residues, the signal peptides and C-terminal extensions were removed. Bioinformatic analyses were performed on 286 fungal genomes sequenced and shared by JGI collaborators. Protein clusters are available thanks to the JGI (https://goo.gl/ZAa2NX) for each of these fungi. A phylogenetic tree has been inferred using 100 cleaned and merged alignment of proteins from selected clusters of proteins. Those clusters are present, as much as possible, in all fungi in 1 copy in order to maximize the score Σ1/n (with n, the number of copy in the genome). Sequences from clusters were aligned with Mafft, trimed with Gblocks and a phylogenetic tree was built with concatenation of alignments with Fasttree. Tree is displayed with Dendroscope and Bio::phylo. See FIG. 6 for the phylogenetic tree and FIGS. 8A & 8B, corresponding to a consensus sequence of the catalytic module.

### A.12. Copper-Loading Protocol

AAxx enzymes were copper loaded using copper salts (sulphate or acetate) during or after the purification. Proteins were incubated with ten molar equivalents of copper salts between two hours and overnight at 4° C. and excess of copper was removed using diafiltration with a 3-kDa centricon or with a gel filtration chromatography step. The presence of copper can be assessed by inductively coupled plasma mass spectrometry (ICP-MS), as described here after.

### A.13. ICP/MS Analysis

Prior to the analysis, samples are mineralized in a mixture containing ⅔ of nitric acid (Sigma-Aldrich, 65% Purissime) and ⅓ of hydrochloric acid (Fluka, 37%, Trace Select), at 120° C. The residues are diluted in ultra-pure water (2 mL) before ICP/MS analysis. The ICP-MS instrument is an ICAP Q (ThermoElectron, Les Ullis, France), equipped with a collision cell. The calibration curve is obtained by dilution of a certified multi-element solution (Sigma-Aldrich). Copper concentrations are determined using Plasmalab software (Thermo-Electron), at a mass of interest m/z=63.

### Example 1: Recombinant Production of the Protein of SEQ ID NO. 1, SEQ ID NO.2 and SEQ ID NO. 3

Proteins of SEQ ID NO. 1, SEQ ID NO. 2 and SEQ ID NO.3 were produced using the heterologous expression system P. pastoris. The native signal peptide of each of the two proteins was conserved allowing for the Histidine residue to be at the N-term position after signal peptide processing in SEQ ID No. 1, SEQ ID No. 2 and SEQ ID No. 3.

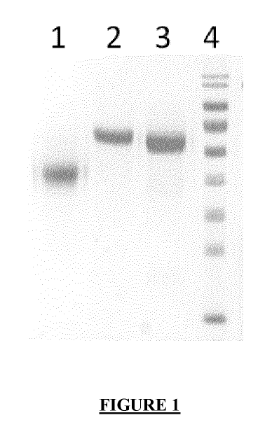

Electrophoretic analysis of the recombinant protein of SEQ ID NO. 1, the recombinant protein of SEQ ID NO. 2 and the recombinant protein of SEQ ID NO. 3 after purification revealed a single band (FIG. 1).

### Example 2: Production of Hydrogen Peroxide by the Proteins of SEQ ID NO. 1, SEQ ID NO. 2 and SEQ ID NO.3

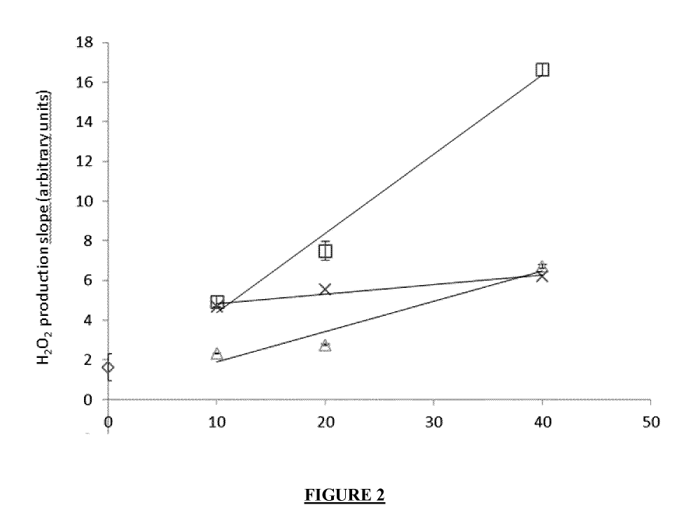

To assess the functionality of the proteins of SEQ ID NO. 1, SEQ ID NO. 2 and SEQ ID NO.3, their capacity to produce H₂O₂ in the presence of oxygen and electron donor (ascorbate) was evaluated. Significant H₂O₂ production was detected (FIG. 2,).

H₂O₂ production was in the similar range as for LPMOs from other families described in the literature such as AA9 LPMOs from P. anserina (Bennati-Granier et al., 2015, Biotechnol. Biofuels 8, 90). This assay reveals that the proteins of SEQ ID NO. 1, SEQ ID NO. 2 and SEQ ID NO.3 bears a copper ion at the catalytic center as described for LPMOs.

### Example 3: Polysaccharide-Degrading Activity of the Proteins of SEQ ID NO. 1 and SEQ ID NO.2 in a Sequential Lignocellulose Degradation Assay

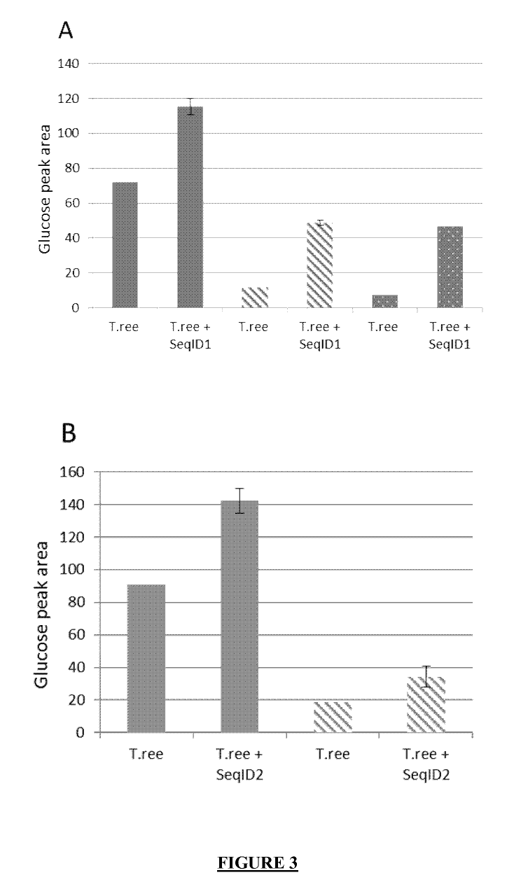

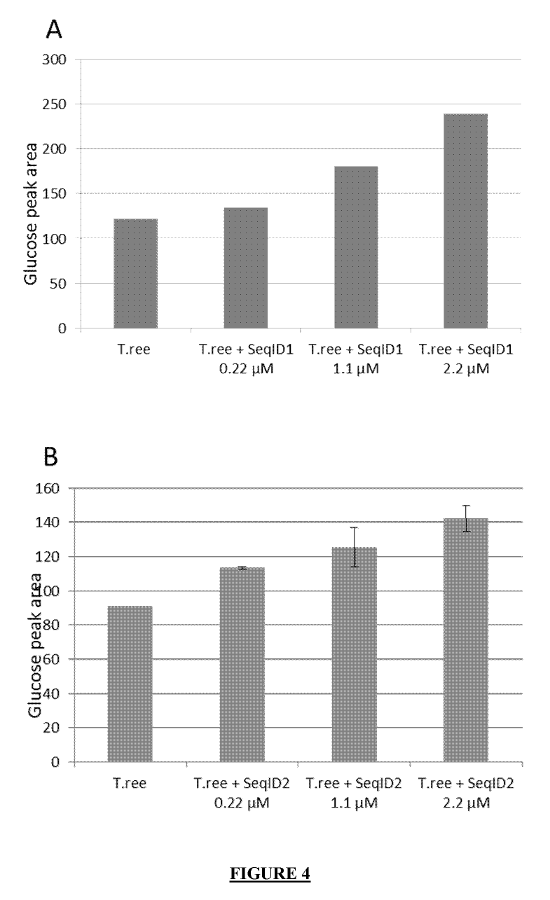

Degradation of lignocellulosic biomass with proteins of SEQ ID NO. 1 or SEQ ID NO.2 and T. reesei cellulase cocktail were tested in sequential reactions. Pretreated poplar was first incubated with 2.2 μM (equivalent to 70 μg) protein of SEQ ID NO. 1 or 2.2 μM (equivalent to 70 μg) protein SEQ ID NO.2 for 48 hours after which 10 μg of T. reesei TR3012 cellulase cocktail was added. The reactions were further incubated for 24 hours. Analyses of soluble sugar release using several methodologies (DNS assay, RTU assay and HPAEC) showed an improvement of released glucose and cello-oligosaccharides (FIG. 3). Addition of increasing amount of the proteins of SEQ ID NO. 1 or SEQ ID NO.2 resulted in proportional increase of glucose release (FIG. 4).

### Example 4: Polysaccharide-Degrading Activity of the Protein of SEQ ID NO. 1 and SEQ ID NO.2 in a Sequential Lignocellulose Degradation Assay in the Presence of AA9 LPMO

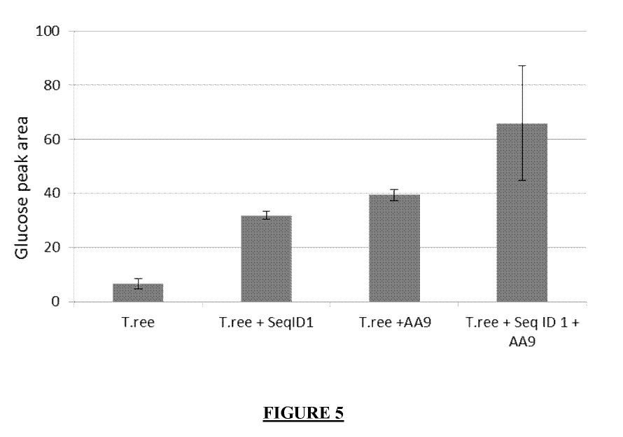

Degradation of lignocellulosic biomass with protein of SEQ ID NO. 1 and the AA9 LPMO was tested in sequential reactions. Pretreated poplar was first incubated with (i) Control medium, (ii) 2.2 μM (equivalent to 35 μg) protein of SEQ ID NO. 1, (iii) 2.2. μM of AA9 LPMO and (iv) 1.1 μM protein of SEQ ID NO. 1 and 1.1 μM of AA9 LPMO for 48 hours after which 10 μg of T. reesei TR3012 cellulase cocktail was added. The reactions were further incubated for 24 hours. Analyses of soluble sugar release using several methodologies (DNS assay, RTU assay and HPAEC) showed an improvement of released glucose and cello-oligosaccharides (FIG. 5).

### Example 5: A Crystal Structure of the PcAAxxB (JGI ID 1372210; GenBank ID #KY769370) Catalytic Module, Refined at a 3.0 Å Resolution, Reveals a Folded Core Protein and an Active Site Formed by a Canonical Histidine Brace Exposed at the Surface

PcAAxxB (#KY769370) was produced to high yield in Pichia pastoris and purified to homogeneity.

The structure of PcAAxxB was solved by multiple-wavelength anomalous dispersion data recorded at the gadolinium edge, and refined at 3.0 Å resolution. The core of the protein folds into a largely antiparallel β-sandwich, a fold globally similar to that seen in LPMOs from other families.

The active site of PcAAxxB constituted by His1, His99 and Tyr176 forming the canonical histidine brace is exposed at the surface (FIG. 2B).

In contrast to the flat substrate-binding surfaces observed in AA9 LPMOs, the PcAAxxB surface has a rippled shape with a clamp formed by two prominent surface loops.herein (see further herebelow) in a pdb (Protein Data Bank) format. Five N-glycans are attached in the crystal structure to PcAAxxB, through asparagine residues Asn13, Asn76, Asn133, Asn183 and Asn217.

The crystal structure further provides evidence of 10 cysteine residues involved in five disulfure bonds, at the following coupled positions: Cys67 & Cys90; Cys109 & Cys136; Cys153 & Cys158; Cys160 & Cys182; Cys202 & Cys218.

The crystallized structure includes two molecules per assymetric unit. Chain A coordinates are disclosed herein (see further herebelow) in a pdb (Protein Data Bank) file format (see content of the crystal structure, further herebelow). Chain B, which is also part of the assymetric unit is not represented herein.

When viewed under PyMOL© Viewer 1.7.4.5 Edu (Schrodinger, LLC), boundaries of all the 6 β-sheets forming the core antiparallel β-sandwich consist of (by reference to SEQ ID No. 2):
from residue Phe53 to Glu64;
from residue Cys109 to Ala114;
from residue Thr128 to Asn133;
from residue Phe141 to Val146;
from residue Cys158 to Trp164;
from residue Met177 to Thr185.

The sequence SEQ ID No. 7 corresponds to the minimal fragment of SEQ ID No. 2 comprising the three amino acids which are involved in the copper-binding catalytic triade (which includes the N-terminal histidine residue); and further comprising the antiparallel β-sandwich, up to residue Thr185.

The following residues comprised within SEQ ID No. 2 can be further positioned in the consensus sequence derived from FIG. 8 as follows:

TABLE 2A
Consensus Sequence and position in SEQ ID No2

| | Consensus Sequence | | | | | | |
|---|---|---|---|---|---|---|---|
| | 1 | 20-35 | 42-60 | 78-151 | 136-226 | 146-241 | 193-338 |
| Amino Acid | H | P | P | H | W | Y | Y |
| Position in | 1 | 28 | 47 | 99 | 164 | 176 | 246 |
| SEQ ID 2 | | | | | | | |

TABLE 2B
Consensus Sequence and position
in SEQ ID No2 (Cysteine Residues)

| | Consensus Sequence | | | | | |
|---|---|---|---|---|---|---|
| | 1 | 20-35 | 42-60 | 78-151 | 136-226 | 146-241 |
| Amino Acid | C | C | C | C | C | C |
| Position in | 153 | 158 | 160 | 182 | 202 | 218 |
| SEQ ID 2 | | | | | | |

### Example 6: Saccharification Assay on Pretreated Biomass Including Poplar, Pine and Wheat Straw

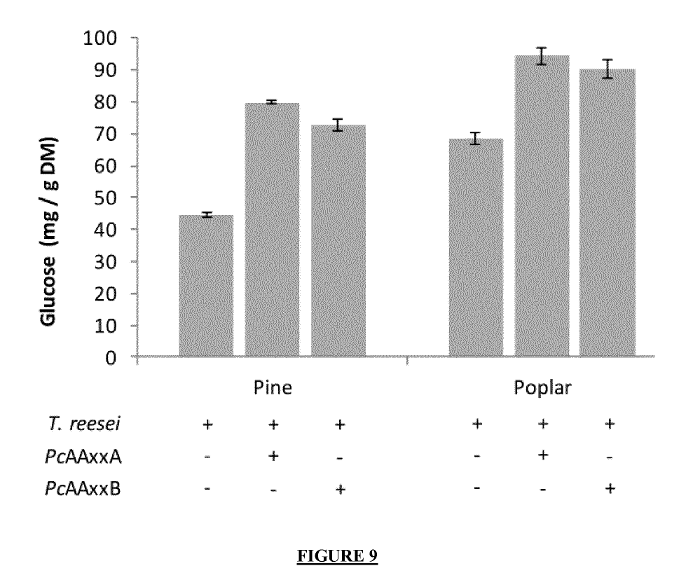

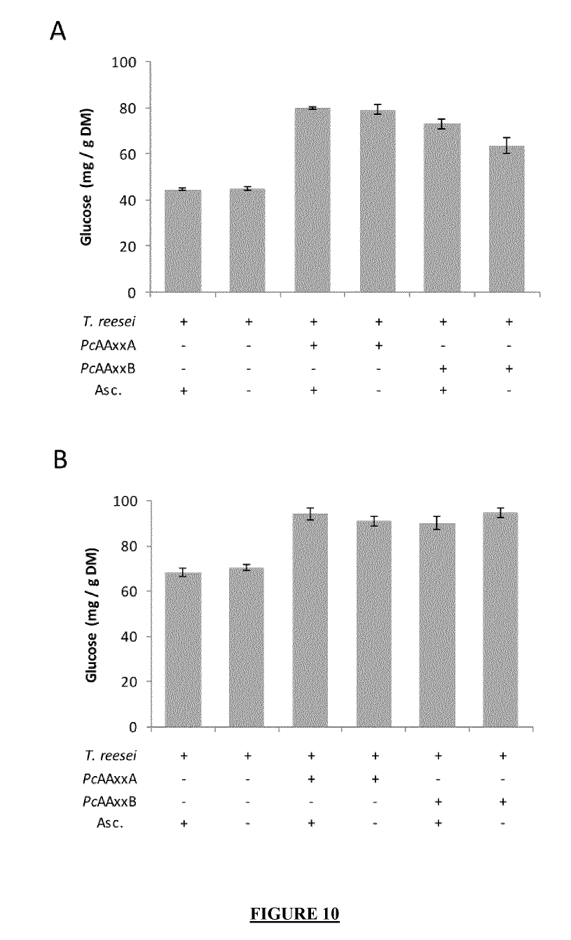

We performed saccharification assays on pretreated biomass including poplar and pine using a Trichoderma reesei CL847 cocktal mainly composed of cellulases and xylanases. A boost of glucose release from poplar and pine was observed upon addition of any of the AAxx enzymes to the cocktail (see FIG. 9).

When the reactions were conducted in the absence of a reductant the boost effect was also maintained, suggesting that one of the components from the biomass (e.g. lignin) may act as an electron donor. In a finding with important consequences for biorefinery use of woody biomass as feedstock, the T. reesei CL847 cocktail enriched in AA9 LPMO acting on cellulose was also boosted by PcAAxxA (of SEQ ID NO. 1), suggesting that AA9 and AAxx enzymes may act on different regions within the lignocellulosic matrix.

Because native AAxx members do not harbor any CBM module, we artificially attached a fungal CBM1 module targeting crystalline cellulose to PcAAxxAA. The resulting modular PcAAxxA-CBM1 enzyme performed less efficiently than the catalytic module alone (data not shown), suggesting that AAxx enzymes do not require specific binding to the flat crystalline cellulose surface.

Overall, those experiments provide evidence that enzymes belonging to the AAxx family:
remain efficient, as polysaccharide-oxidizing enzymes, for targeting a variety of material, and in particular a variety of lignocellulosic material; and
when used in polysaccharide-degrading compositions in combination with other enzymes, they may provide a surprisingly synergic activity, for instance by having a distinct attack on a lignocellulosic substrate, and especially on a xylan-comprising lignocellulosic substrate.

SEQUENCE LISTING
SEQ ID NO. 1
HAAFWDKSMYGFNVTAQTFPYDNRPQVPLYNMTFDQWWFHGHKDYPPNEGDFFELPAGGEVNSIISCDKGATP
FYESSPGGDSGYGSNSPCPGQPMSEYHTTGIDDVKGCCMAIAYKPDVNDVQPDDFVVFSCNSTCVWEMNTKFE
IPKLPACPEGGCHCAWFWIHSYDSGAEQIYMNGFKCKVTGDVGTQPLGKPAVPRRCGADPDHGKPDPTPGNCT
IGAKTPMYWYQREGNNMFEDTYDAPYYNPLYGFNDGAQNDIFMDGVIASLA
SEQ ID NO. 2
HIAFWHNSMYGFNVTEQTFPYDNRPVVPLQYMTFQEWWFHNHLDYPPHPGDFFDFPAGKAATAELACNKGATT
WFNSSEGGNIQNGNDPCPGSPPSEYHTTGIDDVKGCAMAIAYESDVRKIKPEDFTVFSVNQTCVWYRFTDFQV
PERMPPCPPGGCHCAWFWIHSPDSGGEQIYMNGFQCNITGSTSHVPLAKPKVARRCGADPDHGKPDAVPGNCT
YGAKQPLYWLQKEGNNEFDDYIAPPFYNDLYNFKDGAQNDIFVDSYPDGI
SEQ ID NO. 3
HVAAFVKGMYCEGGPDPNNYNPNSNTPVNPLWDLPFEQWWMQADRGCNKAPPPDGASVALPAGGQFTVELAHN
QAQTTLSFNGQFAGEWPDGQPHPENWSGPGSPPDCIQDDGAMHTNNQTMAAGTAWAISYNDDISKVTMDNLVV
FSVLEHTPWKRIATYDVPKDLPACPAGGCYCAWLWVPNGCGEPNMYMANYRCHVTNTTSTKQLAQAKPPTWCG
GDSSKCTKGAKQMIAWNQATGNNVQVPNGASPGYNINMGWAPGAQNDIFA
SEQ ID NO. 4
CACGCCGCGTTCTGGGACAAGTCCATGTACGGCTTCAACGTCACCGCACAGACCTTCCCCTACGACAACCGGC
CCCAGGTTCCGCTCTACAACATGACTTTCGACCAATGGTGGTTCCACGGTCACAAAGACTACCCACCCAACGA
GGGCGATTTCTTCGAACTCCCTGCGGGTGGAGAGGTGAACAGCATCATCTCCTGCGATAAGGGTGCGACTCCG
TTCTACGAGTCGTCTCCGGGCGGAGATTCGGGCTATGGCAGCAACTCTCCTTGCCCCGGGCAGCCCATGTCCG
AGTACCACACGACGGGCATTGATGATGTCAAGGGCTGCTGCATGGCCATCGCGTACAAGCCCGATGTCAACGA
TGTGCAGCCAGACGACTTCGTGGTCTTCTCCTGCAACTCGACGTGCGTTTGGGAGATGAACACCAAGTTTGAG
ATCCCGAAGCTCCCTGCCTGCCCCGAAGGTGGTTGCCATTGCGCCTGGTTCTGGATTCACTCCTACGATAGCG
GTGCTGAGCAGATCTATATGAATGGTTTCAAGTGCAAAGTAACCGGCGACGTAGGCACTCAGCCTCTCGGCAA
GCCTGCCGTCCCGCGCAGGTGCGGTGCCGACCCTGATCACGGCAAGCCTGATCCTACTCCCGGTAATTGTACT
ATCGGGGCCAAGACGCCCATGTACTGGTACCAGCGGGAGGGTAACAACATGTTCGAAGACACTTATGACGCCC
CGTACTATAACCCACTCTACGGCTTCAACGATGGGGCGCAGAACGACATTTTCATGGATGGTGTCATCGCGTC
CCTCGCA
SEQ ID NO. 5
CACATCGCCTTCTGGCATAACAGCATGTACGGGTTCAATGTGACGGAACAGACGTTCCCTTACGACAACCGCC
CCGTCGTCCCGCTTCAGTACATGACCTTCCAAGAATGGTGGTTCCACAACCACCTCGACTACCCGCCCCACCC
GGGCGACTTCTTCGACTTCCCGGCCGGCAAGGCCGCGACGGCGGAGCTCGCGTGCAACAAGGGCGCGACCACC
TGGTTCAACTCCTCCGAGGGCGGCAACATCCAGAACGGCAACGACCCGTGCCCGGGGAGCCCCCCGAGCGAGT
ACCACACGACGGGCATCGACGACGTGAAGGGCTGCGCGATGGCGATCGCGTACGAGTCCGACGTCAGGAAGAT
CAAGCCCGAGGACTTCACCGTCTTCAGCGTGAACCAGACGTGCGTCTGGTACCGCTTCACGGACTTCCAGGTC
CCCGAGCGCATGCCGCCGTGCCCTCCTGGCGGCTGTCACTGCGCGTGGTTCTGGATCCACTCGCCCGATAGCG
GCGGTGAGCAGATCTACATGAACGGCTTCCAGTGCAACATCACCGGCTCCACGTCCCACGTCCCGCTCGCAAA
GCCCAAAGTCGCTCGCCGCTGCGGTGCGGACCCGGACCACGGCAAGCCCGACGCCGTCCCCGGCAACTGCACA
TACGGTGCGAAGCAGCCCCTCTACTGGCTCCAGAAGGAGGGCAACAACGAGTTCGACGACTACATCGCGCCGC
CGTTCTACAACGACCTGTACAACTTCAAGGACGGCGCGCAGAACGACATCTTCGTCGACTCGTACCCCGACGG
CATC
SEQ ID NO. 6
ATGTTCTGGACGGTTCCCGTTACACTGGCTCTCGCCTCCGGGGCCTCTGCCCATGTTGCCGCCTTCGTGAAGG
GCATGTACTGCGAGGGTGGCCCCGACCCTAACAACTACAACCCCAACTCCAACACTCCCGTCAACCCTCTCTG
GGATCTTCCTTTCGAGCAGTGGTGGATGCAGGCCGACCGTGGCTGCAACAAGGCTCCTCCTCCTGACGGTGCC
TCCGTTGCTCTGCCTGCCGGCGGCCAGTTCACTGTTGAGCTCGCCCACAACCAGGCCCAGACGACCTTATCCT
TCAATGGACAGTTCGCTGGCGAGTGGCCTGATGGCCAGCCTCACCCAGAGAACTGGAGCGGCCCCGGTAGCCC
CCCTGACTGCATCCAGGACGACGGTGCCATGCATACCAACAACCAGACAATGGCCGCTGGTACCGCTTGGGCC
ATTTCCTACAATGACGACATCTCCAAGGTGACCATGGACAACCTGGTTGTTTTCTCTGTCCTTGAGCACACCC
CTTGGAAGCGCATCGCCACCTATGATGTCCCCAAGGATCTCCCTGCTTGCCCTGCTGGCGGCTGCTACTGCGC
CTGGCTTTGGGTTCCCAACGGGTGCGGCGAGCCCAACATGTACATGGCCAACTACAGGTGCCATGTCACCAAC
ACCACCTCGACCAAGCAGCTTGCTCAAGCCAAGCCCCCCACCTGGTGTGGCGGTGACTCTTCCAAGTGCACCA
AGGGCGCTAAGCAGATGATTGCTTGGAACCAGGCCACAGGCAACAATGTCCAAGTTCCCAATGGCGCTTCCCC
CGGCTACAACATCAACATGGGCTGGGCTCCCGGTGCTCAGAACGACATCTTCGCGTAG
SEQ ID NO. 7
HIAFWHNSMYGFNVTEQTFPYDNRPVVPLQYMTFQEWWFHNHLDYPPHPGDFFDFPAGKAATAELACNKGATT
WFNSSEGGNIQNGNDPCPGSPPSEYHTTGIDDVKGCAMAIAYESDVRKIKPEDFTVFSVNQTCVWYRFTDFQV
PERMPPCPPGGCHCAWFWIHSPDSGGEQIYMNGFQCNIT
SEQ ID No 8
MFWTVPVTLALASGASA
SEQ ID NO. 9
MFWTVPVTLALASGASAHVAAFVKGMYCEGGPDPNNYNPNSNTPVNPLWDLPFEQWWMQADRGCNKAPPPDGA
SVALPAGGQFTVELAHNQAQTTLSFNGQFAGEWPDGQPHPENWSGPGSPPDCIQDDGAMHTNNQTMAAGTAWA
ISYNDDISKVTMDNLVVFSVLEHTPWKRIATYDVPKDLPACPAGGCYCAWLWVPNGCGEPNMYMANYRCHVTN
TTSTKQLAQAKPPTWCGGDSSKCTKGAKQMIAWNQATGNNVQVPNGASPGYNINMGWAPGAQNDIFA

CRYSTAL STRUCTURE OF AAxx (PcAAxxB) of SEQ ID No2

| | | | | | |
|---|---|---|---|---|---|
| MODRES | NAG | A | 300 | NAG-b-D | RENAME |
| MODRES | NAG | A | 301 | NAG-b-D | RENAME |
| MODRES | NAG | A | 310 | NAG-b-D | RENAME |
| MODRES | NAG | A | 311 | NAG-b-D | RENAME |
| MODRES | MAN | A | 313 | MAN-a-D | RENAME |
| MODRES | MAN | A | 314 | MAN-a-D | RENAME |
| MODRES | MAN | A | 315 | MAN-a-D | RENAME |
| MODRES | NAG | A | 320 | NAG-b-D | RENAME |
| MODRES | NAG | A | 321 | NAG-b-D | RENAME |
| MODRES | MAN | A | 323 | MAN-a-D | RENAME |
| MODRES | MAN | A | 324 | MAN-a-D | RENAME |
| MODRES | NAG | A | 330 | NAG-b-D | RENAME |
| MODRES | NAG | A | 331 | NAG-b-D | RENAME |
| MODRES | MAN | A | 333 | MAN-a-D | RENAME |
| MODRES | NAG | A | 340 | NAG-b-D | RENAME |
| MODRES | NAG | A | 341 | NAG-b-D | RENAME |
| MODRES | MAN | A | 343 | MAN-a-D | RENAME |
| MODRES | MAN | A | 344 | MAN-a-D | RENAME |
| MODRES | MAN | A | 345 | MAN-a-D | RENAME |
| MODRES | MAN | A | 346 | MAN-a-D | RENAME |

| | | | | | | | |
|---|---|---|---|---|---|---|---|
| SSBOND | 1 | CYS | A | 67 | CYS | A | 90 |
| SSBOND | 2 | CYS | A | 109 | CYS | A | 136 |
| SSBOND | 3 | CYS | A | 153 | CYS | A | 158 |
| SSBOND | 4 | CYS | A | 160 | CYS | A | 182 |
| SSBOND | 5 | CYS | A | 202 | CYS | A | 218 |

| | | | | | | | | | |
|---|---|---|---|---|---|---|---|---|---|
| LINKR | C1 | NAG | A | 300 | ND2 | ASN | A | 13 | NAG-ASN |
| LINKR | C1 | NAG | A | 310 | ND2 | ASN | A | 76 | NAG-ASN |
| LINKR | C1 | NAG | A | 320 | ND2 | ASN | A | 133 | NAG-ASN |
| LINKR | C1 | NAG | A | 330 | ND2 | ASN | A | 183 | NAG-ASN |
| LINKR | C1 | NAG | A | 340 | ND2 | ASN | A | 217 | NAG-ASN |
| LINKR | | NAG | A | 300 | | NAG | A | 301 | BETA1-4 |
| LINKR | | NAG | A | 301 | | BMA | A | 302 | BETA1-4 |
| LINKR | | NAG | A | 310 | | NAG | A | 311 | BETA1-4 |
| LINKR | | NAG | A | 311 | | BMA | A | 312 | BETA1-4 |
| LINKR | | BMA | A | 312 | | MAN | A | 313 | ALPHA1-6 |
| LINKR | | MAN | A | 313 | | MAN | A | 314 | ALPHA1-3 |
| LINKR | | BMA | A | 312 | | MAN | A | 315 | ALPHA1-3 |
| LINKR | | NAG | A | 320 | | NAG | A | 321 | BETA1-4 |
| LINKR | | NAG | A | 321 | | BMA | A | 322 | BETA1-4 |
| LINKR | | BMA | A | 322 | | MAN | A | 323 | ALPHA1-3 |
| LINKR | | BMA | A | 322 | | MAN | A | 324 | ALPHA1-6 |
| LINKR | | NAG | A | 330 | | NAG | A | 331 | BETA1-4 |
| LINKR | | NAG | A | 331 | | BMA | A | 332 | BETA1-4 |
| LINKR | | BMA | A | 332 | | MAN | A | 333 | ALPHA1-3 |
| LINKR | | NAG | A | 340 | | NAG | A | 341 | BETA1-4 |
| LINKR | | NAG | A | 341 | | BMA | A | 342 | BETA1-4 |
| LINKR | | BMA | A | 342 | | MAN | A | 343 | ALPHA1-3 |
| LINKR | | MAN | A | 343 | | MAN | A | 345 | ALPHA1-2 |
| LINKR | | BMA | A | 342 | | MAN | A | 344 | ALPHA1-6 |
| LINKR | | MAN | A | 343 | | MAN | A | 346 | ALPHA1-6 |

| | | | | | | | | | | |
|---|---|---|---|---|---|---|---|---|---|---|
| CRYST1 | 203.754 | 203.754 | 110.586 | 90.00 | 90.00 | 90.00 | P | 41 | 21 | 2 |

| | | | | |
|---|---|---|---|---|
| SCALE1 | 0.004908 | 0.000000 | 0.000000 | 0.00000 |
| SCALE2 | −0.000000 | 0.004908 | 0.000000 | 0.00000 |
| SCALE3 | 0.000000 | −0.000000 | 0.009043 | 0.00000 |

| | | | | | | | | | | | |
|---|---|---|---|---|---|---|---|---|---|---|---|
| ATOM | 1 | N | HIS | A | 1 | 84.207 | 23.023 | 6.334 | 1.00 | 91.64 | N |
| ATOM | 2 | CA | HIS | A | 1 | 84.466 | 23.579 | 7.697 | 1.00 | 92.54 | C |
| ATOM | 3 | CB | HIS | A | 1 | 83.152 | 23.732 | 8.477 | 1.00 | 96.32 | C |
| ATOM | 4 | CG | HIS | A | 1 | 82.286 | 24.858 | 7.994 | 1.00 | 103.06 | C |
| ATOM | 5 | ND1 | HIS | A | 1 | 81.800 | 24.929 | 6.705 | 1.00 | 107.25 | N |
| ATOM | 6 | CE1 | HIS | A | 1 | 81.067 | 26.021 | 6.570 | 1.00 | 107.65 | C |
| ATOM | 7 | NE2 | HIS | A | 1 | 81.051 | 26.657 | 7.728 | 1.00 | 107.39 | N |
| ATOM | 8 | CD2 | HIS | A | 1 | 81.802 | 25.948 | 8.636 | 1.00 | 107.70 | C |
| ATOM | 9 | C | HIS | A | 1 | 85.475 | 22.714 | 8.468 | 1.00 | 89.46 | C |
| ATOM | 10 | O | HIS | A | 1 | 85.099 | 21.813 | 9.220 | 1.00 | 87.30 | O |
| ATOM | 11 | N | ILE | A | 2 | 86.758 | 22.994 | 8.247 | 1.00 | 85.02 | N |
| ATOM | 12 | CA | ILE | A | 2 | 87.864 | 22.287 | 8.897 | 1.00 | 82.47 | C |
| ATOM | 13 | CB | ILE | A | 2 | 88.504 | 21.243 | 7.951 | 1.00 | 82.74 | C |
| ATOM | 14 | CG1 | ILE | A | 2 | 87.504 | 20.143 | 7.573 | 1.00 | 84.37 | C |
| ATOM | 15 | CD1 | ILE | A | 2 | 87.087 | 19.238 | 8.714 | 1.00 | 84.11 | C |
| ATOM | 16 | CG2 | ILE | A | 2 | 89.744 | 20.610 | 8.573 | 1.00 | 82.26 | C |
| ATOM | 17 | C | ILE | A | 2 | 88.930 | 23.304 | 9.288 | 1.00 | 82.66 | C |
| ATOM | 18 | O | ILE | A | 2 | 89.091 | 24.331 | 8.623 | 1.00 | 81.32 | O |
| ATOM | 19 | N | ALA | A | 3 | 89.659 | 23.015 | 10.364 | 1.00 | 83.36 | N |
| ATOM | 20 | CA | ALA | A | 3 | 90.744 | 23.883 | 10.810 | 1.00 | 83.72 | C |
| ATOM | 21 | CB | ALA | A | 3 | 90.229 | 24.884 | 11.825 | 1.00 | 85.46 | C |
| ATOM | 22 | C | ALA | A | 3 | 91.897 | 23.099 | 11.410 | 1.00 | 82.57 | C |
| ATOM | 23 | O | ALA | A | 3 | 91.711 | 22.006 | 11.943 | 1.00 | 82.43 | O |
| ATOM | 24 | N | PHE | A | 4 | 93.091 | 23.676 | 11.307 | 1.00 | 82.50 | N |
| ATOM | 25 | CA | PHE | A | 4 | 94.289 | 23.131 | 11.939 | 1.00 | 82.14 | C |
| ATOM | 26 | CB | PHE | A | 4 | 95.544 | 23.679 | 11.248 | 1.00 | 81.58 | C |
| ATOM | 27 | CG | PHE | A | 4 | 96.801 | 23.534 | 12.055 | 1.00 | 82.24 | C |
| ATOM | 28 | CD1 | PHE | A | 4 | 97.531 | 22.356 | 12.025 | 1.00 | 84.74 | C |
| ATOM | 29 | CE1 | PHE | A | 4 | 98.696 | 22.226 | 12.763 | 1.00 | 85.69 | C |
| ATOM | 30 | CZ | PHE | A | 4 | 99.145 | 23.281 | 13.536 | 1.00 | 86.18 | C |
| ATOM | 31 | CE2 | PHE | A | 4 | 98.430 | 24.463 | 13.568 | 1.00 | 85.11 | C |
| ATOM | 32 | CD2 | PHE | A | 4 | 97.267 | 24.587 | 12.829 | 1.00 | 82.90 | C |
| ATOM | 33 | C | PHE | A | 4 | 94.243 | 23.540 | 13.408 | 1.00 | 79.27 | C |
| ATOM | 34 | O | PHE | A | 4 | 94.223 | 24.733 | 13.713 | 1.00 | 78.92 | O |
| ATOM | 35 | N | TRP | A | 5 | 94.214 | 22.556 | 14.308 | 1.00 | 76.19 | N |
| ATOM | 36 | CA | TRP | A | 5 | 94.078 | 22.815 | 15.748 | 1.00 | 73.59 | C |
| ATOM | 37 | CB | TRP | A | 5 | 92.972 | 21.947 | 16.342 | 1.00 | 71.23 | C |
| ATOM | 38 | CG | TRP | A | 5 | 91.616 | 22.342 | 15.888 | 1.00 | 71.66 | C |
| ATOM | 39 | CD1 | TRP | A | 5 | 90.794 | 21.643 | 15.057 | 1.00 | 71.88 | C |
| ATOM | 40 | NE1 | TRP | A | 5 | 89.622 | 22.333 | 14.860 | 1.00 | 73.14 | N |
| ATOM | 41 | CE2 | TRP | A | 5 | 89.674 | 23.506 | 15.565 | 1.00 | 73.24 | C |
| ATOM | 42 | CD2 | TRP | A | 5 | 90.919 | 23.547 | 16.223 | 1.00 | 72.48 | C |
| ATOM | 43 | CE3 | TRP | A | 5 | 91.226 | 24.656 | 17.018 | 1.00 | 72.79 | C |
| ATOM | 44 | CZ3 | TRP | A | 5 | 90.291 | 25.675 | 17.132 | 1.00 | 73.08 | C |
| ATOM | 45 | CH2 | TRP | A | 5 | 89.059 | 25.606 | 16.466 | 1.00 | 74.22 | C |
| ATOM | 46 | CZ2 | TRP | A | 5 | 88.733 | 24.533 | 15.679 | 1.00 | 74.18 | C |
| ATOM | 47 | C | TRP | A | 5 | 95.360 | 22.586 | 16.533 | 1.00 | 75.33 | C |
| ATOM | 48 | O | TRP | A | 5 | 95.922 | 21.491 | 16.518 | 1.00 | 74.09 | O |
| ATOM | 49 | N | HIS | A | 6 | 95.804 | 23.632 | 17.227 | 1.00 | 80.24 | N |
| ATOM | 50 | CA | HIS | A | 6 | 96.984 | 23.570 | 18.089 | 1.00 | 80.86 | C |
| ATOM | 51 | CB | HIS | A | 6 | 98.257 | 23.467 | 17.251 | 1.00 | 78.51 | C |
| ATOM | 52 | CG | HIS | A | 6 | 99.432 | 22.941 | 18.008 | 1.00 | 76.30 | C |
| ATOM | 53 | ND1 | HIS | A | 6 | 100.512 | 23.725 | 18.351 | 1.00 | 76.28 | N |
| ATOM | 54 | CE1 | HIS | A | 6 | 101.395 | 22.993 | 19.006 | 1.00 | 76.89 | C |
| ATOM | 55 | NE2 | HIS | A | 6 | 100.923 | 21.763 | 19.104 | 1.00 | 76.64 | N |
| ATOM | 56 | CD2 | HIS | A | 6 | 99.699 | 21.704 | 18.485 | 1.00 | 74.93 | C |
| ATOM | 57 | C | HIS | A | 6 | 97.027 | 24.823 | 18.964 | 1.00 | 83.70 | C |
| ATOM | 58 | O | HIS | A | 6 | 96.542 | 25.872 | 18.553 | 1.00 | 87.03 | O |
| ATOM | 59 | N | ASN | A | 7 | 97.613 | 24.720 | 20.156 | 1.00 | 84.93 | N |
| ATOM | 60 | CA | ASN | A | 7 | 97.683 | 25.864 | 21.083 | 1.00 | 82.64 | C |
| ATOM | 61 | CB | ASN | A | 7 | 98.139 | 25.415 | 22.476 | 1.00 | 82.43 | C |
| ATOM | 62 | CG | ASN | A | 7 | 99.559 | 24.883 | 22.488 | 1.00 | 85.12 | C |
| ATOM | 63 | OD1 | ASN | A | 7 | 100.174 | 24.686 | 21.440 | 1.00 | 88.15 | O |
| ATOM | 64 | ND2 | ASN | A | 7 | 100.089 | 24.647 | 23.682 | 1.00 | 87.40 | N |
| ATOM | 65 | C | ASN | A | 7 | 98.571 | 27.017 | 20.592 | 1.00 | 80.24 | C |
| ATOM | 66 | O | ASN | A | 7 | 98.520 | 28.120 | 21.138 | 1.00 | 81.89 | O |
| ATOM | 67 | N | SER | A | 8 | 99.379 | 26.753 | 19.568 | 1.00 | 76.92 | N |
| ATOM | 68 | CA | SER | A | 8 | 100.251 | 27.760 | 18.971 | 1.00 | 76.31 | C |
| ATOM | 69 | CB | SER | A | 8 | 101.521 | 27.099 | 18.439 | 1.00 | 78.16 | C |
| ATOM | 70 | OG | SER | A | 8 | 101.219 | 26.179 | 17.403 | 1.00 | 76.69 | O |
| ATOM | 71 | C | SER | A | 8 | 99.578 | 28.518 | 17.828 | 1.00 | 74.65 | C |
| ATOM | 72 | O | SER | A | 8 | 100.198 | 29.385 | 17.213 | 1.00 | 74.69 | O |
| ATOM | 73 | N | MET | A | 9 | 98.324 | 28.191 | 17.536 | 1.00 | 73.77 | N |
| ATOM | 74 | CA | MET | A | 9 | 97.611 | 28.834 | 16.441 | 1.00 | 74.84 | C |
| ATOM | 75 | CB | MET | A | 9 | 96.458 | 27.949 | 15.954 | 1.00 | 74.96 | C |
| ATOM | 76 | CG | MET | A | 9 | 95.228 | 27.964 | 16.847 | 1.00 | 74.10 | C |
| ATOM | 77 | SD | MET | A | 9 | 94.042 | 26.680 | 16.421 | 1.00 | 74.41 | S |
| ATOM | 78 | CE | MET | A | 9 | 93.364 | 27.372 | 14.914 | 1.00 | 77.74 | C |
| ATOM | 79 | C | MET | A | 9 | 97.063 | 30.188 | 16.849 | 1.00 | 75.28 | C |
| ATOM | 80 | O | MET | A | 9 | 96.799 | 30.440 | 18.024 | 1.00 | 83.04 | O |
| ATOM | 81 | N | TYR | A | 10 | 96.888 | 31.053 | 15.859 | 1.00 | 74.31 | N |
| ATOM | 82 | CA | TYR | A | 10 | 96.248 | 32.339 | 16.074 | 1.00 | 74.32 | C |
| ATOM | 83 | CB | TYR | A | 10 | 96.636 | 33.330 | 14.973 | 1.00 | 72.98 | C |
| ATOM | 84 | CG | TYR | A | 10 | 98.026 | 33.895 | 15.150 | 1.00 | 71.66 | C |
| ATOM | 85 | CD1 | TYR | A | 10 | 99.150 | 33.108 | 14.941 | 1.00 | 72.01 | C |
| ATOM | 86 | CE1 | TYR | A | 10 | 100.426 | 33.619 | 15.115 | 1.00 | 72.90 | C |
| ATOM | 87 | CZ | TYR | A | 10 | 100.586 | 34.935 | 15.497 | 1.00 | 73.32 | C |
| ATOM | 88 | OH | TYR | A | 10 | 101.845 | 35.457 | 15.668 | 1.00 | 75.71 | O |
| ATOM | 89 | CE2 | TYR | A | 10 | 99.486 | 35.738 | 15.707 | 1.00 | 73.41 | C |
| ATOM | 90 | CD2 | TYR | A | 10 | 98.216 | 35.216 | 15.535 | 1.00 | 73.15 | C |
| ATOM | 91 | C | TYR | A | 10 | 94.740 | 32.106 | 16.111 | 1.00 | 75.52 | C |
| ATOM | 92 | O | TYR | A | 10 | 94.209 | 31.290 | 15.354 | 1.00 | 77.48 | O |
| ATOM | 93 | N | GLY | A | 11 | 94.059 | 32.813 | 17.004 | 1.00 | 76.98 | N |
| ATOM | 94 | CA | GLY | A | 11 | 92.618 | 32.658 | 17.176 | 1.00 | 77.07 | C |
| ATOM | 95 | C | GLY | A | 11 | 92.251 | 31.299 | 17.739 | 1.00 | 77.32 | C |
| ATOM | 96 | O | GLY | A | 11 | 91.217 | 30.735 | 17.388 | 1.00 | 78.34 | O |
| ATOM | 97 | N | PHE | A | 12 | 93.100 | 30.779 | 18.620 | 1.00 | 76.88 | N |
| ATOM | 98 | CA | PHE | A | 12 | 92.893 | 29.466 | 19.228 | 1.00 | 77.53 | C |
| ATOM | 99 | CB | PHE | A | 12 | 94.060 | 29.150 | 20.169 | 1.00 | 79.12 | C |
| ATOM | 100 | CG | PHE | A | 12 | 93.945 | 27.823 | 20.868 | 1.00 | 82.77 | C |
| ATOM | 101 | CD1 | PHE | A | 12 | 93.956 | 26.638 | 20.146 | 1.00 | 83.25 | C |
| ATOM | 102 | CE1 | PHE | A | 12 | 93.863 | 25.415 | 20.785 | 1.00 | 83.58 | C |
| ATOM | 103 | CZ | PHE | A | 12 | 93.771 | 25.363 | 22.166 | 1.00 | 84.84 | C |
| ATOM | 104 | CE2 | PHE | A | 12 | 93.768 | 26.534 | 22.900 | 1.00 | 85.08 | C |
| ATOM | 105 | CD2 | PHE | A | 12 | 93.861 | 27.755 | 22.253 | 1.00 | 85.04 | C |
| ATOM | 106 | C | PHE | A | 12 | 91.560 | 29.370 | 19.972 | 1.00 | 78.05 | C |
| ATOM | 107 | O | PHE | A | 12 | 90.881 | 28.343 | 19.906 | 1.00 | 79.95 | O |
| ATOM | 108 | N | ASN | A | 13 | 91.187 | 30.445 | 20.664 | 1.00 | 79.85 | N |
| ATOM | 109 | CA | ASN | A | 13 | 89.955 | 30.483 | 21.455 | 1.00 | 80.66 | C |
| ATOM | 110 | CB | ASN | A | 13 | 90.259 | 31.013 | 22.860 | 1.00 | 82.92 | C |
| ATOM | 111 | CG | ASN | A | 13 | 91.025 | 30.020 | 23.719 | 1.00 | 85.75 | C |
| ATOM | 112 | OD1 | ASN | A | 13 | 91.179 | 28.849 | 23.368 | 1.00 | 86.69 | O |
| ATOM | 113 | ND2 | ASN | A | 13 | 91.509 | 30.497 | 24.869 | 1.00 | 88.38 | N |
| ATOM | 114 | C | ASN | A | 13 | 88.835 | 31.321 | 20.839 | 1.00 | 80.10 | C |
| ATOM | 115 | O | ASN | A | 13 | 87.829 | 31.582 | 21.499 | 1.00 | 79.99 | O |
| ATOM | 116 | N | VAL | A | 14 | 88.996 | 31.732 | 19.582 | 1.00 | 80.60 | N |
| ATOM | 117 | CA | VAL | A | 14 | 87.972 | 32.528 | 18.898 | 1.00 | 79.17 | C |
| ATOM | 118 | CB | VAL | A | 14 | 88.473 | 33.066 | 17.539 | 1.00 | 78.29 | C |
| ATOM | 119 | CG1 | VAL | A | 14 | 87.331 | 33.669 | 16.731 | 1.00 | 78.86 | C |
| ATOM | 120 | CG2 | VAL | A | 14 | 89.567 | 34.101 | 17.751 | 1.00 | 79.23 | C |
| ATOM | 121 | C | VAL | A | 14 | 86.715 | 31.690 | 18.685 | 1.00 | 79.96 | C |
| ATOM | 122 | O | VAL | A | 14 | 86.795 | 30.543 | 18.244 | 1.00 | 82.35 | O |
| ATOM | 123 | N | THR | A | 15 | 85.562 | 32.271 | 19.007 | 1.00 | 81.16 | N |
| ATOM | 124 | CA | THR | A | 15 | 84.278 | 31.594 | 18.853 | 1.00 | 83.25 | C |
| ATOM | 125 | CB | THR | A | 15 | 83.560 | 31.430 | 20.208 | 1.00 | 82.34 | C |
| ATOM | 126 | OG1 | THR | A | 15 | 83.185 | 32.715 | 20.719 | 1.00 | 83.75 | O |
| ATOM | 127 | CG2 | THR | A | 15 | 84.461 | 30.721 | 21.209 | 1.00 | 80.67 | C |
| ATOM | 128 | C | THR | A | 15 | 83.385 | 32.364 | 17.880 | 1.00 | 86.18 | C |
| ATOM | 129 | O | THR | A | 15 | 83.780 | 33.402 | 17.344 | 1.00 | 86.80 | O |
| ATOM | 130 | N | GLU | A | 16 | 82.185 | 31.839 | 17.656 | 1.00 | 90.27 | N |
| ATOM | 131 | CA | GLU | A | 16 | 81.237 | 32.423 | 16.714 | 1.00 | 95.92 | C |
| ATOM | 132 | CB | GLU | A | 16 | 79.978 | 31.557 | 16.663 | 1.00 | 99.78 | C |
| ATOM | 133 | CG | GLU | A | 16 | 78.944 | 31.990 | 15.636 | 1.00 | 101.53 | C |
| ATOM | 134 | CD | GLU | A | 16 | 77.828 | 30.973 | 15.465 | 1.00 | 104.50 | C |
| ATOM | 135 | OE1 | GLU | A | 16 | 78.104 | 29.755 | 15.519 | 1.00 | 106.60 | O |
| ATOM | 136 | OE2 | GLU | A | 16 | 76.670 | 31.390 | 15.262 | 1.00 | 104.82 | O |
| ATOM | 137 | C | GLU | A | 16 | 80.870 | 33.868 | 17.052 | 1.00 | 98.70 | C |
| ATOM | 138 | O | GLU | A | 16 | 80.799 | 34.715 | 16.159 | 1.00 | 100.81 | O |
| ATOM | 139 | N | GLN | A | 17 | 80.660 | 34.144 | 18.337 | 1.00 | 101.15 | N |
| ATOM | 140 | CA | GLN | A | 17 | 80.240 | 35.477 | 18.794 | 1.00 | 101.61 | C |
| ATOM | 141 | CB | GLN | A | 17 | 79.328 | 35.375 | 20.035 | 1.00 | 106.37 | C |
| ATOM | 142 | CG | GLN | A | 17 | 79.957 | 34.784 | 21.301 | 1.00 | 110.91 | C |
| ATOM | 143 | CD | GLN | A | 17 | 79.913 | 33.260 | 21.369 | 1.00 | 112.06 | C |
| ATOM | 144 | OE1 | GLN | A | 17 | 79.731 | 32.579 | 20.358 | 1.00 | 112.42 | O |
| ATOM | 145 | NE2 | GLN | A | 17 | 80.090 | 32.721 | 22.571 | 1.00 | 111.28 | N |
| ATOM | 146 | C | GLN | A | 17 | 81.395 | 36.460 | 19.063 | 1.00 | 95.82 | C |
| ATOM | 147 | O | GLN | A | 17 | 81.150 | 37.635 | 19.333 | 1.00 | 91.71 | O |
| ATOM | 148 | N | THR | A | 18 | 82.639 | 35.990 | 18.977 | 1.00 | 93.69 | N |
| ATOM | 149 | CA | THR | A | 18 | 83.815 | 36.837 | 19.228 | 1.00 | 91.59 | C |
| ATOM | 150 | CB | THR | A | 18 | 85.127 | 36.039 | 19.082 | 1.00 | 89.62 | C |
| ATOM | 151 | OG1 | THR | A | 18 | 85.113 | 34.926 | 19.982 | 1.00 | 91.84 | O |
| ATOM | 152 | CG2 | THR | A | 18 | 86.335 | 36.912 | 19.388 | 1.00 | 88.68 | C |
| ATOM | 153 | C | THR | A | 18 | 83.873 | 38.056 | 18.303 | 1.00 | 91.59 | C |
| ATOM | 154 | O | THR | A | 18 | 84.147 | 39.165 | 18.756 | 1.00 | 93.09 | O |
| ATOM | 155 | N | PHE | A | 19 | 83.631 | 37.838 | 17.013 | 1.00 | 92.07 | N |
| ATOM | 156 | CA | PHE | A | 19 | 83.607 | 38.916 | 16.022 | 1.00 | 91.00 | C |
| ATOM | 157 | CB | PHE | A | 19 | 84.372 | 38.500 | 14.758 | 1.00 | 89.62 | C |
| ATOM | 158 | CG | PHE | A | 19 | 85.861 | 38.393 | 14.941 | 1.00 | 86.57 | C |
| ATOM | 159 | CD1 | PHE | A | 19 | 86.713 | 39.338 | 14.388 | 1.00 | 84.95 | C |
| ATOM | 160 | CE1 | PHE | A | 19 | 88.085 | 39.234 | 14.543 | 1.00 | 84.49 | C |
| ATOM | 161 | CZ | PHE | A | 19 | 88.621 | 38.176 | 15.253 | 1.00 | 84.63 | C |
| ATOM | 162 | CE2 | PHE | A | 19 | 87.785 | 37.223 | 15.802 | 1.00 | 85.95 | C |
| ATOM | 163 | CD2 | PHE | A | 19 | 86.413 | 37.332 | 15.643 | 1.00 | 86.68 | C |
| ATOM | 164 | C | PHE | A | 19 | 82.154 | 39.213 | 15.652 | 1.00 | 91.80 | C |
| ATOM | 165 | O | PHE | A | 19 | 81.265 | 38.417 | 15.966 | 1.00 | 92.30 | O |
| ATOM | 166 | N | PRO | A | 20 | 81.900 | 40.359 | 14.985 | 1.00 | 92.36 | N |
| ATOM | 167 | CA | PRO | A | 20 | 80.544 | 40.650 | 14.497 | 1.00 | 93.18 | C |
| ATOM | 168 | CB | PRO | A | 20 | 80.708 | 41.983 | 13.761 | 1.00 | 93.72 | C |
| ATOM | 169 | CG | PRO | A | 20 | 81.914 | 42.610 | 14.364 | 1.00 | 93.17 | C |
| ATOM | 170 | CD | PRO | A | 20 | 82.827 | 41.476 | 14.724 | 1.00 | 92.10 | C |
| ATOM | 171 | C | PRO | A | 20 | 80.048 | 39.579 | 13.529 | 1.00 | 92.47 | C |
| ATOM | 172 | O | PRO | A | 20 | 78.861 | 39.253 | 13.516 | 1.00 | 94.83 | O |
| ATOM | 173 | N | TYR | A | 21 | 80.973 | 39.050 | 12.731 | 1.00 | 89.94 | N |
| ATOM | 174 | CA | TYR | A | 21 | 80.694 | 37.973 | 11.791 | 1.00 | 87.48 | C |
| ATOM | 175 | CB | TYR | A | 21 | 81.251 | 38.310 | 10.402 | 1.00 | 86.87 | C |
| ATOM | 176 | CG | TYR | A | 21 | 82.767 | 38.336 | 10.269 | 1.00 | 85.77 | C |
| ATOM | 177 | CD1 | TYR | A | 21 | 83.520 | 39.408 | 10.744 | 1.00 | 85.72 | C |
| ATOM | 178 | CE1 | TYR | A | 21 | 84.900 | 39.438 | 10.599 | 1.00 | 86.03 | C |
| ATOM | 179 | CZ | TYR | A | 21 | 85.545 | 38.393 | 9.961 | 1.00 | 85.88 | C |
| ATOM | 180 | OH | TYR | A | 21 | 86.915 | 38.409 | 9.808 | 1.00 | 85.79 | O |
| ATOM | 181 | CE2 | TYR | A | 21 | 84.817 | 37.327 | 9.472 | 1.00 | 86.31 | C |
| ATOM | 182 | CD2 | TYR | A | 21 | 83.440 | 37.305 | 9.622 | 1.00 | 85.97 | C |
| ATOM | 183 | C | TYR | A | 21 | 81.291 | 36.676 | 12.326 | 1.00 | 85.29 | C |
| ATOM | 184 | O | TYR | A | 21 | 82.034 | 36.690 | 13.307 | 1.00 | 85.15 | O |
| ATOM | 185 | N | ASP | A | 22 | 80.952 | 35.556 | 11.695 | 1.00 | 84.23 | N |
| ATOM | 186 | CA | ASP | A | 22 | 81.479 | 34.259 | 12.112 | 1.00 | 81.39 | C |
| ATOM | 187 | CB | ASP | A | 22 | 80.601 | 33.118 | 11.595 | 1.00 | 79.88 | C |
| ATOM | 188 | CG | ASP | A | 22 | 80.934 | 31.781 | 12.235 | 1.00 | 80.48 | C |
| ATOM | 189 | OD1 | ASP | A | 22 | 81.957 | 31.686 | 12.950 | 1.00 | 80.95 | O |
| ATOM | 190 | OD2 | ASP | A | 22 | 80.167 | 30.817 | 12.025 | 1.00 | 77.89 | O |
| ATOM | 191 | C | ASP | A | 22 | 82.899 | 34.119 | 11.584 | 1.00 | 80.14 | C |
| ATOM | 192 | O | ASP | A | 22 | 83.105 | 33.974 | 10.377 | 1.00 | 83.56 | O |
| ATOM | 193 | N | ASN | A | 23 | 83.871 | 34.176 | 12.490 | 1.00 | 78.77 | N |
| ATOM | 194 | CA | ASN | A | 23 | 85.278 | 34.094 | 12.118 | 1.00 | 78.72 | C |
| ATOM | 195 | CB | ASN | A | 23 | 85.956 | 35.449 | 12.324 | 1.00 | 79.93 | C |
| ATOM | 196 | CG | ASN | A | 23 | 87.323 | 35.515 | 11.656 | 1.00 | 82.32 | C |
| ATOM | 197 | OD1 | ASN | A | 23 | 88.345 | 35.469 | 12.332 | 1.00 | 87.78 | O |
| ATOM | 198 | ND2 | ASN | A | 23 | 87.349 | 35.564 | 10.327 | 1.00 | 82.73 | N |
| ATOM | 199 | C | ASN | A | 23 | 86.021 | 33.010 | 12.901 | 1.00 | 77.85 | C |
| ATOM | 200 | O | ASN | A | 23 | 87.188 | 33.174 | 13.256 | 1.00 | 76.33 | O |
| ATOM | 201 | N | ARG | A | 24 | 85.346 | 31.896 | 13.167 | 1.00 | 77.43 | N |
| ATOM | 202 | CA | ARG | A | 24 | 85.985 | 30.779 | 13.851 | 1.00 | 78.84 | C |
| ATOM | 203 | CB | ARG | A | 24 | 84.961 | 29.708 | 14.215 | 1.00 | 82.46 | C |
| ATOM | 204 | CG | ARG | A | 24 | 83.948 | 30.142 | 15.261 | 1.00 | 87.00 | C |
| ATOM | 205 | CD | ARG | A | 24 | 82.841 | 29.113 | 15.404 | 1.00 | 90.70 | C |
| ATOM | 206 | NE | ARG | A | 24 | 81.997 | 29.063 | 14.213 | 1.00 | 94.41 | N |
| ATOM | 207 | CZ | ARG | A | 24 | 81.062 | 28.146 | 13.978 | 1.00 | 100.00 | C |
| ATOM | 208 | NH1 | ARG | A | 24 | 80.832 | 27.168 | 14.850 | 1.00 | 102.61 | N |
| ATOM | 209 | NH2 | ARG | A | 24 | 80.355 | 28.203 | 12.855 | 1.00 | 102.93 | N |
| ATOM | 210 | C | ARG | A | 24 | 87.049 | 30.181 | 12.936 | 1.00 | 77.88 | C |
| ATOM | 211 | O | ARG | A | 24 | 86.931 | 30.270 | 11.715 | 1.00 | 79.51 | O |
| ATOM | 212 | N | PRO | A | 25 | 88.092 | 29.562 | 13.517 | 1.00 | 76.89 | N |
| ATOM | 213 | CA | PRO | A | 25 | 89.160 | 28.944 | 12.722 | 1.00 | 76.11 | C |
| ATOM | 214 | CB | PRO | A | 25 | 89.920 | 28.115 | 13.758 | 1.00 | 75.95 | C |
| ATOM | 215 | CG | PRO | A | 25 | 89.723 | 28.849 | 15.034 | 1.00 | 75.64 | C |
| ATOM | 216 | CD | PRO | A | 25 | 88.354 | 29.457 | 14.964 | 1.00 | 75.85 | C |
| ATOM | 217 | C | PRO | A | 25 | 88.683 | 28.040 | 11.575 | 1.00 | 76.14 | C |
| ATOM | 218 | O | PRO | A | 25 | 89.365 | 27.944 | 10.553 | 1.00 | 78.16 | O |
| ATOM | 219 | N | VAL | A | 26 | 87.534 | 27.386 | 11.750 | 1.00 | 74.03 | N |
| ATOM | 220 | CA | VAL | A | 26 | 87.001 | 26.463 | 10.742 | 1.00 | 74.16 | C |
| ATOM | 221 | CB | VAL | A | 26 | 86.097 | 25.379 | 11.375 | 1.00 | 75.02 | C |
| ATOM | 222 | CG1 | VAL | A | 26 | 86.876 | 24.575 | 12.405 | 1.00 | 76.87 | C |
| ATOM | 223 | CG2 | VAL | A | 26 | 84.841 | 25.980 | 11.996 | 1.00 | 75.97 | C |
| ATOM | 224 | C | VAL | A | 26 | 86.235 | 27.141 | 9.608 | 1.00 | 76.22 | C |
| ATOM | 225 | O | VAL | A | 26 | 85.985 | 26.517 | 8.578 | 1.00 | 80.23 | O |
| ATOM | 226 | N | VAL | A | 27 | 85.862 | 28.405 | 9.788 | 1.00 | 77.19 | N |
| ATOM | 227 | CA | VAL | A | 27 | 85.106 | 29.132 | 8.766 | 1.00 | 77.16 | C |
| ATOM | 228 | CB | VAL | A | 27 | 84.644 | 30.512 | 9.286 | 1.00 | 78.69 | C |
| ATOM | 229 | CG1 | VAL | A | 27 | 84.097 | 31.382 | 8.159 | 1.00 | 81.69 | C |
| ATOM | 230 | CG2 | VAL | A | 27 | 83.600 | 30.338 | 10.378 | 1.00 | 78.01 | C |
| ATOM | 231 | C | VAL | A | 27 | 85.940 | 29.305 | 7.493 | 1.00 | 75.90 | C |
| ATOM | 232 | O | VAL | A | 27 | 87.110 | 29.679 | 7.569 | 1.00 | 76.15 | O |
| ATOM | 233 | N | PRO | A | 28 | 85.340 | 29.026 | 6.321 | 1.00 | 76.46 | N |
| ATOM | 234 | CA | PRO | A | 28 | 86.056 | 29.172 | 5.053 | 1.00 | 77.60 | C |
| ATOM | 235 | CB | PRO | A | 28 | 85.147 | 28.453 | 4.056 | 1.00 | 76.68 | C |
| ATOM | 236 | CG | PRO | A | 28 | 83.790 | 28.562 | 4.637 | 1.00 | 77.52 | C |
| ATOM | 237 | CD | PRO | A | 28 | 83.994 | 28.461 | 6.119 | 1.00 | 78.17 | C |
| ATOM | 238 | C | PRO | A | 28 | 86.248 | 30.626 | 4.631 | 1.00 | 78.58 | C |
| ATOM | 239 | O | PRO | A | 28 | 85.462 | 31.496 | 5.015 | 1.00 | 81.59 | O |
| ATOM | 240 | N | LEU | A | 29 | 87.292 | 30.865 | 3.841 | 1.00 | 77.97 | N |
| ATOM | 241 | CA | LEU | A | 29 | 87.631 | 32.196 | 3.347 | 1.00 | 79.27 | C |
| ATOM | 242 | CB | LEU | A | 29 | 89.133 | 32.447 | 3.503 | 1.00 | 77.54 | C |
| ATOM | 243 | CG | LEU | A | 29 | 89.675 | 32.775 | 4.904 | 1.00 | 76.54 | C |
| ATOM | 244 | CD1 | LEU | A | 29 | 91.176 | 32.541 | 4.937 | 1.00 | 75.32 | C |
| ATOM | 245 | CD2 | LEU | A | 29 | 89.013 | 31.971 | 6.011 | 1.00 | 76.40 | C |
| ATOM | 246 | C | LEU | A | 29 | 87.228 | 32.296 | 1.880 | 1.00 | 82.98 | C |
| ATOM | 247 | O | LEU | A | 29 | 87.705 | 31.521 | 1.050 | 1.00 | 85.82 | O |
| ATOM | 248 | N | GLN | A | 30 | 86.360 | 33.256 | 1.563 | 1.00 | 85.08 | N |
| ATOM | 249 | CA | GLN | A | 30 | 85.833 | 33.409 | 0.207 | 1.00 | 84.07 | C |
| ATOM | 250 | CB | GLN | A | 30 | 84.644 | 32.461 | 0.010 | 1.00 | 83.65 | C |
| ATOM | 251 | CG | GLN | A | 30 | 83.505 | 32.673 | 1.004 | 1.00 | 85.40 | C |
| ATOM | 252 | CD | GLN | A | 30 | 82.633 | 31.442 | 1.217 | 1.00 | 85.90 | C |
| ATOM | 253 | OE1 | GLN | A | 30 | 83.118 | 30.311 | 1.215 | 1.00 | 83.42 | O |
| ATOM | 254 | NE2 | GLN | A | 30 | 81.341 | 31.665 | 1.432 | 1.00 | 86.12 | N |
| ATOM | 255 | C | GLN | A | 30 | 85.427 | 34.856 | −0.081 | 1.00 | 85.69 | C |
| ATOM | 256 | O | GLN | A | 30 | 84.653 | 35.455 | 0.666 | 1.00 | 86.01 | O |
| ATOM | 257 | N | TYR | A | 31 | 85.965 | 35.405 | −1.167 | 1.00 | 89.33 | N |
| ATOM | 258 | CA | TYR | A | 31 | 85.709 | 36.788 | −1.591 | 1.00 | 91.39 | C |
| ATOM | 259 | CB | TYR | A | 31 | 84.343 | 36.903 | −2.281 | 1.00 | 91.41 | C |
| ATOM | 260 | CG | TYR | A | 31 | 84.183 | 38.164 | −3.108 | 1.00 | 92.21 | C |
| ATOM | 261 | CD1 | TYR | A | 31 | 85.077 | 38.463 | −4.132 | 1.00 | 93.65 | C |
| ATOM | 262 | CE1 | TYR | A | 31 | 84.937 | 39.607 | −4.898 | 1.00 | 94.01 | C |
| ATOM | 263 | CZ | TYR | A | 31 | 83.888 | 40.466 | −4.653 | 1.00 | 94.14 | C |
| ATOM | 264 | OH | TYR | A | 31 | 83.753 | 41.599 | −5.418 | 1.00 | 94.67 | O |
| ATOM | 265 | CE2 | TYR | A | 31 | 82.982 | 40.191 | −3.647 | 1.00 | 94.39 | C |
| ATOM | 266 | CD2 | TYR | A | 31 | 83.131 | 39.043 | −2.884 | 1.00 | 93.28 | C |
| ATOM | 267 | C | TYR | A | 31 | 85.835 | 37.782 | −0.434 | 1.00 | 89.84 | C |
| ATOM | 268 | O | TYR | A | 31 | 84.935 | 38.585 | −0.175 | 1.00 | 91.00 | O |
| ATOM | 269 | N | MET | A | 32 | 86.975 | 37.710 | 0.248 | 1.00 | 87.23 | N |
| ATOM | 270 | CA | MET | A | 32 | 87.272 | 38.560 | 1.394 | 1.00 | 84.81 | C |
| ATOM | 271 | CB | MET | A | 32 | 87.742 | 37.702 | 2.566 | 1.00 | 83.80 | C |
| ATOM | 272 | CG | MET | A | 32 | 86.653 | 36.922 | 3.268 | 1.00 | 84.29 | C |
| ATOM | 273 | SD | MET | A | 32 | 87.372 | 35.892 | 4.557 | 1.00 | 88.43 | S |
| ATOM | 274 | CE | MET | A | 32 | 85.911 | 35.425 | 5.482 | 1.00 | 88.00 | C |
| ATOM | 275 | C | MET | A | 32 | 88.370 | 39.561 | 1.072 | 1.00 | 83.30 | C |
| ATOM | 276 | O | MET | A | 32 | 89.194 | 39.332 | 0.187 | 1.00 | 82.99 | O |
| ATOM | 277 | N | THR | A | 33 | 88.379 | 40.665 | 1.812 | 1.00 | 84.00 | N |
| ATOM | 278 | CA | THR | A | 33 | 89.443 | 41.655 | 1.708 | 1.00 | 85.10 | C |
| ATOM | 279 | CB | THR | A | 33 | 89.066 | 42.968 | 2.408 | 1.00 | 86.62 | C |
| ATOM | 280 | OG1 | THR | A | 33 | 88.682 | 42.694 | 3.761 | 1.00 | 85.39 | O |
| ATOM | 281 | CG2 | THR | A | 33 | 87.911 | 43.640 | 1.687 | 1.00 | 88.78 | C |
| ATOM | 282 | C | THR | A | 33 | 90.679 | 41.072 | 2.374 | 1.00 | 84.97 | C |
| ATOM | 283 | O | THR | A | 33 | 90.570 | 40.134 | 3.167 | 1.00 | 84.64 | O |
| ATOM | 284 | N | PHE | A | 34 | 91.847 | 41.626 | 2.068 | 1.00 | 84.71 | N |
| ATOM | 285 | CA | PHE | A | 34 | 93.096 | 41.070 | 2.584 | 1.00 | 84.09 | C |
| ATOM | 286 | CB | PHE | A | 34 | 94.300 | 41.925 | 2.201 | 1.00 | 82.95 | C |
| ATOM | 287 | CG | PHE | A | 34 | 95.605 | 41.323 | 2.623 | 1.00 | 81.81 | C |
| ATOM | 288 | CD1 | PHE | A | 34 | 96.087 | 40.193 | 1.984 | 1.00 | 81.66 | C |
| ATOM | 289 | CE1 | PHE | A | 34 | 97.283 | 39.618 | 2.369 | 1.00 | 81.94 | C |
| ATOM | 290 | CZ | PHE | A | 34 | 98.014 | 40.170 | 3.406 | 1.00 | 80.79 | C |
| ATOM | 291 | CE2 | PHE | A | 34 | 97.540 | 41.294 | 4.057 | 1.00 | 81.75 | C |
| ATOM | 292 | CD2 | PHE | A | 34 | 96.339 | 41.863 | 3.668 | 1.00 | 82.13 | C |
| ATOM | 293 | C | PHE | A | 34 | 93.078 | 40.861 | 4.094 | 1.00 | 85.55 | C |
| ATOM | 294 | O | PHE | A | 34 | 93.405 | 39.779 | 4.571 | 1.00 | 83.63 | O |
| ATOM | 295 | N | GLN | A | 35 | 92.682 | 41.892 | 4.835 | 1.00 | 89.59 | N |
| ATOM | 296 | CA | GLN | A | 35 | 92.652 | 41.828 | 6.298 | 1.00 | 92.31 | C |
| ATOM | 297 | CB | GLN | A | 35 | 92.176 | 43.159 | 6.884 | 1.00 | 97.58 | C |
| ATOM | 298 | CG | GLN | A | 35 | 92.242 | 43.228 | 8.401 | 1.00 | 100.31 | C |
| ATOM | 299 | CD | GLN | A | 35 | 91.806 | 44.578 | 8.923 | 1.00 | 104.73 | C |
| ATOM | 300 | OE1 | GLN | A | 35 | 90.700 | 45.025 | 8.633 | 1.00 | 108.24 | O |
| ATOM | 301 | NE2 | GLN | A | 35 | 92.663 | 45.233 | 9.703 | 1.00 | 108.47 | N |
| ATOM | 302 | C | GLN | A | 35 | 91.768 | 40.698 | 6.819 | 1.00 | 87.97 | C |
| ATOM | 303 | O | GLN | A | 35 | 92.081 | 40.082 | 7.840 | 1.00 | 88.49 | O |
| ATOM | 304 | N | GLU | A | 36 | 90.672 | 40.432 | 6.115 | 1.00 | 83.87 | N |
| ATOM | 305 | CA | GLU | A | 36 | 89.734 | 39.383 | 6.508 | 1.00 | 81.96 | C |
| ATOM | 306 | CB | GLU | A | 36 | 88.388 | 39.590 | 5.809 | 1.00 | 82.61 | C |
| ATOM | 307 | CG | GLU | A | 36 | 87.670 | 40.854 | 6.258 | 1.00 | 84.88 | C |
| ATOM | 308 | CD | GLU | A | 36 | 86.424 | 41.172 | 5.449 | 1.00 | 85.92 | C |
| ATOM | 309 | OE1 | GLU | A | 36 | 86.237 | 40.599 | 4.356 | 1.00 | 88.20 | O |
| ATOM | 310 | OE2 | GLU | A | 36 | 85.633 | 42.019 | 5.908 | 1.00 | 87.08 | O |
| ATOM | 311 | C | GLU | A | 36 | 90.264 | 37.969 | 6.244 | 1.00 | 78.74 | C |
| ATOM | 312 | O | GLU | A | 36 | 90.151 | 37.099 | 7.110 | 1.00 | 79.13 | O |
| ATOM | 313 | N | TRP | A | 37 | 90.850 | 37.739 | 5.068 | 1.00 | 74.66 | N |
| ATOM | 314 | CA | TRP | A | 37 | 91.333 | 36.397 | 4.715 | 1.00 | 71.51 | C |
| ATOM | 315 | CB | TRP | A | 37 | 91.230 | 36.128 | 3.192 | 1.00 | 70.80 | C |
| ATOM | 316 | CG | TRP | A | 37 | 92.341 | 36.670 | 2.332 | 1.00 | 67.87 | C |
| ATOM | 317 | CD1 | TRP | A | 37 | 92.305 | 37.789 | 1.559 | 1.00 | 68.80 | C |
| ATOM | 318 | NE1 | TRP | A | 37 | 93.506 | 37.958 | 0.912 | 1.00 | 69.34 | N |
| ATOM | 319 | CE2 | TRP | A | 37 | 94.342 | 36.932 | 1.257 | 1.00 | 66.64 | C |
| ATOM | 320 | CD2 | TRP | A | 37 | 93.638 | 36.096 | 2.143 | 1.00 | 65.97 | C |
| ATOM | 321 | CE3 | TRP | A | 37 | 94.277 | 34.962 | 2.649 | 1.00 | 67.44 | C |
| ATOM | 322 | CZ3 | TRP | A | 37 | 95.578 | 34.702 | 2.256 | 1.00 | 68.15 | C |
| ATOM | 323 | CH2 | TRP | A | 37 | 96.251 | 35.555 | 1.371 | 1.00 | 68.14 | C |
| ATOM | 324 | CZ2 | TRP | A | 37 | 95.650 | 36.671 | 0.862 | 1.00 | 67.60 | C |
| ATOM | 325 | C | TRP | A | 37 | 92.741 | 36.106 | 5.246 | 1.00 | 69.80 | C |
| ATOM | 326 | O | TRP | A | 37 | 93.022 | 34.984 | 5.657 | 1.00 | 68.47 | O |
| ATOM | 327 | N | TRP | A | 38 | 93.619 | 37.105 | 5.240 | 1.00 | 72.07 | N |
| ATOM | 328 | CA | TRP | A | 38 | 95.006 | 36.918 | 5.697 | 1.00 | 75.67 | C |
| ATOM | 329 | CB | TRP | A | 38 | 95.836 | 38.173 | 5.417 | 1.00 | 77.42 | C |
| ATOM | 330 | CG | TRP | A | 38 | 97.241 | 38.121 | 5.933 | 1.00 | 78.07 | C |
| ATOM | 331 | CD1 | TRP | A | 38 | 97.750 | 38.830 | 6.979 | 1.00 | 79.20 | C |
| ATOM | 332 | NE1 | TRP | A | 38 | 99.078 | 38.532 | 7.153 | 1.00 | 79.67 | N |
| ATOM | 333 | CE2 | TRP | A | 38 | 99.453 | 37.612 | 6.211 | 1.00 | 78.00 | C |
| ATOM | 334 | CD2 | TRP | A | 38 | 98.318 | 37.326 | 5.426 | 1.00 | 78.69 | C |
| ATOM | 335 | CE3 | TRP | A | 38 | 98.435 | 36.401 | 4.383 | 1.00 | 81.00 | C |
| ATOM | 336 | CZ3 | TRP | A | 38 | 99.666 | 35.795 | 4.164 | 1.00 | 80.23 | C |
| ATOM | 337 | CH2 | TRP | A | 38 | 100.773 | 36.102 | 4.966 | 1.00 | 78.28 | C |
| ATOM | 338 | CZ2 | TRP | A | 38 | 100.686 | 37.002 | 5.992 | 1.00 | 77.56 | C |
| ATOM | 339 | C | TRP | A | 38 | 95.084 | 36.554 | 7.184 | 1.00 | 76.24 | C |
| ATOM | 340 | O | TRP | A | 38 | 94.643 | 37.316 | 8.046 | 1.00 | 72.52 | O |
| ATOM | 341 | N | PHE | A | 39 | 95.648 | 35.378 | 7.461 | 1.00 | 77.18 | N |
| ATOM | 342 | CA | PHE | A | 39 | 95.744 | 34.828 | 8.819 | 1.00 | 76.64 | C |
| ATOM | 343 | CB | PHE | A | 39 | 96.652 | 35.693 | 9.705 | 1.00 | 77.48 | C |
| ATOM | 344 | CG | PHE | A | 39 | 98.059 | 35.181 | 9.814 | 1.00 | 78.53 | C |
| ATOM | 345 | CD1 | PHE | A | 39 | 98.464 | 34.469 | 10.936 | 1.00 | 79.16 | C |
| ATOM | 346 | CE1 | PHE | A | 39 | 99.762 | 33.996 | 11.045 | 1.00 | 79.01 | C |
| ATOM | 347 | CZ | PHE | A | 39 | 100.670 | 34.227 | 10.025 | 1.00 | 79.14 | C |
| ATOM | 348 | CE2 | PHE | A | 39 | 100.278 | 34.935 | 8.902 | 1.00 | 78.45 | C |
| ATOM | 349 | CD2 | PHE | A | 39 | 98.978 | 35.407 | 8.801 | 1.00 | 78.90 | C |
| ATOM | 350 | C | PHE | A | 39 | 94.382 | 34.604 | 9.482 | 1.00 | 76.52 | C |
| ATOM | 351 | O | PHE | A | 39 | 94.301 | 34.448 | 10.702 | 1.00 | 81.02 | O |
| ATOM | 352 | N | HIS | A | 40 | 93.327 | 34.553 | 8.669 | 1.00 | 74.90 | N |
| ATOM | 353 | CA | HIS | A | 40 | 91.956 | 34.369 | 9.146 | 1.00 | 74.06 | C |
| ATOM | 354 | CB | HIS | A | 40 | 91.817 | 33.016 | 9.857 | 1.00 | 69.89 | C |
| ATOM | 355 | CG | HIS | A | 40 | 90.460 | 32.401 | 9.730 | 1.00 | 66.79 | C |
| ATOM | 356 | ND1 | HIS | A | 40 | 89.299 | 33.085 | 10.019 | 1.00 | 66.58 | N |
| ATOM | 357 | CE1 | HIS | A | 40 | 88.262 | 32.290 | 9.821 | 1.00 | 65.80 | C |
| ATOM | 358 | NE2 | HIS | A | 40 | 88.709 | 31.111 | 9.428 | 1.00 | 65.20 | N |
| ATOM | 359 | CD2 | HIS | A | 40 | 90.081 | 31.153 | 9.365 | 1.00 | 65.32 | C |
| ATOM | 360 | C | HIS | A | 40 | 91.552 | 35.513 | 10.078 | 1.00 | 78.21 | C |
| ATOM | 361 | O | HIS | A | 40 | 90.708 | 35.339 | 10.959 | 1.00 | 79.42 | O |
| ATOM | 362 | N | ASN | A | 41 | 92.157 | 36.683 | 9.867 | 1.00 | 82.14 | N |
| ATOM | 363 | CA | ASN | A | 41 | 91.928 | 37.861 | 10.703 | 1.00 | 82.77 | C |
| ATOM | 364 | CB | ASN | A | 41 | 90.579 | 38.500 | 10.345 | 1.00 | 80.83 | C |
| ATOM | 365 | CG | ASN | A | 41 | 90.473 | 39.946 | 10.794 | 1.00 | 79.26 | C |
| ATOM | 366 | OD1 | ASN | A | 41 | 91.480 | 40.641 | 10.938 | 1.00 | 78.80 | O |
| ATOM | 367 | ND2 | ASN | A | 41 | 89.246 | 40.413 | 10.996 | 1.00 | 79.09 | N |
| ATOM | 368 | C | ASN | A | 41 | 92.010 | 37.528 | 12.198 | 1.00 | 84.66 | C |
| ATOM | 369 | O | ASN | A | 41 | 91.164 | 37.952 | 12.989 | 1.00 | 88.85 | O |
| ATOM | 370 | N | HIS | A | 42 | 93.030 | 36.753 | 12.568 | 1.00 | 84.35 | N |
| ATOM | 371 | CA | HIS | A | 42 | 93.252 | 36.351 | 13.959 | 1.00 | 84.97 | C |
| ATOM | 372 | CB | HIS | A | 42 | 93.240 | 34.823 | 14.093 | 1.00 | 83.97 | C |
| ATOM | 373 | CG | HIS | A | 42 | 91.890 | 34.204 | 13.916 | 1.00 | 83.92 | C |
| ATOM | 374 | ND1 | HIS | A | 42 | 91.717 | 32.943 | 13.387 | 1.00 | 86.21 | N |
| ATOM | 375 | CE1 | HIS | A | 42 | 90.428 | 32.654 | 13.355 | 1.00 | 84.60 | C |
| ATOM | 376 | NE2 | HIS | A | 42 | 89.759 | 33.686 | 13.838 | 1.00 | 82.81 | N |
| ATOM | 377 | CD2 | HIS | A | 42 | 90.650 | 34.669 | 14.195 | 1.00 | 82.87 | C |
| ATOM | 378 | C | HIS | A | 42 | 94.569 | 36.885 | 14.530 | 1.00 | 86.81 | C |
| ATOM | 379 | O | HIS | A | 42 | 95.054 | 36.371 | 15.539 | 1.00 | 88.94 | O |
| ATOM | 380 | N | LEU | A | 43 | 95.141 | 37.915 | 13.906 | 1.00 | 87.62 | N |
| ATOM | 381 | CA | LEU | A | 43 | 96.409 | 38.480 | 14.379 | 1.00 | 87.31 | C |
| ATOM | 382 | CB | LEU | A | 43 | 96.950 | 39.518 | 13.390 | 1.00 | 85.45 | C |
| ATOM | 383 | CG | LEU | A | 43 | 97.366 | 38.975 | 12.018 | 1.00 | 84.56 | C |
| ATOM | 384 | CD1 | LEU | A | 43 | 97.778 | 40.109 | 11.092 | 1.00 | 84.45 | C |
| ATOM | 385 | CD2 | LEU | A | 43 | 98.489 | 37.957 | 12.145 | 1.00 | 84.05 | C |
| ATOM | 386 | C | LEU | A | 43 | 96.285 | 39.086 | 15.781 | 1.00 | 90.04 | C |
| ATOM | 387 | O | LEU | A | 43 | 97.224 | 39.010 | 16.576 | 1.00 | 91.88 | O |
| ATOM | 388 | N | ASP | A | 44 | 95.122 | 39.658 | 16.089 | 1.00 | 90.94 | N |
| ATOM | 389 | CA | ASP | A | 44 | 94.872 | 40.239 | 17.413 | 1.00 | 90.81 | C |
| ATOM | 390 | CB | ASP | A | 44 | 93.751 | 41.285 | 17.342 | 1.00 | 91.78 | C |
| ATOM | 391 | CG | ASP | A | 44 | 94.126 | 42.494 | 16.497 | 1.00 | 93.05 | C |
| ATOM | 392 | OD1 | ASP | A | 44 | 95.329 | 42.704 | 16.227 | 1.00 | 93.42 | O |
| ATOM | 393 | OD2 | ASP | A | 44 | 93.210 | 43.246 | 16.105 | 1.00 | 95.74 | O |
| ATOM | 394 | C | ASP | A | 44 | 94.545 | 39.189 | 18.481 | 1.00 | 88.30 | C |
| ATOM | 395 | O | ASP | A | 44 | 94.294 | 39.538 | 19.634 | 1.00 | 92.18 | O |
| ATOM | 396 | N | TYR | A | 45 | 94.553 | 37.912 | 18.102 | 1.00 | 86.07 | N |
| ATOM | 397 | CA | TYR | A | 45 | 94.333 | 36.810 | 19.038 | 1.00 | 86.13 | C |
| ATOM | 398 | CB | TYR | A | 45 | 93.001 | 36.116 | 18.743 | 1.00 | 83.98 | C |
| ATOM | 399 | CG | TYR | A | 45 | 91.804 | 37.000 | 18.991 | 1.00 | 83.67 | C |
| ATOM | 400 | CD1 | TYR | A | 45 | 91.391 | 37.924 | 18.038 | 1.00 | 83.99 | C |
| ATOM | 401 | CE1 | TYR | A | 45 | 90.297 | 38.743 | 18.261 | 1.00 | 83.73 | C |
| ATOM | 402 | CZ | TYR | A | 45 | 89.601 | 38.644 | 19.450 | 1.00 | 83.17 | C |
| ATOM | 403 | OH | TYR | A | 45 | 88.513 | 39.457 | 19.672 | 1.00 | 82.14 | O |
| ATOM | 404 | CE2 | TYR | A | 45 | 89.992 | 37.733 | 20.414 | 1.00 | 83.17 | C |
| ATOM | 405 | CD2 | TYR | A | 45 | 91.089 | 36.920 | 20.182 | 1.00 | 82.66 | C |
| ATOM | 406 | C | TYR | A | 45 | 95.491 | 35.818 | 18.937 | 1.00 | 86.55 | C |
| ATOM | 407 | O | TYR | A | 45 | 95.294 | 34.660 | 18.566 | 1.00 | 86.50 | O |
| ATOM | 408 | N | PRO | A | 46 | 96.709 | 36.270 | 19.281 | 1.00 | 88.41 | N |
| ATOM | 409 | CA | PRO | A | 46 | 97.863 | 35.396 | 19.162 | 1.00 | 88.47 | C |
| ATOM | 410 | CB | PRO | A | 46 | 99.037 | 36.370 | 19.243 | 1.00 | 88.57 | C |
| ATOM | 411 | CG | PRO | A | 46 | 98.546 | 37.474 | 20.115 | 1.00 | 88.79 | C |
| ATOM | 412 | CD | PRO | A | 46 | 97.045 | 37.515 | 19.997 | 1.00 | 89.40 | C |
| ATOM | 413 | C | PRO | A | 46 | 97.936 | 34.393 | 20.304 | 1.00 | 89.24 | C |
| ATOM | 414 | O | PRO | A | 46 | 97.334 | 34.616 | 21.353 | 1.00 | 91.06 | O |
| ATOM | 415 | N | PRO | A | 47 | 98.682 | 33.295 | 20.108 | 1.00 | 89.74 | N |
| ATOM | 416 | CA | PRO | A | 47 | 98.854 | 32.294 | 21.153 | 1.00 | 89.39 | C |
| ATOM | 417 | CB | PRO | A | 47 | 99.518 | 31.142 | 20.405 | 1.00 | 89.86 | C |
| ATOM | 418 | CG | PRO | A | 47 | 100.352 | 31.832 | 19.379 | 1.00 | 89.43 | C |
| ATOM | 419 | CD | PRO | A | 47 | 99.525 | 33.004 | 18.932 | 1.00 | 89.34 | C |
| ATOM | 420 | C | PRO | A | 47 | 99.786 | 32.818 | 22.235 | 1.00 | 90.42 | C |
| ATOM | 421 | O | PRO | A | 47 | 100.293 | 33.936 | 22.120 | 1.00 | 90.12 | O |
| ATOM | 422 | N | HIS | A | 48 | 100.019 | 32.023 | 23.273 | 1.00 | 93.02 | N |
| ATOM | 423 | CA | HIS | A | 48 | 100.965 | 32.424 | 24.307 | 1.00 | 94.38 | C |
| ATOM | 424 | CB | HIS | A | 48 | 100.828 | 31.565 | 25.564 | 1.00 | 95.13 | C |
| ATOM | 425 | CG | HIS | A | 48 | 99.486 | 31.671 | 26.221 | 1.00 | 95.66 | C |
| ATOM | 426 | ND1 | HIS | A | 48 | 98.803 | 32.864 | 26.331 | 1.00 | 97.19 | N |
| ATOM | 427 | CE1 | HIS | A | 48 | 97.659 | 32.658 | 26.959 | 1.00 | 97.52 | C |
| ATOM | 428 | NE2 | HIS | A | 48 | 97.581 | 31.377 | 27.272 | 1.00 | 97.13 | N |
| ATOM | 429 | CD2 | HIS | A | 48 | 98.713 | 30.738 | 26.826 | 1.00 | 95.64 | C |
| ATOM | 430 | C | HIS | A | 48 | 102.376 | 32.351 | 23.718 | 1.00 | 96.28 | C |
| ATOM | 431 | O | HIS | A | 48 | 102.644 | 31.505 | 22.862 | 1.00 | 96.29 | O |
| ATOM | 432 | N | PRO | A | 49 | 103.281 | 33.240 | 24.166 | 1.00 | 97.64 | N |
| ATOM | 433 | CA | PRO | A | 49 | 104.614 | 33.331 | 23.557 | 1.00 | 98.49 | C |
| ATOM | 434 | CB | PRO | A | 49 | 105.323 | 34.391 | 24.414 | 1.00 | 99.83 | C |
| ATOM | 435 | CG | PRO | A | 49 | 104.229 | 35.170 | 25.054 | 1.00 | 98.41 | C |
| ATOM | 436 | CD | PRO | A | 49 | 103.136 | 34.177 | 25.295 | 1.00 | 97.68 | C |
| ATOM | 437 | C | PRO | A | 49 | 105.431 | 32.036 | 23.539 | 1.00 | 97.48 | C |
| ATOM | 438 | O | PRO | A | 49 | 106.244 | 31.849 | 22.636 | 1.00 | 96.07 | O |
| ATOM | 439 | N | GLY | A | 50 | 105.216 | 31.159 | 24.519 | 1.00 | 97.60 | N |
| ATOM | 440 | CA | GLY | A | 50 | 105.978 | 29.912 | 24.628 | 1.00 | 96.66 | C |
| ATOM | 441 | C | GLY | A | 50 | 105.441 | 28.715 | 23.858 | 1.00 | 94.24 | C |
| ATOM | 442 | O | GLY | A | 50 | 106.114 | 27.688 | 23.777 | 1.00 | 96.57 | O |
| ATOM | 443 | N | ASP | A | 51 | 104.238 | 28.835 | 23.296 | 1.00 | 91.17 | N |
| ATOM | 444 | CA | ASP | A | 51 | 103.607 | 27.733 | 22.562 | 1.00 | 88.43 | C |
| ATOM | 445 | CB | ASP | A | 51 | 102.084 | 27.821 | 22.685 | 1.00 | 88.07 | C |
| ATOM | 446 | CG | ASP | A | 51 | 101.602 | 27.681 | 24.121 | 1.00 | 88.98 | C |
| ATOM | 447 | OD1 | ASP | A | 51 | 102.338 | 27.107 | 24.952 | 1.00 | 87.99 | O |
| ATOM | 448 | OD2 | ASP | A | 51 | 100.477 | 28.141 | 24.416 | 1.00 | 88.52 | O |
| ATOM | 449 | C | ASP | A | 51 | 104.007 | 27.717 | 21.081 | 1.00 | 87.45 | C |
| ATOM | 450 | O | ASP | A | 51 | 103.668 | 28.632 | 20.328 | 1.00 | 82.48 | O |
| ATOM | 451 | N | PHE | A | 52 | 104.729 | 26.669 | 20.681 | 1.00 | 86.67 | N |
| ATOM | 452 | CA | PHE | A | 52 | 105.177 | 26.489 | 19.299 | 1.00 | 86.78 | C |
| ATOM | 453 | CB | PHE | A | 52 | 106.681 | 26.747 | 19.178 | 1.00 | 88.12 | C |
| ATOM | 454 | CG | PHE | A | 52 | 107.071 | 28.187 | 19.330 | 1.00 | 89.61 | C |
| ATOM | 455 | CD1 | PHE | A | 52 | 107.381 | 28.709 | 20.577 | 1.00 | 90.55 | C |
| ATOM | 456 | CE1 | PHE | A | 52 | 107.750 | 30.034 | 20.716 | 1.00 | 91.55 | C |
| ATOM | 457 | CZ | PHE | A | 52 | 107.815 | 30.856 | 19.604 | 1.00 | 92.08 | C |
| ATOM | 458 | CE2 | PHE | A | 52 | 107.513 | 30.347 | 18.354 | 1.00 | 90.65 | C |
| ATOM | 459 | CD2 | PHE | A | 52 | 107.148 | 29.019 | 18.222 | 1.00 | 90.14 | C |
| ATOM | 460 | C | PHE | A | 52 | 104.900 | 25.073 | 18.806 | 1.00 | 86.43 | C |
| ATOM | 461 | O | PHE | A | 52 | 104.956 | 24.116 | 19.580 | 1.00 | 89.33 | O |
| ATOM | 462 | N | PHE | A | 53 | 104.608 | 24.955 | 17.514 | 1.00 | 84.53 | N |
| ATOM | 463 | CA | PHE | A | 53 | 104.450 | 23.658 | 16.861 | 1.00 | 82.72 | C |
| ATOM | 464 | CB | PHE | A | 53 | 103.408 | 23.744 | 15.744 | 1.00 | 79.11 | C |
| ATOM | 465 | CG | PHE | A | 53 | 103.052 | 22.415 | 15.135 | 1.00 | 74.50 | C |
| ATOM | 466 | CD1 | PHE | A | 53 | 101.884 | 21.762 | 15.500 | 1.00 | 72.03 | C |
| ATOM | 467 | CE1 | PHE | A | 53 | 101.551 | 20.539 | 14.944 | 1.00 | 70.56 | C |
| ATOM | 468 | CZ | PHE | A | 53 | 102.389 | 19.954 | 14.012 | 1.00 | 71.23 | C |
| ATOM | 469 | CE2 | PHE | A | 53 | 103.556 | 20.594 | 13.636 | 1.00 | 71.89 | C |
| ATOM | 470 | CD2 | PHE | A | 53 | 103.883 | 21.817 | 14.194 | 1.00 | 73.25 | C |
| ATOM | 471 | C | PHE | A | 53 | 105.820 | 23.292 | 16.290 | 1.00 | 83.28 | C |
| ATOM | 472 | O | PHE | A | 53 | 106.380 | 24.038 | 15.488 | 1.00 | 82.27 | O |
| ATOM | 473 | N | ASP | A | 54 | 106.356 | 22.147 | 16.700 | 1.00 | 84.85 | N |
| ATOM | 474 | CA | ASP | A | 54 | 107.699 | 21.737 | 16.288 | 1.00 | 85.30 | C |
| ATOM | 475 | CB | ASP | A | 54 | 108.404 | 21.003 | 17.436 | 1.00 | 87.15 | C |
| ATOM | 476 | CG | ASP | A | 54 | 108.726 | 21.918 | 18.607 | 1.00 | 88.60 | C |
| ATOM | 477 | OD1 | ASP | A | 54 | 109.024 | 23.113 | 18.382 | 1.00 | 87.90 | O |
| ATOM | 478 | OD2 | ASP | A | 54 | 108.698 | 21.435 | 19.758 | 1.00 | 93.39 | O |
| ATOM | 479 | C | ASP | A | 54 | 107.708 | 20.870 | 15.029 | 1.00 | 83.18 | C |
| ATOM | 480 | O | ASP | A | 54 | 106.981 | 19.880 | 14.941 | 1.00 | 83.58 | O |
| ATOM | 481 | N | PHE | A | 55 | 108.541 | 21.258 | 14.065 | 1.00 | 80.53 | N |
| ATOM | 482 | CA | PHE | A | 55 | 108.739 | 20.499 | 12.832 | 1.00 | 78.93 | C |
| ATOM | 483 | CB | PHE | A | 55 | 108.573 | 21.403 | 11.604 | 1.00 | 79.62 | C |
| ATOM | 484 | CG | PHE | A | 55 | 107.140 | 21.707 | 11.263 | 1.00 | 78.94 | C |
| ATOM | 485 | CD1 | PHE | A | 55 | 106.328 | 20.729 | 10.701 | 1.00 | 78.40 | C |
| ATOM | 486 | CE1 | PHE | A | 55 | 105.009 | 20.997 | 10.388 | 1.00 | 76.98 | C |
| ATOM | 487 | CZ | PHE | A | 55 | 104.486 | 22.254 | 10.624 | 1.00 | 77.19 | C |
| ATOM | 488 | CE2 | PHE | A | 55 | 105.283 | 23.242 | 11.175 | 1.00 | 77.28 | C |
| ATOM | 489 | CD2 | PHE | A | 55 | 106.603 | 22.967 | 11.492 | 1.00 | 77.85 | C |
| ATOM | 490 | C | PHE | A | 55 | 110.137 | 19.882 | 12.844 | 1.00 | 77.06 | C |
| ATOM | 491 | O | PHE | A | 55 | 111.126 | 20.594 | 12.670 | 1.00 | 76.91 | O |
| ATOM | 492 | N | PRO | A | 56 | 110.228 | 18.556 | 13.051 | 1.00 | 75.75 | N |
| ATOM | 493 | CA | PRO | A | 56 | 111.536 | 17.916 | 13.097 | 1.00 | 77.64 | C |
| ATOM | 494 | CB | PRO | A | 56 | 111.263 | 16.636 | 13.886 | 1.00 | 77.83 | C |
| ATOM | 495 | CG | PRO | A | 56 | 109.837 | 16.308 | 13.603 | 1.00 | 76.68 | C |
| ATOM | 496 | CD | PRO | A | 56 | 109.136 | 17.579 | 13.209 | 1.00 | 76.19 | C |
| ATOM | 497 | C | PRO | A | 56 | 112.080 | 17.594 | 11.707 | 1.00 | 80.24 | C |
| ATOM | 498 | O | PRO | A | 56 | 111.481 | 16.802 | 10.979 | 1.00 | 83.61 | O |
| ATOM | 499 | N | ALA | A | 57 | 113.214 | 18.200 | 11.359 | 1.00 | 80.81 | N |
| ATOM | 500 | CA | ALA | A | 57 | 113.845 | 17.992 | 10.058 | 1.00 | 79.94 | C |
| ATOM | 501 | CB | ALA | A | 57 | 115.173 | 18.730 | 9.993 | 1.00 | 79.03 | C |
| ATOM | 502 | C | ALA | A | 57 | 114.053 | 16.510 | 9.766 | 1.00 | 81.02 | C |
| ATOM | 503 | O | ALA | A | 57 | 114.479 | 15.753 | 10.636 | 1.00 | 82.40 | O |
| ATOM | 504 | N | GLY | A | 58 | 113.730 | 16.103 | 8.541 | 1.00 | 83.86 | N |
| ATOM | 505 | CA | GLY | A | 58 | 113.888 | 14.716 | 8.109 | 1.00 | 84.69 | C |
| ATOM | 506 | C | GLY | A | 58 | 112.937 | 13.736 | 8.771 | 1.00 | 84.18 | C |
| ATOM | 507 | O | GLY | A | 58 | 113.135 | 12.524 | 8.674 | 1.00 | 85.98 | O |
| ATOM | 508 | N | LYS | A | 59 | 111.899 | 14.255 | 9.427 | 1.00 | 82.88 | N |
| ATOM | 509 | CA | LYS | A | 59 | 110.931 | 13.430 | 10.147 | 1.00 | 83.70 | C |
| ATOM | 510 | CB | LYS | A | 59 | 111.279 | 13.390 | 11.639 | 1.00 | 87.21 | C |
| ATOM | 511 | CG | LYS | A | 59 | 112.457 | 12.494 | 11.985 | 1.00 | 90.93 | C |
| ATOM | 512 | CD | LYS | A | 59 | 112.880 | 12.671 | 13.433 | 1.00 | 95.35 | C |
| ATOM | 513 | CE | LYS | A | 59 | 113.889 | 11.613 | 13.850 | 1.00 | 100.46 | C |
| ATOM | 514 | NZ | LYS | A | 59 | 114.320 | 11.783 | 15.267 | 1.00 | 103.42 | N |
| ATOM | 515 | C | LYS | A | 59 | 109.507 | 13.947 | 9.962 | 1.00 | 81.27 | C |
| ATOM | 516 | O | LYS | A | 59 | 109.292 | 15.090 | 9.554 | 1.00 | 79.60 | O |
| ATOM | 517 | N | ALA | A | 60 | 108.540 | 13.091 | 10.271 | 1.00 | 79.91 | N |
| ATOM | 518 | CA | ALA | A | 60 | 107.128 | 13.437 | 10.156 | 1.00 | 79.46 | C |
| ATOM | 519 | CB | ALA | A | 60 | 106.282 | 12.173 | 10.104 | 1.00 | 81.71 | C |
| ATOM | 520 | C | ALA | A | 60 | 106.678 | 14.321 | 11.315 | 1.00 | 77.96 | C |
| ATOM | 521 | O | ALA | A | 60 | 107.222 | 14.246 | 12.418 | 1.00 | 81.06 | O |
| ATOM | 522 | N | ALA | A | 61 | 105.681 | 15.158 | 11.049 | 1.00 | 74.15 | N |
| ATOM | 523 | CA | ALA | A | 61 | 105.096 | 16.029 | 12.056 | 1.00 | 72.68 | C |
| ATOM | 524 | CB | ALA | A | 61 | 105.499 | 17.470 | 11.806 | 1.00 | 71.45 | C |
| ATOM | 525 | C | ALA | A | 61 | 103.584 | 15.879 | 11.991 | 1.00 | 73.76 | C |
| ATOM | 526 | O | ALA | A | 61 | 102.958 | 16.323 | 11.031 | 1.00 | 76.05 | O |
| ATOM | 527 | N | THR | A | 62 | 102.998 | 15.252 | 13.007 | 1.00 | 74.78 | N |
| ATOM | 528 | CA | THR | A | 62 | 101.556 | 15.021 | 13.029 | 1.00 | 74.22 | C |
| ATOM | 529 | CB | THR | A | 62 | 101.182 | 13.889 | 13.999 | 1.00 | 74.44 | C |
| ATOM | 530 | OG1 | THR | A | 62 | 101.914 | 12.706 | 13.655 | 1.00 | 77.29 | O |
| ATOM | 531 | CG2 | THR | A | 62 | 99.690 | 13.590 | 13.929 | 1.00 | 74.77 | C |
| ATOM | 532 | C | THR | A | 62 | 100.789 | 16.284 | 13.415 | 1.00 | 73.32 | C |
| ATOM | 533 | O | THR | A | 62 | 101.075 | 16.906 | 14.437 | 1.00 | 73.85 | O |
| ATOM | 534 | N | ALA | A | 63 | 99.818 | 16.648 | 12.581 | 1.00 | 71.65 | N |
| ATOM | 535 | CA | ALA | A | 63 | 98.959 | 17.799 | 12.812 | 1.00 | 71.44 | C |
| ATOM | 536 | CB | ALA | A | 63 | 99.055 | 18.763 | 11.646 | 1.00 | 72.36 | C |
| ATOM | 537 | C | ALA | A | 63 | 97.529 | 17.313 | 12.972 | 1.00 | 72.07 | C |
| ATOM | 538 | O | ALA | A | 63 | 97.175 | 16.254 | 12.454 | 1.00 | 72.86 | O |
| ATOM | 539 | N | GLU | A | 64 | 96.713 | 18.080 | 13.691 | 1.00 | 73.76 | N |
| ATOM | 540 | CA | GLU | A | 64 | 95.314 | 17.714 | 13.916 | 1.00 | 74.96 | C |
| ATOM | 541 | CB | GLU | A | 64 | 94.972 | 17.740 | 15.404 | 1.00 | 76.45 | C |
| ATOM | 542 | CG | GLU | A | 64 | 95.780 | 16.747 | 16.224 | 1.00 | 79.88 | C |
| ATOM | 543 | CD | GLU | A | 64 | 95.271 | 16.604 | 17.645 | 1.00 | 83.26 | C |
| ATOM | 544 | OE1 | GLU | A | 64 | 94.084 | 16.260 | 17.822 | 1.00 | 84.32 | O |
| ATOM | 545 | OE2 | GLU | A | 64 | 96.063 | 16.818 | 18.587 | 1.00 | 85.29 | O |
| ATOM | 546 | C | GLU | A | 64 | 94.378 | 18.630 | 13.139 | 1.00 | 74.74 | C |
| ATOM | 547 | O | GLU | A | 64 | 94.387 | 19.850 | 13.323 | 1.00 | 74.92 | O |
| ATOM | 548 | N | LEU | A | 65 | 93.577 | 18.024 | 12.268 | 1.00 | 73.51 | N |
| ATOM | 549 | CA | LEU | A | 65 | 92.629 | 18.744 | 11.431 | 1.00 | 71.37 | C |
| ATOM | 550 | CB | LEU | A | 65 | 92.931 | 18.473 | 9.956 | 1.00 | 71.01 | C |
| ATOM | 551 | CG | LEU | A | 65 | 94.395 | 18.637 | 9.526 | 1.00 | 71.64 | C |
| ATOM | 552 | CD1 | LEU | A | 65 | 94.593 | 18.148 | 8.102 | 1.00 | 73.33 | C |
| ATOM | 553 | CD2 | LEU | A | 65 | 94.859 | 20.078 | 9.664 | 1.00 | 71.74 | C |
| ATOM | 554 | C | LEU | A | 65 | 91.232 | 18.257 | 11.782 | 1.00 | 69.60 | C |
| ATOM | 555 | O | LEU | A | 65 | 90.983 | 17.052 | 11.773 | 1.00 | 68.72 | O |
| ATOM | 556 | N | ALA | A | 66 | 90.328 | 19.181 | 12.104 | 1.00 | 69.80 | N |
| ATOM | 557 | CA | ALA | A | 66 | 88.965 | 18.808 | 12.481 | 1.00 | 71.86 | C |
| ATOM | 558 | CB | ALA | A | 66 | 88.952 | 18.233 | 13.893 | 1.00 | 72.50 | C |
| ATOM | 559 | C | ALA | A | 66 | 87.972 | 19.962 | 12.377 | 1.00 | 75.05 | C |
| ATOM | 560 | O | ALA | A | 66 | 88.357 | 21.130 | 12.313 | 1.00 | 76.83 | O |
| ATOM | 561 | N | CYS | A | 67 | 86.689 | 19.611 | 12.367 | 1.00 | 79.66 | N |
| ATOM | 562 | CA | CYS | A | 67 | 85.606 | 20.587 | 12.285 | 1.00 | 84.59 | C |
| ATOM | 563 | CB | CYS | A | 67 | 84.324 | 19.913 | 11.791 | 1.00 | 89.06 | C |
| ATOM | 564 | SG | CYS | A | 67 | 83.623 | 18.703 | 12.944 | 1.00 | 99.46 | S |
| ATOM | 565 | C | CYS | A | 67 | 85.349 | 21.253 | 13.632 | 1.00 | 85.01 | C |
| ATOM | 566 | O | CYS | A | 67 | 84.738 | 22.321 | 13.691 | 1.00 | 85.07 | O |
| ATOM | 567 | N | ASN | A | 68 | 85.799 | 20.609 | 14.707 | 1.00 | 89.37 | N |
| ATOM | 568 | CA | ASN | A | 68 | 85.644 | 21.151 | 16.053 | 1.00 | 93.19 | C |
| ATOM | 569 | CB | ASN | A | 68 | 84.401 | 20.587 | 16.750 | 1.00 | 98.47 | C |
| ATOM | 570 | CG | ASN | A | 68 | 83.562 | 21.678 | 17.382 | 1.00 | 106.32 | C |
| ATOM | 571 | OD1 | ASN | A | 68 | 84.040 | 22.438 | 18.231 | 1.00 | 111.29 | O |
| ATOM | 572 | ND2 | ASN | A | 68 | 82.315 | 21.785 | 16.947 | 1.00 | 110.61 | N |
| ATOM | 573 | C | ASN | A | 68 | 86.866 | 20.854 | 16.898 | 1.00 | 89.54 | C |
| ATOM | 574 | O | ASN | A | 68 | 87.495 | 19.803 | 16.752 | 1.00 | 91.68 | O |
| ATOM | 575 | N | LYS | A | 69 | 87.186 | 21.778 | 17.795 | 1.00 | 86.26 | N |
| ATOM | 576 | CA | LYS | A | 69 | 88.306 | 21.598 | 18.706 | 1.00 | 84.37 | C |
| ATOM | 577 | CB | LYS | A | 69 | 88.599 | 22.902 | 19.446 | 1.00 | 84.78 | C |
| ATOM | 578 | CG | LYS | A | 69 | 89.835 | 22.845 | 20.325 | 1.00 | 85.39 | C |
| ATOM | 579 | CD | LYS | A | 69 | 90.389 | 24.228 | 20.637 | 1.00 | 86.09 | C |
| ATOM | 580 | CE | LYS | A | 69 | 89.394 | 25.097 | 21.390 | 1.00 | 86.95 | C |
| ATOM | 581 | NZ | LYS | A | 69 | 89.991 | 26.410 | 21.760 | 1.00 | 87.92 | N |
| ATOM | 582 | C | LYS | A | 69 | 88.029 | 20.447 | 19.683 | 1.00 | 82.18 | C |
| ATOM | 583 | O | LYS | A | 69 | 88.958 | 19.835 | 20.203 | 1.00 | 82.98 | O |
| ATOM | 584 | N | GLY | A | 70 | 86.750 | 20.150 | 19.913 | 1.00 | 80.13 | N |
| ATOM | 585 | CA | GLY | A | 70 | 86.350 | 19.014 | 20.742 | 1.00 | 78.14 | C |
| ATOM | 586 | C | GLY | A | 70 | 86.766 | 17.677 | 20.153 | 1.00 | 77.52 | C |
| ATOM | 587 | O | GLY | A | 70 | 86.987 | 16.712 | 20.885 | 1.00 | 76.75 | O |
| ATOM | 588 | N | ALA | A | 71 | 86.878 | 17.622 | 18.826 | 1.00 | 78.12 | N |
| ATOM | 589 | CA | ALA | A | 71 | 87.305 | 16.410 | 18.126 | 1.00 | 75.95 | C |
| ATOM | 590 | CB | ALA | A | 71 | 86.765 | 16.412 | 16.704 | 1.00 | 77.32 | C |
| ATOM | 591 | C | ALA | A | 71 | 88.826 | 16.241 | 18.109 | 1.00 | 75.03 | C |
| ATOM | 592 | O | ALA | A | 71 | 89.329 | 15.288 | 17.521 | 1.00 | 77.75 | O |
| ATOM | 593 | N | THR | A | 72 | 89.552 | 17.157 | 18.748 | 1.00 | 75.74 | N |
| ATOM | 594 | CA | THR | A | 72 | 91.009 | 17.092 | 18.825 | 1.00 | 77.19 | C |
| ATOM | 595 | CB | THR | A | 72 | 91.642 | 18.360 | 18.225 | 1.00 | 77.47 | C |
| ATOM | 596 | OG1 | THR | A | 72 | 91.168 | 19.515 | 18.927 | 1.00 | 74.39 | O |
| ATOM | 597 | CG2 | THR | A | 72 | 91.290 | 18.485 | 16.752 | 1.00 | 80.25 | C |
| ATOM | 598 | C | THR | A | 72 | 91.464 | 16.948 | 20.276 | 1.00 | 80.88 | C |
| ATOM | 599 | O | THR | A | 72 | 90.646 | 16.952 | 21.196 | 1.00 | 82.69 | O |
| ATOM | 600 | N | THR | A | 73 | 92.776 | 16.827 | 20.471 | 1.00 | 83.90 | N |
| ATOM | 601 | CA | THR | A | 73 | 93.373 | 16.708 | 21.805 | 1.00 | 84.61 | C |
| ATOM | 602 | CB | THR | A | 73 | 94.884 | 16.416 | 21.707 | 1.00 | 85.56 | C |
| ATOM | 603 | OG1 | THR | A | 73 | 95.130 | 15.511 | 20.625 | 1.00 | 85.62 | O |
| ATOM | 604 | CG2 | THR | A | 73 | 95.410 | 15.811 | 23.004 | 1.00 | 88.69 | C |
| ATOM | 605 | C | THR | A | 73 | 93.189 | 17.985 | 22.634 | 1.00 | 84.43 | C |
| ATOM | 606 | O | THR | A | 73 | 93.260 | 17.951 | 23.863 | 1.00 | 87.77 | O |
| ATOM | 607 | N | TRP | A | 74 | 92.934 | 19.102 | 21.958 | 1.00 | 82.29 | N |
| ATOM | 608 | CA | TRP | A | 74 | 92.762 | 20.392 | 22.617 | 1.00 | 80.19 | C |
| ATOM | 609 | CB | TRP | A | 74 | 93.267 | 21.503 | 21.692 | 1.00 | 81.10 | C |
| ATOM | 610 | CG | TRP | A | 74 | 94.650 | 21.231 | 21.197 | 1.00 | 81.68 | C |
| ATOM | 611 | CD1 | TRP | A | 74 | 95.000 | 20.674 | 20.003 | 1.00 | 80.85 | C |
| ATOM | 612 | NE1 | TRP | A | 74 | 96.365 | 20.556 | 19.917 | 1.00 | 82.91 | N |
| ATOM | 613 | CE2 | TRP | A | 74 | 96.923 | 21.033 | 21.073 | 1.00 | 83.06 | C |
| ATOM | 614 | CD2 | TRP | A | 74 | 95.870 | 21.458 | 21.907 | 1.00 | 82.46 | C |
| ATOM | 615 | CE3 | TRP | A | 74 | 96.175 | 21.991 | 23.163 | 1.00 | 81.89 | C |
| ATOM | 616 | CZ3 | TRP | A | 74 | 97.504 | 22.080 | 23.541 | 1.00 | 83.00 | C |
| ATOM | 617 | CH2 | TRP | A | 74 | 98.530 | 21.649 | 22.689 | 1.00 | 83.09 | C |
| ATOM | 618 | CZ2 | TRP | A | 74 | 98.262 | 21.124 | 21.456 | 1.00 | 82.68 | C |
| ATOM | 619 | C | TRP | A | 74 | 91.308 | 20.640 | 23.015 | 1.00 | 78.66 | C |
| ATOM | 620 | O | TRP | A | 74 | 90.870 | 21.787 | 23.103 | 1.00 | 77.18 | O |
| ATOM | 621 | N | PHE | A | 75 | 90.576 | 19.562 | 23.290 | 1.00 | 79.48 | N |
| ATOM | 622 | CA | PHE | A | 75 | 89.155 | 19.645 | 23.638 | 1.00 | 81.86 | C |
| ATOM | 623 | CB | PHE | A | 75 | 88.525 | 18.248 | 23.675 | 1.00 | 81.03 | C |
| ATOM | 624 | CG | PHE | A | 75 | 89.025 | 17.382 | 24.800 | 1.00 | 81.49 | C |
| ATOM | 625 | CD1 | PHE | A | 75 | 88.464 | 17.474 | 26.067 | 1.00 | 82.81 | C |
| ATOM | 626 | CE1 | PHE | A | 75 | 88.920 | 16.679 | 27.106 | 1.00 | 82.06 | C |
| ATOM | 627 | CZ | PHE | A | 75 | 89.945 | 15.776 | 26.886 | 1.00 | 81.50 | C |
| ATOM | 628 | CE2 | PHE | A | 75 | 90.513 | 15.675 | 25.629 | 1.00 | 82.26 | C |
| ATOM | 629 | CD2 | PHE | A | 75 | 90.052 | 16.473 | 24.593 | 1.00 | 81.87 | C |
| ATOM | 630 | C | PHE | A | 75 | 88.883 | 20.350 | 24.966 | 1.00 | 84.27 | C |
| ATOM | 631 | O | PHE | A | 75 | 87.794 | 20.879 | 25.167 | 1.00 | 83.13 | O |
| ATOM | 632 | N | ASN | A | 76 | 89.857 | 20.344 | 25.874 | 1.00 | 88.11 | N |
| ATOM | 633 | CA | ASN | A | 76 | 89.682 | 20.985 | 27.186 | 1.00 | 89.63 | C |
| ATOM | 634 | CB | ASN | A | 76 | 90.845 | 20.651 | 28.129 | 1.00 | 92.20 | C |
| ATOM | 635 | CG | ASN | A | 76 | 92.193 | 21.063 | 27.577 | 1.00 | 94.54 | C |
| ATOM | 636 | OD1 | ASN | A | 76 | 92.288 | 21.819 | 26.609 | 1.00 | 96.72 | O |
| ATOM | 637 | ND2 | ASN | A | 76 | 93.249 | 20.564 | 28.198 | 1.00 | 98.59 | N |
| ATOM | 638 | C | ASN | A | 76 | 89.463 | 22.498 | 27.114 | 1.00 | 88.24 | C |
| ATOM | 639 | O | ASN | A | 76 | 88.887 | 23.084 | 28.029 | 1.00 | 90.63 | O |
| ATOM | 640 | N | SER | A | 77 | 89.933 | 23.123 | 26.036 | 1.00 | 87.91 | N |
| ATOM | 641 | CA | SER | A | 77 | 89.700 | 24.548 | 25.801 | 1.00 | 88.76 | C |
| ATOM | 642 | CB | SER | A | 77 | 90.924 | 25.195 | 25.151 | 1.00 | 87.82 | C |
| ATOM | 643 | OG | SER | A | 77 | 91.255 | 24.559 | 23.934 | 1.00 | 88.97 | O |
| ATOM | 644 | C | SER | A | 77 | 88.456 | 24.775 | 24.934 | 1.00 | 89.89 | C |
| ATOM | 645 | O | SER | A | 77 | 88.060 | 25.916 | 24.702 | 1.00 | 90.07 | O |
| ATOM | 646 | N | SER | A | 78 | 87.850 | 23.689 | 24.453 | 1.00 | 94.92 | N |
| ATOM | 647 | CA | SER | A | 78 | 86.629 | 23.761 | 23.649 | 1.00 | 99.03 | C |
| ATOM | 648 | CB | SER | A | 78 | 86.590 | 22.628 | 22.623 | 1.00 | 99.62 | C |
| ATOM | 649 | OG | SER | A | 78 | 86.188 | 21.405 | 23.222 | 1.00 | 97.38 | O |
| ATOM | 650 | C | SER | A | 78 | 85.422 | 23.637 | 24.574 | 1.00 | 102.60 | C |
| ATOM | 651 | O | SER | A | 78 | 85.491 | 22.969 | 25.600 | 1.00 | 103.92 | O |
| ATOM | 652 | N | GLU | A | 79 | 84.313 | 24.267 | 24.207 | 1.00 | 108.55 | N |
| ATOM | 653 | CA | GLU | A | 79 | 83.107 | 24.256 | 25.060 | 1.00 | 114.49 | C |
| ATOM | 654 | CB | GLU | A | 79 | 82.200 | 25.440 | 24.691 | 1.00 | 123.71 | C |
| ATOM | 655 | CG | GLU | A | 79 | 80.904 | 25.532 | 25.489 | 1.00 | 132.76 | C |
| ATOM | 656 | CD | GLU | A | 79 | 80.009 | 26.677 | 25.043 | 1.00 | 140.84 | C |
| ATOM | 657 | OE1 | GLU | A | 79 | 80.450 | 27.508 | 24.217 | 1.00 | 147.37 | O |
| ATOM | 658 | OE2 | GLU | A | 79 | 78.857 | 26.748 | 25.519 | 1.00 | 143.38 | O |
| ATOM | 659 | C | GLU | A | 79 | 82.293 | 22.953 | 25.091 | 1.00 | 109.97 | C |
| ATOM | 660 | O | GLU | A | 79 | 81.727 | 22.609 | 26.129 | 1.00 | 106.29 | O |
| ATOM | 661 | N | GLY | A | 80 | 82.220 | 22.242 | 23.971 | 1.00 | 110.25 | N |
| ATOM | 662 | CA | GLY | A | 80 | 81.446 | 20.997 | 23.891 | 1.00 | 107.47 | C |
| ATOM | 663 | C | GLY | A | 80 | 81.995 | 19.802 | 24.660 | 1.00 | 104.12 | C |
| ATOM | 664 | O | GLY | A | 80 | 81.264 | 18.857 | 24.953 | 1.00 | 104.62 | O |
| ATOM | 665 | N | GLY | A | 81 | 83.275 | 19.851 | 25.012 | 1.00 | 100.72 | N |
| ATOM | 666 | CA | GLY | A | 81 | 83.952 | 18.716 | 25.629 | 1.00 | 97.65 | C |
| ATOM | 667 | C | GLY | A | 81 | 84.530 | 17.772 | 24.592 | 1.00 | 93.95 | C |
| ATOM | 668 | O | GLY | A | 81 | 84.514 | 18.060 | 23.394 | 1.00 | 92.80 | O |
| ATOM | 669 | N | ASN | A | 82 | 85.027 | 16.633 | 25.063 | 1.00 | 92.19 | N |
| ATOM | 670 | CA | ASN | A | 82 | 85.684 | 15.651 | 24.207 | 1.00 | 90.58 | C |
| ATOM | 671 | CB | ASN | A | 82 | 86.543 | 14.713 | 25.058 | 1.00 | 90.22 | C |
| ATOM | 672 | CG | ASN | A | 82 | 87.370 | 13.761 | 24.221 | 1.00 | 90.16 | C |
| ATOM | 673 | OD1 | ASN | A | 82 | 87.340 | 13.811 | 22.994 | 1.00 | 93.93 | O |
| ATOM | 674 | ND2 | ASN | A | 82 | 88.116 | 12.889 | 24.882 | 1.00 | 90.44 | N |
| ATOM | 675 | C | ASN | A | 82 | 84.721 | 14.806 | 23.376 | 1.00 | 91.56 | C |
| ATOM | 676 | O | ASN | A | 82 | 83.892 | 14.076 | 23.922 | 1.00 | 92.50 | O |
| ATOM | 677 | N | ILE | A | 83 | 84.850 | 14.902 | 22.054 | 1.00 | 91.59 | N |
| ATOM | 678 | CA | ILE | A | 83 | 84.067 | 14.083 | 21.128 | 1.00 | 92.28 | C |
| ATOM | 679 | CB | ILE | A | 83 | 83.002 | 14.924 | 20.383 | 1.00 | 92.95 | C |
| ATOM | 680 | CG1 | ILE | A | 83 | 83.655 | 15.994 | 19.494 | 1.00 | 93.81 | C |
| ATOM | 681 | CD1 | ILE | A | 83 | 82.671 | 16.825 | 18.698 | 1.00 | 94.08 | C |
| ATOM | 682 | CG2 | ILE | A | 83 | 82.048 | 15.565 | 21.378 | 1.00 | 93.58 | C |
| ATOM | 683 | C | ILE | A | 83 | 84.953 | 13.371 | 20.103 | 1.00 | 92.93 | C |
| ATOM | 684 | O | ILE | A | 83 | 84.443 | 12.795 | 19.141 | 1.00 | 95.28 | O |
| ATOM | 685 | N | GLN | A | 84 | 86.269 | 13.390 | 20.316 | 1.00 | 91.26 | N |
| ATOM | 686 | CA | GLN | A | 84 | 87.202 | 12.832 | 19.337 | 1.00 | 91.95 | C |
| ATOM | 687 | CB | GLN | A | 84 | 88.656 | 13.183 | 19.686 | 1.00 | 93.42 | C |
| ATOM | 688 | CG | GLN | A | 84 | 89.228 | 12.543 | 20.940 | 1.00 | 92.49 | C |
| ATOM | 689 | CD | GLN | A | 84 | 90.513 | 13.214 | 21.412 | 1.00 | 91.92 | C |
| ATOM | 690 | OE1 | GLN | A | 84 | 90.641 | 13.518 | 22.588 | 1.00 | 93.06 | O |
| ATOM | 691 | NE2 | GLN | A | 84 | 91.453 | 13.469 | 20.497 | 1.00 | 89.11 | N |
| ATOM | 692 | C | GLN | A | 84 | 87.021 | 11.331 | 19.159 | 1.00 | 88.78 | C |
| ATOM | 693 | O | GLN | A | 84 | 86.706 | 10.617 | 20.110 | 1.00 | 87.28 | O |
| ATOM | 694 | N | ASN | A | 85 | 87.207 | 10.875 | 17.923 | 1.00 | 88.53 | N |
| ATOM | 695 | CA | ASN | A | 85 | 86.994 | 9.478 | 17.560 | 1.00 | 86.85 | C |
| ATOM | 696 | CB | ASN | A | 85 | 85.559 | 9.296 | 17.050 | 1.00 | 88.39 | C |
| ATOM | 697 | CG | ASN | A | 85 | 85.231 | 7.852 | 16.728 | 1.00 | 90.44 | C |
| ATOM | 698 | OD1 | ASN | A | 85 | 85.629 | 6.938 | 17.448 | 1.00 | 91.39 | O |
| ATOM | 699 | ND2 | ASN | A | 85 | 84.495 | 7.640 | 15.644 | 1.00 | 95.33 | N |
| ATOM | 700 | C | ASN | A | 85 | 88.000 | 9.008 | 16.507 | 1.00 | 82.78 | C |
| ATOM | 701 | O | ASN | A | 85 | 87.668 | 8.860 | 15.331 | 1.00 | 82.04 | O |
| ATOM | 702 | N | GLY | A | 86 | 89.235 | 8.784 | 16.946 | 1.00 | 80.60 | N |
| ATOM | 703 | CA | GLY | A | 86 | 90.294 | 8.289 | 16.072 | 1.00 | 78.14 | C |
| ATOM | 704 | C | GLY | A | 86 | 90.655 | 9.265 | 14.972 | 1.00 | 76.72 | C |
| ATOM | 705 | O | GLY | A | 86 | 90.790 | 10.462 | 15.217 | 1.00 | 78.98 | O |
| ATOM | 706 | N | ASN | A | 87 | 90.792 | 8.751 | 13.753 | 1.00 | 76.52 | N |
| ATOM | 707 | CA | ASN | A | 87 | 91.156 | 9.571 | 12.591 | 1.00 | 76.73 | C |
| ATOM | 708 | CB | ASN | A | 87 | 91.836 | 8.710 | 11.516 | 1.00 | 76.38 | C |
| ATOM | 709 | CG | ASN | A | 87 | 93.330 | 8.566 | 11.734 | 1.00 | 75.91 | C |
| ATOM | 710 | OD1 | ASN | A | 87 | 94.020 | 9.537 | 12.034 | 1.00 | 72.57 | O |
| ATOM | 711 | ND2 | ASN | A | 87 | 93.842 | 7.354 | 11.552 | 1.00 | 78.45 | N |
| ATOM | 712 | C | ASN | A | 87 | 90.006 | 10.355 | 11.953 | 1.00 | 77.29 | C |
| ATOM | 713 | O | ASN | A | 87 | 90.212 | 11.019 | 10.940 | 1.00 | 78.45 | O |
| ATOM | 714 | N | ASP | A | 88 | 88.811 | 10.293 | 12.535 | 1.00 | 77.80 | N |
| ATOM | 715 | CA | ASP | A | 88 | 87.659 | 11.015 | 11.994 | 1.00 | 79.67 | C |
| ATOM | 716 | CB | ASP | A | 88 | 86.369 | 10.517 | 12.654 | 1.00 | 79.19 | C |
| ATOM | 717 | CG | ASP | A | 88 | 85.114 | 11.001 | 11.945 | 1.00 | 81.31 | C |
| ATOM | 718 | OD1 | ASP | A | 88 | 85.179 | 11.989 | 11.182 | 1.00 | 84.32 | O |
| ATOM | 719 | OD2 | ASP | A | 88 | 84.050 | 10.382 | 12.154 | 1.00 | 84.65 | O |
| ATOM | 720 | C | ASP | A | 88 | 87.826 | 12.530 | 12.209 | 1.00 | 81.10 | C |
| ATOM | 721 | O | ASP | A | 88 | 87.862 | 12.985 | 13.354 | 1.00 | 85.52 | O |
| ATOM | 722 | N | PRO | A | 89 | 87.937 | 13.311 | 11.112 | 1.00 | 80.28 | N |
| ATOM | 723 | CA | PRO | A | 89 | 88.069 | 14.765 | 11.244 | 1.00 | 80.13 | C |
| ATOM | 724 | CB | PRO | A | 89 | 88.398 | 15.231 | 9.816 | 1.00 | 81.36 | C |
| ATOM | 725 | CG | PRO | A | 89 | 88.773 | 14.005 | 9.063 | 1.00 | 81.57 | C |
| ATOM | 726 | CD | PRO | A | 89 | 87.996 | 12.898 | 9.700 | 1.00 | 81.53 | C |
| ATOM | 727 | C | PRO | A | 89 | 86.802 | 15.462 | 11.738 | 1.00 | 81.61 | C |
| ATOM | 728 | O | PRO | A | 89 | 86.882 | 16.565 | 12.271 | 1.00 | 82.89 | O |
| ATOM | 729 | N | CYS | A | 90 | 85.643 | 14.842 | 11.545 | 1.00 | 83.76 | N |
| ATOM | 730 | CA | CYS | A | 90 | 84.397 | 15.408 | 12.052 | 1.00 | 87.12 | C |
| ATOM | 731 | CB | CYS | A | 90 | 83.756 | 16.336 | 11.019 | 1.00 | 89.21 | C |
| ATOM | 732 | SG | CYS | A | 90 | 82.536 | 17.452 | 11.749 | 1.00 | 97.73 | S |
| ATOM | 733 | C | CYS | A | 90 | 83.441 | 14.291 | 12.475 | 1.00 | 87.04 | C |
| ATOM | 734 | O | CYS | A | 90 | 82.627 | 13.831 | 11.676 | 1.00 | 88.42 | O |
| ATOM | 735 | N | PRO | A | 91 | 83.560 | 13.834 | 13.736 | 1.00 | 88.68 | N |
| ATOM | 736 | CA | PRO | A | 91 | 82.711 | 12.766 | 14.265 | 1.00 | 87.95 | C |
| ATOM | 737 | CB | PRO | A | 91 | 83.210 | 12.595 | 15.706 | 1.00 | 88.48 | C |
| ATOM | 738 | CG | PRO | A | 91 | 84.600 | 13.125 | 15.697 | 1.00 | 89.41 | C |
| ATOM | 739 | CD | PRO | A | 91 | 84.582 | 14.252 | 14.712 | 1.00 | 89.53 | C |
| ATOM | 740 | C | PRO | A | 91 | 81.231 | 13.133 | 14.254 | 1.00 | 87.89 | C |
| ATOM | 741 | O | PRO | A | 91 | 80.864 | 14.231 | 14.670 | 1.00 | 88.66 | O |
| ATOM | 742 | N | GLY | A | 92 | 80.400 | 12.216 | 13.768 | 1.00 | 88.83 | N |
| ATOM | 743 | CA | GLY | A | 92 | 78.956 | 12.427 | 13.711 | 1.00 | 88.73 | C |
| ATOM | 744 | C | GLY | A | 92 | 78.455 | 13.014 | 12.404 | 1.00 | 87.94 | C |
| ATOM | 745 | O | GLY | A | 92 | 77.247 | 13.115 | 12.200 | 1.00 | 92.01 | O |
| ATOM | 746 | N | SER | A | 93 | 79.372 | 13.401 | 11.519 | 1.00 | 85.11 | N |
| ATOM | 747 | CA | SER | A | 93 | 79.009 | 13.950 | 10.211 | 1.00 | 83.68 | C |
| ATOM | 748 | CB | SER | A | 93 | 79.288 | 15.455 | 10.156 | 1.00 | 83.96 | C |
| ATOM | 749 | OG | SER | A | 93 | 80.668 | 15.729 | 10.309 | 1.00 | 86.87 | O |
| ATOM | 750 | C | SER | A | 93 | 79.765 | 13.203 | 9.102 | 1.00 | 81.71 | C |
| ATOM | 751 | O | SER | A | 93 | 80.841 | 12.653 | 9.346 | 1.00 | 79.60 | O |
| ATOM | 752 | N | PRO | A | 94 | 79.195 | 13.171 | 7.881 | 1.00 | 81.27 | N |
| ATOM | 753 | CA | PRO | A | 94 | 79.796 | 12.420 | 6.781 | 1.00 | 79.29 | C |
| ATOM | 754 | CB | PRO | A | 94 | 78.625 | 12.248 | 5.816 | 1.00 | 81.13 | C |
| ATOM | 755 | CG | PRO | A | 94 | 77.800 | 13.467 | 6.022 | 1.00 | 80.76 | C |
| ATOM | 756 | CD | PRO | A | 94 | 77.957 | 13.858 | 7.462 | 1.00 | 80.51 | C |
| ATOM | 757 | C | PRO | A | 94 | 80.948 | 13.170 | 6.107 | 1.00 | 78.51 | C |
| ATOM | 758 | O | PRO | A | 94 | 81.217 | 14.320 | 6.464 | 1.00 | 77.25 | O |
| ATOM | 759 | N | PRO | A | 95 | 81.628 | 12.527 | 5.134 | 1.00 | 76.99 | N |
| ATOM | 760 | CA | PRO | A | 95 | 82.752 | 13.168 | 4.438 | 1.00 | 75.51 | C |
| ATOM | 761 | CB | PRO | A | 95 | 83.280 | 12.063 | 3.515 | 1.00 | 74.85 | C |
| ATOM | 762 | CG | PRO | A | 95 | 82.794 | 10.793 | 4.113 | 1.00 | 76.11 | C |
| ATOM | 763 | CD | PRO | A | 95 | 81.460 | 11.127 | 4.709 | 1.00 | 76.90 | C |
| ATOM | 764 | C | PRO | A | 95 | 82.383 | 14.411 | 3.622 | 1.00 | 76.85 | C |
| ATOM | 765 | O | PRO | A | 95 | 83.273 | 15.177 | 3.255 | 1.00 | 73.77 | O |
| ATOM | 766 | N | SER | A | 96 | 81.094 | 14.606 | 3.337 | 1.00 | 79.96 | N |
| ATOM | 767 | CA | SER | A | 96 | 80.635 | 15.806 | 2.632 | 1.00 | 81.01 | C |
| ATOM | 768 | CB | SER | A | 96 | 79.130 | 15.742 | 2.356 | 1.00 | 82.03 | C |
| ATOM | 769 | OG | SER | A | 96 | 78.380 | 15.812 | 3.556 | 1.00 | 81.00 | O |
| ATOM | 770 | C | SER | A | 96 | 80.958 | 17.063 | 3.436 | 1.00 | 81.78 | C |
| ATOM | 771 | O | SER | A | 96 | 81.075 | 18.153 | 2.876 | 1.00 | 82.50 | O |
| ATOM | 772 | N | GLU | A | 97 | 81.095 | 16.898 | 4.751 | 1.00 | 84.85 | N |
| ATOM | 773 | CA | GLU | A | 97 | 81.482 | 17.987 | 5.638 | 1.00 | 85.15 | C |
| ATOM | 774 | CB | GLU | A | 97 | 81.290 | 17.571 | 7.102 | 1.00 | 87.89 | C |
| ATOM | 775 | CG | GLU | A | 97 | 81.230 | 18.728 | 8.093 | 1.00 | 89.90 | C |
| ATOM | 776 | CD | GLU | A | 97 | 82.591 | 19.298 | 8.462 | 1.00 | 90.87 | C |
| ATOM | 777 | OE1 | GLU | A | 97 | 82.633 | 20.470 | 8.886 | 1.00 | 90.84 | O |
| ATOM | 778 | OE2 | GLU | A | 97 | 83.615 | 18.586 | 8.340 | 1.00 | 90.58 | O |
| ATOM | 779 | C | GLU | A | 97 | 82.932 | 18.407 | 5.363 | 1.00 | 82.63 | C |
| ATOM | 780 | O | GLU | A | 97 | 83.256 | 19.591 | 5.449 | 1.00 | 80.59 | O |
| ATOM | 781 | N | TYR | A | 98 | 83.795 | 17.451 | 5.010 | 1.00 | 81.53 | N |
| ATOM | 782 | CA | TYR | A | 98 | 85.179 | 17.784 | 4.653 | 1.00 | 79.61 | C |
| ATOM | 783 | CB | TYR | A | 98 | 86.205 | 16.673 | 4.983 | 1.00 | 79.95 | C |
| ATOM | 784 | CG | TYR | A | 98 | 85.804 | 15.496 | 5.847 | 1.00 | 80.21 | C |
| ATOM | 785 | CD1 | TYR | A | 98 | 85.165 | 15.671 | 7.065 | 1.00 | 83.17 | C |
| ATOM | 786 | CE1 | TYR | A | 98 | 84.821 | 14.580 | 7.851 | 1.00 | 86.71 | C |
| ATOM | 787 | CZ | TYR | A | 98 | 85.150 | 13.294 | 7.436 | 1.00 | 85.74 | C |
| ATOM | 788 | OH | TYR | A | 98 | 84.815 | 12.204 | 8.217 | 1.00 | 82.99 | O |
| ATOM | 789 | CE2 | TYR | A | 98 | 85.816 | 13.102 | 6.243 | 1.00 | 81.75 | C |
| ATOM | 790 | CD2 | TYR | A | 98 | 86.147 | 14.197 | 5.464 | 1.00 | 79.31 | C |
| ATOM | 791 | C | TYR | A | 98 | 85.281 | 18.111 | 3.148 | 1.00 | 78.58 | C |
| ATOM | 792 | O | TYR | A | 98 | 86.383 | 18.165 | 2.602 | 1.00 | 75.58 | O |
| ATOM | 793 | N | HIS | A | 99 | 84.138 | 18.308 | 2.488 | 1.00 | 78.17 | N |
| ATOM | 794 | CA | HIS | A | 99 | 84.070 | 18.625 | 1.056 | 1.00 | 78.49 | C |
| ATOM | 795 | CB | HIS | A | 99 | 84.615 | 20.029 | 0.775 | 1.00 | 80.65 | C |
| ATOM | 796 | CG | HIS | A | 99 | 83.839 | 21.122 | 1.438 | 1.00 | 84.17 | C |
| ATOM | 797 | ND1 | HIS | A | 99 | 82.534 | 21.414 | 1.104 | 1.00 | 86.58 | N |
| ATOM | 798 | CE1 | HIS | A | 99 | 82.108 | 22.423 | 1.841 | 1.00 | 87.71 | C |
| ATOM | 799 | NE2 | HIS | A | 99 | 83.091 | 22.802 | 2.637 | 1.00 | 87.96 | N |
| ATOM | 800 | CD2 | HIS | A | 99 | 84.187 | 22.006 | 2.402 | 1.00 | 85.49 | C |
| ATOM | 801 | C | HIS | A | 99 | 84.760 | 17.591 | 0.157 | 1.00 | 77.06 | C |
| ATOM | 802 | O | HIS | A | 99 | 85.602 | 17.933 | −0.678 | 1.00 | 75.08 | O |
| ATOM | 803 | N | THR | A | 100 | 84.385 | 16.328 | 0.333 | 1.00 | 76.26 | N |
| ATOM | 804 | CA | THR | A | 100 | 84.905 | 15.243 | −0.494 | 1.00 | 75.33 | C |
| ATOM | 805 | CB | THR | A | 100 | 86.259 | 14.719 | 0.026 | 1.00 | 75.69 | C |
| ATOM | 806 | OG1 | THR | A | 100 | 86.691 | 13.618 | −0.781 | 1.00 | 73.34 | O |
| ATOM | 807 | CG2 | THR | A | 100 | 86.161 | 14.268 | 1.487 | 1.00 | 76.67 | C |
| ATOM | 808 | C | THR | A | 100 | 83.904 | 14.094 | −0.565 | 1.00 | 77.28 | C |
| ATOM | 809 | O | THR | A | 100 | 83.156 | 13.852 | 0.381 | 1.00 | 78.28 | O |
| ATOM | 810 | N | THR | A | 101 | 83.893 | 13.401 | −1.700 | 1.00 | 79.81 | N |
| ATOM | 811 | CA | THR | A | 101 | 83.006 | 12.257 | −1.908 | 1.00 | 78.80 | C |
| ATOM | 812 | CB | THR | A | 101 | 82.726 | 12.026 | −3.405 | 1.00 | 78.80 | C |
| ATOM | 813 | OG1 | THR | A | 101 | 83.949 | 11.727 | −4.091 | 1.00 | 80.62 | O |
| ATOM | 814 | CG2 | THR | A | 101 | 82.095 | 13.259 | −4.025 | 1.00 | 78.50 | C |
| ATOM | 815 | C | THR | A | 101 | 83.630 | 10.999 | −1.314 | 1.00 | 77.63 | C |
| ATOM | 816 | O | THR | A | 101 | 82.924 | 10.127 | −0.809 | 1.00 | 79.35 | O |
| ATOM | 817 | N | GLY | A | 102 | 84.957 | 10.912 | −1.389 | 1.00 | 74.75 | N |
| ATOM | 818 | CA | GLY | A | 102 | 85.700 | 9.780 | −0.843 | 1.00 | 73.71 | C |
| ATOM | 819 | C | GLY | A | 102 | 87.185 | 10.075 | −0.760 | 1.00 | 72.34 | C |
| ATOM | 820 | O | GLY | A | 102 | 87.616 | 11.198 | −1.013 | 1.00 | 71.11 | O |
| ATOM | 821 | N | ILE | A | 103 | 87.971 | 9.062 | −0.411 | 1.00 | 73.72 | N |
| ATOM | 822 | CA | ILE | A | 103 | 89.422 | 9.218 | −0.295 | 1.00 | 77.88 | C |
| ATOM | 823 | CB | ILE | A | 103 | 90.077 | 7.954 | 0.313 | 1.00 | 78.60 | C |
| ATOM | 824 | CG1 | ILE | A | 103 | 91.526 | 8.238 | 0.723 | 1.00 | 79.37 | C |
| ATOM | 825 | CD1 | ILE | A | 103 | 92.155 | 7.125 | 1.531 | 1.00 | 81.18 | C |
| ATOM | 826 | CG2 | ILE | A | 103 | 90.005 | 6.774 | −0.650 | 1.00 | 79.39 | C |
| ATOM | 827 | C | ILE | A | 103 | 90.080 | 9.560 | −1.638 | 1.00 | 81.78 | C |
| ATOM | 828 | O | ILE | A | 103 | 91.135 | 10.193 | −1.677 | 1.00 | 83.03 | O |
| ATOM | 829 | N | ASP | A | 104 | 89.454 | 9.131 | −2.730 | 1.00 | 88.15 | N |
| ATOM | 830 | CA | ASP | A | 104 | 89.956 | 9.401 | −4.083 | 1.00 | 91.83 | C |
| ATOM | 831 | CB | ASP | A | 104 | 89.357 | 8.399 | −5.089 | 1.00 | 98.41 | C |
| ATOM | 832 | CG | ASP | A | 104 | 87.828 | 8.292 | −4.998 | 1.00 | 106.90 | C |
| ATOM | 833 | OD1 | ASP | A | 104 | 87.191 | 9.093 | −4.276 | 1.00 | 108.74 | O |
| ATOM | 834 | OD2 | ASP | A | 104 | 87.263 | 7.390 | −5.653 | 1.00 | 115.22 | O |
| ATOM | 835 | C | ASP | A | 104 | 89.704 | 10.844 | −4.547 | 1.00 | 87.09 | C |
| ATOM | 836 | O | ASP | A | 104 | 90.380 | 11.332 | −5.454 | 1.00 | 85.40 | O |
| ATOM | 837 | N | ASP | A | 105 | 88.747 | 11.520 | −3.913 | 1.00 | 84.52 | N |
| ATOM | 838 | CA | ASP | A | 105 | 88.347 | 12.878 | −4.300 | 1.00 | 83.78 | C |
| ATOM | 839 | CB | ASP | A | 105 | 86.818 | 12.984 | −4.220 | 1.00 | 83.39 | C |
| ATOM | 840 | CG | ASP | A | 105 | 86.278 | 14.287 | −4.782 | 1.00 | 83.83 | C |
| ATOM | 841 | OD1 | ASP | A | 105 | 86.840 | 14.809 | −5.772 | 1.00 | 84.71 | O |
| ATOM | 842 | OD2 | ASP | A | 105 | 85.271 | 14.779 | −4.231 | 1.00 | 83.05 | O |
| ATOM | 843 | C | ASP | A | 105 | 89.006 | 13.990 | −3.462 | 1.00 | 82.38 | C |
| ATOM | 844 | O | ASP | A | 105 | 88.697 | 15.169 | −3.637 | 1.00 | 84.48 | O |
| ATOM | 845 | N | VAL | A | 106 | 89.915 | 13.623 | −2.562 | 1.00 | 80.21 | N |
| ATOM | 846 | CA | VAL | A | 106 | 90.636 | 14.616 | −1.758 | 1.00 | 76.79 | C |
| ATOM | 847 | CB | VAL | A | 106 | 91.240 | 14.007 | −0.473 | 1.00 | 76.30 | C |
| ATOM | 848 | CG1 | VAL | A | 106 | 90.152 | 13.342 | 0.361 | 1.00 | 75.86 | C |
| ATOM | 849 | CG2 | VAL | A | 106 | 92.364 | 13.028 | −0.792 | 1.00 | 75.64 | C |
| ATOM | 850 | C | VAL | A | 106 | 91.732 | 15.252 | −2.613 | 1.00 | 77.16 | C |
| ATOM | 851 | O | VAL | A | 106 | 92.360 | 14.571 | −3.425 | 1.00 | 78.52 | O |
| ATOM | 852 | N | LYS | A | 107 | 91.964 | 16.550 | −2.423 | 1.00 | 76.96 | N |
| ATOM | 853 | CA | LYS | A | 107 | 92.907 | 17.302 | −3.262 | 1.00 | 76.64 | C |
| ATOM | 854 | CB | LYS | A | 107 | 92.202 | 18.528 | −3.844 | 1.00 | 78.36 | C |
| ATOM | 855 | CG | LYS | A | 107 | 90.971 | 18.201 | −4.675 | 1.00 | 80.98 | C |
| ATOM | 856 | CD | LYS | A | 107 | 91.330 | 17.445 | −5.943 | 1.00 | 83.14 | C |
| ATOM | 857 | CE | LYS | A | 107 | 90.093 | 17.097 | −6.748 | 1.00 | 85.25 | C |
| ATOM | 858 | NZ | LYS | A | 107 | 90.459 | 16.404 | −8.012 | 1.00 | 89.06 | N |
| ATOM | 859 | C | LYS | A | 107 | 94.197 | 17.739 | −2.568 | 1.00 | 75.69 | C |
| ATOM | 860 | O | LYS | A | 107 | 95.007 | 18.443 | −3.166 | 1.00 | 76.63 | O |
| ATOM | 861 | N | GLY | A | 108 | 94.386 | 17.345 | −1.313 | 1.00 | 75.76 | N |
| ATOM | 862 | CA | GLY | A | 108 | 95.621 | 17.655 | −0.597 | 1.00 | 74.81 | C |
| ATOM | 863 | C | GLY | A | 108 | 95.705 | 19.048 | 0.000 | 1.00 | 74.07 | C |
| ATOM | 864 | O | GLY | A | 108 | 95.023 | 19.975 | −0.442 | 1.00 | 74.11 | O |
| ATOM | 865 | N | CYS | A | 109 | 96.561 | 19.179 | 1.012 | 1.00 | 73.65 | N |
| ATOM | 866 | CA | CYS | A | 109 | 96.774 | 20.438 | 1.719 | 1.00 | 74.38 | C |
| ATOM | 867 | CB | CYS | A | 109 | 95.952 | 20.472 | 3.003 | 1.00 | 77.23 | C |
| ATOM | 868 | SG | CYS | A | 109 | 96.511 | 19.304 | 4.250 | 1.00 | 86.71 | S |
| ATOM | 869 | C | CYS | A | 109 | 98.258 | 20.590 | 2.034 | 1.00 | 70.37 | C |
| ATOM | 870 | O | CYS | A | 109 | 99.021 | 19.636 | 1.884 | 1.00 | 70.18 | O |
| ATOM | 871 | N | ALA | A | 110 | 98.661 | 21.774 | 2.492 | 1.00 | 68.05 | N |
| ATOM | 872 | CA | ALA | A | 110 | 100.080 | 22.073 | 2.693 | 1.00 | 67.71 | C |
| ATOM | 873 | CB | ALA | A | 110 | 100.606 | 22.832 | 1.484 | 1.00 | 66.87 | C |
| ATOM | 874 | C | ALA | A | 110 | 100.396 | 22.864 | 3.960 | 1.00 | 67.08 | C |
| ATOM | 875 | O | ALA | A | 110 | 99.507 | 23.404 | 4.615 | 1.00 | 65.06 | O |
| ATOM | 876 | N | MET | A | 111 | 101.687 | 22.911 | 4.283 | 1.00 | 66.62 | N |
| ATOM | 877 | CA | MET | A | 111 | 102.214 | 23.716 | 5.376 | 1.00 | 68.59 | C |
| ATOM | 878 | CB | MET | A | 111 | 102.953 | 22.855 | 6.408 | 1.00 | 69.75 | C |
| ATOM | 879 | CG | MET | A | 111 | 102.147 | 21.746 | 7.071 | 1.00 | 71.01 | C |
| ATOM | 880 | SD | MET | A | 111 | 100.703 | 22.240 | 8.040 | 1.00 | 71.63 | S |
| ATOM | 881 | CE | MET | A | 111 | 101.331 | 23.608 | 8.969 | 1.00 | 71.26 | C |
| ATOM | 882 | C | MET | A | 111 | 103.216 | 24.694 | 4.768 | 1.00 | 70.65 | C |
| ATOM | 883 | O | MET | A | 111 | 104.177 | 24.273 | 4.118 | 1.00 | 69.63 | O |
| ATOM | 884 | N | ALA | A | 112 | 102.993 | 25.989 | 4.972 | 1.00 | 72.73 | N |
| ATOM | 885 | CA | ALA | A | 112 | 103.926 | 27.013 | 4.511 | 1.00 | 73.22 | C |
| ATOM | 886 | CB | ALA | A | 112 | 103.182 | 28.138 | 3.816 | 1.00 | 74.60 | C |
| ATOM | 887 | C | ALA | A | 112 | 104.715 | 27.550 | 5.699 | 1.00 | 75.00 | C |
| ATOM | 888 | O | ALA | A | 112 | 104.319 | 27.354 | 6.849 | 1.00 | 74.54 | O |
| ATOM | 889 | N | ILE | A | 113 | 105.829 | 28.223 | 5.417 | 1.00 | 75.76 | N |
| ATOM | 890 | CA | ILE | A | 113 | 106.684 | 28.781 | 6.467 | 1.00 | 75.57 | C |
| ATOM | 891 | CB | ILE | A | 113 | 107.799 | 27.787 | 6.871 | 1.00 | 74.30 | C |
| ATOM | 892 | CG1 | ILE | A | 113 | 108.525 | 28.264 | 8.132 | 1.00 | 72.52 | C |
| ATOM | 893 | CD1 | ILE | A | 113 | 109.418 | 27.209 | 8.747 | 1.00 | 71.73 | C |
| ATOM | 894 | CG2 | ILE | A | 113 | 108.787 | 27.567 | 5.729 | 1.00 | 74.42 | C |
| ATOM | 895 | C | ILE | A | 113 | 107.318 | 30.108 | 6.057 | 1.00 | 78.06 | C |
| ATOM | 896 | O | ILE | A | 113 | 107.704 | 30.299 | 4.903 | 1.00 | 78.43 | O |
| ATOM | 897 | N | ALA | A | 114 | 107.418 | 31.017 | 7.023 | 1.00 | 81.36 | N |
| ATOM | 898 | CA | ALA | A | 114 | 108.054 | 32.312 | 6.827 | 1.00 | 82.37 | C |
| ATOM | 899 | CB | ALA | A | 114 | 107.010 | 33.411 | 6.755 | 1.00 | 82.04 | C |
| ATOM | 900 | C | ALA | A | 114 | 109.006 | 32.554 | 7.992 | 1.00 | 84.16 | C |
| ATOM | 901 | O | ALA | A | 114 | 108.574 | 32.630 | 9.144 | 1.00 | 85.37 | O |
| ATOM | 902 | N | TYR | A | 115 | 110.297 | 32.668 | 7.688 | 1.00 | 84.64 | N |
| ATOM | 903 | CA | TYR | A | 115 | 111.326 | 32.873 | 8.709 | 1.00 | 84.66 | C |
| ATOM | 904 | CB | TYR | A | 115 | 112.710 | 32.503 | 8.165 | 1.00 | 82.76 | C |
| ATOM | 905 | CG | TYR | A | 115 | 112.967 | 31.012 | 8.120 | 1.00 | 81.09 | C |
| ATOM | 906 | CD1 | TYR | A | 115 | 114.000 | 30.445 | 8.856 | 1.00 | 80.03 | C |
| ATOM | 907 | CE1 | TYR | A | 115 | 114.239 | 29.082 | 8.820 | 1.00 | 79.47 | C |
| ATOM | 908 | CZ | TYR | A | 115 | 113.434 | 28.265 | 8.053 | 1.00 | 77.13 | C |
| ATOM | 909 | OH | TYR | A | 115 | 113.668 | 26.915 | 8.019 | 1.00 | 73.14 | O |
| ATOM | 910 | CE2 | TYR | A | 115 | 112.398 | 28.800 | 7.319 | 1.00 | 79.12 | C |
| ATOM | 911 | CD2 | TYR | A | 115 | 112.167 | 30.165 | 7.358 | 1.00 | 81.10 | C |
| ATOM | 912 | C | TYR | A | 115 | 111.304 | 34.313 | 9.219 | 1.00 | 86.07 | C |
| ATOM | 913 | O | TYR | A | 115 | 112.121 | 35.147 | 8.821 | 1.00 | 85.81 | O |
| ATOM | 914 | N | GLU | A | 116 | 110.346 | 34.586 | 10.100 | 1.00 | 87.27 | N |
| ATOM | 915 | CA | GLU | A | 116 | 110.166 | 35.905 | 10.685 | 1.00 | 91.13 | C |
| ATOM | 916 | CB | GLU | A | 116 | 109.296 | 36.769 | 9.766 | 1.00 | 93.22 | C |
| ATOM | 917 | CG | GLU | A | 116 | 108.943 | 38.140 | 10.326 | 1.00 | 97.30 | C |
| ATOM | 918 | CD | GLU | A | 116 | 110.159 | 39.000 | 10.616 | 1.00 | 101.54 | C |
| ATOM | 919 | OE1 | GLU | A | 116 | 111.055 | 39.082 | 9.748 | 1.00 | 107.29 | O |
| ATOM | 920 | OE2 | GLU | A | 116 | 110.211 | 39.609 | 11.706 | 1.00 | 102.79 | O |
| ATOM | 921 | C | GLU | A | 116 | 109.524 | 35.765 | 12.065 | 1.00 | 93.80 | C |
| ATOM | 922 | O | GLU | A | 116 | 108.479 | 35.132 | 12.210 | 1.00 | 92.53 | O |
| ATOM | 923 | N | SER | A | 117 | 110.159 | 36.358 | 13.071 | 1.00 | 97.53 | N |
| ATOM | 924 | CA | SER | A | 117 | 109.664 | 36.303 | 14.448 | 1.00 | 97.24 | C |
| ATOM | 925 | CB | SER | A | 117 | 110.806 | 36.585 | 15.435 | 1.00 | 100.11 | C |
| ATOM | 926 | OG | SER | A | 117 | 111.643 | 37.633 | 14.972 | 1.00 | 105.40 | O |
| ATOM | 927 | C | SER | A | 117 | 108.489 | 37.255 | 14.694 | 1.00 | 95.90 | C |
| ATOM | 928 | O | SER | A | 117 | 107.653 | 36.990 | 15.558 | 1.00 | 94.55 | O |
| ATOM | 929 | N | ASP | A | 118 | 108.431 | 38.358 | 13.946 | 1.00 | 96.01 | N |
| ATOM | 930 | CA | ASP | A | 118 | 107.349 | 39.340 | 14.078 | 1.00 | 97.66 | C |
| ATOM | 931 | CB | ASP | A | 118 | 107.911 | 40.765 | 13.984 | 1.00 | 99.77 | C |
| ATOM | 932 | CG | ASP | A | 118 | 106.853 | 41.842 | 14.212 | 1.00 | 101.82 | C |
| ATOM | 933 | OD1 | ASP | A | 118 | 105.746 | 41.528 | 14.704 | 1.00 | 103.70 | O |
| ATOM | 934 | OD2 | ASP | A | 118 | 107.141 | 43.018 | 13.904 | 1.00 | 104.07 | O |
| ATOM | 935 | C | ASP | A | 118 | 106.288 | 39.116 | 12.999 | 1.00 | 97.06 | C |
| ATOM | 936 | O | ASP | A | 118 | 106.510 | 39.421 | 11.829 | 1.00 | 98.49 | O |
| ATOM | 937 | N | VAL | A | 119 | 105.130 | 38.604 | 13.408 | 1.00 | 96.94 | N |
| ATOM | 938 | CA | VAL | A | 119 | 104.030 | 38.294 | 12.483 | 1.00 | 97.43 | C |
| ATOM | 939 | CB | VAL | A | 119 | 102.807 | 37.719 | 13.242 | 1.00 | 99.06 | C |
| ATOM | 940 | CG1 | VAL | A | 119 | 102.134 | 38.786 | 14.098 | 1.00 | 99.23 | C |
| ATOM | 941 | CG2 | VAL | A | 119 | 101.810 | 37.093 | 12.276 | 1.00 | 100.11 | C |
| ATOM | 942 | C | VAL | A | 119 | 103.581 | 39.492 | 11.637 | 1.00 | 96.97 | C |
| ATOM | 943 | O | VAL | A | 119 | 103.140 | 39.322 | 10.500 | 1.00 | 96.72 | O |
| ATOM | 944 | N | ARG | A | 120 | 103.705 | 40.695 | 12.193 | 1.00 | 97.92 | N |
| ATOM | 945 | CA | ARG | A | 120 | 103.284 | 41.919 | 11.508 | 1.00 | 99.51 | C |
| ATOM | 946 | CB | ARG | A | 120 | 103.353 | 43.115 | 12.466 | 1.00 | 103.11 | C |
| ATOM | 947 | CG | ARG | A | 120 | 102.446 | 43.008 | 13.686 | 1.00 | 105.30 | C |
| ATOM | 948 | CD | ARG | A | 120 | 100.973 | 43.100 | 13.315 | 1.00 | 105.70 | C |
| ATOM | 949 | NE | ARG | A | 120 | 100.099 | 42.829 | 14.454 | 1.00 | 105.98 | N |
| ATOM | 950 | CZ | ARG | A | 120 | 98.769 | 42.875 | 14.415 | 1.00 | 106.94 | C |
| ATOM | 951 | NH1 | ARG | A | 120 | 98.134 | 43.189 | 13.290 | 1.00 | 107.36 | N |
| ATOM | 952 | NH2 | ARG | A | 120 | 98.066 | 42.610 | 15.511 | 1.00 | 108.34 | N |
| ATOM | 953 | C | ARG | A | 120 | 104.108 | 42.222 | 10.255 | 1.00 | 98.14 | C |
| ATOM | 954 | O | ARG | A | 120 | 103.621 | 42.887 | 9.341 | 1.00 | 98.42 | O |
| ATOM | 955 | N | LYS | A | 121 | 105.348 | 41.739 | 10.217 | 1.00 | 96.66 | N |
| ATOM | 956 | CA | LYS | A | 121 | 106.230 | 41.955 | 9.069 | 1.00 | 97.00 | C |
| ATOM | 957 | CB | LYS | A | 121 | 107.695 | 41.967 | 9.517 | 1.00 | 100.76 | C |
| ATOM | 958 | CG | LYS | A | 121 | 108.074 | 43.177 | 10.355 | 1.00 | 105.33 | C |
| ATOM | 959 | CD | LYS | A | 121 | 109.582 | 43.288 | 10.515 | 1.00 | 108.74 | C |
| ATOM | 960 | CE | LYS | A | 121 | 109.983 | 44.630 | 11.105 | 1.00 | 111.26 | C |
| ATOM | 961 | NZ | LYS | A | 121 | 111.458 | 44.828 | 11.062 | 1.00 | 114.12 | N |
| ATOM | 962 | C | LYS | A | 121 | 106.052 | 40.929 | 7.948 | 1.00 | 95.26 | C |
| ATOM | 963 | O | LYS | A | 121 | 106.583 | 41.120 | 6.854 | 1.00 | 97.97 | O |
| ATOM | 964 | N | ILE | A | 122 | 105.310 | 39.853 | 8.207 | 1.00 | 93.34 | N |
| ATOM | 965 | CA | ILE | A | 122 | 105.126 | 38.788 | 7.214 | 1.00 | 91.06 | C |
| ATOM | 966 | CB | ILE | A | 122 | 104.603 | 37.482 | 7.850 | 1.00 | 89.31 | C |
| ATOM | 967 | CG1 | ILE | A | 122 | 105.591 | 36.969 | 8.900 | 1.00 | 90.30 | C |
| ATOM | 968 | CD1 | ILE | A | 122 | 105.068 | 35.813 | 9.722 | 1.00 | 90.61 | C |
| ATOM | 969 | CG2 | ILE | A | 122 | 104.398 | 36.411 | 6.784 | 1.00 | 87.46 | C |
| ATOM | 970 | C | ILE | A | 122 | 104.167 | 39.215 | 6.107 | 1.00 | 91.89 | C |
| ATOM | 971 | O | ILE | A | 122 | 103.072 | 39.711 | 6.378 | 1.00 | 92.60 | O |
| ATOM | 972 | N | LYS | A | 123 | 104.593 | 39.000 | 4.864 | 1.00 | 93.11 | N |
| ATOM | 973 | CA | LYS | A | 123 | 103.795 | 39.318 | 3.684 | 1.00 | 95.03 | C |
| ATOM | 974 | CB | LYS | A | 123 | 104.570 | 40.271 | 2.776 | 1.00 | 98.86 | C |
| ATOM | 975 | CG | LYS | A | 123 | 104.764 | 41.651 | 3.385 | 1.00 | 102.64 | C |
| ATOM | 976 | CD | LYS | A | 123 | 105.857 | 42.439 | 2.684 | 1.00 | 105.78 | C |
| ATOM | 977 | CE | LYS | A | 123 | 106.091 | 43.771 | 3.377 | 1.00 | 108.87 | C |
| ATOM | 978 | NZ | LYS | A | 123 | 107.317 | 44.453 | 2.879 | 1.00 | 111.78 | N |
| ATOM | 979 | C | LYS | A | 123 | 103.439 | 38.027 | 2.945 | 1.00 | 92.62 | C |
| ATOM | 980 | O | LYS | A | 123 | 104.125 | 37.017 | 3.103 | 1.00 | 91.55 | O |
| ATOM | 981 | N | PRO | A | 124 | 102.368 | 38.053 | 2.129 | 1.00 | 89.62 | N |
| ATOM | 982 | CA | PRO | A | 124 | 101.920 | 36.841 | 1.431 | 1.00 | 89.50 | C |
| ATOM | 983 | CB | PRO | A | 124 | 100.746 | 37.338 | 0.581 | 1.00 | 91.29 | C |
| ATOM | 984 | CG | PRO | A | 124 | 100.301 | 38.592 | 1.241 | 1.00 | 90.38 | C |
| ATOM | 985 | CD | PRO | A | 124 | 101.553 | 39.220 | 1.756 | 1.00 | 89.45 | C |
| ATOM | 986 | C | PRO | A | 124 | 102.984 | 36.211 | 0.536 | 1.00 | 89.46 | C |
| ATOM | 987 | O | PRO | A | 124 | 102.998 | 34.994 | 0.358 | 1.00 | 89.19 | O |
| ATOM | 988 | N | GLU | A | 125 | 103.867 | 37.041 | −0.011 | 1.00 | 89.63 | N |
| ATOM | 989 | CA | GLU | A | 125 | 104.938 | 36.569 | −0.881 | 1.00 | 89.62 | C |
| ATOM | 990 | CB | GLU | A | 125 | 105.574 | 37.735 | −1.655 | 1.00 | 95.09 | C |
| ATOM | 991 | CG | GLU | A | 125 | 104.612 | 38.588 | −2.484 | 1.00 | 99.97 | C |
| ATOM | 992 | CD | GLU | A | 125 | 103.940 | 39.715 | −1.704 | 1.00 | 102.20 | C |
| ATOM | 993 | OE1 | GLU | A | 125 | 104.465 | 40.128 | −0.647 | 1.00 | 102.51 | O |
| ATOM | 994 | OE2 | GLU | A | 125 | 102.882 | 40.202 | −2.161 | 1.00 | 103.21 | O |
| ATOM | 995 | C | GLU | A | 125 | 106.033 | 35.846 | −0.096 | 1.00 | 86.13 | C |
| ATOM | 996 | O | GLU | A | 125 | 106.828 | 35.119 | −0.683 | 1.00 | 82.74 | O |
| ATOM | 997 | N | ASP | A | 126 | 106.077 | 36.053 | 1.221 | 1.00 | 86.04 | N |
| ATOM | 998 | CA | ASP | A | 126 | 107.130 | 35.479 | 2.067 | 1.00 | 83.80 | C |
| ATOM | 999 | CB | ASP | A | 126 | 107.259 | 36.269 | 3.375 | 1.00 | 86.37 | C |
| ATOM | 1000 | CG | ASP | A | 126 | 107.607 | 37.735 | 3.148 | 1.00 | 89.43 | C |
| ATOM | 1001 | OD1 | ASP | A | 126 | 108.014 | 38.095 | 2.023 | 1.00 | 90.53 | O |
| ATOM | 1002 | OD2 | ASP | A | 126 | 107.479 | 38.529 | 4.105 | 1.00 | 91.56 | O |
| ATOM | 1003 | C | ASP | A | 126 | 106.937 | 33.997 | 2.385 | 1.00 | 79.71 | C |
| ATOM | 1004 | O | ASP | A | 126 | 107.919 | 33.277 | 2.575 | 1.00 | 76.75 | O |
| ATOM | 1005 | N | PHE | A | 127 | 105.687 | 33.539 | 2.440 | 1.00 | 77.69 | N |
| ATOM | 1006 | CA | PHE | A | 127 | 105.406 | 32.127 | 2.739 | 1.00 | 76.03 | C |
| ATOM | 1007 | CB | PHE | A | 127 | 103.907 | 31.876 | 2.959 | 1.00 | 74.67 | C |
| ATOM | 1008 | CG | PHE | A | 127 | 103.461 | 32.070 | 4.377 | 1.00 | 74.73 | C |
| ATOM | 1009 | CD1 | PHE | A | 127 | 103.999 | 31.292 | 5.392 | 1.00 | 75.69 | C |
| ATOM | 1010 | CE1 | PHE | A | 127 | 103.590 | 31.452 | 6.703 | 1.00 | 75.83 | C |
| ATOM | 1011 | CZ | PHE | A | 127 | 102.619 | 32.386 | 7.014 | 1.00 | 75.24 | C |
| ATOM | 1012 | CE2 | PHE | A | 127 | 102.064 | 33.159 | 6.012 | 1.00 | 75.56 | C |
| ATOM | 1013 | CD2 | PHE | A | 127 | 102.481 | 32.998 | 4.700 | 1.00 | 74.80 | C |
| ATOM | 1014 | C | PHE | A | 127 | 105.929 | 31.164 | 1.670 | 1.00 | 76.28 | C |
| ATOM | 1015 | O | PHE | A | 127 | 105.753 | 31.386 | 0.471 | 1.00 | 74.14 | O |
| ATOM | 1016 | N | THR | A | 128 | 106.567 | 30.093 | 2.139 | 1.00 | 75.69 | N |
| ATOM | 1017 | CA | THR | A | 128 | 107.117 | 29.044 | 1.291 | 1.00 | 74.38 | C |
| ATOM | 1018 | CB | THR | A | 128 | 108.643 | 28.953 | 1.456 | 1.00 | 74.05 | C |
| ATOM | 1019 | OG1 | THR | A | 128 | 109.244 | 30.200 | 1.090 | 1.00 | 74.56 | O |
| ATOM | 1020 | CG2 | THR | A | 128 | 109.220 | 27.834 | 0.598 | 1.00 | 74.60 | C |
| ATOM | 1021 | C | THR | A | 128 | 106.526 | 27.704 | 1.708 | 1.00 | 74.11 | C |
| ATOM | 1022 | O | THR | A | 128 | 106.618 | 27.328 | 2.875 | 1.00 | 74.98 | O |
| ATOM | 1023 | N | VAL | A | 129 | 105.926 | 26.987 | 0.761 | 1.00 | 72.30 | N |
| ATOM | 1024 | CA | VAL | A | 129 | 105.366 | 25.665 | 1.040 | 1.00 | 70.81 | C |
| ATOM | 1025 | CB | VAL | A | 129 | 104.408 | 25.200 | −0.075 | 1.00 | 69.00 | C |
| ATOM | 1026 | CG1 | VAL | A | 129 | 103.927 | 23.778 | 0.179 | 1.00 | 68.01 | C |
| ATOM | 1027 | CG2 | VAL | A | 129 | 103.226 | 26.152 | −0.189 | 1.00 | 69.56 | C |
| ATOM | 1028 | C | VAL | A | 129 | 106.525 | 24.682 | 1.177 | 1.00 | 72.26 | C |
| ATOM | 1029 | O | VAL | A | 129 | 107.254 | 24.448 | 0.211 | 1.00 | 74.48 | O |
| ATOM | 1030 | N | PHE | A | 130 | 106.701 | 24.129 | 2.378 | 1.00 | 71.94 | N |
| ATOM | 1031 | CA | PHE | A | 130 | 107.815 | 23.214 | 2.656 | 1.00 | 71.73 | C |
| ATOM | 1032 | CB | PHE | A | 130 | 108.617 | 23.687 | 3.876 | 1.00 | 73.22 | C |
| ATOM | 1033 | CG | PHE | A | 130 | 107.899 | 23.525 | 5.185 | 1.00 | 73.63 | C |
| ATOM | 1034 | CD1 | PHE | A | 130 | 107.111 | 24.548 | 5.692 | 1.00 | 74.85 | C |
| ATOM | 1035 | CE1 | PHE | A | 130 | 106.451 | 24.402 | 6.902 | 1.00 | 76.17 | C |
| ATOM | 1036 | CZ | PHE | A | 130 | 106.577 | 23.226 | 7.620 | 1.00 | 75.83 | C |
| ATOM | 1037 | CE2 | PHE | A | 130 | 107.363 | 22.200 | 7.125 | 1.00 | 75.44 | C |
| ATOM | 1038 | CD2 | PHE | A | 130 | 108.022 | 22.354 | 5.918 | 1.00 | 74.02 | C |
| ATOM | 1039 | C | PHE | A | 130 | 107.387 | 21.760 | 2.844 | 1.00 | 71.33 | C |
| ATOM | 1040 | O | PHE | A | 130 | 108.229 | 20.862 | 2.806 | 1.00 | 70.69 | O |
| ATOM | 1041 | N | SER | A | 131 | 106.094 | 21.525 | 3.053 | 1.00 | 70.85 | N |
| ATOM | 1042 | CA | SER | A | 131 | 105.587 | 20.165 | 3.209 | 1.00 | 71.30 | C |
| ATOM | 1043 | CB | SER | A | 131 | 105.675 | 19.720 | 4.669 | 1.00 | 73.51 | C |
| ATOM | 1044 | OG | SER | A | 131 | 105.301 | 18.360 | 4.805 | 1.00 | 74.55 | O |
| ATOM | 1045 | C | SER | A | 131 | 104.149 | 20.060 | 2.719 | 1.00 | 69.79 | C |
| ATOM | 1046 | O | SER | A | 131 | 103.352 | 20.980 | 2.901 | 1.00 | 67.79 | O |
| ATOM | 1047 | N | VAL | A | 132 | 103.834 | 18.924 | 2.103 | 1.00 | 70.57 | N |
| ATOM | 1048 | CA | VAL | A | 132 | 102.519 | 18.674 | 1.518 | 1.00 | 70.58 | C |
| ATOM | 1049 | CB | VAL | A | 132 | 102.561 | 18.867 | −0.020 | 1.00 | 69.36 | C |
| ATOM | 1050 | CG1 | VAL | A | 132 | 101.234 | 18.487 | −0.664 | 1.00 | 69.12 | C |
| ATOM | 1051 | CG2 | VAL | A | 132 | 102.931 | 20.302 | −0.365 | 1.00 | 69.14 | C |
| ATOM | 1052 | C | VAL | A | 132 | 102.070 | 17.248 | 1.819 | 1.00 | 70.20 | C |
| ATOM | 1053 | O | VAL | A | 132 | 102.897 | 16.343 | 1.946 | 1.00 | 72.95 | O |
| ATOM | 1054 | N | ASN | A | 133 | 100.760 | 17.067 | 1.957 | 1.00 | 68.16 | N |
| ATOM | 1055 | CA | ASN | A | 133 | 100.165 | 15.743 | 2.071 | 1.00 | 70.49 | C |
| ATOM | 1056 | CB | ASN | A | 133 | 99.778 | 15.417 | 3.514 | 1.00 | 72.09 | C |
| ATOM | 1057 | CG | ASN | A | 133 | 99.396 | 13.956 | 3.699 | 1.00 | 72.52 | C |
| ATOM | 1058 | OD1 | ASN | A | 133 | 98.600 | 13.412 | 2.932 | 1.00 | 72.44 | O |
| ATOM | 1059 | ND2 | ASN | A | 133 | 99.962 | 13.316 | 4.724 | 1.00 | 74.04 | N |
| ATOM | 1060 | C | ASN | A | 133 | 98.955 | 15.720 | 1.148 | 1.00 | 72.68 | C |
| ATOM | 1061 | O | ASN | A | 133 | 97.956 | 16.379 | 1.414 | 1.00 | 73.03 | O |
| ATOM | 1062 | N | GLN | A | 134 | 99.057 | 14.969 | 0.056 | 1.00 | 74.63 | N |
| ATOM | 1063 | CA | GLN | A | 134 | 98.015 | 14.954 | −0.969 | 1.00 | 74.97 | C |
| ATOM | 1064 | CB | GLN | A | 134 | 98.548 | 14.351 | −2.269 | 1.00 | 76.21 | C |
| ATOM | 1065 | CG | GLN | A | 134 | 99.637 | 15.191 | −2.909 | 1.00 | 77.57 | C |
| ATOM | 1066 | CD | GLN | A | 134 | 100.043 | 14.681 | −4.275 | 1.00 | 80.24 | C |
| ATOM | 1067 | OE1 | GLN | A | 134 | 99.592 | 13.623 | −4.718 | 1.00 | 82.97 | O |
| ATOM | 1068 | NE2 | GLN | A | 134 | 100.902 | 15.434 | −4.952 | 1.00 | 81.02 | N |
| ATOM | 1069 | C | GLN | A | 134 | 96.732 | 14.247 | −0.554 | 1.00 | 74.40 | C |
| ATOM | 1070 | O | GLN | A | 134 | 95.684 | 14.473 | −1.157 | 1.00 | 80.04 | O |
| ATOM | 1071 | N | THR | A | 135 | 96.812 | 13.384 | 0.452 | 1.00 | 74.31 | N |
| ATOM | 1072 | CA | THR | A | 135 | 95.631 | 12.701 | 0.965 | 1.00 | 77.03 | C |
| ATOM | 1073 | CB | THR | A | 135 | 95.939 | 11.235 | 1.327 | 1.00 | 75.26 | C |
| ATOM | 1074 | OG1 | THR | A | 135 | 96.708 | 10.634 | 0.280 | 1.00 | 74.35 | O |
| ATOM | 1075 | CG2 | THR | A | 135 | 94.652 | 10.444 | 1.522 | 1.00 | 75.12 | C |
| ATOM | 1076 | C | THR | A | 135 | 95.165 | 13.486 | 2.190 | 1.00 | 82.39 | C |
| ATOM | 1077 | O | THR | A | 135 | 95.116 | 12.959 | 3.307 | 1.00 | 82.05 | O |
| ATOM | 1078 | N | CYS | A | 136 | 94.824 | 14.756 | 1.972 | 1.00 | 84.83 | N |
| ATOM | 1079 | CA | CYS | A | 136 | 94.499 | 15.645 | 3.077 | 1.00 | 86.52 | C |
| ATOM | 1080 | CB | CYS | A | 136 | 95.175 | 16.990 | 2.920 | 1.00 | 86.38 | C |
| ATOM | 1081 | SG | CYS | A | 136 | 94.984 | 17.997 | 4.401 | 1.00 | 83.29 | S |
| ATOM | 1082 | C | CYS | A | 136 | 93.022 | 15.895 | 3.277 | 1.00 | 90.46 | C |
| ATOM | 1083 | O | CYS | A | 136 | 92.241 | 15.963 | 2.326 | 1.00 | 85.99 | O |
| ATOM | 1084 | N | VAL | A | 137 | 92.688 | 16.096 | 4.548 | 1.00 | 94.32 | N |
| ATOM | 1085 | CA | VAL | A | 137 | 91.324 | 16.244 | 5.012 | 1.00 | 87.90 | C |
| ATOM | 1086 | CB | VAL | A | 137 | 90.656 | 17.503 | 4.443 | 1.00 | 88.76 | C |
| ATOM | 1087 | CG1 | VAL | A | 137 | 89.238 | 17.622 | 4.957 | 1.00 | 94.38 | C |
| ATOM | 1088 | CG2 | VAL | A | 137 | 91.460 | 18.733 | 4.840 | 1.00 | 86.92 | C |
| ATOM | 1089 | C | VAL | A | 137 | 90.590 | 14.932 | 4.727 | 1.00 | 85.62 | C |
| ATOM | 1090 | O | VAL | A | 137 | 89.553 | 14.884 | 4.065 | 1.00 | 85.99 | O |
| ATOM | 1091 | N | TRP | A | 138 | 91.219 | 13.862 | 5.207 | 1.00 | 81.26 | N |
| ATOM | 1092 | CA | TRP | A | 138 | 90.633 | 12.533 | 5.250 | 1.00 | 80.06 | C |
| ATOM | 1093 | CB | TRP | A | 138 | 91.206 | 11.622 | 4.161 | 1.00 | 78.20 | C |
| ATOM | 1094 | CG | TRP | A | 138 | 90.416 | 10.367 | 4.023 | 1.00 | 74.91 | C |
| ATOM | 1095 | CD1 | TRP | A | 138 | 90.803 | 9.111 | 4.369 | 1.00 | 74.39 | C |
| ATOM | 1096 | NE1 | TRP | A | 138 | 89.790 | 8.218 | 4.115 | 1.00 | 76.74 | N |
| ATOM | 1097 | CE2 | TRP | A | 138 | 88.714 | 8.900 | 3.612 | 1.00 | 76.01 | C |
| ATOM | 1098 | CD2 | TRP | A | 138 | 89.073 | 10.259 | 3.545 | 1.00 | 74.70 | C |
| ATOM | 1099 | CE3 | TRP | A | 138 | 88.142 | 11.181 | 3.055 | 1.00 | 74.89 | C |
| ATOM | 1100 | CZ3 | TRP | A | 138 | 86.896 | 10.722 | 2.655 | 1.00 | 76.10 | C |
| ATOM | 1101 | CH2 | TRP | A | 138 | 86.567 | 9.361 | 2.733 | 1.00 | 77.44 | C |
| ATOM | 1102 | CZ2 | TRP | A | 138 | 87.460 | 8.436 | 3.207 | 1.00 | 77.43 | C |
| ATOM | 1103 | C | TRP | A | 138 | 90.904 | 11.958 | 6.643 | 1.00 | 80.76 | C |
| ATOM | 1104 | O | TRP | A | 138 | 89.991 | 11.459 | 7.303 | 1.00 | 79.36 | O |
| ATOM | 1105 | N | TYR | A | 139 | 92.166 | 12.034 | 7.071 | 1.00 | 82.51 | N |
| ATOM | 1106 | CA | TYR | A | 139 | 92.581 | 11.601 | 8.401 | 1.00 | 81.56 | C |
| ATOM | 1107 | CB | TYR | A | 139 | 93.916 | 10.861 | 8.339 | 1.00 | 80.89 | C |
| ATOM | 1108 | CG | TYR | A | 139 | 94.018 | 9.811 | 7.257 | 1.00 | 80.17 | C |
| ATOM | 1109 | CD1 | TYR | A | 139 | 93.330 | 8.606 | 7.359 | 1.00 | 80.27 | C |
| ATOM | 1110 | CE1 | TYR | A | 139 | 93.437 | 7.637 | 6.374 | 1.00 | 82.65 | C |
| ATOM | 1111 | CZ | TYR | A | 139 | 94.248 | 7.865 | 5.272 | 1.00 | 81.80 | C |
| ATOM | 1112 | OH | TYR | A | 139 | 94.359 | 6.909 | 4.287 | 1.00 | 81.17 | O |
| ATOM | 1113 | CE2 | TYR | A | 139 | 94.946 | 9.050 | 5.155 | 1.00 | 80.06 | C |
| ATOM | 1114 | CD2 | TYR | A | 139 | 94.832 | 10.011 | 6.144 | 1.00 | 79.78 | C |
| ATOM | 1115 | C | TYR | A | 139 | 92.750 | 12.826 | 9.296 | 1.00 | 81.52 | C |
| ATOM | 1116 | O | TYR | A | 139 | 93.288 | 13.844 | 8.860 | 1.00 | 82.26 | O |
| ATOM | 1117 | N | ARG | A | 140 | 92.303 | 12.717 | 10.546 | 1.00 | 81.61 | N |
| ATOM | 1118 | CA | ARG | A | 140 | 92.458 | 13.798 | 11.522 | 1.00 | 80.81 | C |
| ATOM | 1119 | CB | ARG | A | 140 | 91.682 | 13.503 | 12.802 | 1.00 | 80.92 | C |
| ATOM | 1120 | CG | ARG | A | 140 | 91.965 | 14.496 | 13.916 | 1.00 | 81.79 | C |
| ATOM | 1121 | CD | ARG | A | 140 | 90.903 | 14.448 | 14.997 | 1.00 | 85.18 | C |
| ATOM | 1122 | NE | ARG | A | 140 | 90.964 | 13.227 | 15.803 | 1.00 | 86.47 | N |
| ATOM | 1123 | CZ | ARG | A | 140 | 91.715 | 13.062 | 16.892 | 1.00 | 84.38 | C |
| ATOM | 1124 | NH1 | ARG | A | 140 | 91.672 | 11.905 | 17.545 | 1.00 | 85.39 | N |
| ATOM | 1125 | NH2 | ARG | A | 140 | 92.506 | 14.034 | 17.339 | 1.00 | 83.04 | N |
| ATOM | 1126 | C | ARG | A | 140 | 93.927 | 14.003 | 11.857 | 1.00 | 79.50 | C |
| ATOM | 1127 | O | ARG | A | 140 | 94.402 | 15.133 | 11.904 | 1.00 | 79.49 | O |
| ATOM | 1128 | N | PHE | A | 141 | 94.631 | 12.903 | 12.110 | 1.00 | 78.69 | N |
| ATOM | 1129 | CA | PHE | A | 141 | 96.063 | 12.951 | 12.362 | 1.00 | 78.46 | C |
| ATOM | 1130 | CB | PHE | A | 141 | 96.504 | 11.793 | 13.256 | 1.00 | 79.59 | C |
| ATOM | 1131 | CG | PHE | A | 141 | 95.953 | 11.862 | 14.650 | 1.00 | 80.07 | C |
| ATOM | 1132 | CD1 | PHE | A | 141 | 96.507 | 12.721 | 15.588 | 1.00 | 80.90 | C |
| ATOM | 1133 | CE1 | PHE | A | 141 | 96.005 | 12.785 | 16.875 | 1.00 | 83.01 | C |
| ATOM | 1134 | CZ | PHE | A | 141 | 94.941 | 11.980 | 17.240 | 1.00 | 82.71 | C |
| ATOM | 1135 | CE2 | PHE | A | 141 | 94.382 | 11.119 | 16.314 | 1.00 | 82.15 | C |
| ATOM | 1136 | CD2 | PHE | A | 141 | 94.888 | 11.062 | 15.029 | 1.00 | 81.39 | C |
| ATOM | 1137 | C | PHE | A | 141 | 96.782 | 12.898 | 11.022 | 1.00 | 77.36 | C |
| ATOM | 1138 | O | PHE | A | 141 | 97.094 | 11.821 | 10.518 | 1.00 | 77.71 | O |
| ATOM | 1139 | N | THR | A | 142 | 97.023 | 14.072 | 10.447 | 1.00 | 76.07 | N |
| ATOM | 1140 | CA | THR | A | 142 | 97.678 | 14.188 | 9.150 | 1.00 | 76.31 | C |
| ATOM | 1141 | CB | THR | A | 142 | 96.990 | 15.256 | 8.278 | 1.00 | 76.58 | C |
| ATOM | 1142 | OG1 | THR | A | 142 | 95.643 | 14.850 | 7.996 | 1.00 | 77.62 | O |
| ATOM | 1143 | CG2 | THR | A | 142 | 97.737 | 15.450 | 6.964 | 1.00 | 77.49 | C |
| ATOM | 1144 | C | THR | A | 142 | 99.147 | 14.545 | 9.334 | 1.00 | 76.47 | C |
| ATOM | 1145 | O | THR | A | 142 | 99.472 | 15.623 | 9.829 | 1.00 | 76.20 | O |
| ATOM | 1146 | N | ASP | A | 143 | 100.027 | 13.633 | 8.927 | 1.00 | 77.81 | N |
| ATOM | 1147 | CA | ASP | A | 143 | 101.468 | 13.842 | 9.038 | 1.00 | 78.46 | C |
| ATOM | 1148 | CB | ASP | A | 143 | 102.212 | 12.500 | 9.032 | 1.00 | 81.65 | C |
| ATOM | 1149 | CG | ASP | A | 143 | 101.879 | 11.633 | 10.239 | 1.00 | 83.58 | C |
| ATOM | 1150 | OD1 | ASP | A | 143 | 101.038 | 12.040 | 11.070 | 1.00 | 85.51 | O |
| ATOM | 1151 | OD2 | ASP | A | 143 | 102.467 | 10.539 | 10.354 | 1.00 | 84.80 | O |
| ATOM | 1152 | C | ASP | A | 143 | 101.993 | 14.713 | 7.903 | 1.00 | 74.97 | C |
| ATOM | 1153 | O | ASP | A | 143 | 101.479 | 14.670 | 6.785 | 1.00 | 77.14 | O |
| ATOM | 1154 | N | PHE | A | 144 | 103.021 | 15.498 | 8.210 | 1.00 | 71.79 | N |
| ATOM | 1155 | CA | PHE | A | 144 | 103.688 | 16.348 | 7.237 | 1.00 | 71.00 | C |
| ATOM | 1156 | CB | PHE | A | 144 | 103.379 | 17.820 | 7.507 | 1.00 | 69.59 | C |
| ATOM | 1157 | CG | PHE | A | 144 | 101.965 | 18.210 | 7.182 | 1.00 | 69.05 | C |
| ATOM | 1158 | CD1 | PHE | A | 144 | 100.983 | 18.185 | 8.158 | 1.00 | 69.25 | C |
| ATOM | 1159 | CE1 | PHE | A | 144 | 99.678 | 18.543 | 7.860 | 1.00 | 69.31 | C |
| ATOM | 1160 | CZ | PHE | A | 144 | 99.341 | 18.931 | 6.577 | 1.00 | 67.47 | C |
| ATOM | 1161 | CE2 | PHE | A | 144 | 100.311 | 18.962 | 5.593 | 1.00 | 67.42 | C |
| ATOM | 1162 | CD2 | PHE | A | 144 | 101.614 | 18.600 | 5.896 | 1.00 | 68.29 | C |
| ATOM | 1163 | C | PHE | A | 144 | 105.190 | 16.100 | 7.311 | 1.00 | 73.12 | C |
| ATOM | 1164 | O | PHE | A | 144 | 105.820 | 16.379 | 8.328 | 1.00 | 71.85 | O |
| ATOM | 1165 | N | GLN | A | 145 | 105.755 | 15.570 | 6.227 | 1.00 | 78.05 | N |
| ATOM | 1166 | CA | GLN | A | 145 | 107.182 | 15.262 | 6.170 | 1.00 | 77.96 | C |
| ATOM | 1167 | CB | GLN | A | 145 | 107.504 | 14.360 | 4.975 | 1.00 | 80.55 | C |
| ATOM | 1168 | CG | GLN | A | 145 | 106.704 | 13.064 | 4.915 | 1.00 | 82.81 | C |
| ATOM | 1169 | CD | GLN | A | 145 | 106.892 | 12.190 | 6.142 | 1.00 | 85.81 | C |
| ATOM | 1170 | OE1 | GLN | A | 145 | 107.994 | 12.091 | 6.688 | 1.00 | 87.14 | O |
| ATOM | 1171 | NE2 | GLN | A | 145 | 105.816 | 11.537 | 6.575 | 1.00 | 87.99 | N |
| ATOM | 1172 | C | GLN | A | 145 | 107.965 | 16.559 | 6.056 | 1.00 | 76.90 | C |
| ATOM | 1173 | O | GLN | A | 145 | 107.702 | 17.368 | 5.168 | 1.00 | 77.08 | O |
| ATOM | 1174 | N | VAL | A | 146 | 108.920 | 16.754 | 6.961 | 1.00 | 75.97 | N |
| ATOM | 1175 | CA | VAL | A | 146 | 109.732 | 17.964 | 6.982 | 1.00 | 75.59 | C |
| ATOM | 1176 | CB | VAL | A | 146 | 110.064 | 18.389 | 8.425 | 1.00 | 75.15 | C |
| ATOM | 1177 | CG1 | VAL | A | 146 | 110.804 | 19.720 | 8.442 | 1.00 | 75.39 | C |
| ATOM | 1178 | CG2 | VAL | A | 146 | 108.795 | 18.483 | 9.259 | 1.00 | 74.27 | C |
| ATOM | 1179 | C | VAL | A | 146 | 111.029 | 17.690 | 6.231 | 1.00 | 76.30 | C |
| ATOM | 1180 | O | VAL | A | 146 | 111.608 | 16.613 | 6.385 | 1.00 | 76.96 | O |
| ATOM | 1181 | N | PRO | A | 147 | 111.487 | 18.649 | 5.404 | 1.00 | 78.68 | N |
| ATOM | 1182 | CA | PRO | A | 147 | 112.755 | 18.431 | 4.701 | 1.00 | 81.84 | C |
| ATOM | 1183 | CB | PRO | A | 147 | 112.881 | 19.659 | 3.787 | 1.00 | 80.60 | C |
| ATOM | 1184 | CG | PRO | A | 147 | 111.513 | 20.234 | 3.693 | 1.00 | 79.84 | C |
| ATOM | 1185 | CD | PRO | A | 147 | 110.823 | 19.894 | 4.977 | 1.00 | 79.51 | C |
| ATOM | 1186 | C | PRO | A | 147 | 113.941 | 18.347 | 5.661 | 1.00 | 86.08 | C |
| ATOM | 1187 | O | PRO | A | 147 | 113.938 | 19.000 | 6.706 | 1.00 | 86.09 | O |
| ATOM | 1188 | N | GLU | A | 148 | 114.939 | 17.545 | 5.307 | 1.00 | 91.96 | N |
| ATOM | 1189 | CA | GLU | A | 148 | 116.120 | 17.368 | 6.150 | 1.00 | 97.64 | C |
| ATOM | 1190 | CB | GLU | A | 148 | 116.968 | 16.200 | 5.636 | 1.00 | 101.74 | C |
| ATOM | 1191 | CG | GLU | A | 148 | 118.213 | 15.911 | 6.463 | 1.00 | 107.11 | C |
| ATOM | 1192 | CD | GLU | A | 148 | 119.079 | 14.827 | 5.853 | 1.00 | 113.41 | C |
| ATOM | 1193 | OE1 | GLU | A | 148 | 118.525 | 13.806 | 5.393 | 1.00 | 120.31 | O |
| ATOM | 1194 | OE2 | GLU | A | 148 | 120.318 | 14.993 | 5.842 | 1.00 | 118.08 | O |
| ATOM | 1195 | C | GLU | A | 148 | 116.971 | 18.637 | 6.223 | 1.00 | 98.80 | C |
| ATOM | 1196 | O | GLU | A | 148 | 117.497 | 18.973 | 7.284 | 1.00 | 99.60 | O |
| ATOM | 1197 | N | ARG | A | 149 | 117.092 | 19.345 | 5.102 | 1.00 | 100.31 | N |
| ATOM | 1198 | CA | ARG | A | 149 | 117.944 | 20.535 | 5.026 | 1.00 | 102.05 | C |
| ATOM | 1199 | CB | ARG | A | 149 | 118.496 | 20.691 | 3.602 | 1.00 | 109.61 | C |
| ATOM | 1200 | CG | ARG | A | 149 | 119.367 | 19.534 | 3.140 | 1.00 | 116.76 | C |
| ATOM | 1201 | CD | ARG | A | 149 | 119.858 | 19.745 | 1.716 | 1.00 | 125.71 | C |
| ATOM | 1202 | NE | ARG | A | 149 | 120.677 | 18.626 | 1.239 | 1.00 | 133.21 | N |
| ATOM | 1203 | CZ | ARG | A | 149 | 121.985 | 18.479 | 1.460 | 1.00 | 133.30 | C |
| ATOM | 1204 | NH1 | ARG | A | 149 | 122.666 | 19.375 | 2.173 | 1.00 | 135.95 | N |
| ATOM | 1205 | NH2 | ARG | A | 149 | 122.619 | 17.417 | 0.970 | 1.00 | 129.76 | N |
| ATOM | 1206 | C | ARG | A | 149 | 117.261 | 21.839 | 5.471 | 1.00 | 96.88 | C |
| ATOM | 1207 | O | ARG | A | 149 | 117.676 | 22.925 | 5.055 | 1.00 | 95.81 | O |
| ATOM | 1208 | N | MET | A | 150 | 116.233 | 21.746 | 6.316 | 1.00 | 90.98 | N |
| ATOM | 1209 | CA | MET | A | 150 | 115.560 | 22.943 | 6.824 | 1.00 | 89.04 | C |
| ATOM | 1210 | CB | MET | A | 150 | 114.235 | 22.594 | 7.512 | 1.00 | 88.98 | C |
| ATOM | 1211 | CG | MET | A | 150 | 113.137 | 22.104 | 6.582 | 1.00 | 89.61 | C |
| ATOM | 1212 | SD | MET | A | 150 | 112.456 | 23.348 | 5.461 | 1.00 | 90.13 | S |
| ATOM | 1213 | CE | MET | A | 150 | 111.604 | 24.446 | 6.585 | 1.00 | 88.70 | C |
| ATOM | 1214 | C | MET | A | 150 | 116.472 | 23.659 | 7.818 | 1.00 | 88.28 | C |
| ATOM | 1215 | O | MET | A | 150 | 116.906 | 23.054 | 8.797 | 1.00 | 89.76 | O |
| ATOM | 1216 | N | PRO | A | 151 | 116.768 | 24.949 | 7.570 | 1.00 | 87.50 | N |
| ATOM | 1217 | CA | PRO | A | 151 | 117.634 | 25.717 | 8.464 | 1.00 | 88.75 | C |
| ATOM | 1218 | CB | PRO | A | 151 | 117.986 | 26.949 | 7.630 | 1.00 | 87.34 | C |
| ATOM | 1219 | CG | PRO | A | 151 | 116.809 | 27.138 | 6.747 | 1.00 | 87.38 | C |
| ATOM | 1220 | CD | PRO | A | 151 | 116.305 | 25.760 | 6.430 | 1.00 | 87.98 | C |
| ATOM | 1221 | C | PRO | A | 151 | 116.922 | 26.131 | 9.753 | 1.00 | 90.96 | C |
| ATOM | 1222 | O | PRO | A | 151 | 115.692 | 26.071 | 9.816 | 1.00 | 91.16 | O |
| ATOM | 1223 | N | PRO | A | 152 | 117.688 | 26.558 | 10.775 | 1.00 | 91.63 | N |
| ATOM | 1224 | CA | PRO | A | 152 | 117.083 | 26.959 | 12.038 | 1.00 | 91.53 | C |
| ATOM | 1225 | CB | PRO | A | 152 | 118.278 | 27.020 | 12.986 | 1.00 | 92.45 | C |
| ATOM | 1226 | CG | PRO | A | 152 | 119.412 | 27.417 | 12.110 | 1.00 | 92.47 | C |
| ATOM | 1227 | CD | PRO | A | 152 | 119.150 | 26.757 | 10.786 | 1.00 | 92.22 | C |
| ATOM | 1228 | C | PRO | A | 152 | 116.417 | 28.324 | 11.934 | 1.00 | 91.29 | C |
| ATOM | 1229 | O | PRO | A | 152 | 116.912 | 29.203 | 11.227 | 1.00 | 87.61 | O |
| ATOM | 1230 | N | CYS | A | 153 | 115.303 | 28.490 | 12.638 | 1.00 | 95.22 | N |
| ATOM | 1231 | CA | CYS | A | 153 | 114.562 | 29.747 | 12.624 | 1.00 | 100.58 | C |
| ATOM | 1232 | CB | CYS | A | 153 | 113.097 | 29.506 | 13.001 | 1.00 | 105.48 | C |
| ATOM | 1233 | SG | CYS | A | 153 | 112.131 | 28.865 | 11.616 | 1.00 | 110.80 | S |
| ATOM | 1234 | C | CYS | A | 153 | 115.197 | 30.783 | 13.546 | 1.00 | 99.11 | C |
| ATOM | 1235 | O | CYS | A | 153 | 115.962 | 30.426 | 14.440 | 1.00 | 100.03 | O |
| ATOM | 1236 | N | PRO | A | 154 | 114.883 | 32.075 | 13.328 | 1.00 | 99.89 | N |
| ATOM | 1237 | CA | PRO | A | 154 | 115.453 | 33.146 | 14.152 | 1.00 | 103.15 | C |
| ATOM | 1238 | CB | PRO | A | 154 | 114.838 | 34.426 | 13.556 | 1.00 | 102.45 | C |
| ATOM | 1239 | CG | PRO | A | 154 | 113.722 | 33.979 | 12.679 | 1.00 | 101.24 | C |
| ATOM | 1240 | CD | PRO | A | 154 | 114.096 | 32.614 | 12.206 | 1.00 | 101.08 | C |
| ATOM | 1241 | C | PRO | A | 154 | 115.136 | 33.030 | 15.655 | 1.00 | 106.30 | C |
| ATOM | 1242 | O | PRO | A | 154 | 114.291 | 32.223 | 16.043 | 1.00 | 103.59 | O |
| ATOM | 1243 | N | PRO | A | 155 | 115.820 | 33.835 | 16.494 | 1.00 | 109.83 | N |
| ATOM | 1244 | CA | PRO | A | 155 | 115.691 | 33.817 | 17.956 | 1.00 | 112.03 | C |
| ATOM | 1245 | CB | PRO | A | 155 | 116.332 | 35.143 | 18.369 | 1.00 | 114.04 | C |
| ATOM | 1246 | CG | PRO | A | 155 | 117.382 | 35.371 | 17.341 | 1.00 | 113.75 | C |
| ATOM | 1247 | CD | PRO | A | 155 | 116.841 | 34.809 | 16.056 | 1.00 | 111.64 | C |
| ATOM | 1248 | C | PRO | A | 155 | 114.265 | 33.733 | 18.509 | 1.00 | 111.44 | C |
| ATOM | 1249 | O | PRO | A | 155 | 114.016 | 32.954 | 19.434 | 1.00 | 112.99 | O |
| ATOM | 1250 | N | GLY | A | 156 | 113.345 | 34.518 | 17.951 | 1.00 | 108.40 | N |
| ATOM | 1251 | CA | GLY | A | 156 | 111.954 | 34.533 | 18.417 | 1.00 | 107.86 | C |
| ATOM | 1252 | C | GLY | A | 156 | 111.032 | 33.536 | 17.729 | 1.00 | 106.15 | C |
| ATOM | 1253 | O | GLY | A | 156 | 109.811 | 33.688 | 17.773 | 1.00 | 101.23 | O |
| ATOM | 1254 | N | GLY | A | 157 | 111.609 | 32.511 | 17.105 | 1.00 | 106.69 | N |
| ATOM | 1255 | CA | GLY | A | 157 | 110.837 | 31.524 | 16.355 | 1.00 | 104.57 | C |
| ATOM | 1256 | C | GLY | A | 157 | 110.410 | 32.056 | 14.998 | 1.00 | 101.90 | C |
| ATOM | 1257 | O | GLY | A | 157 | 110.762 | 33.174 | 14.618 | 1.00 | 104.11 | O |
| ATOM | 1258 | N | CYS | A | 158 | 109.652 | 31.247 | 14.266 | 1.00 | 98.35 | N |
| ATOM | 1259 | CA | CYS | A | 158 | 109.128 | 31.629 | 12.955 | 1.00 | 98.02 | C |
| ATOM | 1260 | CB | CYS | A | 158 | 109.898 | 30.915 | 11.840 | 1.00 | 99.92 | C |
| ATOM | 1261 | SG | CYS | A | 158 | 110.210 | 29.171 | 12.180 | 1.00 | 111.88 | S |
| ATOM | 1262 | C | CYS | A | 158 | 107.637 | 31.313 | 12.888 | 1.00 | 93.44 | C |
| ATOM | 1263 | O | CYS | A | 158 | 107.072 | 30.765 | 13.837 | 1.00 | 94.72 | O |
| ATOM | 1264 | N | HIS | A | 159 | 107.001 | 31.678 | 11.779 | 1.00 | 89.00 | N |
| ATOM | 1265 | CA | HIS | A | 159 | 105.567 | 31.461 | 11.600 | 1.00 | 86.06 | C |
| ATOM | 1266 | CB | HIS | A | 159 | 104.862 | 32.797 | 11.384 | 1.00 | 86.11 | C |
| ATOM | 1267 | CG | HIS | A | 159 | 104.714 | 33.602 | 12.634 | 1.00 | 85.86 | C |
| ATOM | 1268 | ND1 | HIS | A | 159 | 105.771 | 34.260 | 13.224 | 1.00 | 85.50 | N |
| ATOM | 1269 | CE1 | HIS | A | 159 | 105.349 | 34.875 | 14.314 | 1.00 | 87.12 | C |
| ATOM | 1270 | NE2 | HIS | A | 159 | 104.056 | 34.642 | 14.451 | 1.00 | 85.96 | N |
| ATOM | 1271 | CD2 | HIS | A | 159 | 103.634 | 33.850 | 13.412 | 1.00 | 86.34 | C |
| ATOM | 1272 | C | HIS | A | 159 | 105.258 | 30.514 | 10.447 | 1.00 | 83.23 | C |
| ATOM | 1273 | O | HIS | A | 159 | 105.976 | 30.474 | 9.448 | 1.00 | 81.54 | O |
| ATOM | 1274 | N | CYS | A | 160 | 104.185 | 29.746 | 10.617 | 1.00 | 80.95 | N |
| ATOM | 1275 | CA | CYS | A | 160 | 103.723 | 28.792 | 9.620 | 1.00 | 80.06 | C |
| ATOM | 1276 | CB | CYS | A | 160 | 103.953 | 27.364 | 10.107 | 1.00 | 82.18 | C |
| ATOM | 1277 | SG | CYS | A | 160 | 105.680 | 26.861 | 10.200 | 1.00 | 88.40 | S |
| ATOM | 1278 | C | CYS | A | 160 | 102.240 | 28.985 | 9.361 | 1.00 | 78.26 | C |
| ATOM | 1279 | O | CYS | A | 160 | 101.560 | 29.715 | 10.086 | 1.00 | 76.85 | O |
| ATOM | 1280 | N | ALA | A | 161 | 101.743 | 28.316 | 8.325 | 1.00 | 76.08 | N |
| ATOM | 1281 | CA | ALA | A | 161 | 100.328 | 28.373 | 7.982 | 1.00 | 73.79 | C |
| ATOM | 1282 | CB | ALA | A | 161 | 100.049 | 29.573 | 7.094 | 1.00 | 73.37 | C |
| ATOM | 1283 | C | ALA | A | 161 | 99.888 | 27.088 | 7.295 | 1.00 | 71.21 | C |
| ATOM | 1284 | O | ALA | A | 161 | 100.631 | 26.515 | 6.500 | 1.00 | 72.15 | O |
| ATOM | 1285 | N | TRP | A | 162 | 98.683 | 26.637 | 7.625 | 1.00 | 69.66 | N |
| ATOM | 1286 | CA | TRP | A | 162 | 98.103 | 25.446 | 7.022 | 1.00 | 68.18 | C |
| ATOM | 1287 | CB | TRP | A | 162 | 97.434 | 24.575 | 8.083 | 1.00 | 66.71 | C |
| ATOM | 1288 | CG | TRP | A | 162 | 96.645 | 23.429 | 7.522 | 1.00 | 64.24 | C |
| ATOM | 1289 | CD1 | TRP | A | 162 | 97.124 | 22.215 | 7.134 | 1.00 | 63.87 | C |
| ATOM | 1290 | NE1 | TRP | A | 162 | 96.099 | 21.421 | 6.686 | 1.00 | 62.98 | N |
| ATOM | 1291 | CE2 | TRP | A | 162 | 94.926 | 22.119 | 6.779 | 1.00 | 62.55 | C |
| ATOM | 1292 | CD2 | TRP | A | 162 | 95.232 | 23.390 | 7.304 | 1.00 | 62.40 | C |
| ATOM | 1293 | CE3 | TRP | A | 162 | 94.194 | 24.305 | 7.505 | 1.00 | 63.45 | C |
| ATOM | 1294 | CZ3 | TRP | A | 162 | 92.898 | 23.927 | 7.176 | 1.00 | 64.28 | C |
| ATOM | 1295 | CH2 | TRP | A | 162 | 92.626 | 22.655 | 6.651 | 1.00 | 64.06 | C |
| ATOM | 1296 | CZ2 | TRP | A | 162 | 93.625 | 21.739 | 6.448 | 1.00 | 63.81 | C |
| ATOM | 1297 | C | TRP | A | 162 | 97.083 | 25.886 | 5.988 | 1.00 | 67.79 | C |
| ATOM | 1298 | O | TRP | A | 162 | 96.149 | 26.617 | 6.309 | 1.00 | 67.02 | O |
| ATOM | 1299 | N | PHE | A | 163 | 97.266 | 25.439 | 4.750 | 1.00 | 67.99 | N |
| ATOM | 1300 | CA | PHE | A | 163 | 96.366 | 25.793 | 3.657 | 1.00 | 65.65 | C |
| ATOM | 1301 | CB | PHE | A | 163 | 97.140 | 26.477 | 2.538 | 1.00 | 62.67 | C |
| ATOM | 1302 | CG | PHE | A | 163 | 97.753 | 27.784 | 2.935 | 1.00 | 59.96 | C |
| ATOM | 1303 | CD1 | PHE | A | 163 | 99.060 | 27.843 | 3.393 | 1.00 | 57.85 | C |
| ATOM | 1304 | CE1 | PHE | A | 163 | 99.628 | 29.055 | 3.745 | 1.00 | 58.02 | C |
| ATOM | 1305 | CZ | PHE | A | 163 | 98.889 | 30.224 | 3.644 | 1.00 | 58.37 | C |
| ATOM | 1306 | CE2 | PHE | A | 163 | 97.584 | 30.177 | 3.188 | 1.00 | 58.49 | C |
| ATOM | 1307 | CD2 | PHE | A | 163 | 97.023 | 28.961 | 2.836 | 1.00 | 59.66 | C |
| ATOM | 1308 | C | PHE | A | 163 | 95.669 | 24.561 | 3.102 | 1.00 | 65.65 | C |
| ATOM | 1309 | O | PHE | A | 163 | 96.204 | 23.462 | 3.175 | 1.00 | 64.46 | O |
| ATOM | 1310 | N | TRP | A | 164 | 94.484 | 24.762 | 2.530 | 1.00 | 66.84 | N |
| ATOM | 1311 | CA | TRP | A | 164 | 93.709 | 23.672 | 1.948 | 1.00 | 66.24 | C |
| ATOM | 1312 | CB | TRP | A | 164 | 93.004 | 22.883 | 3.057 | 1.00 | 66.14 | C |
| ATOM | 1313 | CG | TRP | A | 164 | 92.024 | 21.853 | 2.559 | 1.00 | 64.96 | C |
| ATOM | 1314 | CD1 | TRP | A | 164 | 92.256 | 20.891 | 1.626 | 1.00 | 64.45 | C |
| ATOM | 1315 | NE1 | TRP | A | 164 | 91.129 | 20.124 | 1.444 | 1.00 | 64.11 | N |
| ATOM | 1316 | CE2 | TRP | A | 164 | 90.145 | 20.573 | 2.282 | 1.00 | 62.02 | C |
| ATOM | 1317 | CD2 | TRP | A | 164 | 90.674 | 21.661 | 3.006 | 1.00 | 64.79 | C |
| ATOM | 1318 | CE3 | TRP | A | 164 | 89.861 | 22.304 | 3.948 | 1.00 | 66.51 | C |
| ATOM | 1319 | CZ3 | TRP | A | 164 | 88.559 | 21.847 | 4.128 | 1.00 | 64.78 | C |
| ATOM | 1320 | CH2 | TRP | A | 164 | 88.065 | 20.761 | 3.389 | 1.00 | 61.88 | C |
| ATOM | 1321 | CZ2 | TRP | A | 164 | 88.842 | 20.112 | 2.467 | 1.00 | 60.20 | C |
| ATOM | 1322 | C | TRP | A | 164 | 92.684 | 24.174 | 0.937 | 1.00 | 65.82 | C |
| ATOM | 1323 | O | TRP | A | 164 | 91.957 | 25.128 | 1.202 | 1.00 | 65.09 | O |
| ATOM | 1324 | N | ILE | A | 165 | 92.654 | 23.531 | −0.227 | 1.00 | 68.37 | N |
| ATOM | 1325 | CA | ILE | A | 165 | 91.652 | 23.797 | −1.261 | 1.00 | 68.83 | C |
| ATOM | 1326 | CB | ILE | A | 165 | 92.247 | 24.510 | −2.490 | 1.00 | 67.90 | C |
| ATOM | 1327 | CG1 | ILE | A | 165 | 92.757 | 25.897 | −2.091 | 1.00 | 67.98 | C |
| ATOM | 1328 | CD1 | ILE | A | 165 | 93.456 | 26.654 | −3.199 | 1.00 | 69.23 | C |
| ATOM | 1329 | CG2 | ILE | A | 165 | 91.195 | 24.624 | −3.588 | 1.00 | 69.24 | C |
| ATOM | 1330 | C | ILE | A | 165 | 91.067 | 22.451 | −1.664 | 1.00 | 68.08 | C |
| ATOM | 1331 | O | ILE | A | 165 | 91.808 | 21.515 | −1.962 | 1.00 | 69.04 | O |
| ATOM | 1332 | N | HIS | A | 166 | 89.742 | 22.366 | −1.682 | 1.00 | 68.83 | N |
| ATOM | 1333 | CA | HIS | A | 166 | 89.053 | 21.102 | −1.927 | 1.00 | 71.85 | C |
| ATOM | 1334 | CB | HIS | A | 166 | 87.972 | 20.887 | −0.864 | 1.00 | 71.51 | C |
| ATOM | 1335 | CG | HIS | A | 166 | 86.856 | 21.878 | −0.925 | 1.00 | 72.05 | C |
| ATOM | 1336 | ND1 | HIS | A | 166 | 85.731 | 21.680 | −1.693 | 1.00 | 75.81 | N |
| ATOM | 1337 | CE1 | HIS | A | 166 | 84.918 | 22.711 | −1.555 | 1.00 | 76.33 | C |
| ATOM | 1338 | NE2 | HIS | A | 166 | 85.478 | 23.574 | −0.727 | 1.00 | 75.99 | N |
| ATOM | 1339 | CD2 | HIS | A | 166 | 86.693 | 23.077 | −0.320 | 1.00 | 74.28 | C |
| ATOM | 1340 | C | HIS | A | 166 | 88.446 | 21.000 | −3.331 | 1.00 | 73.81 | C |
| ATOM | 1341 | O | HIS | A | 166 | 88.442 | 21.962 | −4.096 | 1.00 | 72.40 | O |
| ATOM | 1342 | N | SER | A | 167 | 87.926 | 19.817 | −3.644 | 1.00 | 75.65 | N |
| ATOM | 1343 | CA | SER | A | 167 | 87.361 | 19.532 | −4.953 | 1.00 | 78.55 | C |
| ATOM | 1344 | CB | SER | A | 167 | 87.006 | 18.047 | −5.042 | 1.00 | 82.08 | C |
| ATOM | 1345 | OG | SER | A | 167 | 86.547 | 17.699 | −6.338 | 1.00 | 86.01 | O |
| ATOM | 1346 | C | SER | A | 167 | 86.119 | 20.376 | −5.238 | 1.00 | 78.52 | C |
| ATOM | 1347 | O | SER | A | 167 | 85.413 | 20.761 | −4.307 | 1.00 | 79.61 | O |
| ATOM | 1348 | N | PRO | A | 168 | 85.852 | 20.677 | −6.526 | 1.00 | 78.75 | N |
| ATOM | 1349 | CA | PRO | A | 168 | 84.641 | 21.425 | −6.881 | 1.00 | 78.88 | C |
| ATOM | 1350 | CB | PRO | A | 168 | 84.899 | 21.874 | −8.326 | 1.00 | 78.02 | C |
| ATOM | 1351 | CG | PRO | A | 168 | 86.326 | 21.574 | −8.603 | 1.00 | 78.49 | C |
| ATOM | 1352 | CD | PRO | A | 168 | 86.703 | 20.447 | −7.704 | 1.00 | 78.14 | C |
| ATOM | 1353 | C | PRO | A | 168 | 83.367 | 20.588 | −6.835 | 1.00 | 80.35 | C |
| ATOM | 1354 | O | PRO | A | 168 | 82.273 | 21.146 | −6.742 | 1.00 | 81.61 | O |
| ATOM | 1355 | N | ASP | A | 169 | 83.510 | 19.266 | −6.896 | 1.00 | 80.49 | N |
| ATOM | 1356 | CA | ASP | A | 169 | 82.354 | 18.370 | −6.936 | 1.00 | 82.01 | C |
| ATOM | 1357 | CB | ASP | A | 169 | 82.756 | 17.006 | −7.512 | 1.00 | 84.94 | C |
| ATOM | 1358 | CG | ASP | A | 169 | 83.697 | 16.240 | −6.606 | 1.00 | 87.76 | C |
| ATOM | 1359 | OD1 | ASP | A | 169 | 84.189 | 16.833 | −5.623 | 1.00 | 92.11 | O |
| ATOM | 1360 | OD2 | ASP | A | 169 | 83.948 | 15.046 | −6.877 | 1.00 | 90.58 | O |
| ATOM | 1361 | C | ASP | A | 169 | 81.649 | 18.172 | −5.591 | 1.00 | 80.62 | C |
| ATOM | 1362 | O | ASP | A | 169 | 80.693 | 17.404 | −5.515 | 1.00 | 80.75 | O |
| ATOM | 1363 | N | SER | A | 170 | 82.113 | 18.839 | −4.536 | 1.00 | 81.20 | N |
| ATOM | 1364 | CA | SER | A | 170 | 81.455 | 18.742 | −3.232 | 1.00 | 83.20 | C |
| ATOM | 1365 | CB | SER | A | 170 | 82.153 | 17.712 | −2.346 | 1.00 | 82.93 | C |
| ATOM | 1366 | OG | SER | A | 170 | 81.443 | 17.534 | −1.132 | 1.00 | 81.13 | O |
| ATOM | 1367 | C | SER | A | 170 | 81.408 | 20.090 | −2.522 | 1.00 | 83.08 | C |
| ATOM | 1368 | O | SER | A | 170 | 82.436 | 20.610 | −2.093 | 1.00 | 82.18 | O |
| ATOM | 1369 | N | GLY | A | 171 | 80.206 | 20.652 | −2.424 | 1.00 | 84.35 | N |
| ATOM | 1370 | CA | GLY | A | 171 | 79.982 | 21.907 | −1.718 | 1.00 | 86.21 | C |
| ATOM | 1371 | C | GLY | A | 171 | 80.536 | 23.145 | −2.399 | 1.00 | 85.87 | C |
| ATOM | 1372 | O | GLY | A | 171 | 81.128 | 23.072 | −3.480 | 1.00 | 85.11 | O |
| ATOM | 1373 | N | GLY | A | 172 | 80.336 | 24.287 | −1.745 | 1.00 | 86.38 | N |
| ATOM | 1374 | CA | GLY | A | 172 | 80.805 | 25.574 | −2.247 | 1.00 | 84.86 | C |
| ATOM | 1375 | C | GLY | A | 172 | 82.315 | 25.662 | −2.207 | 1.00 | 81.45 | C |
| ATOM | 1376 | O | GLY | A | 172 | 82.956 | 25.046 | −1.357 | 1.00 | 81.76 | O |
| ATOM | 1377 | N | GLU | A | 173 | 82.880 | 26.442 | −3.120 | 1.00 | 79.46 | N |
| ATOM | 1378 | CA | GLU | A | 173 | 84.325 | 26.534 | −3.252 | 1.00 | 80.95 | C |
| ATOM | 1379 | CB | GLU | A | 173 | 84.712 | 26.590 | −4.732 | 1.00 | 82.70 | C |
| ATOM | 1380 | CG | GLU | A | 173 | 86.160 | 26.234 | −5.000 | 1.00 | 83.57 | C |
| ATOM | 1381 | CD | GLU | A | 173 | 86.508 | 24.803 | −4.637 | 1.00 | 86.05 | C |
| ATOM | 1382 | OE1 | GLU | A | 173 | 85.904 | 23.875 | −5.209 | 1.00 | 88.87 | O |
| ATOM | 1383 | OE2 | GLU | A | 173 | 87.404 | 24.608 | −3.790 | 1.00 | 89.20 | O |
| ATOM | 1384 | C | GLU | A | 173 | 84.884 | 27.736 | −2.490 | 1.00 | 80.15 | C |
| ATOM | 1385 | O | GLU | A | 173 | 84.255 | 28.792 | −2.417 | 1.00 | 77.51 | O |
| ATOM | 1386 | N | GLN | A | 174 | 86.078 | 27.551 | −1.931 | 1.00 | 81.16 | N |
| ATOM | 1387 | CA | GLN | A | 174 | 86.731 | 28.546 | −1.085 | 1.00 | 78.07 | C |
| ATOM | 1388 | CB | GLN | A | 174 | 85.999 | 28.623 | 0.256 | 1.00 | 76.04 | C |
| ATOM | 1389 | CG | GLN | A | 174 | 85.976 | 27.296 | 1.025 | 1.00 | 75.95 | C |
| ATOM | 1390 | CD | GLN | A | 174 | 84.636 | 26.569 | 1.067 | 1.00 | 75.19 | C |
| ATOM | 1391 | OE1 | GLN | A | 174 | 84.600 | 25.370 | 1.346 | 1.00 | 78.72 | O |
| ATOM | 1392 | NE2 | GLN | A | 174 | 83.537 | 27.274 | 0.816 | 1.00 | 75.03 | N |
| ATOM | 1393 | C | GLN | A | 174 | 88.182 | 28.127 | −0.826 | 1.00 | 78.53 | C |
| ATOM | 1394 | O | GLN | A | 174 | 88.661 | 27.152 | −1.412 | 1.00 | 83.04 | O |
| ATOM | 1395 | N | ILE | A | 175 | 88.873 | 28.855 | 0.051 | 1.00 | 76.75 | N |
| ATOM | 1396 | CA | ILE | A | 175 | 90.186 | 28.425 | 0.545 | 1.00 | 76.57 | C |
| ATOM | 1397 | CB | ILE | A | 175 | 91.345 | 29.321 | 0.057 | 1.00 | 76.05 | C |
| ATOM | 1398 | CG1 | ILE | A | 175 | 91.363 | 30.668 | 0.788 | 1.00 | 75.36 | C |
| ATOM | 1399 | CD1 | ILE | A | 175 | 92.640 | 31.458 | 0.585 | 1.00 | 74.21 | C |
| ATOM | 1400 | CG2 | ILE | A | 175 | 91.255 | 29.508 | −1.451 | 1.00 | 77.02 | C |
| ATOM | 1401 | C | ILE | A | 175 | 90.161 | 28.378 | 2.070 | 1.00 | 76.43 | C |
| ATOM | 1402 | O | ILE | A | 175 | 89.246 | 28.910 | 2.700 | 1.00 | 77.44 | O |
| ATOM | 1403 | N | TYR | A | 176 | 91.169 | 27.731 | 2.649 | 1.00 | 77.61 | N |
| ATOM | 1404 | CA | TYR | A | 176 | 91.292 | 27.601 | 4.101 | 1.00 | 77.05 | C |
| ATOM | 1405 | CB | TYR | A | 176 | 90.988 | 26.167 | 4.531 | 1.00 | 76.53 | C |
| ATOM | 1406 | CG | TYR | A | 176 | 89.539 | 25.780 | 4.364 | 1.00 | 76.89 | C |
| ATOM | 1407 | CD1 | TYR | A | 176 | 89.042 | 25.385 | 3.130 | 1.00 | 77.36 | C |
| ATOM | 1408 | CE1 | TYR | A | 176 | 87.714 | 25.029 | 2.976 | 1.00 | 77.51 | C |
| ATOM | 1409 | CZ | TYR | A | 176 | 86.865 | 25.067 | 4.063 | 1.00 | 76.94 | C |
| ATOM | 1410 | OH | TYR | A | 176 | 85.547 | 24.719 | 3.911 | 1.00 | 79.19 | O |
| ATOM | 1411 | CE2 | TYR | A | 176 | 87.332 | 25.456 | 5.299 | 1.00 | 76.88 | C |
| ATOM | 1412 | CD2 | TYR | A | 176 | 88.663 | 25.809 | 5.445 | 1.00 | 78.44 | C |
| ATOM | 1413 | C | TYR | A | 176 | 92.696 | 27.988 | 4.546 | 1.00 | 77.51 | C |
| ATOM | 1414 | O | TYR | A | 176 | 93.678 | 27.575 | 3.932 | 1.00 | 75.08 | O |
| ATOM | 1415 | N | MET | A | 177 | 92.784 | 28.783 | 5.610 | 1.00 | 80.01 | N |
| ATOM | 1416 | CA | MET | A | 177 | 94.074 | 29.209 | 6.154 | 1.00 | 77.80 | C |
| ATOM | 1417 | CB | MET | A | 177 | 94.488 | 30.561 | 5.577 | 1.00 | 78.47 | C |
| ATOM | 1418 | CG | MET | A | 177 | 95.844 | 31.040 | 6.070 | 1.00 | 77.88 | C |
| ATOM | 1419 | SD | MET | A | 177 | 96.289 | 32.658 | 5.429 | 1.00 | 79.52 | S |
| ATOM | 1420 | CE | MET | A | 177 | 97.914 | 32.856 | 6.160 | 1.00 | 79.52 | C |
| ATOM | 1421 | C | MET | A | 177 | 94.028 | 29.309 | 7.674 | 1.00 | 74.45 | C |
| ATOM | 1422 | O | MET | A | 177 | 93.098 | 29.879 | 8.239 | 1.00 | 73.78 | O |
| ATOM | 1423 | N | ASN | A | 178 | 95.050 | 28.750 | 8.317 | 1.00 | 72.63 | N |
| ATOM | 1424 | CA | ASN | A | 178 | 95.213 | 28.808 | 9.762 | 1.00 | 68.48 | C |
| ATOM | 1425 | CB | ASN | A | 178 | 94.727 | 27.510 | 10.410 | 1.00 | 68.50 | C |
| ATOM | 1426 | CG | ASN | A | 178 | 93.215 | 27.425 | 10.498 | 1.00 | 71.24 | C |
| ATOM | 1427 | OD1 | ASN | A | 178 | 92.615 | 26.424 | 10.105 | 1.00 | 73.50 | O |
| ATOM | 1428 | ND2 | ASN | A | 178 | 92.588 | 28.473 | 11.026 | 1.00 | 72.32 | N |
| ATOM | 1429 | C | ASN | A | 178 | 96.680 | 29.037 | 10.096 | 1.00 | 68.03 | C |
| ATOM | 1430 | O | ASN | A | 178 | 97.489 | 28.114 | 10.006 | 1.00 | 67.06 | O |
| ATOM | 1431 | N | GLY | A | 179 | 97.022 | 30.268 | 10.467 | 1.00 | 69.28 | N |
| ATOM | 1432 | CA | GLY | A | 179 | 98.395 | 30.613 | 10.837 | 1.00 | 72.01 | C |
| ATOM | 1433 | C | GLY | A | 179 | 98.720 | 30.206 | 12.266 | 1.00 | 75.63 | C |
| ATOM | 1434 | O | GLY | A | 179 | 97.822 | 30.091 | 13.108 | 1.00 | 76.00 | O |
| ATOM | 1435 | N | PHE | A | 180 | 100.002 | 29.988 | 12.547 | 1.00 | 76.09 | N |
| ATOM | 1436 | CA | PHE | A | 180 | 100.427 | 29.579 | 13.883 | 1.00 | 77.15 | C |
| ATOM | 1437 | CB | PHE | A | 180 | 100.021 | 28.126 | 14.143 | 1.00 | 78.41 | C |
| ATOM | 1438 | CG | PHE | A | 180 | 100.700 | 27.136 | 13.248 | 1.00 | 79.99 | C |
| ATOM | 1439 | CD1 | PHE | A | 180 | 100.268 | 26.952 | 11.945 | 1.00 | 79.59 | C |
| ATOM | 1440 | CE1 | PHE | A | 180 | 100.890 | 26.037 | 11.122 | 1.00 | 77.76 | C |
| ATOM | 1441 | CZ | PHE | A | 180 | 101.948 | 25.286 | 11.600 | 1.00 | 76.93 | C |
| ATOM | 1442 | CE2 | PHE | A | 180 | 102.382 | 25.456 | 12.892 | 1.00 | 79.56 | C |
| ATOM | 1443 | CD2 | PHE | A | 180 | 101.759 | 26.375 | 13.714 | 1.00 | 80.71 | C |
| ATOM | 1444 | C | PHE | A | 180 | 101.930 | 29.735 | 14.095 | 1.00 | 79.15 | C |
| ATOM | 1445 | O | PHE | A | 180 | 102.698 | 29.831 | 13.136 | 1.00 | 78.34 | O |
| ATOM | 1446 | N | GLN | A | 181 | 102.337 | 29.759 | 15.363 | 1.00 | 81.63 | N |
| ATOM | 1447 | CA | GLN | A | 181 | 103.748 | 29.842 | 15.723 | 1.00 | 82.49 | C |
| ATOM | 1448 | CB | GLN | A | 181 | 103.922 | 30.335 | 17.160 | 1.00 | 85.63 | C |
| ATOM | 1449 | CG | GLN | A | 181 | 103.581 | 31.800 | 17.376 | 1.00 | 87.98 | C |
| ATOM | 1450 | CD | GLN | A | 181 | 103.922 | 32.281 | 18.779 | 1.00 | 87.06 | C |
| ATOM | 1451 | OE1 | GLN | A | 181 | 104.026 | 31.487 | 19.715 | 1.00 | 84.92 | O |
| ATOM | 1452 | NE2 | GLN | A | 181 | 104.095 | 33.590 | 18.928 | 1.00 | 86.83 | N |
| ATOM | 1453 | C | GLN | A | 181 | 104.379 | 28.467 | 15.588 | 1.00 | 82.49 | C |
| ATOM | 1454 | O | GLN | A | 181 | 103.909 | 27.505 | 16.197 | 1.00 | 81.59 | O |
| ATOM | 1455 | N | CYS | A | 182 | 105.438 | 28.377 | 14.789 | 1.00 | 82.33 | N |
| ATOM | 1456 | CA | CYS | A | 182 | 106.152 | 27.123 | 14.597 | 1.00 | 82.80 | C |
| ATOM | 1457 | CB | CYS | A | 182 | 105.926 | 26.586 | 13.189 | 1.00 | 85.08 | C |
| ATOM | 1458 | SG | CYS | A | 182 | 106.333 | 27.768 | 11.889 | 1.00 | 88.27 | S |
| ATOM | 1459 | C | CYS | A | 182 | 107.642 | 27.302 | 14.822 | 1.00 | 82.53 | C |
| ATOM | 1460 | O | CYS | A | 182 | 108.136 | 28.420 | 14.963 | 1.00 | 80.43 | O |
| ATOM | 1461 | N | ASN | A | 183 | 108.350 | 26.180 | 14.858 | 1.00 | 83.98 | N |
| ATOM | 1462 | CA | ASN | A | 183 | 109.789 | 26.178 | 15.038 | 1.00 | 87.53 | C |
| ATOM | 1463 | CB | ASN | A | 183 | 110.127 | 26.372 | 16.515 | 1.00 | 94.35 | C |
| ATOM | 1464 | CG | ASN | A | 183 | 111.599 | 26.621 | 16.752 | 1.00 | 99.90 | C |
| ATOM | 1465 | OD1 | ASN | A | 183 | 112.340 | 27.000 | 15.837 | 1.00 | 102.98 | O |
| ATOM | 1466 | ND2 | ASN | A | 183 | 112.035 | 26.415 | 17.987 | 1.00 | 102.09 | N |
| ATOM | 1467 | C | ASN | A | 183 | 110.364 | 24.867 | 14.518 | 1.00 | 86.07 | C |
| ATOM | 1468 | O | ASN | A | 183 | 109.675 | 23.846 | 14.509 | 1.00 | 84.50 | O |
| ATOM | 1469 | N | ILE | A | 184 | 111.621 | 24.899 | 14.084 | 1.00 | 85.52 | N |
| ATOM | 1470 | CA | ILE | A | 184 | 112.261 | 23.724 | 13.500 | 1.00 | 85.50 | C |
| ATOM | 1471 | CB | ILE | A | 184 | 112.902 | 24.067 | 12.145 | 1.00 | 86.78 | C |
| ATOM | 1472 | CG1 | ILE | A | 184 | 111.855 | 24.741 | 11.257 | 1.00 | 88.90 | C |
| ATOM | 1473 | CD1 | ILE | A | 184 | 112.291 | 24.944 | 9.829 | 1.00 | 91.71 | C |
| ATOM | 1474 | CG2 | ILE | A | 184 | 113.462 | 22.813 | 11.477 | 1.00 | 86.77 | C |
| ATOM | 1475 | C | ILE | A | 184 | 113.305 | 23.121 | 14.431 | 1.00 | 85.05 | C |
| ATOM | 1476 | O | ILE | A | 184 | 114.204 | 23.814 | 14.906 | 1.00 | 87.23 | O |
| ATOM | 1477 | N | THR | A | 185 | 113.172 | 21.819 | 14.673 | 1.00 | 84.65 | N |
| ATOM | 1478 | CA | THR | A | 185 | 114.090 | 21.070 | 15.519 | 1.00 | 84.52 | C |
| ATOM | 1479 | CB | THR | A | 185 | 113.324 | 20.290 | 16.607 | 1.00 | 83.63 | C |
| ATOM | 1480 | OG1 | THR | A | 185 | 112.565 | 19.231 | 16.010 | 1.00 | 82.31 | O |
| ATOM | 1481 | CG2 | THR | A | 185 | 112.382 | 21.212 | 17.373 | 1.00 | 82.88 | C |
| ATOM | 1482 | C | THR | A | 185 | 114.884 | 20.089 | 14.660 | 1.00 | 87.43 | C |
| ATOM | 1483 | O | THR | A | 185 | 114.461 | 19.731 | 13.562 | 1.00 | 92.86 | O |
| ATOM | 1484 | N | GLY | A | 186 | 116.033 | 19.654 | 15.163 | 1.00 | 89.40 | N |
| ATOM | 1485 | CA | GLY | A | 186 | 116.885 | 18.702 | 14.449 | 1.00 | 89.10 | C |
| ATOM | 1486 | C | GLY | A | 186 | 117.567 | 19.281 | 13.222 | 1.00 | 87.83 | C |
| ATOM | 1487 | O | GLY | A | 186 | 117.929 | 18.544 | 12.304 | 1.00 | 86.14 | O |
| ATOM | 1488 | N | SER | A | 187 | 117.760 | 20.599 | 13.215 | 1.00 | 87.16 | N |
| ATOM | 1489 | CA | SER | A | 187 | 118.376 | 21.288 | 12.084 | 1.00 | 87.75 | C |
| ATOM | 1490 | CB | SER | A | 187 | 118.233 | 22.803 | 12.249 | 1.00 | 86.93 | C |
| ATOM | 1491 | OG | SER | A | 187 | 118.824 | 23.495 | 11.163 | 1.00 | 87.00 | O |
| ATOM | 1492 | C | SER | A | 187 | 119.852 | 20.926 | 11.941 | 1.00 | 88.80 | C |
| ATOM | 1493 | O | SER | A | 187 | 120.620 | 21.057 | 12.892 | 1.00 | 91.38 | O |
| ATOM | 1494 | N | THR | A | 188 | 120.234 | 20.467 | 10.750 | 1.00 | 91.89 | N |
| ATOM | 1495 | CA | THR | A | 188 | 121.626 | 20.126 | 10.444 | 1.00 | 93.80 | C |
| ATOM | 1496 | CB | THR | A | 188 | 121.803 | 18.608 | 10.225 | 1.00 | 94.36 | C |
| ATOM | 1497 | OG1 | THR | A | 188 | 121.083 | 18.199 | 9.055 | 1.00 | 97.73 | O |
| ATOM | 1498 | CG2 | THR | A | 188 | 121.299 | 17.827 | 11.429 | 1.00 | 95.01 | C |
| ATOM | 1499 | C | THR | A | 188 | 122.085 | 20.876 | 9.193 | 1.00 | 95.01 | C |
| ATOM | 1500 | O | THR | A | 188 | 122.790 | 20.323 | 8.349 | 1.00 | 94.25 | O |
| ATOM | 1501 | N | SER | A | 189 | 121.677 | 22.139 | 9.084 | 1.00 | 97.50 | N |
| ATOM | 1502 | CA | SER | A | 189 | 122.010 | 22.974 | 7.930 | 1.00 | 98.32 | C |
| ATOM | 1503 | CB | SER | A | 189 | 121.191 | 22.547 | 6.711 | 1.00 | 97.46 | C |
| ATOM | 1504 | OG | SER | A | 189 | 121.468 | 23.375 | 5.596 | 1.00 | 96.80 | O |
| ATOM | 1505 | C | SER | A | 189 | 121.746 | 24.447 | 8.228 | 1.00 | 100.26 | C |
| ATOM | 1506 | O | SER | A | 189 | 120.976 | 24.773 | 9.128 | 1.00 | 100.80 | O |
| ATOM | 1507 | N | HIS | A | 190 | 122.402 | 25.327 | 7.475 | 1.00 | 100.89 | N |
| ATOM | 1508 | CA | HIS | A | 190 | 122.232 | 26.775 | 7.622 | 1.00 | 101.15 | C |
| ATOM | 1509 | CB | HIS | A | 190 | 123.489 | 27.394 | 8.244 | 1.00 | 104.71 | C |
| ATOM | 1510 | CG | HIS | A | 190 | 123.642 | 27.110 | 9.705 | 1.00 | 109.21 | C |
| ATOM | 1511 | ND1 | HIS | A | 190 | 124.242 | 25.965 | 10.184 | 1.00 | 110.75 | N |
| ATOM | 1512 | CE1 | HIS | A | 190 | 124.233 | 25.986 | 11.505 | 1.00 | 112.14 | C |
| ATOM | 1513 | NE2 | HIS | A | 190 | 123.651 | 27.104 | 11.900 | 1.00 | 113.10 | N |
| ATOM | 1514 | CD2 | HIS | A | 190 | 123.272 | 27.825 | 10.794 | 1.00 | 111.06 | C |
| ATOM | 1515 | C | HIS | A | 190 | 121.908 | 27.475 | 6.301 | 1.00 | 98.77 | C |
| ATOM | 1516 | O | HIS | A | 190 | 121.777 | 28.700 | 6.269 | 1.00 | 99.11 | O |
| ATOM | 1517 | N | VAL | A | 191 | 121.765 | 26.708 | 5.222 | 1.00 | 96.73 | N |
| ATOM | 1518 | CA | VAL | A | 191 | 121.453 | 27.279 | 3.913 | 1.00 | 92.97 | C |
| ATOM | 1519 | CB | VAL | A | 191 | 121.734 | 26.286 | 2.765 | 1.00 | 93.24 | C |
| ATOM | 1520 | CG1 | VAL | A | 191 | 121.399 | 26.919 | 1.420 | 1.00 | 94.47 | C |
| ATOM | 1521 | CG2 | VAL | A | 191 | 123.188 | 25.832 | 2.791 | 1.00 | 92.73 | C |
| ATOM | 1522 | C | VAL | A | 191 | 119.975 | 27.671 | 3.907 | 1.00 | 89.35 | C |
| ATOM | 1523 | O | VAL | A | 191 | 119.106 | 26.804 | 4.023 | 1.00 | 88.41 | O |
| ATOM | 1524 | N | PRO | A | 192 | 119.685 | 28.977 | 3.763 | 1.00 | 85.73 | N |
| ATOM | 1525 | CA | PRO | A | 192 | 118.304 | 29.454 | 3.837 | 1.00 | 85.92 | C |
| ATOM | 1526 | CB | PRO | A | 192 | 118.465 | 30.977 | 3.882 | 1.00 | 85.56 | C |
| ATOM | 1527 | CG | PRO | A | 192 | 119.733 | 31.239 | 3.152 | 1.00 | 85.62 | C |
| ATOM | 1528 | CD | PRO | A | 192 | 120.625 | 30.062 | 3.424 | 1.00 | 85.73 | C |
| ATOM | 1529 | C | PRO | A | 192 | 117.431 | 29.038 | 2.650 | 1.00 | 87.68 | C |
| ATOM | 1530 | O | PRO | A | 192 | 117.916 | 28.426 | 1.694 | 1.00 | 87.51 | O |
| ATOM | 1531 | N | LEU | A | 193 | 116.145 | 29.368 | 2.734 | 1.00 | 88.68 | N |
| ATOM | 1532 | CA | LEU | A | 193 | 115.191 | 29.059 | 1.675 | 1.00 | 88.77 | C |
| ATOM | 1533 | CB | LEU | A | 193 | 113.762 | 28.982 | 2.228 | 1.00 | 89.66 | C |
| ATOM | 1534 | CG | LEU | A | 193 | 113.369 | 27.730 | 3.019 | 1.00 | 91.71 | C |
| ATOM | 1535 | CD1 | LEU | A | 193 | 114.294 | 27.487 | 4.203 | 1.00 | 93.84 | C |
| ATOM | 1536 | CD2 | LEU | A | 193 | 111.925 | 27.843 | 3.485 | 1.00 | 90.96 | C |
| ATOM | 1537 | C | LEU | A | 193 | 115.258 | 30.117 | 0.582 | 1.00 | 90.31 | C |
| ATOM | 1538 | O | LEU | A | 193 | 115.552 | 31.282 | 0.852 | 1.00 | 90.47 | O |
| ATOM | 1539 | N | ALA | A | 194 | 114.984 | 29.701 | −0.651 | 1.00 | 91.88 | N |
| ATOM | 1540 | CA | ALA | A | 194 | 114.947 | 30.620 | −1.785 | 1.00 | 92.62 | C |
| ATOM | 1541 | CB | ALA | A | 194 | 115.054 | 29.851 | −3.092 | 1.00 | 94.08 | C |
| ATOM | 1542 | C | ALA | A | 194 | 113.647 | 31.413 | −1.744 | 1.00 | 92.46 | C |
| ATOM | 1543 | O | ALA | A | 194 | 112.663 | 30.965 | −1.151 | 1.00 | 92.11 | O |
| ATOM | 1544 | N | LYS | A | 195 | 113.645 | 32.593 | −2.361 | 1.00 | 93.79 | N |
| ATOM | 1545 | CA | LYS | A | 195 | 112.437 | 33.415 | −2.412 | 1.00 | 96.51 | C |
| ATOM | 1546 | CB | LYS | A | 195 | 112.718 | 34.780 | −3.041 | 1.00 | 101.11 | C |
| ATOM | 1547 | CG | LYS | A | 195 | 113.591 | 35.670 | −2.171 | 1.00 | 108.63 | C |
| ATOM | 1548 | CD | LYS | A | 195 | 113.770 | 37.056 | −2.769 | 1.00 | 116.33 | C |
| ATOM | 1549 | CE | LYS | A | 195 | 114.626 | 37.937 | −1.870 | 1.00 | 118.72 | C |
| ATOM | 1550 | NZ | LYS | A | 195 | 114.852 | 39.289 | −2.453 | 1.00 | 120.76 | N |
| ATOM | 1551 | C | LYS | A | 195 | 111.364 | 32.666 | −3.196 | 1.00 | 94.08 | C |
| ATOM | 1552 | O | LYS | A | 195 | 111.553 | 32.383 | −4.380 | 1.00 | 94.57 | O |
| ATOM | 1553 | N | PRO | A | 196 | 110.239 | 32.335 | −2.537 | 1.00 | 89.68 | N |
| ATOM | 1554 | CA | PRO | A | 196 | 109.214 | 31.518 | −3.174 | 1.00 | 89.92 | C |
| ATOM | 1555 | CB | PRO | A | 196 | 108.264 | 31.195 | −2.021 | 1.00 | 90.30 | C |
| ATOM | 1556 | CG | PRO | A | 196 | 108.366 | 32.383 | −1 .137 | 1.00 | 89.69 | C |
| ATOM | 1557 | CD | PRO | A | 196 | 109.794 | 32.837 | −1.224 | 1.00 | 89.48 | C |
| ATOM | 1558 | C | PRO | A | 196 | 108.480 | 32.251 | −4.291 | 1.00 | 88.68 | C |
| ATOM | 1559 | O | PRO | A | 196 | 108.298 | 33.467 | −4.217 | 1.00 | 84.56 | O |
| ATOM | 1560 | N | LYS | A | 197 | 108.062 | 31.498 | −5.308 | 1.00 | 89.24 | N |
| ATOM | 1561 | CA | LYS | A | 197 | 107.406 | 32.056 | −6.486 | 1.00 | 90.03 | C |
| ATOM | 1562 | CB | LYS | A | 197 | 108.300 | 31.857 | −7.708 | 1.00 | 94.64 | C |
| ATOM | 1563 | CG | LYS | A | 197 | 109.639 | 32.561 | −7.567 | 1.00 | 102.22 | C |
| ATOM | 1564 | CD | LYS | A | 197 | 110.545 | 32.364 | −8.768 | 1.00 | 108.84 | C |
| ATOM | 1565 | CE | LYS | A | 197 | 111.819 | 33.181 | −8.599 | 1.00 | 114.08 | C |
| ATOM | 1566 | NZ | LYS | A | 197 | 112.776 | 33.008 | −9.726 | 1.00 | 117.94 | N |
| ATOM | 1567 | C | LYS | A | 197 | 106.035 | 31.434 | −6.711 | 1.00 | 87.01 | C |
| ATOM | 1568 | O | LYS | A | 197 | 105.739 | 30.359 | −6.192 | 1.00 | 86.28 | O |
| ATOM | 1569 | N | VAL | A | 198 | 105.205 | 32.120 | −7.492 | 1.00 | 85.50 | N |
| ATOM | 1570 | CA | VAL | A | 198 | 103.827 | 31.689 | −7.739 | 1.00 | 84.79 | C |
| ATOM | 1571 | CB | VAL | A | 198 | 103.047 | 32.737 | −8.567 | 1.00 | 84.93 | C |
| ATOM | 1572 | CG1 | VAL | A | 198 | 101.643 | 32.239 | −8.896 | 1.00 | 84.34 | C |
| ATOM | 1573 | CG2 | VAL | A | 198 | 102.983 | 34.067 | −7.822 | 1.00 | 84.73 | C |
| ATOM | 1574 | C | VAL | A | 198 | 103.783 | 30.337 | −8.452 | 1.00 | 83.78 | C |
| ATOM | 1575 | O | VAL | A | 198 | 104.502 | 30.119 | −9.426 | 1.00 | 82.25 | O |
| ATOM | 1576 | N | ALA | A | 199 | 102.938 | 29.438 | −7.952 | 1.00 | 85.82 | N |
| ATOM | 1577 | CA | ALA | A | 199 | 102.791 | 28.103 | −8.529 | 1.00 | 87.01 | C |
| ATOM | 1578 | CB | ALA | A | 199 | 102.153 | 27.159 | −7.521 | 1.00 | 87.79 | C |
| ATOM | 1579 | C | ALA | A | 199 | 101.950 | 28.165 | −9.799 | 1.00 | 86.64 | C |
| ATOM | 1580 | O | ALA | A | 199 | 100.884 | 28.778 | −9.811 | 1.00 | 85.61 | O |
| ATOM | 1581 | N | ARG | A | 200 | 102.434 | 27.526 | −10.862 | 1.00 | 87.46 | N |
| ATOM | 1582 | CA | ARG | A | 200 | 101.745 | 27.524 | −12.153 | 1.00 | 85.78 | C |
| ATOM | 1583 | CB | ARG | A | 200 | 102.754 | 27.716 | −13.284 | 1.00 | 87.93 | C |
| ATOM | 1584 | CG | ARG | A | 200 | 103.507 | 29.037 | −13.218 | 1.00 | 90.52 | C |
| ATOM | 1585 | CD | ARG | A | 200 | 104.596 | 29.114 | −14.276 | 1.00 | 92.57 | C |
| ATOM | 1586 | NE | ARG | A | 200 | 104.039 | 29.134 | −15.626 | 1.00 | 94.90 | N |
| ATOM | 1587 | CZ | ARG | A | 200 | 103.594 | 30.223 | −16.251 | 1.00 | 97.54 | C |
| ATOM | 1588 | NH1 | ARG | A | 200 | 103.104 | 30.118 | −17.481 | 1.00 | 100.03 | N |
| ATOM | 1589 | NH2 | ARG | A | 200 | 103.630 | 31.416 | −15.663 | 1.00 | 98.92 | N |
| ATOM | 1590 | C | ARG | A | 200 | 100.978 | 26.224 | −12.355 | 1.00 | 83.28 | C |
| ATOM | 1591 | O | ARG | A | 200 | 101.387 | 25.176 | −11.855 | 1.00 | 84.83 | O |
| ATOM | 1592 | N | ARG | A | 201 | 99.866 | 26.299 | −13.087 | 1.00 | 82.76 | N |
| ATOM | 1593 | CA | ARG | A | 201 | 99.032 | 25.127 | −13.374 | 1.00 | 84.44 | C |
| ATOM | 1594 | CB | ARG | A | 201 | 97.675 | 25.557 | −13.940 | 1.00 | 82.09 | C |
| ATOM | 1595 | CG | ARG | A | 201 | 96.632 | 24.456 | −13.958 | 1.00 | 79.77 | C |
| ATOM | 1596 | CD | ARG | A | 201 | 95.331 | 24.954 | −14.554 | 1.00 | 79.28 | C |
| ATOM | 1597 | NE | ARG | A | 201 | 94.242 | 23.990 | −14.392 | 1.00 | 80.17 | N |
| ATOM | 1598 | CZ | ARG | A | 201 | 93.447 | 23.899 | −13.323 | 1.00 | 79.41 | C |
| ATOM | 1599 | NH1 | ARG | A | 201 | 92.485 | 22.982 | −13.298 | 1.00 | 79.97 | N |
| ATOM | 1600 | NH2 | ARG | A | 201 | 93.600 | 24.707 | −12.279 | 1.00 | 79.24 | N |
| ATOM | 1601 | C | ARG | A | 201 | 99.786 | 24.225 | −14.352 | 1.00 | 87.56 | C |
| ATOM | 1602 | O | ARG | A | 201 | 99.591 | 24.278 | −15.563 | 1.00 | 85.14 | O |
| ATOM | 1603 | N | CYS | A | 202 | 100.640 | 23.380 | −13.792 | 1.00 | 94.65 | N |
| ATOM | 1604 | CA | CYS | A | 202 | 101.609 | 22.608 | −14.559 | 1.00 | 95.70 | C |
| ATOM | 1605 | CB | CYS | A | 202 | 102.867 | 22.449 | −13.708 | 1.00 | 99.41 | C |
| ATOM | 1606 | SG | CYS | A | 202 | 104.213 | 21.618 | −14.557 | 1.00 | 111.34 | S |
| ATOM | 1607 | C | CYS | A | 202 | 101.142 | 21.228 | −15.009 | 1.00 | 93.03 | C |
| ATOM | 1608 | O | CYS | A | 202 | 101.151 | 20.921 | −16.198 | 1.00 | 90.38 | O |
| ATOM | 1609 | N | GLY | A | 203 | 100.750 | 20.400 | −14.046 | 1.00 | 94.84 | N |
| ATOM | 1610 | CA | GLY | A | 203 | 100.396 | 19.003 | −14.295 | 1.00 | 95.40 | C |
| ATOM | 1611 | C | GLY | A | 203 | 99.189 | 18.741 | −15.174 | 1.00 | 94.49 | C |
| ATOM | 1612 | O | GLY | A | 203 | 98.573 | 19.662 | −15.708 | 1.00 | 95.78 | O |
| ATOM | 1613 | N | ALA | A | 204 | 98.860 | 17.462 | −15.312 | 1.00 | 93.19 | N |
| ATOM | 1614 | CA | ALA | A | 204 | 97.767 | 17.027 | −16.167 | 1.00 | 94.27 | C |
| ATOM | 1615 | CB | ALA | A | 204 | 98.058 | 15.637 | −16.707 | 1.00 | 94.46 | C |
| ATOM | 1616 | C | ALA | A | 204 | 96.443 | 17.021 | −15.425 | 1.00 | 98.11 | C |
| ATOM | 1617 | O | ALA | A | 204 | 96.405 | 16.857 | −14.206 | 1.00 | 102.18 | O |
| ATOM | 1618 | N | ASP | A | 205 | 95.361 | 17.206 | −16.176 | 1.00 | 103.96 | N |
| ATOM | 1619 | CA | ASP | A | 205 | 94.000 | 17.120 | −15.642 | 1.00 | 110.17 | C |
| ATOM | 1620 | CB | ASP | A | 205 | 93.586 | 18.402 | −14.895 | 1.00 | 112.33 | C |
| ATOM | 1621 | CG | ASP | A | 205 | 93.754 | 19.665 | −15.729 | 1.00 | 113.97 | C |
| ATOM | 1622 | OD1 | ASP | A | 205 | 94.538 | 19.648 | −16.705 | 1.00 | 121.66 | O |
| ATOM | 1623 | OD2 | ASP | A | 205 | 93.104 | 20.682 | −15.395 | 1.00 | 108.73 | O |
| ATOM | 1624 | C | ASP | A | 205 | 93.024 | 16.790 | −16.777 | 1.00 | 110.88 | C |
| ATOM | 1625 | O | ASP | A | 205 | 92.516 | 17.692 | −17.446 | 1.00 | 112.06 | O |
| ATOM | 1626 | N | PRO | A | 206 | 92.781 | 15.487 | −17.013 | 1.00 | 110.76 | N |
| ATOM | 1627 | CA | PRO | A | 206 | 91.870 | 15.035 | −18.068 | 1.00 | 113.60 | C |
| ATOM | 1628 | CB | PRO | A | 206 | 91.710 | 13.543 | −17.767 | 1.00 | 113.91 | C |
| ATOM | 1629 | CG | PRO | A | 206 | 92.992 | 13.157 | −17.117 | 1.00 | 113.31 | C |
| ATOM | 1630 | CD | PRO | A | 206 | 93.432 | 14.354 | −16.327 | 1.00 | 111.03 | C |
| ATOM | 1631 | C | PRO | A | 206 | 90.500 | 15.726 | −18.064 | 1.00 | 115.91 | C |
| ATOM | 1632 | O | PRO | A | 206 | 89.961 | 16.030 | −19.131 | 1.00 | 114.76 | O |
| ATOM | 1633 | N | ASP | A | 207 | 89.960 | 15.974 | −16.872 | 1.00 | 115.90 | N |
| ATOM | 1634 | CA | ASP | A | 207 | 88.638 | 16.587 | −16.709 | 1.00 | 115.10 | C |
| ATOM | 1635 | CB | ASP | A | 207 | 88.322 | 16.779 | −15.220 | 1.00 | 116.51 | C |
| ATOM | 1636 | CG | ASP | A | 207 | 88.249 | 15.466 | −14.456 | 1.00 | 118.65 | C |
| ATOM | 1637 | OD1 | ASP | A | 207 | 88.492 | 14.399 | −15.061 | 1.00 | 120.29 | O |
| ATOM | 1638 | OD2 | ASP | A | 207 | 87.950 | 15.505 | −13.244 | 1.00 | 117.90 | O |
| ATOM | 1639 | C | ASP | A | 207 | 88.484 | 17.927 | −17.428 | 1.00 | 114.57 | C |
| ATOM | 1640 | O | ASP | A | 207 | 87.439 | 18.192 | −18.023 | 1.00 | 120.87 | O |
| ATOM | 1641 | N | HIS | A | 208 | 89.521 | 18.760 | −17.376 | 1.00 | 113.32 | N |
| ATOM | 1642 | CA | HIS | A | 208 | 89.483 | 20.096 | −17.988 | 1.00 | 113.45 | C |
| ATOM | 1643 | CB | HIS | A | 208 | 90.067 | 21.132 | −17.017 | 1.00 | 115.37 | C |
| ATOM | 1644 | CG | HIS | A | 208 | 89.172 | 21.444 | −15.857 | 1.00 | 117.34 | C |
| ATOM | 1645 | ND1 | HIS | A | 208 | 88.893 | 20.531 | −14.863 | 1.00 | 120.15 | N |
| ATOM | 1646 | CE1 | HIS | A | 208 | 88.082 | 21.080 | −13.975 | 1.00 | 119.71 | C |
| ATOM | 1647 | NE2 | HIS | A | 208 | 87.829 | 22.320 | −14.355 | 1.00 | 121.26 | N |
| ATOM | 1648 | CD2 | HIS | A | 208 | 88.499 | 22.573 | −15.528 | 1.00 | 119.91 | C |
| ATOM | 1649 | C | HIS | A | 208 | 90.182 | 20.175 | −19.356 | 1.00 | 112.20 | C |
| ATOM | 1650 | O | HIS | A | 208 | 90.581 | 21.259 | −19.788 | 1.00 | 107.64 | O |
| ATOM | 1651 | N | GLY | A | 209 | 90.323 | 19.037 | −20.036 | 1.00 | 114.07 | N |
| ATOM | 1652 | CA | GLY | A | 209 | 90.876 | 19.007 | −21.392 | 1.00 | 116.91 | C |
| ATOM | 1653 | C | GLY | A | 209 | 92.220 | 18.320 | −21.533 | 1.00 | 121.22 | C |
| ATOM | 1654 | O | GLY | A | 209 | 92.299 | 17.200 | −22.040 | 1.00 | 125.14 | O |
| ATOM | 1655 | N | LYS | A | 210 | 93.276 | 18.995 | −21.081 | 1.00 | 123.83 | N |
| ATOM | 1656 | CA | LYS | A | 210 | 94.650 | 18.501 | −21.232 | 1.00 | 123.76 | C |
| ATOM | 1657 | CB | LYS | A | 210 | 95.645 | 19.588 | −20.823 | 1.00 | 129.30 | C |
| ATOM | 1658 | CG | LYS | A | 210 | 95.595 | 20.797 | −21.742 | 1.00 | 137.32 | C |
| ATOM | 1659 | CD | LYS | A | 210 | 96.602 | 21.857 | −21.349 | 1.00 | 141.35 | C |
| ATOM | 1660 | CE | LYS | A | 210 | 96.470 | 23.076 | −22.249 | 1.00 | 142.74 | C |
| ATOM | 1661 | NZ | LYS | A | 210 | 97.345 | 24.190 | −21.804 | 1.00 | 145.82 | N |
| ATOM | 1662 | C | LYS | A | 210 | 94.936 | 17.227 | −20.440 | 1.00 | 118.22 | C |
| ATOM | 1663 | O | LYS | A | 210 | 94.935 | 17.261 | −19.210 | 1.00 | 115.67 | O |
| ATOM | 1664 | N | PRO | A | 211 | 95.191 | 16.103 | −21.144 | 1.00 | 112.92 | N |
| ATOM | 1665 | CA | PRO | A | 211 | 95.491 | 14.842 | −20.476 | 1.00 | 111.37 | C |
| ATOM | 1666 | CB | PRO | A | 211 | 94.974 | 13.806 | −21.474 | 1.00 | 111.45 | C |
| ATOM | 1667 | CG | PRO | A | 211 | 95.191 | 14.441 | −22.807 | 1.00 | 112.22 | C |
| ATOM | 1668 | CD | PRO | A | 211 | 95.187 | 15.936 | −22.612 | 1.00 | 112.40 | C |
| ATOM | 1669 | C | PRO | A | 211 | 96.986 | 14.615 | −20.204 | 1.00 | 108.39 | C |
| ATOM | 1670 | O | PRO | A | 211 | 97.336 | 13.704 | −19.453 | 1.00 | 106.62 | O |
| ATOM | 1671 | N | ASP | A | 212 | 97.850 | 15.431 | −20.808 | 1.00 | 106.42 | N |
| ATOM | 1672 | CA | ASP | A | 212 | 99.298 | 15.296 | −20.656 | 1.00 | 105.63 | C |
| ATOM | 1673 | CB | ASP | A | 212 | 99.967 | 15.171 | −22.029 | 1.00 | 108.37 | C |
| ATOM | 1674 | CG | ASP | A | 212 | 99.534 | 13.927 | −22.786 | 1.00 | 111.39 | C |
| ATOM | 1675 | OD1 | ASP | A | 212 | 99.239 | 12.896 | −22.141 | 1.00 | 110.82 | O |
| ATOM | 1676 | OD2 | ASP | A | 212 | 99.505 | 13.982 | −24.034 | 1.00 | 113.97 | O |
| ATOM | 1677 | C | ASP | A | 212 | 99.883 | 16.499 | −19.930 | 1.00 | 101.27 | C |
| ATOM | 1678 | O | ASP | A | 212 | 99.555 | 17.644 | −20.248 | 1.00 | 99.99 | O |
| ATOM | 1679 | N | ALA | A | 213 | 100.766 | 16.234 | −18.970 | 1.00 | 96.39 | N |
| ATOM | 1680 | CA | ALA | A | 213 | 101.421 | 17.292 | −18.205 | 1.00 | 92.95 | C |
| ATOM | 1681 | CB | ALA | A | 213 | 102.143 | 16.702 | −17.007 | 1.00 | 93.67 | C |
| ATOM | 1682 | C | ALA | A | 213 | 102.404 | 18.066 | −19.077 | 1.00 | 90.86 | C |
| ATOM | 1683 | O | ALA | A | 213 | 102.891 | 17.548 | −20.081 | 1.00 | 91.44 | O |
| ATOM | 1684 | N | VAL | A | 214 | 102.694 | 19.303 | −18.683 | 1.00 | 89.07 | N |
| ATOM | 1685 | CA | VAL | A | 214 | 103.615 | 20.160 | −19.426 | 1.00 | 90.36 | C |
| ATOM | 1686 | CB | VAL | A | 214 | 102.894 | 21.402 | −19.986 | 1.00 | 90.82 | C |
| ATOM | 1687 | CG1 | VAL | A | 214 | 103.848 | 22.256 | −20.817 | 1.00 | 92.38 | C |
| ATOM | 1688 | CG2 | VAL | A | 214 | 101.700 | 20.974 | −20.828 | 1.00 | 92.12 | C |
| ATOM | 1689 | C | VAL | A | 214 | 104.780 | 20.571 | −18.519 | 1.00 | 92.80 | C |
| ATOM | 1690 | O | VAL | A | 214 | 104.756 | 21.652 | −17.931 | 1.00 | 94.19 | O |
| ATOM | 1691 | N | PRO | A | 215 | 105.809 | 19.705 | −18.408 | 1.00 | 93.71 | N |
| ATOM | 1692 | CA | PRO | A | 215 | 106.980 | 19.938 | −17.548 | 1.00 | 93.49 | C |
| ATOM | 1693 | CB | PRO | A | 215 | 108.004 | 18.906 | −18.037 | 1.00 | 93.83 | C |
| ATOM | 1694 | CG | PRO | A | 215 | 107.254 | 17.909 | −18.850 | 1.00 | 94.23 | C |
| ATOM | 1695 | CD | PRO | A | 215 | 105.857 | 18.391 | −19.077 | 1.00 | 93.70 | C |
| ATOM | 1696 | C | PRO | A | 215 | 107.585 | 21.339 | −17.653 | 1.00 | 94.44 | C |
| ATOM | 1697 | O | PRO | A | 215 | 108.031 | 21.893 | −16.649 | 1.00 | 95.00 | O |
| ATOM | 1698 | N | GLY | A | 216 | 107.604 | 21.896 | −18.861 | 1.00 | 93.59 | N |
| ATOM | 1699 | CA | GLY | A | 216 | 108.176 | 23.218 | −19.100 | 1.00 | 91.57 | C |
| ATOM | 1700 | C | GLY | A | 216 | 107.431 | 24.382 | −18.468 | 1.00 | 91.34 | C |
| ATOM | 1701 | O | GLY | A | 216 | 107.971 | 25.484 | −18.381 | 1.00 | 93.81 | O |
| ATOM | 1702 | N | ASN | A | 217 | 106.196 | 24.145 | −18.030 | 1.00 | 90.53 | N |
| ATOM | 1703 | CA | ASN | A | 217 | 105.355 | 25.193 | −17.451 | 1.00 | 89.29 | C |
| ATOM | 1704 | CB | ASN | A | 217 | 103.918 | 24.992 | −17.933 | 1.00 | 87.44 | C |
| ATOM | 1705 | CG | ASN | A | 217 | 103.098 | 26.271 | −17.916 | 1.00 | 86.60 | C |
| ATOM | 1706 | OD1 | ASN | A | 217 | 103.606 | 27.361 | −18.182 | 1.00 | 82.24 | O |
| ATOM | 1707 | ND2 | ASN | A | 217 | 101.812 | 26.133 | −17.613 | 1.00 | 88.48 | N |
| ATOM | 1708 | C | ASN | A | 217 | 105.377 | 25.209 | −15.921 | 1.00 | 90.89 | C |
| ATOM | 1709 | O | ASN | A | 217 | 104.729 | 26.049 | −15.301 | 1.00 | 93.27 | O |
| ATOM | 1710 | N | CYS | A | 218 | 106.122 | 24.285 | −15.321 | 1.00 | 92.57 | N |
| ATOM | 1711 | CA | CYS | A | 218 | 106.186 | 24.151 | −13.861 | 1.00 | 92.13 | C |
| ATOM | 1712 | CB | CYS | A | 218 | 106.800 | 22.797 | −13.471 | 1.00 | 95.46 | C |
| ATOM | 1713 | SG | CYS | A | 218 | 105.647 | 21.446 | −13.139 | 1.00 | 101.99 | S |
| ATOM | 1714 | C | CYS | A | 218 | 106.993 | 25.242 | −13.171 | 1.00 | 89.73 | C |
| ATOM | 1715 | O | CYS | A | 218 | 107.875 | 25.855 | −13.769 | 1.00 | 90.36 | O |
| ATOM | 1716 | N | THR | A | 219 | 106.663 | 25.468 | −11.902 | 1.00 | 89.47 | N |
| ATOM | 1717 | CA | THR | A | 219 | 107.444 | 26.320 | −11.015 | 1.00 | 89.67 | C |
| ATOM | 1718 | CB | THR | A | 219 | 106.547 | 27.210 | −10.139 | 1.00 | 89.31 | C |
| ATOM | 1719 | OG1 | THR | A | 219 | 105.719 | 28.023 | −10.977 | 1.00 | 89.54 | O |
| ATOM | 1720 | CG2 | THR | A | 219 | 107.386 | 28.109 | −9.241 | 1.00 | 89.94 | C |
| ATOM | 1721 | C | THR | A | 219 | 108.222 | 25.325 | −10.157 | 1.00 | 89.67 | C |
| ATOM | 1722 | O | THR | A | 219 | 107.624 | 24.568 | −9.388 | 1.00 | 88.45 | O |
| ATOM | 1723 | N | TYR | A | 220 | 109.546 | 25.319 | −10.295 | 1.00 | 88.06 | N |
| ATOM | 1724 | CA | TYR | A | 220 | 110.376 | 24.283 | −9.673 | 1.00 | 87.99 | C |
| ATOM | 1725 | CB | TYR | A | 220 | 111.469 | 23.834 | −10.651 | 1.00 | 85.51 | C |
| ATOM | 1726 | CG | TYR | A | 220 | 110.940 | 22.896 | −11.707 | 1.00 | 84.82 | C |
| ATOM | 1727 | CD1 | TYR | A | 220 | 110.797 | 21.540 | −11.438 | 1.00 | 85.64 | C |
| ATOM | 1728 | CE1 | TYR | A | 220 | 110.303 | 20.669 | −12.390 | 1.00 | 87.79 | C |
| ATOM | 1729 | CZ | TYR | A | 220 | 109.938 | 21.150 | −13.629 | 1.00 | 87.64 | C |
| ATOM | 1730 | OH | TYR | A | 220 | 109.447 | 20.273 | −14.565 | 1.00 | 92.31 | O |
| ATOM | 1731 | CE2 | TYR | A | 220 | 110.066 | 22.495 | −13.923 | 1.00 | 85.64 | C |
| ATOM | 1732 | CD2 | TYR | A | 220 | 110.560 | 23.361 | −12.963 | 1.00 | 84.72 | C |
| ATOM | 1733 | C | TYR | A | 220 | 110.982 | 24.613 | −8.309 | 1.00 | 89.61 | C |
| ATOM | 1734 | O | TYR | A | 220 | 111.157 | 23.715 | −7.484 | 1.00 | 90.52 | O |
| ATOM | 1735 | N | GLY | A | 221 | 111.296 | 25.879 | −8.060 | 1.00 | 88.76 | N |
| ATOM | 1736 | CA | GLY | A | 221 | 111.916 | 26.265 | −6.791 | 1.00 | 88.20 | C |
| ATOM | 1737 | C | GLY | A | 221 | 110.953 | 26.231 | −5.612 | 1.00 | 84.48 | C |
| ATOM | 1738 | O | GLY | A | 221 | 110.070 | 25.375 | −5.532 | 1.00 | 80.92 | O |
| ATOM | 1739 | N | ALA | A | 222 | 111.142 | 27.164 | −4.684 | 1.00 | 81.28 | N |
| ATOM | 1740 | CA | ALA | A | 222 | 110.228 | 27.326 | −3.564 | 1.00 | 79.28 | C |
| ATOM | 1741 | CB | ALA | A | 222 | 110.833 | 28.242 | −2.510 | 1.00 | 80.09 | C |
| ATOM | 1742 | C | ALA | A | 222 | 108.935 | 27.919 | −4.105 | 1.00 | 76.65 | C |
| ATOM | 1743 | O | ALA | A | 222 | 108.970 | 28.817 | −4.947 | 1.00 | 77.00 | O |
| ATOM | 1744 | N | LYS | A | 223 | 107.799 | 27.420 | −3.628 | 1.00 | 75.17 | N |
| ATOM | 1745 | CA | LYS | A | 223 | 106.499 | 27.890 | −4.102 | 1.00 | 73.94 | C |
| ATOM | 1746 | CB | LYS | A | 223 | 105.717 | 26.727 | −4.719 | 1.00 | 74.06 | C |
| ATOM | 1747 | CG | LYS | A | 223 | 106.187 | 26.356 | −6.117 | 1.00 | 72.01 | C |
| ATOM | 1748 | CD | LYS | A | 223 | 105.382 | 25.206 | −6.701 | 1.00 | 70.96 | C |
| ATOM | 1749 | CE | LYS | A | 223 | 105.802 | 23.859 | −6.137 | 1.00 | 70.20 | C |
| ATOM | 1750 | NZ | LYS | A | 223 | 107.178 | 23.471 | −6.561 | 1.00 | 69.98 | N |
| ATOM | 1751 | C | LYS | A | 223 | 105.666 | 28.563 | −3.014 | 1.00 | 72.76 | C |
| ATOM | 1752 | O | LYS | A | 223 | 105.661 | 28.132 | −1.863 | 1.00 | 77.03 | O |
| ATOM | 1753 | N | GLN | A | 224 | 104.959 | 29.622 | −3.399 | 1.00 | 71.94 | N |
| ATOM | 1754 | CA | GLN | A | 224 | 104.062 | 30.330 | −2.490 | 1.00 | 71.49 | C |
| ATOM | 1755 | CB | GLN | A | 224 | 103.700 | 31.707 | −3.047 | 1.00 | 69.85 | C |
| ATOM | 1756 | CG | GLN | A | 224 | 104.875 | 32.633 | −3.299 | 1.00 | 67.77 | C |
| ATOM | 1757 | CD | GLN | A | 224 | 104.429 | 34.012 | −3.742 | 1.00 | 67.40 | C |
| ATOM | 1758 | OE1 | GLN | A | 224 | 103.238 | 34.263 | −3.939 | 1.00 | 68.00 | O |
| ATOM | 1759 | NE2 | GLN | A | 224 | 105.384 | 34.919 | −3.894 | 1.00 | 68.81 | N |
| ATOM | 1760 | C | GLN | A | 224 | 102.772 | 29.537 | −2.320 | 1.00 | 71.05 | C |
| ATOM | 1761 | O | GLN | A | 224 | 102.475 | 28.664 | −3.132 | 1.00 | 72.16 | O |
| ATOM | 1762 | N | PRO | A | 225 | 101.989 | 29.846 | −1.270 | 1.00 | 72.77 | N |
| ATOM | 1763 | CA | PRO | A | 225 | 100.696 | 29.177 | −1.136 | 1.00 | 72.88 | C |
| ATOM | 1764 | CB | PRO | A | 225 | 100.196 | 29.624 | 0.248 | 1.00 | 72.02 | C |
| ATOM | 1765 | CG | PRO | A | 225 | 101.396 | 30.150 | 0.956 | 1.00 | 73.36 | C |
| ATOM | 1766 | CD | PRO | A | 225 | 102.278 | 30.714 | −0.116 | 1.00 | 73.96 | C |
| ATOM | 1767 | C | PRO | A | 225 | 99.735 | 29.633 | −2.230 | 1.00 | 72.69 | C |
| ATOM | 1768 | O | PRO | A | 225 | 99.995 | 30.629 | −2.903 | 1.00 | 73.43 | O |
| ATOM | 1769 | N | LEU | A | 226 | 98.637 | 28.904 | −2.397 | 1.00 | 72.76 | N |
| ATOM | 1770 | CA | LEU | A | 226 | 97.638 | 29.223 | −3.411 | 1.00 | 71.49 | C |
| ATOM | 1771 | CB | LEU | A | 226 | 96.976 | 27.935 | −3.908 | 1.00 | 71.67 | C |
| ATOM | 1772 | CG | LEU | A | 226 | 97.921 | 26.901 | −4.523 | 1.00 | 72.47 | C |
| ATOM | 1773 | CD1 | LEU | A | 226 | 97.232 | 25.551 | −4.637 | 1.00 | 72.82 | C |
| ATOM | 1774 | CD2 | LEU | A | 226 | 98.436 | 27.365 | −5.879 | 1.00 | 73.04 | C |
| ATOM | 1775 | C | LEU | A | 226 | 96.579 | 30.175 | −2.857 | 1.00 | 71.78 | C |
| ATOM | 1776 | O | LEU | A | 226 | 95.613 | 29.740 | −2.222 | 1.00 | 75.71 | O |
| ATOM | 1777 | N | TYR | A | 227 | 96.773 | 31.474 | −3.083 | 1.00 | 69.55 | N |
| ATOM | 1778 | CA | TYR | A | 227 | 95.798 | 32.485 | −2.666 | 1.00 | 70.32 | C |
| ATOM | 1779 | CB | TYR | A | 227 | 96.469 | 33.819 | −2.358 | 1.00 | 70.40 | C |
| ATOM | 1780 | CG | TYR | A | 227 | 97.631 | 33.712 | −1.404 | 1.00 | 70.85 | C |
| ATOM | 1781 | CD1 | TYR | A | 227 | 97.499 | 33.054 | −0.183 | 1.00 | 72.80 | C |
| ATOM | 1782 | CE1 | TYR | A | 227 | 98.562 | 32.964 | 0.702 | 1.00 | 71.55 | C |
| ATOM | 1783 | CZ | TYR | A | 227 | 99.771 | 33.541 | 0.371 | 1.00 | 71.70 | C |
| ATOM | 1784 | OH | TYR | A | 227 | 100.833 | 33.460 | 1.236 | 1.00 | 71.35 | O |
| ATOM | 1785 | CE2 | TYR | A | 227 | 99.925 | 34.200 | −0.833 | 1.00 | 73.08 | C |
| ATOM | 1786 | CD2 | TYR | A | 227 | 98.858 | 34.285 | −1.710 | 1.00 | 71.37 | C |
| ATOM | 1787 | C | TYR | A | 227 | 94.801 | 32.649 | −3.800 | 1.00 | 69.55 | C |
| ATOM | 1788 | O | TYR | A | 227 | 95.157 | 33.101 | −4.890 | 1.00 | 66.57 | O |
| ATOM | 1789 | N | TRP | A | 228 | 93.548 | 32.300 | −3.531 | 1.00 | 70.33 | N |
| ATOM | 1790 | CA | TRP | A | 228 | 92.557 | 32.191 | −4.585 | 1.00 | 70.72 | C |
| ATOM | 1791 | CB | TRP | A | 228 | 92.648 | 30.765 | −5.144 | 1.00 | 70.69 | C |
| ATOM | 1792 | CG | TRP | A | 228 | 91.719 | 30.467 | −6.247 | 1.00 | 70.07 | C |
| ATOM | 1793 | CD1 | TRP | A | 228 | 91.780 | 30.943 | −7.518 | 1.00 | 71.56 | C |
| ATOM | 1794 | NE1 | TRP | A | 228 | 90.750 | 30.431 | −8.263 | 1.00 | 71.58 | N |
| ATOM | 1795 | CE2 | TRP | A | 228 | 90.004 | 29.601 | −7.472 | 1.00 | 68.67 | C |
| ATOM | 1796 | CD2 | TRP | A | 228 | 90.592 | 29.598 | −6.194 | 1.00 | 68.31 | C |
| ATOM | 1797 | CE3 | TRP | A | 228 | 90.020 | 28.817 | −5.188 | 1.00 | 68.07 | C |
| ATOM | 1798 | CZ3 | TRP | A | 228 | 88.899 | 28.087 | −5.481 | 1.00 | 68.67 | C |
| ATOM | 1799 | CH2 | TRP | A | 228 | 88.329 | 28.114 | −6.767 | 1.00 | 69.73 | C |
| ATOM | 1800 | CZ2 | TRP | A | 228 | 88.871 | 28.862 | −7.770 | 1.00 | 69.10 | C |
| ATOM | 1801 | C | TRP | A | 228 | 91.139 | 32.504 | −4.106 | 1.00 | 71.61 | C |
| ATOM | 1802 | O | TRP | A | 228 | 90.819 | 32.346 | −2.928 | 1.00 | 70.99 | O |
| ATOM | 1803 | N | LEU | A | 229 | 90.312 | 32.980 | −5.035 | 1.00 | 74.68 | N |
| ATOM | 1804 | CA | LEU | A | 229 | 88.887 | 33.246 | −4.803 | 1.00 | 75.33 | C |
| ATOM | 1805 | CB | LEU | A | 229 | 88.181 | 31.963 | −4.351 | 1.00 | 74.13 | C |
| ATOM | 1806 | CG | LEU | A | 229 | 86.713 | 31.815 | −4.751 | 1.00 | 75.62 | C |
| ATOM | 1807 | CD1 | LEU | A | 229 | 86.613 | 31.384 | −6.209 | 1.00 | 75.47 | C |
| ATOM | 1808 | CD2 | LEU | A | 229 | 86.015 | 30.802 | −3.857 | 1.00 | 76.28 | C |
| ATOM | 1809 | C | LEU | A | 229 | 88.607 | 34.376 | −3.803 | 1.00 | 76.75 | C |
| ATOM | 1810 | O | LEU | A | 229 | 87.518 | 34.435 | −3.232 | 1.00 | 78.50 | O |
| ATOM | 1811 | N | GLN | A | 230 | 89.568 | 35.277 | −3.607 | 1.00 | 78.20 | N |
| ATOM | 1812 | CA | GLN | A | 230 | 89.400 | 36.389 | −2.668 | 1.00 | 81.19 | C |
| ATOM | 1813 | CB | GLN | A | 230 | 90.621 | 36.510 | −1.748 | 1.00 | 80.60 | C |
| ATOM | 1814 | CG | GLN | A | 230 | 90.956 | 35.236 | −0.995 | 1.00 | 80.41 | C |
| ATOM | 1815 | CD | GLN | A | 230 | 89.756 | 34.657 | −0.267 | 1.00 | 80.81 | C |
| ATOM | 1816 | OE1 | GLN | A | 230 | 89.005 | 35.378 | 0.385 | 1.00 | 81.10 | O |
| ATOM | 1817 | NE2 | GLN | A | 230 | 89.567 | 33.350 | −0.383 | 1.00 | 80.45 | N |
| ATOM | 1818 | C | GLN | A | 230 | 89.173 | 37.707 | −3.393 | 1.00 | 83.69 | C |
| ATOM | 1819 | O | GLN | A | 230 | 89.290 | 37.785 | −4.615 | 1.00 | 85.42 | O |
| ATOM | 1820 | N | LYS | A | 231 | 88.852 | 38.741 | −2.625 | 1.00 | 86.49 | N |
| ATOM | 1821 | CA | LYS | A | 231 | 88.640 | 40.070 | −3.173 | 1.00 | 87.59 | C |
| ATOM | 1822 | CB | LYS | A | 231 | 87.760 | 40.892 | −2.231 | 1.00 | 91.33 | C |
| ATOM | 1823 | CG | LYS | A | 231 | 87.261 | 42.196 | −2.825 | 1.00 | 95.93 | C |
| ATOM | 1824 | CD | LYS | A | 231 | 86.089 | 42.745 | −2.028 | 1.00 | 98.90 | C |
| ATOM | 1825 | CE | LYS | A | 231 | 85.572 | 44.044 | −2.621 | 1.00 | 102.14 | C |
| ATOM | 1826 | NZ | LYS | A | 231 | 84.321 | 44.487 | −1.950 | 1.00 | 104.65 | N |
| ATOM | 1827 | C | LYS | A | 231 | 89.971 | 40.778 | −3.412 | 1.00 | 87.28 | C |
| ATOM | 1828 | O | LYS | A | 231 | 90.073 | 41.614 | −4.307 | 1.00 | 88.94 | O |
| ATOM | 1829 | N | GLU | A | 232 | 90.986 | 40.440 | −2.615 | 1.00 | 89.06 | N |
| ATOM | 1830 | CA | GLU | A | 232 | 92.305 | 41.067 | −2.724 | 1.00 | 90.80 | C |
| ATOM | 1831 | CB | GLU | A | 232 | 92.448 | 42.184 | −1.683 | 1.00 | 94.95 | C |
| ATOM | 1832 | CG | GLU | A | 232 | 91.340 | 43.231 | −1.678 | 1.00 | 98.24 | C |
| ATOM | 1833 | CD | GLU | A | 232 | 91.492 | 44.250 | −0.559 | 1.00 | 99.79 | C |
| ATOM | 1834 | OE1 | GLU | A | 232 | 92.363 | 44.064 | 0.319 | 1.00 | 100.02 | O |
| ATOM | 1835 | OE2 | GLU | A | 232 | 90.727 | 45.237 | −0.549 | 1.00 | 101.71 | O |
| ATOM | 1836 | C | GLU | A | 232 | 93.449 | 40.074 | −2.508 | 1.00 | 88.75 | C |
| ATOM | 1837 | O | GLU | A | 232 | 93.289 | 39.063 | −1.825 | 1.00 | 87.80 | O |
| ATOM | 1838 | N | GLY | A | 233 | 94.604 | 40.383 | −3.095 | 1.00 | 87.81 | N |
| ATOM | 1839 | CA | GLY | A | 233 | 95.838 | 39.625 | −2.874 | 1.00 | 86.80 | C |
| ATOM | 1840 | C | GLY | A | 233 | 95.893 | 38.203 | −3.401 | 1.00 | 85.56 | C |
| ATOM | 1841 | O | GLY | A | 233 | 96.456 | 37.325 | −2.750 | 1.00 | 86.63 | O |
| ATOM | 1842 | N | ASN | A | 234 | 95.327 | 37.978 | −4.582 | 1.00 | 85.54 | N |
| ATOM | 1843 | CA | ASN | A | 234 | 95.341 | 36.658 | −5.210 | 1.00 | 85.24 | C |
| ATOM | 1844 | CB | ASN | A | 234 | 94.073 | 36.455 | −6.043 | 1.00 | 85.03 | C |
| ATOM | 1845 | CG | ASN | A | 234 | 92.838 | 36.264 | −5.192 | 1.00 | 84.34 | C |
| ATOM | 1846 | OD1 | ASN | A | 234 | 92.732 | 35.297 | −4.442 | 1.00 | 86.46 | O |
| ATOM | 1847 | ND2 | ASN | A | 234 | 91.886 | 37.171 | −5.322 | 1.00 | 85.12 | N |
| ATOM | 1848 | C | ASN | A | 234 | 96.554 | 36.467 | −6.115 | 1.00 | 86.89 | C |
| ATOM | 1849 | O | ASN | A | 234 | 96.942 | 37.384 | −6.843 | 1.00 | 87.53 | O |
| ATOM | 1850 | N | ASN | A | 235 | 97.151 | 35.278 | −6.060 | 1.00 | 86.38 | N |
| ATOM | 1851 | CA | ASN | A | 235 | 98.233 | 34.899 | −6.979 | 1.00 | 86.26 | C |
| ATOM | 1852 | CB | ASN | A | 235 | 99.465 | 34.387 | −6.221 | 1.00 | 86.78 | C |
| ATOM | 1853 | CG | ASN | A | 235 | 99.156 | 33.219 | −5.310 | 1.00 | 89.65 | C |
| ATOM | 1854 | OD1 | ASN | A | 235 | 98.019 | 32.754 | −5.232 | 1.00 | 91.82 | O |
| ATOM | 1855 | ND2 | ASN | A | 235 | 100.173 | 32.742 | −4.603 | 1.00 | 93.67 | N |
| ATOM | 1856 | C | ASN | A | 235 | 97.753 | 33.875 | −8.019 | 1.00 | 86.12 | C |
| ATOM | 1857 | O | ASN | A | 235 | 98.416 | 33.661 | −9.035 | 1.00 | 86.94 | O |
| ATOM | 1858 | N | GLU | A | 236 | 96.610 | 33.240 | −7.749 | 1.00 | 84.08 | N |
| ATOM | 1859 | CA | GLU | A | 236 | 95.962 | 32.338 | −8.697 | 1.00 | 84.15 | C |
| ATOM | 1860 | CB | GLU | A | 236 | 95.678 | 30.980 | −8.058 | 1.00 | 86.71 | C |
| ATOM | 1861 | CG | GLU | A | 236 | 96.901 | 30.285 | −7.479 | 1.00 | 87.83 | C |
| ATOM | 1862 | CD | GLU | A | 236 | 97.991 | 30.039 | −8.502 | 1.00 | 88.15 | C |
| ATOM | 1863 | OE1 | GLU | A | 236 | 97.673 | 29.826 | −9.691 | 1.00 | 90.60 | O |
| ATOM | 1864 | OE2 | GLU | A | 236 | 99.171 | 30.051 | −8.107 | 1.00 | 91.39 | O |
| ATOM | 1865 | C | GLU | A | 236 | 94.652 | 32.977 | −9.136 | 1.00 | 83.78 | C |
| ATOM | 1866 | O | GLU | A | 236 | 93.927 | 33.542 | −8.314 | 1.00 | 82.36 | O |
| ATOM | 1867 | N | PHE | A | 237 | 94.351 | 32.889 | −10.431 | 1.00 | 82.30 | N |
| ATOM | 1868 | CA | PHE | A | 237 | 93.163 | 33.530 | −10.991 | 1.00 | 79.56 | C |
| ATOM | 1869 | CB | PHE | A | 237 | 93.587 | 34.682 | −11.900 | 1.00 | 78.27 | C |
| ATOM | 1870 | CG | PHE | A | 237 | 94.560 | 35.626 | −11.260 | 1.00 | 77.48 | C |
| ATOM | 1871 | CD1 | PHE | A | 237 | 94.115 | 36.639 | −10.424 | 1.00 | 78.26 | C |
| ATOM | 1872 | CE1 | PHE | A | 237 | 95.011 | 37.512 | −9.830 | 1.00 | 79.61 | C |
| ATOM | 1873 | CZ | PHE | A | 237 | 96.368 | 37.378 | −10.069 | 1.00 | 80.36 | C |
| ATOM | 1874 | CE2 | PHE | A | 237 | 96.825 | 36.370 | −10.900 | 1.00 | 79.83 | C |
| ATOM | 1875 | CD2 | PHE | A | 237 | 95.922 | 35.499 | −11.489 | 1.00 | 78.69 | C |
| ATOM | 1876 | C | PHE | A | 237 | 92.275 | 32.558 | −11.763 | 1.00 | 79.29 | C |
| ATOM | 1877 | O | PHE | A | 237 | 91.539 | 32.967 | −12.664 | 1.00 | 81.99 | O |
| ATOM | 1878 | N | ASP | A | 238 | 92.327 | 31.279 | −11.400 | 1.00 | 77.44 | N |
| ATOM | 1879 | CA | ASP | A | 238 | 91.510 | 30.265 | −12.062 | 1.00 | 77.10 | C |
| ATOM | 1880 | CB | ASP | A | 238 | 91.932 | 28.856 | −11.639 | 1.00 | 78.33 | C |
| ATOM | 1881 | CG | ASP | A | 238 | 93.362 | 28.523 | −12.033 | 1.00 | 79.57 | C |
| ATOM | 1882 | OD1 | ASP | A | 238 | 94.279 | 29.312 | −11.712 | 1.00 | 79.76 | O |
| ATOM | 1883 | OD2 | ASP | A | 238 | 93.567 | 27.455 | −12.648 | 1.00 | 82.19 | O |
| ATOM | 1884 | C | ASP | A | 238 | 90.031 | 30.473 | −11.746 | 1.00 | 75.52 | C |
| ATOM | 1885 | O | ASP | A | 238 | 89.684 | 31.143 | −10.775 | 1.00 | 73.58 | O |
| ATOM | 1886 | N | ASP | A | 239 | 89.164 | 29.900 | −12.572 | 1.00 | 77.44 | N |
| ATOM | 1887 | CA | ASP | A | 239 | 87.727 | 30.036 | −12.373 | 1.00 | 78.86 | C |
| ATOM | 1888 | CB | ASP | A | 239 | 86.955 | 29.654 | −13.642 | 1.00 | 81.52 | C |
| ATOM | 1889 | CG | ASP | A | 239 | 85.477 | 30.011 | −13.560 | 1.00 | 82.95 | C |
| ATOM | 1890 | OD1 | ASP | A | 239 | 84.643 | 29.085 | −13.509 | 1.00 | 78.59 | O |
| ATOM | 1891 | OD2 | ASP | A | 239 | 85.153 | 31.218 | −13.519 | 1.00 | 85.56 | O |
| ATOM | 1892 | C | ASP | A | 239 | 87.260 | 29.164 | −11.216 | 1.00 | 77.00 | C |
| ATOM | 1893 | O | ASP | A | 239 | 87.864 | 28.137 | −10.916 | 1.00 | 77.36 | O |
| ATOM | 1894 | N | TYR | A | 240 | 86.175 | 29.599 | −10.584 | 1.00 | 77.15 | N |
| ATOM | 1895 | CA | TYR | A | 240 | 85.514 | 28.897 | −9.484 | 1.00 | 78.20 | C |
| ATOM | 1896 | CB | TYR | A | 240 | 84.096 | 29.465 | −9.351 | 1.00 | 78.56 | C |
| ATOM | 1897 | CG | TYR | A | 240 | 83.250 | 28.928 | −8.219 | 1.00 | 78.85 | C |
| ATOM | 1898 | CD1 | TYR | A | 240 | 82.506 | 27.762 | −8.372 | 1.00 | 80.37 | C |
| ATOM | 1899 | CE1 | TYR | A | 240 | 81.712 | 27.279 | −7.344 | 1.00 | 81.09 | C |
| ATOM | 1900 | CZ | TYR | A | 240 | 81.641 | 27.975 | −6.150 | 1.00 | 79.15 | C |
| ATOM | 1901 | OH | TYR | A | 240 | 80.854 | 27.504 | −5.126 | 1.00 | 80.96 | O |
| ATOM | 1902 | CE2 | TYR | A | 240 | 82.359 | 29.141 | −5.979 | 1.00 | 77.95 | C |
| ATOM | 1903 | CD2 | TYR | A | 240 | 83.152 | 29.616 | −7.011 | 1.00 | 77.64 | C |
| ATOM | 1904 | C | TYR | A | 240 | 85.456 | 27.376 | −9.667 | 1.00 | 80.18 | C |
| ATOM | 1905 | O | TYR | A | 240 | 85.729 | 26.629 | −8.727 | 1.00 | 81.92 | O |
| ATOM | 1906 | N | ILE | A | 241 | 85.119 | 26.926 | −10.874 | 1.00 | 83.74 | N |
| ATOM | 1907 | CA | ILE | A | 241 | 84.960 | 25.487 | −11.153 | 1.00 | 86.17 | C |
| ATOM | 1908 | CB | ILE | A | 241 | 83.893 | 25.231 | −12.245 | 1.00 | 88.43 | C |
| ATOM | 1909 | CG1 | ILE | A | 241 | 84.370 | 25.716 | −13.623 | 1.00 | 93.04 | C |
| ATOM | 1910 | CD1 | ILE | A | 241 | 83.371 | 25.475 | −14.738 | 1.00 | 94.29 | C |
| ATOM | 1911 | CG2 | ILE | A | 241 | 82.582 | 25.897 | −11.854 | 1.00 | 88.01 | C |
| ATOM | 1912 | C | ILE | A | 241 | 86.254 | 24.744 | −11.523 | 1.00 | 83.81 | C |
| ATOM | 1913 | O | ILE | A | 241 | 86.244 | 23.519 | −11.653 | 1.00 | 82.49 | O |
| ATOM | 1914 | N | ALA | A | 242 | 87.356 | 25.474 | −11.682 | 1.00 | 81.78 | N |
| ATOM | 1915 | CA | ALA | A | 242 | 88.653 | 24.872 | −12.006 | 1.00 | 82.72 | C |
| ATOM | 1916 | CB | ALA | A | 242 | 89.058 | 25.243 | −13.426 | 1.00 | 83.77 | C |
| ATOM | 1917 | C | ALA | A | 242 | 89.720 | 25.330 | −11.007 | 1.00 | 81.87 | C |
| ATOM | 1918 | O | ALA | A | 242 | 90.746 | 25.886 | −11.403 | 1.00 | 86.24 | O |
| ATOM | 1919 | N | PRO | A | 243 | 89.491 | 25.078 | −9.706 | 1.00 | 78.01 | N |
| ATOM | 1920 | CA | PRO | A | 243 | 90.380 | 25.570 | −8.659 | 1.00 | 76.24 | C |
| ATOM | 1921 | CB | PRO | A | 243 | 89.671 | 25.135 | −7.374 | 1.00 | 76.42 | C |
| ATOM | 1922 | CG | PRO | A | 243 | 88.922 | 23.922 | −7.762 | 1.00 | 76.32 | C |
| ATOM | 1923 | CD | PRO | A | 243 | 88.454 | 24.189 | −9.159 | 1.00 | 77.94 | C |
| ATOM | 1924 | C | PRO | A | 243 | 91.775 | 24.963 | −8.688 | 1.00 | 76.10 | C |
| ATOM | 1925 | O | PRO | A | 243 | 91.943 | 23.840 | −9.163 | 1.00 | 79.70 | O |
| ATOM | 1926 | N | PRO | A | 244 | 92.774 | 25.704 | −8.180 | 1.00 | 75.98 | N |
| ATOM | 1927 | CA | PRO | A | 244 | 94.118 | 25.170 | −8.030 | 1.00 | 76.31 | C |
| ATOM | 1928 | CB | PRO | A | 244 | 94.962 | 26.420 | −7.789 | 1.00 | 76.74 | C |
| ATOM | 1929 | CG | PRO | A | 244 | 94.036 | 27.354 | −7.101 | 1.00 | 78.02 | C |
| ATOM | 1930 | CD | PRO | A | 244 | 92.691 | 27.103 | −7.721 | 1.00 | 78.51 | C |
| ATOM | 1931 | C | PRO | A | 244 | 94.177 | 24.244 | −6.818 | 1.00 | 76.54 | C |
| ATOM | 1932 | O | PRO | A | 244 | 93.361 | 24.386 | −5.906 | 1.00 | 77.43 | O |
| ATOM | 1933 | N | PHE | A | 245 | 95.121 | 23.306 | −6.813 | 1.00 | 74.44 | N |
| ATOM | 1934 | CA | PHE | A | 245 | 95.264 | 22.362 | −5.706 | 1.00 | 73.61 | C |
| ATOM | 1935 | CB | PHE | A | 245 | 94.775 | 20.975 | −6.115 | 1.00 | 74.52 | C |
| ATOM | 1936 | CG | PHE | A | 245 | 93.368 | 20.944 | −6.629 | 1.00 | 73.98 | C |
| ATOM | 1937 | CD1 | PHE | A | 245 | 92.323 | 21.415 | −5.855 | 1.00 | 74.62 | C |
| ATOM | 1938 | CE1 | PHE | A | 245 | 91.024 | 21.367 | −6.321 | 1.00 | 75.77 | C |
| ATOM | 1939 | CZ | PHE | A | 245 | 90.755 | 20.825 | −7.565 | 1.00 | 75.22 | C |
| ATOM | 1940 | CE2 | PHE | A | 245 | 91.788 | 20.338 | −8.340 | 1.00 | 74.75 | C |
| ATOM | 1941 | CD2 | PHE | A | 245 | 93.084 | 20.393 | −7.869 | 1.00 | 74.29 | C |
| ATOM | 1942 | C | PHE | A | 245 | 96.709 | 22.221 | −5.263 | 1.00 | 72.53 | C |
| ATOM | 1943 | O | PHE | A | 245 | 97.627 | 22.606 | −5.982 | 1.00 | 73.25 | O |
| ATOM | 1944 | N | TYR | A | 246 | 96.897 | 21.653 | −4.074 | 1.00 | 72.91 | N |
| ATOM | 1945 | CA | TYR | A | 246 | 98.229 | 21.308 | −3.579 | 1.00 | 72.22 | C |
| ATOM | 1946 | CB | TYR | A | 246 | 98.327 | 21.497 | −2.059 | 1.00 | 68.60 | C |
| ATOM | 1947 | CG | TYR | A | 246 | 98.162 | 22.946 | −1.635 | 1.00 | 64.47 | C |
| ATOM | 1948 | CD1 | TYR | A | 246 | 99.206 | 23.855 | −1.774 | 1.00 | 62.58 | C |
| ATOM | 1949 | CE1 | TYR | A | 246 | 99.059 | 25.179 | −1.400 | 1.00 | 61.70 | C |
| ATOM | 1950 | CZ | TYR | A | 246 | 97.859 | 25.611 | −0.878 | 1.00 | 63.67 | C |
| ATOM | 1951 | OH | TYR | A | 246 | 97.704 | 26.932 | −0.505 | 1.00 | 65.65 | O |
| ATOM | 1952 | CE2 | TYR | A | 246 | 96.807 | 24.728 | −0.731 | 1.00 | 64.18 | C |
| ATOM | 1953 | CD2 | TYR | A | 246 | 96.963 | 23.406 | −1.110 | 1.00 | 63.71 | C |
| ATOM | 1954 | C | TYR | A | 246 | 98.484 | 19.868 | −4.014 | 1.00 | 75.37 | C |
| ATOM | 1955 | O | TYR | A | 246 | 98.487 | 18.939 | −3.204 | 1.00 | 74.50 | O |
| ATOM | 1956 | N | ASN | A | 247 | 98.678 | 19.720 | −5.325 | 1.00 | 79.83 | N |
| ATOM | 1957 | CA | ASN | A | 247 | 98.830 | 18.434 | −6.003 | 1.00 | 80.51 | C |
| ATOM | 1958 | CB | ASN | A | 247 | 97.544 | 18.107 | −6.774 | 1.00 | 80.27 | C |
| ATOM | 1959 | CG | ASN | A | 247 | 96.464 | 17.522 | −5.903 | 1.00 | 83.93 | C |
| ATOM | 1960 | OD1 | ASN | A | 247 | 96.733 | 16.963 | −4.840 | 1.00 | 93.05 | O |
| ATOM | 1961 | ND2 | ASN | A | 247 | 95.225 | 17.621 | −6.366 | 1.00 | 85.79 | N |
| ATOM | 1962 | C | ASN | A | 247 | 99.952 | 18.480 | −7.020 | 1.00 | 82.01 | C |
| ATOM | 1963 | O | ASN | A | 247 | 100.646 | 19.491 | −7.160 | 1.00 | 78.89 | O |
| ATOM | 1964 | N | ASP | A | 248 | 100.112 | 17.371 | −7.739 | 1.00 | 84.71 | N |
| ATOM | 1965 | CA | ASP | A | 248 | 101.017 | 17.314 | −8.880 | 1.00 | 84.53 | C |
| ATOM | 1966 | CB | ASP | A | 248 | 100.996 | 15.918 | −9.516 | 1.00 | 87.09 | C |
| ATOM | 1967 | CG | ASP | A | 248 | 101.481 | 14.828 | −8.570 | 1.00 | 88.71 | C |
| ATOM | 1968 | OD1 | ASP | A | 248 | 102.396 | 15.092 | −7.764 | 1.00 | 90.98 | O |
| ATOM | 1969 | OD2 | ASP | A | 248 | 100.952 | 13.698 | −8.642 | 1.00 | 92.33 | O |
| ATOM | 1970 | C | ASP | A | 248 | 100.591 | 18.374 | −9.902 | 1.00 | 81.24 | C |
| ATOM | 1971 | O | ASP | A | 248 | 101.433 | 18.957 | −10.579 | 1.00 | 81.25 | O |
| ATOM | 1972 | N | LEU | A | 249 | 99.286 | 18.641 | −9.978 | 1.00 | 77.42 | N |
| ATOM | 1973 | CA | LEU | A | 249 | 98.732 | 19.660 | −10.873 | 1.00 | 75.67 | C |
| ATOM | 1974 | CB | LEU | A | 249 | 97.238 | 19.847 | −10.598 | 1.00 | 75.24 | C |
| ATOM | 1975 | CG | LEU | A | 249 | 96.473 | 20.856 | −11.463 | 1.00 | 74.40 | C |
| ATOM | 1976 | CD1 | LEU | A | 249 | 96.534 | 20.491 | −12.938 | 1.00 | 74.67 | C |
| ATOM | 1977 | CD2 | LEU | A | 249 | 95.028 | 20.947 | −11.005 | 1.00 | 74.24 | C |
| ATOM | 1978 | C | LEU | A | 249 | 99.442 | 21.014 | −10.787 | 1.00 | 76.45 | C |
| ATOM | 1979 | O | LEU | A | 249 | 99.513 | 21.734 | −11.782 | 1.00 | 77.11 | O |
| ATOM | 1980 | N | TYR | A | 250 | 99.941 | 21.366 | −9.604 | 1.00 | 77.44 | N |
| ATOM | 1981 | CA | TYR | A | 250 | 100.709 | 22.604 | −9.418 | 1.00 | 76.72 | C |
| ATOM | 1982 | CB | TYR | A | 250 | 99.971 | 23.544 | −8.460 | 1.00 | 76.13 | C |
| ATOM | 1983 | CG | TYR | A | 250 | 98.875 | 24.317 | −9.151 | 1.00 | 77.89 | C |
| ATOM | 1984 | CD1 | TYR | A | 250 | 99.041 | 25.661 | −9.470 | 1.00 | 78.85 | C |
| ATOM | 1985 | CE1 | TYR | A | 250 | 98.046 | 26.370 | −10.121 | 1.00 | 79.13 | C |
| ATOM | 1986 | CZ | TYR | A | 250 | 96.873 | 25.733 | −10.478 | 1.00 | 77.99 | C |
| ATOM | 1987 | OH | TYR | A | 250 | 95.886 | 26.441 | −11.125 | 1.00 | 78.48 | O |
| ATOM | 1988 | CE2 | TYR | A | 250 | 96.689 | 24.397 | −10.183 | 1.00 | 78.51 | C |
| ATOM | 1989 | CD2 | TYR | A | 250 | 97.690 | 23.696 | −9.527 | 1.00 | 78.94 | C |
| ATOM | 1990 | C | TYR | A | 250 | 102.139 | 22.314 | −8.949 | 1.00 | 77.37 | C |
| ATOM | 1991 | O | TYR | A | 250 | 102.786 | 23.154 | −8.319 | 1.00 | 79.16 | O |
| ATOM | 1992 | N | ASN | A | 251 | 102.627 | 21.124 | −9.302 | 1.00 | 75.93 | N |
| ATOM | 1993 | CA | ASN | A | 251 | 103.972 | 20.654 | −8.959 | 1.00 | 71.67 | C |
| ATOM | 1994 | CB | ASN | A | 251 | 105.034 | 21.535 | −9.629 | 1.00 | 68.52 | C |
| ATOM | 1995 | CG | ASN | A | 251 | 106.359 | 20.814 | −9.825 | 1.00 | 66.38 | C |
| ATOM | 1996 | OD1 | ASN | A | 251 | 106.408 | 19.585 | −9.907 | 1.00 | 63.22 | O |
| ATOM | 1997 | ND2 | ASN | A | 251 | 107.446 | 21.583 | −9.909 | 1.00 | 65.44 | N |
| ATOM | 1998 | C | ASN | A | 251 | 104.234 | 20.545 | −7.451 | 1.00 | 72.30 | C |
| ATOM | 1999 | O | ASN | A | 251 | 105.375 | 20.673 | −6.999 | 1.00 | 71.50 | O |
| ATOM | 2000 | N | PHE | A | 252 | 103.179 | 20.306 | −6.678 | 1.00 | 72.57 | N |
| ATOM | 2001 | CA | PHE | A | 252 | 103.325 | 20.072 | −5.247 | 1.00 | 73.19 | C |
| ATOM | 2002 | CB | PHE | A | 252 | 102.166 | 20.686 | −4.453 | 1.00 | 71.29 | C |
| ATOM | 2003 | CG | PHE | A | 252 | 102.198 | 22.192 | −4.393 | 1.00 | 70.12 | C |
| ATOM | 2004 | CD1 | PHE | A | 252 | 103.206 | 22.851 | −3.699 | 1.00 | 69.07 | C |
| ATOM | 2005 | CE1 | PHE | A | 252 | 103.239 | 24.236 | −3.636 | 1.00 | 67.34 | C |
| ATOM | 2006 | CZ | PHE | A | 252 | 102.253 | 24.980 | −4.258 | 1.00 | 66.91 | C |
| ATOM | 2007 | CE2 | PHE | A | 252 | 101.236 | 24.338 | −4.944 | 1.00 | 67.96 | C |
| ATOM | 2008 | CD2 | PHE | A | 252 | 101.209 | 22.952 | −5.006 | 1.00 | 69.30 | C |
| ATOM | 2009 | C | PHE | A | 252 | 103.407 | 18.563 | −5.028 | 1.00 | 75.80 | C |
| ATOM | 2010 | O | PHE | A | 252 | 102.383 | 17.886 | −4.913 | 1.00 | 72.23 | O |
| ATOM | 2011 | N | LYS | A | 253 | 104.634 | 18.045 | −4.987 | 1.00 | 81.32 | N |
| ATOM | 2012 | CA | LYS | A | 253 | 104.864 | 16.615 | −4.785 | 1.00 | 85.77 | C |
| ATOM | 2013 | CB | LYS | A | 253 | 106.331 | 16.241 | −5.026 | 1.00 | 92.98 | C |
| ATOM | 2014 | CG | LYS | A | 253 | 106.767 | 16.408 | −6.472 | 1.00 | 99.97 | C |
| ATOM | 2015 | CD | LYS | A | 253 | 108.226 | 16.049 | −6.675 | 1.00 | 106.09 | C |
| ATOM | 2016 | CE | LYS | A | 253 | 109.129 | 17.247 | −6.439 | 1.00 | 111.70 | C |
| ATOM | 2017 | NZ | LYS | A | 253 | 110.541 | 16.971 | −6.825 | 1.00 | 115.13 | N |
| ATOM | 2018 | C | LYS | A | 253 | 104.424 | 16.191 | −3.387 | 1.00 | 86.29 | C |
| ATOM | 2019 | O | LYS | A | 253 | 104.542 | 16.954 | −2.421 | 1.00 | 88.83 | O |
| ATOM | 2020 | N | ASP | A | 254 | 103.890 | 14.979 | −3.291 | 1.00 | 84.69 | N |
| ATOM | 2021 | CA | ASP | A | 254 | 103.447 | 14.441 | −2.014 | 1.00 | 79.32 | C |
| ATOM | 2022 | CB | ASP | A | 254 | 102.777 | 13.076 | −2.216 | 1.00 | 79.03 | C |
| ATOM | 2023 | CG | ASP | A | 254 | 101.821 | 12.712 | −1.095 | 1.00 | 79.95 | C |
| ATOM | 2024 | OD1 | ASP | A | 254 | 101.391 | 13.611 | −0.345 | 1.00 | 79.56 | O |
| ATOM | 2025 | OD2 | ASP | A | 254 | 101.476 | 11.517 | −0.979 | 1.00 | 83.79 | O |
| ATOM | 2026 | C | ASP | A | 254 | 104.684 | 14.321 | −1.131 | 1.00 | 77.94 | C |
| ATOM | 2027 | O | ASP | A | 254 | 105.735 | 13.878 | −1.592 | 1.00 | 77.26 | O |
| ATOM | 2028 | N | GLY | A | 255 | 104.570 | 14.750 | 0.122 | 1.00 | 77.82 | N |
| ATOM | 2029 | CA | GLY | A | 255 | 105.690 | 14.703 | 1.061 | 1.00 | 75.19 | C |
| ATOM | 2030 | C | GLY | A | 255 | 106.428 | 16.024 | 1.162 | 1.00 | 73.47 | C |
| ATOM | 2031 | O | GLY | A | 255 | 105.882 | 17.078 | 0.847 | 1.00 | 73.15 | O |
| ATOM | 2032 | N | ALA | A | 256 | 107.681 | 15.961 | 1.599 | 1.00 | 73.11 | N |
| ATOM | 2033 | CA | ALA | A | 256 | 108.483 | 17.159 | 1.825 | 1.00 | 73.60 | C |
| ATOM | 2034 | CB | ALA | A | 256 | 109.684 | 16.829 | 2.703 | 1.00 | 72.94 | C |
| ATOM | 2035 | C | ALA | A | 256 | 108.948 | 17.803 | 0.522 | 1.00 | 74.79 | C |
| ATOM | 2036 | O | ALA | A | 256 | 109.271 | 17.110 | −0.443 | 1.00 | 75.91 | O |
| ATOM | 2037 | N | GLN | A | 257 | 108.985 | 19.132 | 0.514 | 1.00 | 74.46 | N |
| ATOM | 2038 | CA | GLN | A | 257 | 109.469 | 19.893 | −0.628 | 1.00 | 74.65 | C |
| ATOM | 2039 | CB | GLN | A | 257 | 108.696 | 21.213 | −0.770 | 1.00 | 74.41 | C |
| ATOM | 2040 | CG | GLN | A | 257 | 107.178 | 21.068 | −0.734 | 1.00 | 74.48 | C |
| ATOM | 2041 | CD | GLN | A | 257 | 106.627 | 20.175 | −1.841 | 1.00 | 75.54 | C |
| ATOM | 2042 | OE1 | GLN | A | 257 | 106.881 | 20.398 | −3.028 | 1.00 | 72.96 | O |
| ATOM | 2043 | NE2 | GLN | A | 257 | 105.852 | 19.169 | −1.456 | 1.00 | 76.19 | N |
| ATOM | 2044 | C | GLN | A | 257 | 110.958 | 20.148 | −0.401 | 1.00 | 75.86 | C |
| ATOM | 2045 | O | GLN | A | 257 | 111.344 | 21.135 | 0.226 | 1.00 | 78.46 | O |
| ATOM | 2046 | N | ASN | A | 258 | 111.786 | 19.244 | −0.918 | 1.00 | 77.15 | N |
| ATOM | 2047 | CA | ASN | A | 258 | 113.235 | 19.270 | −0.685 | 1.00 | 78.64 | C |
| ATOM | 2048 | CB | ASN | A | 258 | 113.801 | 17.857 | −0.862 | 1.00 | 79.34 | C |
| ATOM | 2049 | CG | ASN | A | 258 | 113.222 | 16.867 | 0.135 | 1.00 | 80.33 | C |
| ATOM | 2050 | OD1 | ASN | A | 258 | 112.547 | 15.911 | −0.245 | 1.00 | 81.10 | O |
| ATOM | 2051 | ND2 | ASN | A | 258 | 113.475 | 17.098 | 1.417 | 1.00 | 80.45 | N |
| ATOM | 2052 | C | ASN | A | 258 | 114.042 | 20.244 | −1.549 | 1.00 | 79.55 | C |
| ATOM | 2053 | O | ASN | A | 258 | 115.225 | 20.467 | −1.285 | 1.00 | 79.77 | O |
| ATOM | 2054 | N | ASP | A | 259 | 113.409 | 20.832 | −2.560 | 1.00 | 81.95 | N |
| ATOM | 2055 | CA | ASP | A | 259 | 114.106 | 21.715 | −3.508 | 1.00 | 84.57 | C |
| ATOM | 2056 | CB | ASP | A | 259 | 113.758 | 21.302 | −4.946 | 1.00 | 85.64 | C |
| ATOM | 2057 | CG | ASP | A | 259 | 112.263 | 21.364 | −5.237 | 1.00 | 86.82 | C |
| ATOM | 2058 | OD1 | ASP | A | 259 | 111.458 | 21.428 | −4.282 | 1.00 | 84.66 | O |
| ATOM | 2059 | OD2 | ASP | A | 259 | 111.893 | 21.328 | −6.427 | 1.00 | 87.99 | O |
| ATOM | 2060 | C | ASP | A | 259 | 113.808 | 23.204 | −3.304 | 1.00 | 84.43 | C |
| ATOM | 2061 | O | ASP | A | 259 | 113.875 | 23.990 | −4.254 | 1.00 | 89.08 | O |
| ATOM | 2062 | N | ILE | A | 260 | 113.514 | 23.590 | −2.064 | 1.00 | 81.70 | N |
| ATOM | 2063 | CA | ILE | A | 260 | 113.126 | 24.971 | −1.751 | 1.00 | 79.45 | C |
| ATOM | 2064 | CB | ILE | A | 260 | 112.008 | 25.012 | −0.679 | 1.00 | 78.65 | C |
| ATOM | 2065 | CG1 | ILE | A | 260 | 112.502 | 24.469 | 0.671 | 1.00 | 79.64 | C |
| ATOM | 2066 | CD1 | ILE | A | 260 | 111.509 | 24.642 | 1.801 | 1.00 | 79.25 | C |
| ATOM | 2067 | CG2 | ILE | A | 260 | 110.794 | 24.227 | −1.154 | 1.00 | 78.74 | C |
| ATOM | 2068 | C | ILE | A | 260 | 114.287 | 25.851 | −1.286 | 1.00 | 78.23 | C |
| ATOM | 2069 | O | ILE | A | 260 | 114.076 | 27.007 | −0.919 | 1.00 | 78.94 | O |
| ATOM | 2070 | N | PHE | A | 261 | 115.505 | 25.319 | −1.321 | 1.00 | 78.47 | N |
| ATOM | 2071 | CA | PHE | A | 261 | 116.668 | 26.030 | −0.788 | 1.00 | 81.03 | C |
| ATOM | 2072 | CB | PHE | A | 261 | 117.508 | 25.080 | 0.067 | 1.00 | 79.65 | C |
| ATOM | 2073 | CG | PHE | A | 261 | 116.687 | 24.280 | 1.040 | 1.00 | 77.23 | C |
| ATOM | 2074 | CD1 | PHE | A | 261 | 116.206 | 24.862 | 2.201 | 1.00 | 76.79 | C |
| ATOM | 2075 | CE1 | PHE | A | 261 | 115.435 | 24.136 | 3.089 | 1.00 | 75.75 | C |
| ATOM | 2076 | CZ | PHE | A | 261 | 115.130 | 22.814 | 2.818 | 1.00 | 75.19 | C |
| ATOM | 2077 | CE2 | PHE | A | 261 | 115.597 | 22.224 | 1.660 | 1.00 | 75.46 | C |
| ATOM | 2078 | CD2 | PHE | A | 261 | 116.367 | 22.957 | 0.776 | 1.00 | 76.57 | C |
| ATOM | 2079 | C | PHE | A | 261 | 117.507 | 26.667 | −1.884 | 1.00 | 83.24 | C |
| ATOM | 2080 | O | PHE | A | 261 | 117.356 | 26.345 | −3.063 | 1.00 | 86.36 | O |
| ATOM | 2081 | N | VAL | A | 262 | 118.393 | 27.575 | −1.481 | 1.00 | 86.71 | N |
| ATOM | 2082 | CA | VAL | A | 262 | 119.223 | 28.325 | −2.422 | 1.00 | 88.45 | C |
| ATOM | 2083 | CB | VAL | A | 262 | 120.044 | 29.424 | −1.709 | 1.00 | 89.79 | C |
| ATOM | 2084 | CG1 | VAL | A | 262 | 120.955 | 30.146 | −2.697 | 1.00 | 91.60 | C |
| ATOM | 2085 | CG2 | VAL | A | 262 | 119.120 | 30.418 | −1.016 | 1.00 | 90.85 | C |
| ATOM | 2086 | C | VAL | A | 262 | 120.166 | 27.404 | −3.198 | 1.00 | 90.80 | C |
| ATOM | 2087 | O | VAL | A | 262 | 120.316 | 27.547 | −4.413 | 1.00 | 95.70 | O |
| ATOM | 2088 | N | ASP | A | 263 | 120.784 | 26.457 | −2.497 | 1.00 | 90.45 | N |
| ATOM | 2089 | CA | ASP | A | 263 | 121.726 | 25.519 | −3.118 | 1.00 | 88.68 | C |
| ATOM | 2090 | CB | ASP | A | 263 | 122.763 | 25.043 | −2.087 | 1.00 | 89.04 | C |
| ATOM | 2091 | CG | ASP | A | 263 | 122.158 | 24.179 | −0.984 | 1.00 | 88.58 | C |
| ATOM | 2092 | OD1 | ASP | A | 263 | 120.943 | 24.289 | −0.721 | 1.00 | 89.30 | O |
| ATOM | 2093 | OD2 | ASP | A | 263 | 122.909 | 23.391 | −0.373 | 1.00 | 89.57 | O |
| ATOM | 2094 | C | ASP | A | 263 | 121.055 | 24.306 | −3.779 | 1.00 | 87.09 | C |
| ATOM | 2095 | O | ASP | A | 263 | 121.750 | 23.446 | −4.325 | 1.00 | 85.49 | O |
| ATOM | 2096 | N | SER | A | 264 | 119.723 | 24.235 | −3.731 | 1.00 | 87.15 | N |
| ATOM | 2097 | CA | SER | A | 264 | 118.981 | 23.097 | −4.287 | 1.00 | 87.74 | C |
| ATOM | 2098 | CB | SER | A | 264 | 117.471 | 23.291 | −4.125 | 1.00 | 91.73 | C |
| ATOM | 2099 | OG | SER | A | 264 | 117.102 | 23.275 | −2.756 | 1.00 | 94.76 | O |
| ATOM | 2100 | C | SER | A | 264 | 119.312 | 22.849 | −5.749 | 1.00 | 85.13 | C |
| ATOM | 2101 | O | SER | A | 264 | 119.709 | 21.745 | −6.108 | 1.00 | 82.83 | O |
| ATOM | 2102 | N | TYR | A | 265 | 119.149 | 23.876 | −6.579 | 1.00 | 86.01 | N |
| ATOM | 2103 | CA | TYR | A | 265 | 119.461 | 23.775 | −8.008 | 1.00 | 87.83 | C |
| ATOM | 2104 | CB | TYR | A | 265 | 118.352 | 24.418 | −8.842 | 1.00 | 84.66 | C |
| ATOM | 2105 | CG | TYR | A | 265 | 117.039 | 23.670 | −8.788 | 1.00 | 81.02 | C |
| ATOM | 2106 | CD1 | TYR | A | 265 | 116.038 | 24.033 | −7.890 | 1.00 | 80.85 | C |
| ATOM | 2107 | CE1 | TYR | A | 265 | 114.836 | 23.343 | −7.840 | 1.00 | 78.37 | C |
| ATOM | 2108 | CZ | TYR | A | 265 | 114.629 | 22.274 | −8.691 | 1.00 | 77.16 | C |
| ATOM | 2109 | OH | TYR | A | 265 | 113.448 | 21.578 | −8.654 | 1.00 | 73.76 | O |
| ATOM | 2110 | CE2 | TYR | A | 265 | 115.605 | 21.897 | −9.589 | 1.00 | 77.96 | C |
| ATOM | 2111 | CD2 | TYR | A | 265 | 116.801 | 22.592 | −9.633 | 1.00 | 79.46 | C |
| ATOM | 2112 | C | TYR | A | 265 | 120.810 | 24.444 | −8.309 | 1.00 | 93.04 | C |
| ATOM | 2113 | O | TYR | A | 265 | 120.899 | 25.672 | −8.296 | 1.00 | 95.54 | O |
| ATOM | 2114 | N | PRO | A | 266 | 121.863 | 23.643 | −8.588 | 1.00 | 97.30 | N |
| ATOM | 2115 | CA | PRO | A | 266 | 123.201 | 24.199 | −8.834 | 1.00 | 98.80 | C |
| ATOM | 2116 | CB | PRO | A | 266 | 124.093 | 22.954 | −8.953 | 1.00 | 99.77 | C |
| ATOM | 2117 | CG | PRO | A | 266 | 123.288 | 21.837 | −8.389 | 1.00 | 99.49 | C |
| ATOM | 2118 | CD | PRO | A | 266 | 121.873 | 22.178 | −8.724 | 1.00 | 98.32 | C |
| ATOM | 2119 | C | PRO | A | 266 | 123.274 | 25.015 | −10.113 | 1.00 | 99.63 | C |
| ATOM | 2120 | O | PRO | A | 266 | 123.742 | 26.152 | −10.089 | 1.00 | 100.28 | O |
| ATOM | 2121 | N | ASP | A | 267 | 122.797 | 24.431 | −11.210 | 1.00 | 102.03 | N |
| ATOM | 2122 | CA | ASP | A | 267 | 122.783 | 25.090 | −12.511 | 1.00 | 104.39 | C |
| ATOM | 2123 | CB | ASP | A | 267 | 122.805 | 24.049 | −13.637 | 1.00 | 107.27 | C |
| ATOM | 2124 | CG | ASP | A | 267 | 124.012 | 23.135 | −13.566 | 1.00 | 110.11 | C |
| ATOM | 2125 | OD1 | ASP | A | 267 | 125.147 | 23.647 | −13.457 | 1.00 | 111.81 | O |
| ATOM | 2126 | OD2 | ASP | A | 267 | 123.821 | 21.904 | −13.637 | 1.00 | 110.35 | O |
| ATOM | 2127 | C | ASP | A | 267 | 121.564 | 26.002 | −12.678 | 1.00 | 103.12 | C |
| ATOM | 2128 | O | ASP | A | 267 | 121.354 | 26.534 | −13.764 | 1.00 | 102.02 | O |
| ATOM | 2129 | N | GLY | A | 268 | 120.760 | 26.172 | −11.625 | 1.00 | 101.76 | N |
| ATOM | 2130 | CA | GLY | A | 268 | 119.619 | 27.079 | −11.662 | 1.00 | 100.79 | C |
| ATOM | 2131 | C | GLY | A | 268 | 118.421 | 26.487 | −12.372 | 1.00 | 100.45 | C |
| ATOM | 2132 | O | GLY | A | 268 | 118.362 | 25.281 | −12.615 | 1.00 | 101.51 | O |
| ATOM | 2133 | N | ILE | A | 269 | 117.471 | 27.354 | −12.712 | 1.00 | 99.07 | N |
| ATOM | 2134 | CA | ILE | A | 269 | 116.263 | 26.968 | −13.431 | 1.00 | 97.44 | C |
| ATOM | 2135 | CB | ILE | A | 269 | 114.996 | 27.165 | −12.561 | 1.00 | 97.79 | C |
| ATOM | 2136 | CG1 | ILE | A | 269 | 115.128 | 26.365 | −11.255 | 1.00 | 96.99 | C |
| ATOM | 2137 | CD1 | ILE | A | 269 | 115.244 | 27.208 | −10.004 | 1.00 | 97.80 | C |
| ATOM | 2138 | CG2 | ILE | A | 269 | 113.731 | 26.752 | −13.309 | 1.00 | 99.53 | C |
| ATOM | 2139 | C | ILE | A | 269 | 116.176 | 27.798 | −14.713 | 1.00 | 97.53 | C |
| ATOM | 2140 | O | ILE | A | 269 | 115.593 | 28.878 | −14.708 | 1.00 | 98.28 | O |
| ATOM | 2141 | N | PRO | A | 270 | 116.793 | 27.312 | −15.808 | 1.00 | 98.11 | N |
| ATOM | 2142 | CA | PRO | A | 270 | 116.708 | 27.974 | −17.115 | 1.00 | 98.64 | C |
| ATOM | 2143 | CB | PRO | A | 270 | 117.383 | 26.973 | −18.050 | 1.00 | 99.56 | C |
| ATOM | 2144 | CG | PRO | A | 270 | 118.402 | 26.311 | −17.193 | 1.00 | 100.10 | C |
| ATOM | 2145 | CD | PRO | A | 270 | 117.822 | 26.255 | −15.806 | 1.00 | 99.54 | C |
| ATOM | 2146 | C | PRO | A | 270 | 115.277 | 28.264 | −17.590 | 1.00 | 97.88 | C |
| ATOM | 2147 | O | PRO | A | 270 | 115.063 | 29.227 | −18.329 | 1.00 | 99.51 | O |
| ATOM | 2148 | N | LEU | A | 271 | 114.317 | 27.437 | −17.170 | 1.00 | 95.54 | N |
| ATOM | 2149 | CA | LEU | A | 271 | 112.901 | 27.631 | −17.509 | 1.00 | 95.53 | C |
| ATOM | 2150 | CB | LEU | A | 271 | 112.035 | 26.515 | −16.919 | 1.00 | 93.02 | C |
| ATOM | 2151 | CG | LEU | A | 271 | 111.789 | 25.274 | −17.775 | 1.00 | 91.39 | C |
| ATOM | 2152 | CD1 | LEU | A | 271 | 113.066 | 24.535 | −18.101 | 1.00 | 89.61 | C |
| ATOM | 2153 | CD2 | LEU | A | 271 | 110.834 | 24.345 | −17.046 | 1.00 | 93.51 | C |
| ATOM | 2154 | C | LEU | A | 271 | 112.338 | 28.975 | −17.055 | 1.00 | 100.00 | C |
| ATOM | 2155 | O | LEU | A | 271 | 111.328 | 29.430 | −17.586 | 1.00 | 103.24 | O |
| ATOM | 2156 | N | GLU | A | 272 | 112.974 | 29.598 | −16.068 | 1.00 | 105.60 | N |
| ATOM | 2157 | CA | GLU | A | 272 | 112.547 | 30.912 | −15.595 | 1.00 | 111.95 | C |
| ATOM | 2158 | CB | GLU | A | 272 | 113.237 | 31.255 | −14.272 | 1.00 | 115.04 | C |
| ATOM | 2159 | CG | GLU | A | 272 | 112.796 | 30.355 | −13.123 | 1.00 | 115.05 | C |
| ATOM | 2160 | CD | GLU | A | 272 | 113.542 | 30.617 | −11.825 | 1.00 | 116.07 | C |
| ATOM | 2161 | OE1 | GLU | A | 272 | 114.662 | 31.175 | −11.865 | 1.00 | 115.23 | O |
| ATOM | 2162 | OE2 | GLU | A | 272 | 113.005 | 30.252 | −10.757 | 1.00 | 113.02 | O |
| ATOM | 2163 | C | GLU | A | 272 | 112.778 | 32.014 | −16.640 | 1.00 | 114.41 | C |
| ATOM | 2164 | O | GLU | A | 272 | 112.257 | 33.118 | −16.497 | 1.00 | 115.45 | O |
| ATOM | 2165 | N | GLN | A | 273 | 113.557 | 31.715 | −17.681 | 1.00 | 118.50 | N |
| ATOM | 2166 | CA | GLN | A | 273 | 113.747 | 32.640 | −18.800 | 1.00 | 125.42 | C |
| ATOM | 2167 | CB | GLN | A | 273 | 114.790 | 32.092 | −19.781 | 1.00 | 125.39 | C |
| ATOM | 2168 | CG | GLN | A | 273 | 115.017 | 32.962 | −21.010 | 1.00 | 125.94 | C |
| ATOM | 2169 | CD | GLN | A | 273 | 116.022 | 32.366 | −21.977 | 1.00 | 127.86 | C |
| ATOM | 2170 | OE1 | GLN | A | 273 | 115.685 | 32.049 | −23.118 | 1.00 | 127.00 | O |
| ATOM | 2171 | NE2 | GLN | A | 273 | 117.259 | 32.198 | −21.522 | 1.00 | 129.88 | N |
| ATOM | 2172 | C | GLN | A | 273 | 112.422 | 32.879 | −19.529 | 1.00 | 132.03 | C |
| ATOM | 2173 | O | GLN | A | 273 | 112.161 | 33.984 | −20.010 | 1.00 | 133.87 | O |
| ATOM | 2174 | N | LYS | A | 274 | 111.592 | 31.840 | −19.601 | 1.00 | 138.59 | N |
| ATOM | 2175 | CA | LYS | A | 274 | 110.282 | 31.923 | −20.253 | 1.00 | 141.98 | C |
| ATOM | 2176 | CB | LYS | A | 274 | 109.711 | 30.519 | −20.480 | 1.00 | 144.49 | C |
| ATOM | 2177 | CG | LYS | A | 274 | 110.568 | 29.656 | −21.397 | 1.00 | 147.46 | C |
| ATOM | 2178 | CD | LYS | A | 274 | 110.051 | 28.230 | −21.495 | 1.00 | 149.98 | C |
| ATOM | 2179 | CE | LYS | A | 274 | 110.973 | 27.373 | −22.348 | 1.00 | 151.33 | C |
| ATOM | 2180 | NZ | LYS | A | 274 | 110.500 | 25.966 | −22.455 | 1.00 | 152.26 | N |
| ATOM | 2181 | C | LYS | A | 274 | 109.295 | 32.783 | −19.456 | 1.00 | 143.02 | C |
| ATOM | 2182 | O | LYS | A | 274 | 108.337 | 33.311 | −20.018 | 1.00 | 141.96 | O |
| ATOM | 2183 | N | LEU | A | 275 | 109.533 | 32.916 | −18.152 | 1.00 | 149.30 | N |
| ATOM | 2184 | CA | LEU | A | 275 | 108.716 | 33.777 | −17.291 | 1.00 | 154.74 | C |
| ATOM | 2185 | CB | LEU | A | 275 | 108.867 | 33.374 | −15.819 | 1.00 | 158.31 | C |
| ATOM | 2186 | CG | LEU | A | 275 | 108.535 | 31.925 | −15.445 | 1.00 | 162.45 | C |
| ATOM | 2187 | CD1 | LEU | A | 275 | 108.845 | 31.673 | −13.976 | 1.00 | 161.31 | C |
| ATOM | 2188 | CD2 | LEU | A | 275 | 107.082 | 31.590 | −15.755 | 1.00 | 162.69 | C |
| ATOM | 2189 | C | LEU | A | 275 | 109.105 | 35.249 | −17.455 | 1.00 | 154.01 | C |
| ATOM | 2190 | O | LEU | A | 275 | 108.273 | 36.138 | −17.273 | 1.00 | 153.38 | O |
| ATOM | 2191 | N | ILE | A | 276 | 110.371 | 35.496 | −17.792 | 1.00 | 155.71 | N |
| ATOM | 2192 | CA | ILE | A | 276 | 110.884 | 36.858 | −17.988 | 1.00 | 155.92 | C |
| ATOM | 2193 | CB | ILE | A | 276 | 112.436 | 36.874 | −18.018 | 1.00 | 156.41 | C |
| ATOM | 2194 | CG1 | ILE | A | 276 | 112.996 | 36.460 | −16.651 | 1.00 | 155.75 | C |
| ATOM | 2195 | CD1 | ILE | A | 276 | 114.482 | 36.168 | −16.645 | 1.00 | 153.90 | C |
| ATOM | 2196 | CG2 | ILE | A | 276 | 112.967 | 38.256 | −18.385 | 1.00 | 155.85 | C |
| ATOM | 2197 | C | ILE | A | 276 | 110.315 | 37.506 | −19.259 | 1.00 | 154.59 | C |
| ATOM | 2198 | O | ILE | A | 276 | 110.170 | 38.728 | −19.320 | 1.00 | 152.55 | O |
| ATOM | 2199 | N | SER | A | 277 | 109.987 | 36.690 | −20.261 | 1.00 | 154.98 | N |
| ATOM | 2200 | CA | SER | A | 277 | 109.430 | 37.192 | −21.524 | 1.00 | 153.95 | C |
| ATOM | 2201 | CB | SER | A | 277 | 109.402 | 36.082 | −22.582 | 1.00 | 150.69 | C |
| ATOM | 2202 | OG | SER | A | 277 | 108.569 | 35.007 | −22.185 | 1.00 | 147.26 | O |
| ATOM | 2203 | C | SER | A | 277 | 108.027 | 37.791 | −21.363 | 1.00 | 156.19 | C |
| ATOM | 2204 | O | SER | A | 277 | 107.674 | 38.743 | −22.061 | 1.00 | 157.19 | O |
| ATOM | 2205 | N | GLU | A | 278 | 107.239 | 37.231 | −20.446 | 1.00 | 156.49 | N |
| ATOM | 2206 | CA | GLU | A | 278 | 105.871 | 37.700 | −20.194 | 1.00 | 152.92 | C |
| ATOM | 2207 | CB | GLU | A | 278 | 105.094 | 36.667 | −19.369 | 1.00 | 151.29 | C |
| ATOM | 2208 | CG | GLU | A | 278 | 104.885 | 35.335 | −20.078 | 1.00 | 150.66 | C |
| ATOM | 2209 | CD | GLU | A | 278 | 104.176 | 34.299 | −19.219 | 1.00 | 149.23 | C |
| ATOM | 2210 | OE1 | GLU | A | 278 | 104.099 | 34.478 | −17.984 | 1.00 | 145.23 | O |
| ATOM | 2211 | OE2 | GLU | A | 278 | 103.700 | 33.292 | −19.784 | 1.00 | 148.65 | O |
| ATOM | 2212 | C | GLU | A | 278 | 105.854 | 39.051 | −19.480 | 1.00 | 149.61 | C |
| ATOM | 2213 | O | GLU | A | 278 | 106.758 | 39.374 | −18.709 | 1.00 | 145.55 | O |
| HETATM | 2214 | C1 | NAG | A | 300 | 92.231 | 29.653 | 25.803 | 1.00 | 88.06 | C |
| HETATM | 2215 | C2 | NAG | A | 300 | 93.546 | 29.976 | 26.521 | 1.00 | 88.70 | C |
| HETATM | 2216 | N2 | NAG | A | 300 | 94.628 | 30.106 | 25.559 | 1.00 | 88.07 | N |
| HETATM | 2217 | C7 | NAG | A | 300 | 94.728 | 31.148 | 24.730 | 1.00 | 88.36 | C |
| HETATM | 2218 | O7 | NAG | A | 300 | 93.922 | 32.075 | 24.698 | 1.00 | 86.86 | O |
| HETATM | 2219 | C8 | NAG | A | 300 | 95.899 | 31.162 | 23.792 | 1.00 | 89.47 | C |
| HETATM | 2220 | C3 | NAG | A | 300 | 93.936 | 28.982 | 27.618 | 1.00 | 93.40 | C |
| HETATM | 2221 | O3 | NAG | A | 300 | 94.906 | 29.554 | 28.468 | 1.00 | 91.90 | O |
| HETATM | 2222 | C4 | NAG | A | 300 | 92.725 | 28.576 | 28.442 | 1.00 | 97.32 | C |
| HETATM | 2223 | O4 | NAG | A | 300 | 93.069 | 27.586 | 29.395 | 1.00 | 107.70 | O |
| HETATM | 2224 | C5 | NAG | A | 300 | 91.667 | 28.059 | 27.480 | 1.00 | 94.56 | C |
| HETATM | 2225 | C6 | NAG | A | 300 | 90.444 | 27.489 | 28.189 | 1.00 | 95.47 | C |
| HETATM | 2226 | O6 | NAG | A | 300 | 89.905 | 28.462 | 29.053 | 1.00 | 98.19 | O |
| HETATM | 2227 | O5 | NAG | A | 300 | 91.261 | 29.151 | 26.700 | 1.00 | 88.89 | O |
| HETATM | 2228 | C1 | NAG | A | 301 | 93.078 | 28.120 | 30.733 | 1.00 | 119.47 | C |
| HETATM | 2229 | C2 | NAG | A | 301 | 92.849 | 26.984 | 31.739 | 1.00 | 125.81 | C |
| HETATM | 2230 | N2 | NAG | A | 301 | 91.499 | 26.413 | 31.646 | 1.00 | 126.51 | N |
| HETATM | 2231 | C7 | NAG | A | 301 | 90.352 | 27.055 | 31.942 | 1.00 | 121.91 | C |
| HETATM | 2232 | O7 | NAG | A | 301 | 90.283 | 28.222 | 32.329 | 1.00 | 120.28 | O |
| HETATM | 2233 | C8 | NAG | A | 301 | 89.070 | 26.285 | 31.782 | 1.00 | 117.84 | C |
| HETATM | 2234 | C3 | NAG | A | 301 | 93.193 | 27.361 | 33.192 | 1.00 | 131.36 | C |
| HETATM | 2235 | O3 | NAG | A | 301 | 93.482 | 26.189 | 33.923 | 1.00 | 130.30 | O |
| HETATM | 2236 | C4 | NAG | A | 301 | 94.367 | 28.330 | 33.322 | 1.00 | 133.81 | C |
| HETATM | 2237 | O4 | NAG | A | 301 | 94.322 | 28.954 | 34.594 | 1.00 | 141.80 | O |
| HETATM | 2238 | C5 | NAG | A | 301 | 94.319 | 29.408 | 32.244 | 1.00 | 130.44 | C |
| HETATM | 2239 | C6 | NAG | A | 301 | 95.511 | 30.359 | 32.319 | 1.00 | 130.22 | C |
| HETATM | 2240 | O6 | NAG | A | 301 | 96.715 | 29.646 | 32.140 | 1.00 | 133.23 | O |
| HETATM | 2241 | O5 | NAG | A | 301 | 94.303 | 28.769 | 30.987 | 1.00 | 122.75 | O |
| HETATM | 2242 | C1 | BMA | A | 302 | 95.375 | 28.514 | 35.477 | 1.00 | 149.86 | C |
| HETATM | 2243 | O5 | BMA | A | 302 | 94.924 | 27.535 | 36.414 | 1.00 | 151.87 | O |
| HETATM | 2244 | C5 | BMA | A | 302 | 95.978 | 27.001 | 37.222 | 1.00 | 152.13 | C |
| HETATM | 2245 | C6 | BMA | A | 302 | 95.407 | 25.922 | 38.135 | 1.00 | 149.41 | C |
| HETATM | 2246 | O6 | BMA | A | 302 | 96.469 | 25.123 | 38.669 | 1.00 | 146.07 | O |
| HETATM | 2247 | C4 | BMA | A | 302 | 96.644 | 28.114 | 38.030 | 1.00 | 154.34 | C |
| HETATM | 2248 | O4 | BMA | A | 302 | 97.780 | 27.590 | 38.726 | 1.00 | 158.54 | O |
| HETATM | 2249 | C3 | BMA | A | 302 | 97.086 | 29.262 | 37.127 | 1.00 | 152.99 | C |
| HETATM | 2250 | O3 | BMA | A | 302 | 97.557 | 30.359 | 37.919 | 1.00 | 152.44 | O |
| HETATM | 2251 | C2 | BMA | A | 302 | 95.929 | 29.708 | 36.241 | 1.00 | 151.92 | C |
| HETATM | 2252 | O2 | BMA | A | 302 | 94.893 | 30.288 | 37.041 | 1.00 | 152.03 | O |
| HETATM | 2253 | C1 | NAG | A | 310 | 94.583 | 20.869 | 27.777 | 1.00 | 109.65 | C |
| HETATM | 2254 | C2 | NAG | A | 310 | 95.465 | 21.038 | 29.004 | 1.00 | 117.28 | C |
| HETATM | 2255 | N2 | NAG | A | 310 | 94.903 | 22.024 | 29.913 | 1.00 | 119.62 | N |
| HETATM | 2256 | C7 | NAG | A | 310 | 94.647 | 21.772 | 31.201 | 1.00 | 120.08 | C |
| HETATM | 2257 | O7 | NAG | A | 310 | 94.862 | 20.689 | 31.749 | 1.00 | 116.82 | O |
| HETATM | 2258 | C8 | NAG | A | 310 | 94.062 | 22.903 | 31.995 | 1.00 | 120.22 | C |
| HETATM | 2259 | C3 | NAG | A | 310 | 96.852 | 21.445 | 28.534 | 1.00 | 119.67 | C |
| HETATM | 2260 | O3 | NAG | A | 310 | 97.726 | 21.550 | 29.634 | 1.00 | 119.10 | O |
| HETATM | 2261 | C4 | NAG | A | 310 | 97.370 | 20.405 | 27.550 | 1.00 | 122.93 | C |
| HETATM | 2262 | O4 | NAG | A | 310 | 98.524 | 20.918 | 26.929 | 1.00 | 135.19 | O |
| HETATM | 2263 | C5 | NAG | A | 310 | 96.349 | 20.102 | 26.453 | 1.00 | 118.12 | C |
| HETATM | 2264 | C6 | NAG | A | 310 | 96.764 | 18.929 | 25.573 | 1.00 | 118.70 | C |
| HETATM | 2265 | O6 | NAG | A | 310 | 96.949 | 17.778 | 26.368 | 1.00 | 116.86 | O |
| HETATM | 2266 | O5 | NAG | A | 310 | 95.092 | 19.805 | 27.013 | 1.00 | 111.69 | O |
| HETATM | 2267 | C1 | NAG | A | 311 | 99.746 | 20.381 | 27.460 | 1.00 | 146.81 | C |
| HETATM | 2268 | C2 | NAG | A | 311 | 100.639 | 20.109 | 26.250 | 1.00 | 148.11 | C |
| HETATM | 2269 | N2 | NAG | A | 311 | 100.161 | 18.923 | 25.555 | 1.00 | 144.86 | N |
| HETATM | 2270 | C7 | NAG | A | 311 | 100.351 | 18.733 | 24.248 | 1.00 | 143.26 | C |
| HETATM | 2271 | O7 | NAG | A | 311 | 100.947 | 19.539 | 23.536 | 1.00 | 143.90 | O |
| HETATM | 2272 | C8 | NAG | A | 311 | 99.804 | 17.470 | 23.646 | 1.00 | 142.28 | C |
| HETATM | 2273 | C3 | NAG | A | 311 | 102.113 | 19.945 | 26.600 | 1.00 | 155.81 | C |
| HETATM | 2274 | O3 | NAG | A | 311 | 102.863 | 20.077 | 25.415 | 1.00 | 155.79 | O |
| HETATM | 2275 | C4 | NAG | A | 311 | 102.529 | 20.992 | 27.627 | 1.00 | 163.01 | C |
| HETATM | 2276 | O4 | NAG | A | 311 | 103.872 | 20.824 | 28.030 | 1.00 | 170.20 | O |
| HETATM | 2277 | C5 | NAG | A | 311 | 101.598 | 20.888 | 28.828 | 1.00 | 162.22 | C |
| HETATM | 2278 | C6 | NAG | A | 311 | 102.043 | 21.767 | 29.996 | 1.00 | 160.93 | C |
| HETATM | 2279 | O6 | NAG | A | 311 | 101.095 | 21.690 | 31.037 | 1.00 | 156.34 | O |
| HETATM | 2280 | O5 | NAG | A | 311 | 100.320 | 21.283 | 28.383 | 1.00 | 155.90 | O |
| HETATM | 2281 | C1 | BMA | A | 312 | 104.801 | 21.457 | 27.128 | 1.00 | 173.77 | C |
| HETATM | 2282 | O5 | BMA | A | 312 | 105.738 | 20.504 | 26.637 | 1.00 | 175.56 | O |
| HETATM | 2283 | C5 | BMA | A | 312 | 106.642 | 21.022 | 25.661 | 1.00 | 174.38 | C |
| HETATM | 2284 | C6 | BMA | A | 312 | 107.457 | 19.854 | 25.107 | 1.00 | 173.09 | C |
| HETATM | 2285 | O6 | BMA | A | 312 | 106.897 | 19.386 | 23.867 | 1.00 | 173.14 | O |
| HETATM | 2286 | C4 | BMA | A | 312 | 107.488 | 22.118 | 26.308 | 1.00 | 174.90 | C |
| HETATM | 2287 | O4 | BMA | A | 312 | 108.372 | 22.697 | 25.341 | 1.00 | 170.26 | O |
| HETATM | 2288 | C3 | BMA | A | 312 | 106.560 | 23.196 | 26.872 | 1.00 | 176.32 | C |
| HETATM | 2289 | O3 | BMA | A | 312 | 107.308 | 24.319 | 27.392 | 1.00 | 179.24 | O |
| HETATM | 2290 | C2 | BMA | A | 312 | 105.549 | 22.571 | 27.845 | 1.00 | 175.80 | C |
| HETATM | 2291 | O2 | BMA | A | 312 | 106.163 | 21.983 | 28.997 | 1.00 | 179.19 | O |
| HETATM | 2292 | C1 | MAN | A | 313 | 105.668 | 18.645 | 24.052 | 1.00 | 173.82 | C |
| HETATM | 2293 | C2 | MAN | A | 313 | 105.951 | 17.157 | 24.240 | 1.00 | 173.85 | C |
| HETATM | 2294 | O2 | MAN | A | 313 | 104.749 | 16.562 | 24.671 | 1.00 | 170.58 | O |
| HETATM | 2295 | C3 | MAN | A | 313 | 106.456 | 16.548 | 22.927 | 1.00 | 175.11 | C |
| HETATM | 2296 | O3 | MAN | A | 313 | 106.892 | 15.191 | 23.021 | 1.00 | 179.56 | O |
| HETATM | 2297 | C4 | MAN | A | 313 | 105.468 | 16.852 | 21.794 | 1.00 | 173.27 | C |
| HETATM | 2298 | O4 | MAN | A | 313 | 106.007 | 16.404 | 20.573 | 1.00 | 171.08 | O |
| HETATM | 2299 | C5 | MAN | A | 313 | 105.196 | 18.355 | 21.719 | 1.00 | 173.04 | C |
| HETATM | 2300 | C6 | MAN | A | 313 | 104.164 | 18.707 | 20.645 | 1.00 | 169.96 | C |
| HETATM | 2301 | O6 | MAN | A | 313 | 102.944 | 18.034 | 20.866 | 1.00 | 163.68 | O |
| HETATM | 2302 | O5 | MAN | A | 313 | 104.756 | 18.830 | 22.980 | 1.00 | 175.64 | O |
| HETATM | 2303 | C1 | MAN | A | 314 | 105.922 | 14.184 | 23.394 | 1.00 | 178.69 | C |
| HETATM | 2304 | C2 | MAN | A | 314 | 106.171 | 13.732 | 24.832 | 1.00 | 175.17 | C |
| HETATM | 2305 | O2 | MAN | A | 314 | 105.094 | 12.927 | 25.259 | 1.00 | 167.65 | O |
| HETATM | 2306 | C3 | MAN | A | 314 | 107.477 | 12.949 | 24.950 | 1.00 | 175.89 | C |
| HETATM | 2307 | O3 | MAN | A | 314 | 107.610 | 12.413 | 26.248 | 1.00 | 175.01 | O |
| HETATM | 2308 | C4 | MAN | A | 314 | 107.523 | 11.824 | 23.924 | 1.00 | 177.70 | C |
| HETATM | 2309 | O4 | MAN | A | 314 | 108.804 | 11.232 | 23.946 | 1.00 | 175.31 | O |
| HETATM | 2310 | C5 | MAN | A | 314 | 107.221 | 12.378 | 22.532 | 1.00 | 179.10 | C |
| HETATM | 2311 | C6 | MAN | A | 314 | 107.184 | 11.279 | 21.474 | 1.00 | 177.74 | C |
| HETATM | 2312 | O6 | MAN | A | 314 | 106.145 | 10.367 | 21.754 | 1.00 | 177.31 | O |
| HETATM | 2313 | O5 | MAN | A | 314 | 105.974 | 13.053 | 22.545 | 1.00 | 180.90 | O |
| HETATM | 2314 | C1 | MAN | A | 315 | 107.996 | 24.177 | 28.656 | 1.00 | 178.04 | C |
| HETATM | 2315 | C2 | MAN | A | 315 | 107.861 | 25.503 | 29.400 | 1.00 | 176.71 | C |
| HETATM | 2316 | O2 | MAN | A | 315 | 108.273 | 25.337 | 30.738 | 1.00 | 176.35 | O |
| HETATM | 2317 | C3 | MAN | A | 315 | 108.691 | 26.587 | 28.720 | 1.00 | 174.62 | C |
| HETATM | 2318 | O3 | MAN | A | 315 | 108.650 | 27.779 | 29.472 | 1.00 | 174.02 | O |
| HETATM | 2319 | C4 | MAN | A | 315 | 110.134 | 26.123 | 28.555 | 1.00 | 174.24 | C |
| HETATM | 2320 | O4 | MAN | A | 315 | 110.840 | 27.087 | 27.806 | 1.00 | 169.00 | O |
| HETATM | 2321 | C5 | MAN | A | 315 | 110.186 | 24.763 | 27.856 | 1.00 | 175.46 | C |
| HETATM | 2322 | C6 | MAN | A | 315 | 111.599 | 24.182 | 27.804 | 1.00 | 172.54 | C |
| HETATM | 2323 | O6 | MAN | A | 315 | 112.577 | 25.196 | 27.752 | 1.00 | 170.89 | O |
| HETATM | 2324 | O5 | MAN | A | 315 | 109.361 | 23.825 | 28.526 | 1.00 | 178.20 | O |
| HETATM | 2325 | C1 | NAG | A | 320 | 99.704 | 11.932 | 5.032 | 1.00 | 78.00 | C |
| HETATM | 2326 | C2 | NAG | A | 320 | 101.063 | 11.242 | 5.063 | 1.00 | 79.63 | C |
| HETATM | 2327 | N2 | NAG | A | 320 | 101.690 | 11.279 | 3.754 | 1.00 | 81.27 | N |
| HETATM | 2328 | C7 | NAG | A | 320 | 102.732 | 12.057 | 3.458 | 1.00 | 83.86 | C |
| HETATM | 2329 | O7 | NAG | A | 320 | 103.272 | 12.831 | 4.251 | 1.00 | 85.89 | O |
| HETATM | 2330 | C8 | NAG | A | 320 | 103.254 | 11.957 | 2.055 | 1.00 | 84.99 | C |
| HETATM | 2331 | C3 | NAG | A | 320 | 100.933 | 9.788 | 5.507 | 1.00 | 79.53 | C |
| HETATM | 2332 | O3 | NAG | A | 320 | 102.213 | 9.258 | 5.761 | 1.00 | 78.00 | O |
| HETATM | 2333 | C4 | NAG | A | 320 | 100.100 | 9.685 | 6.772 | 1.00 | 80.67 | C |
| HETATM | 2334 | O4 | NAG | A | 320 | 99.818 | 8.327 | 7.056 | 1.00 | 85.62 | O |
| HETATM | 2335 | C5 | NAG | A | 320 | 98.796 | 10.457 | 6.615 | 1.00 | 78.92 | C |
| HETATM | 2336 | C6 | NAG | A | 320 | 97.984 | 10.436 | 7.902 | 1.00 | 81.04 | C |
| HETATM | 2337 | O6 | NAG | A | 320 | 98.777 | 10.903 | 8.973 | 1.00 | 82.77 | O |
| HETATM | 2338 | O5 | NAG | A | 320 | 99.095 | 11.795 | 6.297 | 1.00 | 77.52 | O |
| HETATM | 2339 | C1 | NAG | A | 321 | 100.443 | 7.908 | 8.284 | 1.00 | 90.18 | C |
| HETATM | 2340 | C2 | NAG | A | 321 | 99.708 | 6.704 | 8.850 | 1.00 | 90.56 | C |
| HETATM | 2341 | N2 | NAG | A | 321 | 98.319 | 7.015 | 9.118 | 1.00 | 86.90 | N |
| HETATM | 2342 | C7 | NAG | A | 321 | 97.320 | 6.575 | 8.360 | 1.00 | 86.04 | C |
| HETATM | 2343 | O7 | NAG | A | 321 | 97.469 | 5.880 | 7.356 | 1.00 | 86.02 | O |
| HETATM | 2344 | C8 | NAG | A | 321 | 95.949 | 6.988 | 8.795 | 1.00 | 86.14 | C |
| HETATM | 2345 | C3 | NAG | A | 321 | 100.336 | 6.267 | 10.162 | 1.00 | 95.00 | C |
| HETATM | 2346 | O3 | NAG | A | 321 | 99.774 | 5.031 | 10.540 | 1.00 | 94.11 | O |
| HETATM | 2347 | C4 | NAG | A | 321 | 101.852 | 6.146 | 10.078 | 1.00 | 99.90 | C |
| HETATM | 2348 | O4 | NAG | A | 321 | 102.343 | 6.206 | 11.401 | 1.00 | 111.46 | O |
| HETATM | 2349 | C5 | NAG | A | 321 | 102.493 | 7.282 | 9.281 | 1.00 | 97.44 | C |
| HETATM | 2350 | C6 | NAG | A | 321 | 103.947 | 6.969 | 8.938 | 1.00 | 97.10 | C |
| HETATM | 2351 | O6 | NAG | A | 321 | 103.999 | 6.031 | 7.886 | 1.00 | 97.63 | O |
| HETATM | 2352 | O5 | NAG | A | 321 | 101.790 | 7.546 | 8.083 | 1.00 | 94.30 | O |
| HETATM | 2353 | C1 | BMA | A | 322 | 102.661 | 4.906 | 11.925 | 1.00 | 122.99 | C |
| HETATM | 2354 | O5 | BMA | A | 322 | 101.525 | 4.109 | 12.255 | 1.00 | 127.83 | O |
| HETATM | 2355 | C5 | BMA | A | 322 | 101.884 | 2.771 | 12.618 | 1.00 | 133.51 | C |
| HETATM | 2356 | C6 | BMA | A | 322 | 100.615 | 1.981 | 12.924 | 1.00 | 137.04 | C |
| HETATM | 2357 | O6 | BMA | A | 322 | 99.842 | 2.620 | 13.953 | 1.00 | 142.80 | O |
| HETATM | 2358 | C4 | BMA | A | 322 | 102.860 | 2.751 | 13.801 | 1.00 | 134.12 | C |
| HETATM | 2359 | O4 | BMA | A | 322 | 103.375 | 1.426 | 13.966 | 1.00 | 134.46 | O |
| HETATM | 2360 | C3 | BMA | A | 322 | 104.027 | 3.719 | 13.595 | 1.00 | 134.38 | C |
| HETATM | 2361 | O3 | BMA | A | 322 | 104.824 | 3.820 | 14.788 | 1.00 | 140.14 | O |
| HETATM | 2362 | C2 | BMA | A | 322 | 103.480 | 5.082 | 13.190 | 1.00 | 128.00 | C |
| HETATM | 2363 | O2 | BMA | A | 322 | 102.656 | 5.617 | 14.234 | 1.00 | 123.99 | O |
| HETATM | 2364 | C1 | MAN | A | 323 | 106.165 | 4.289 | 14.504 | 1.00 | 144.86 | C |
| HETATM | 2365 | C2 | MAN | A | 323 | 107.113 | 3.102 | 14.303 | 1.00 | 145.83 | C |
| HETATM | 2366 | O2 | MAN | A | 323 | 108.221 | 3.519 | 13.532 | 1.00 | 142.79 | O |
| HETATM | 2367 | C3 | MAN | A | 323 | 107.599 | 2.476 | 15.614 | 1.00 | 148.36 | C |
| HETATM | 2368 | O3 | MAN | A | 323 | 108.743 | 1.684 | 15.376 | 1.00 | 147.23 | O |
| HETATM | 2369 | C4 | MAN | A | 323 | 107.926 | 3.522 | 16.674 | 1.00 | 148.74 | C |
| HETATM | 2370 | O4 | MAN | A | 323 | 108.112 | 2.888 | 17.920 | 1.00 | 149.10 | O |
| HETATM | 2371 | C5 | MAN | A | 323 | 106.804 | 4.549 | 16.777 | 1.00 | 147.56 | C |
| HETATM | 2372 | C6 | MAN | A | 323 | 107.115 | 5.600 | 17.840 | 1.00 | 145.14 | C |
| HETATM | 2373 | O6 | MAN | A | 323 | 106.520 | 6.836 | 17.507 | 1.00 | 142.88 | O |
| HETATM | 2374 | O5 | MAN | A | 323 | 106.646 | 5.160 | 15.511 | 1.00 | 147.26 | O |
| HETATM | 2375 | C1 | MAN | A | 324 | 98.715 | 1.788 | 14.313 | 1.00 | 148.42 | C |
| HETATM | 2376 | C2 | MAN | A | 324 | 97.413 | 2.512 | 13.959 | 1.00 | 148.60 | C |
| HETATM | 2377 | O2 | MAN | A | 324 | 96.396 | 1.554 | 13.757 | 1.00 | 150.22 | O |
| HETATM | 2378 | C3 | MAN | A | 324 | 96.973 | 3.530 | 15.015 | 1.00 | 149.37 | C |
| HETATM | 2379 | O3 | MAN | A | 324 | 95.624 | 3.891 | 14.809 | 1.00 | 147.64 | O |
| HETATM | 2380 | C4 | MAN | A | 324 | 97.141 | 2.995 | 16.432 | 1.00 | 149.92 | C |
| HETATM | 2381 | O4 | MAN | A | 324 | 96.913 | 4.039 | 17.352 | 1.00 | 148.37 | O |
| HETATM | 2382 | C5 | MAN | A | 324 | 98.542 | 2.423 | 16.616 | 1.00 | 150.46 | C |
| HETATM | 2383 | C6 | MAN | A | 324 | 98.735 | 1.846 | 18.015 | 1.00 | 148.18 | C |
| HETATM | 2384 | O6 | MAN | A | 324 | 98.707 | 2.883 | 18.969 | 1.00 | 148.30 | O |
| HETATM | 2385 | O5 | MAN | A | 324 | 98.741 | 1.389 | 15.673 | 1.00 | 152.09 | O |
| HETATM | 2386 | C1 | NAG | A | 330 | 113.415 | 26.632 | 18.299 | 1.00 | 110.44 | C |
| HETATM | 2387 | C2 | NAG | A | 330 | 113.451 | 27.224 | 19.699 | 1.00 | 114.33 | C |
| HETATM | 2388 | N2 | NAG | A | 330 | 112.698 | 28.467 | 19.720 | 1.00 | 110.83 | N |
| HETATM | 2389 | C7 | NAG | A | 330 | 111.815 | 28.817 | 20.662 | 1.00 | 109.92 | C |
| HETATM | 2390 | O7 | NAG | A | 330 | 111.216 | 29.888 | 20.603 | 1.00 | 108.90 | O |
| HETATM | 2391 | C8 | NAG | A | 330 | 111.543 | 27.895 | 21.819 | 1.00 | 109.21 | C |
| HETATM | 2392 | C3 | NAG | A | 330 | 114.898 | 27.468 | 20.090 | 1.00 | 118.83 | C |
| HETATM | 2393 | O3 | NAG | A | 330 | 114.967 | 27.874 | 21.436 | 1.00 | 118.84 | O |
| HETATM | 2394 | C4 | NAG | A | 330 | 115.703 | 26.186 | 19.892 | 1.00 | 123.15 | C |
| HETATM | 2395 | O4 | NAG | A | 330 | 117.068 | 26.467 | 20.115 | 1.00 | 134.68 | O |
| HETATM | 2396 | C5 | NAG | A | 330 | 115.502 | 25.610 | 18.490 | 1.00 | 117.64 | C |
| HETATM | 2397 | C6 | NAG | A | 330 | 116.192 | 24.267 | 18.296 | 1.00 | 115.10 | C |
| HETATM | 2398 | O6 | NAG | A | 330 | 115.595 | 23.304 | 19.137 | 1.00 | 110.28 | O |
| HETATM | 2399 | O5 | NAG | A | 330 | 114.125 | 25.419 | 18.271 | 1.00 | 113.09 | O |
| HETATM | 2400 | C1 | NAG | A | 331 | 117.623 | 25.577 | 21.098 | 1.00 | 141.38 | C |
| HETATM | 2401 | C2 | NAG | A | 331 | 119.097 | 25.938 | 21.284 | 1.00 | 142.10 | C |
| HETATM | 2402 | N2 | NAG | A | 331 | 119.820 | 25.923 | 20.014 | 1.00 | 137.18 | N |
| HETATM | 2403 | C7 | NAG | A | 331 | 120.034 | 24.827 | 19.278 | 1.00 | 132.94 | C |
| HETATM | 2404 | O7 | NAG | A | 331 | 119.651 | 23.703 | 19.593 | 1.00 | 130.56 | O |
| HETATM | 2405 | C8 | NAG | A | 331 | 120.795 | 25.006 | 17.995 | 1.00 | 132.22 | C |
| HETATM | 2406 | C3 | NAG | A | 331 | 119.740 | 25.076 | 22.372 | 1.00 | 146.26 | C |
| HETATM | 2407 | O3 | NAG | A | 331 | 121.036 | 25.546 | 22.661 | 1.00 | 145.00 | O |
| HETATM | 2408 | C4 | NAG | A | 331 | 118.870 | 25.127 | 23.623 | 1.00 | 152.12 | C |
| HETATM | 2409 | O4 | NAG | A | 331 | 119.398 | 24.297 | 24.638 | 1.00 | 162.28 | O |
| HETATM | 2410 | C5 | NAG | A | 331 | 117.443 | 24.712 | 23.273 | 1.00 | 149.81 | C |
| HETATM | 2411 | C6 | NAG | A | 331 | 116.516 | 24.666 | 24.486 | 1.00 | 149.59 | C |
| HETATM | 2412 | O6 | NAG | A | 331 | 116.467 | 25.929 | 25.110 | 1.00 | 150.33 | O |
| HETATM | 2413 | O5 | NAG | A | 331 | 116.951 | 25.649 | 22.340 | 1.00 | 144.92 | O |
| HETATM | 2414 | C1 | BMA | A | 332 | 119.957 | 25.093 | 25.701 | 1.00 | 168.28 | C |
| HETATM | 2415 | O5 | BMA | A | 332 | 121.230 | 25.636 | 25.363 | 1.00 | 170.32 | O |
| HETATM | 2416 | C5 | BMA | A | 332 | 121.709 | 26.581 | 26.321 | 1.00 | 168.05 | C |
| HETATM | 2417 | C6 | BMA | A | 332 | 123.071 | 27.095 | 25.858 | 1.00 | 163.68 | C |
| HETATM | 2418 | O6 | BMA | A | 332 | 122.918 | 28.350 | 25.187 | 1.00 | 159.66 | O |
| HETATM | 2419 | C4 | BMA | A | 332 | 121.806 | 25.969 | 27.721 | 1.00 | 171.45 | C |
| HETATM | 2420 | O4 | BMA | A | 332 | 121.940 | 27.054 | 28.648 | 1.00 | 169.83 | O |
| HETATM | 2421 | C3 | BMA | A | 332 | 120.602 | 25.100 | 28.112 | 1.00 | 173.51 | C |
| HETATM | 2422 | O3 | BMA | A | 332 | 120.940 | 24.206 | 29.186 | 1.00 | 179.72 | O |
| HETATM | 2423 | C2 | BMA | A | 332 | 120.106 | 24.243 | 26.951 | 1.00 | 170.13 | C |
| HETATM | 2424 | O2 | BMA | A | 332 | 121.018 | 23.165 | 26.702 | 1.00 | 165.46 | O |
| HETATM | 2425 | C1 | MAN | A | 333 | 120.952 | 24.832 | 30.488 | 1.00 | 181.80 | C |
| HETATM | 2426 | C2 | MAN | A | 333 | 122.387 | 24.853 | 31.001 | 1.00 | 181.64 | C |
| HETATM | 2427 | O2 | MAN | A | 333 | 122.457 | 25.624 | 32.180 | 1.00 | 179.83 | O |
| HETATM | 2428 | C3 | MAN | A | 333 | 122.877 | 23.433 | 31.267 | 1.00 | 180.97 | C |
| HETATM | 2429 | O3 | MAN | A | 333 | 124.163 | 23.465 | 31.847 | 1.00 | 178.86 | O |
| HETATM | 2430 | C4 | MAN | A | 333 | 121.903 | 22.692 | 32.178 | 1.00 | 181.14 | C |
| HETATM | 2431 | O4 | MAN | A | 333 | 122.278 | 21.333 | 32.241 | 1.00 | 179.06 | O |
| HETATM | 2432 | C5 | MAN | A | 333 | 120.464 | 22.813 | 31.673 | 1.00 | 179.97 | C |
| HETATM | 2433 | C6 | MAN | A | 333 | 119.472 | 22.252 | 32.688 | 1.00 | 175.98 | C |
| HETATM | 2434 | O6 | MAN | A | 333 | 118.313 | 21.812 | 32.017 | 1.00 | 171.57 | O |
| HETATM | 2435 | O5 | MAN | A | 333 | 120.127 | 24.167 | 31.426 | 1.00 | 181.86 | O |
| HETATM | 2436 | C1 | NAG | A | 340 | 100.888 | 27.243 | −17.586 | 1.00 | 88.88 | C |
| HETATM | 2437 | C2 | NAG | A | 340 | 99.518 | 26.573 | −17.707 | 1.00 | 90.13 | C |
| HETATM | 2438 | N2 | NAG | A | 340 | 99.446 | 25.405 | −18.592 | 1.00 | 92.46 | N |
| HETATM | 2439 | C7 | NAG | A | 340 | 99.882 | 25.283 | −19.848 | 1.00 | 92.58 | C |
| HETATM | 2440 | O7 | NAG | A | 340 | 99.757 | 24.207 | −20.432 | 1.00 | 91.08 | O |
| HETATM | 2441 | C8 | NAG | A | 340 | 100.517 | 26.438 | −20.572 | 1.00 | 94.41 | C |
| HETATM | 2442 | C3 | NAG | A | 340 | 98.440 | 27.624 | −17.910 | 1.00 | 89.36 | C |
| HETATM | 2443 | O3 | NAG | A | 340 | 97.185 | 26.988 | −17.974 | 1.00 | 89.82 | O |
| HETATM | 2444 | C4 | NAG | A | 340 | 98.497 | 28.556 | −16.702 | 1.00 | 89.59 | C |
| HETATM | 2445 | O4 | NAG | A | 340 | 97.691 | 29.694 | −16.929 | 1.00 | 94.05 | O |
| HETATM | 2446 | C5 | NAG | A | 340 | 99.924 | 29.020 | −16.373 | 1.00 | 88.48 | C |
| HETATM | 2447 | C6 | NAG | A | 340 | 99.977 | 29.712 | −15.011 | 1.00 | 87.61 | C |
| HETATM | 2448 | O6 | NAG | A | 340 | 99.455 | 28.875 | −14.001 | 1.00 | 84.45 | O |
| HETATM | 2449 | O5 | NAG | A | 340 | 100.874 | 27.967 | −16.379 | 1.00 | 88.47 | O |
| HETATM | 2450 | C1 | NAG | A | 341 | 96.435 | 29.594 | −16.232 | 1.00 | 96.35 | C |
| HETATM | 2451 | C2 | NAG | A | 341 | 95.794 | 30.976 | −16.163 | 1.00 | 96.82 | C |
| HETATM | 2452 | N2 | NAG | A | 341 | 96.637 | 31.906 | −15.427 | 1.00 | 95.78 | N |
| HETATM | 2453 | C7 | NAG | A | 341 | 97.606 | 32.617 | −16.010 | 1.00 | 95.58 | C |
| HETATM | 2454 | O7 | NAG | A | 341 | 97.874 | 32.543 | −17.210 | 1.00 | 95.58 | O |
| HETATM | 2455 | C8 | NAG | A | 341 | 98.401 | 33.540 | −15.131 | 1.00 | 95.35 | C |
| HETATM | 2456 | C3 | NAG | A | 341 | 94.416 | 30.892 | −15.520 | 1.00 | 99.78 | C |
| HETATM | 2457 | O3 | NAG | A | 341 | 93.789 | 32.152 | −15.588 | 1.00 | 99.67 | O |
| HETATM | 2458 | C4 | NAG | A | 341 | 93.591 | 29.840 | −16.252 | 1.00 | 104.00 | C |
| HETATM | 2459 | O4 | NAG | A | 341 | 92.301 | 29.715 | −15.684 | 1.00 | 111.33 | O |
| HETATM | 2460 | C5 | NAG | A | 341 | 94.327 | 28.504 | −16.243 | 1.00 | 102.77 | C |
| HETATM | 2461 | C6 | NAG | A | 341 | 93.552 | 27.414 | −16.979 | 1.00 | 104.49 | C |
| HETATM | 2462 | O6 | NAG | A | 341 | 93.598 | 27.650 | −18.370 | 1.00 | 105.18 | O |
| HETATM | 2463 | O5 | NAG | A | 341 | 95.577 | 28.676 | −16.876 | 1.00 | 98.21 | O |
| HETATM | 2464 | C1 | BMA | A | 342 | 91.328 | 30.498 | −16.408 | 1.00 | 119.64 | C |
| HETATM | 2465 | O5 | BMA | A | 342 | 91.085 | 31.765 | −15.791 | 1.00 | 120.34 | O |
| HETATM | 2466 | C5 | BMA | A | 342 | 90.208 | 32.602 | −16.547 | 1.00 | 121.32 | C |
| HETATM | 2467 | C6 | BMA | A | 342 | 90.160 | 33.985 | −15.906 | 1.00 | 121.40 | C |
| HETATM | 2468 | O6 | BMA | A | 342 | 91.453 | 34.603 | −16.035 | 1.00 | 124.56 | O |
| HETATM | 2469 | C4 | BMA | A | 342 | 88.838 | 31.936 | −16.610 | 1.00 | 123.92 | C |
| HETATM | 2470 | O4 | BMA | A | 342 | 87.945 | 32.698 | −17.428 | 1.00 | 121.93 | O |
| HETATM | 2471 | C3 | BMA | A | 342 | 88.953 | 30.537 | −17.203 | 1.00 | 128.89 | C |
| HETATM | 2472 | O3 | BMA | A | 342 | 87.672 | 29.893 | −17.113 | 1.00 | 140.69 | O |
| HETATM | 2473 | C2 | BMA | A | 342 | 90.035 | 29.699 | −16.517 | 1.00 | 124.67 | C |
| HETATM | 2474 | O2 | BMA | A | 342 | 89.623 | 29.265 | −15.215 | 1.00 | 123.88 | O |
| HETATM | 2475 | C1 | MAN | A | 343 | 87.247 | 29.408 | −18.403 | 1.00 | 151.30 | C |
| HETATM | 2476 | C2 | MAN | A | 343 | 85.721 | 29.263 | −18.399 | 1.00 | 154.03 | C |
| HETATM | 2477 | O2 | MAN | A | 343 | 85.137 | 28.979 | −19.667 | 1.00 | 161.06 | O |
| HETATM | 2478 | C3 | MAN | A | 343 | 85.324 | 28.143 | −17.439 | 1.00 | 150.64 | C |
| HETATM | 2479 | O3 | MAN | A | 343 | 83.935 | 27.917 | −17.508 | 1.00 | 147.95 | O |
| HETATM | 2480 | C4 | MAN | A | 343 | 86.065 | 26.864 | −17.811 | 1.00 | 151.44 | C |
| HETATM | 2481 | O4 | MAN | A | 343 | 85.807 | 25.852 | −16.867 | 1.00 | 146.63 | O |
| HETATM | 2482 | C5 | MAN | A | 343 | 87.571 | 27.112 | −17.856 | 1.00 | 153.70 | C |
| HETATM | 2483 | C6 | MAN | A | 343 | 88.343 | 25.860 | −18.278 | 1.00 | 155.05 | C |
| HETATM | 2484 | O6 | MAN | A | 343 | 87.459 | 24.892 | −18.810 | 1.00 | 158.77 | O |
| HETATM | 2485 | O5 | MAN | A | 343 | 87.851 | 28.175 | −18.751 | 1.00 | 154.50 | O |
| HETATM | 2486 | C1 | MAN | A | 345 | 85.695 | 29.622 | −20.841 | 1.00 | 166.37 | C |
| HETATM | 2487 | C2 | MAN | A | 345 | 85.634 | 31.158 | −20.781 | 1.00 | 167.59 | C |
| HETATM | 2488 | O2 | MAN | A | 345 | 86.580 | 31.692 | −21.683 | 1.00 | 167.20 | O |
| HETATM | 2489 | C3 | MAN | A | 345 | 84.245 | 31.731 | −21.075 | 1.00 | 166.74 | C |
| HETATM | 2490 | O3 | MAN | A | 345 | 84.322 | 33.124 | −21.288 | 1.00 | 162.83 | O |
| HETATM | 2491 | C4 | MAN | A | 345 | 83.617 | 31.055 | −22.288 | 1.00 | 167.34 | C |
| HETATM | 2492 | O4 | MAN | A | 345 | 82.280 | 31.482 | −22.423 | 1.00 | 162.36 | O |
| HETATM | 2493 | C5 | MAN | A | 345 | 83.684 | 29.542 | −22.101 | 1.00 | 168.99 | C |
| HETATM | 2494 | C6 | MAN | A | 345 | 83.040 | 28.783 | −23.255 | 1.00 | 168.07 | C |
| HETATM | 2495 | O6 | MAN | A | 345 | 83.134 | 27.399 | −22.995 | 1.00 | 164.56 | O |
| HETATM | 2496 | O5 | MAN | A | 345 | 85.042 | 29.160 | −22.008 | 1.00 | 168.63 | O |
| HETATM | 2497 | C1 | MAN | A | 344 | 91.467 | 35.742 | −16.924 | 1.00 | 128.05 | C |
| HETATM | 2498 | C2 | MAN | A | 344 | 92.582 | 35.574 | −17.955 | 1.00 | 130.27 | C |
| HETATM | 2499 | O2 | MAN | A | 344 | 92.454 | 36.565 | −18.952 | 1.00 | 128.74 | O |
| HETATM | 2500 | C3 | MAN | A | 344 | 93.959 | 35.658 | −17.296 | 1.00 | 130.86 | C |
| HETATM | 2501 | O3 | MAN | A | 344 | 94.974 | 35.677 | −18.278 | 1.00 | 129.03 | O |
| HETATM | 2502 | C4 | MAN | A | 344 | 94.078 | 36.901 | −16.419 | 1.00 | 129.88 | C |
| HETATM | 2503 | O4 | MAN | A | 344 | 95.251 | 36.794 | −15.641 | 1.00 | 125.41 | O |
| HETATM | 2504 | C5 | MAN | A | 344 | 92.861 | 37.074 | −15.505 | 1.00 | 129.52 | C |
| HETATM | 2505 | C6 | MAN | A | 344 | 92.875 | 38.441 | −14.825 | 1.00 | 129.77 | C |
| HETATM | 2506 | O6 | MAN | A | 344 | 93.749 | 38.437 | −13.718 | 1.00 | 128.61 | O |
| HETATM | 2507 | O5 | MAN | A | 344 | 91.648 | 36.961 | −16.230 | 1.00 | 127.47 | O |
| HETATM | 2508 | C1 | MAN | A | 346 | 88.141 | 23.896 | −19.600 | 1.00 | 161.77 | C |
| HETATM | 2509 | C2 | MAN | A | 346 | 88.219 | 24.347 | −21.060 | 1.00 | 161.25 | C |
| HETATM | 2510 | O2 | MAN | A | 346 | 89.132 | 23.525 | −21.752 | 1.00 | 159.91 | O |
| HETATM | 2511 | C3 | MAN | A | 346 | 86.856 | 24.287 | −21.743 | 1.00 | 161.31 | C |
| HETATM | 2512 | O3 | MAN | A | 346 | 86.990 | 24.544 | −23.123 | 1.00 | 161.03 | O |
| HETATM | 2513 | C4 | MAN | A | 346 | 86.221 | 22.919 | −21.532 | 1.00 | 159.90 | C |
| HETATM | 2514 | O4 | MAN | A | 346 | 84.913 | 22.924 | −22.053 | 1.00 | 159.25 | O |
| HETATM | 2515 | C5 | MAN | A | 346 | 86.185 | 22.604 | −20.039 | 1.00 | 159.17 | C |
| HETATM | 2516 | C6 | MAN | A | 346 | 85.541 | 21.251 | −19.747 | 1.00 | 155.90 | C |
| HETATM | 2517 | O6 | MAN | A | 346 | 86.162 | 20.229 | −20.493 | 1.00 | 152.47 | O |
| HETATM | 2518 | O5 | MAN | A | 346 | 87.505 | 22.631 | −19.523 | 1.00 | 163.55 | O |

1. A polysaccharide-oxidizing composition comprising a polysaccharide-oxidizing enzyme wherein, when the polysaccharide-oxidizing enzyme is compared to the reference polypeptide of SEQ ID NO. 1 by using the BLAST-P comparison method, (i) the polysaccharide-oxidizing enzyme possesses an amino acid identity of 20% or more with the reference polypeptide and (ii) the BLAST-P comparison method results in an E-value of 10 e⁻³ or less.

2. The polysaccharide-oxidizing composition according to claim 1, wherein the polysaccharide-oxidizing enzyme has at least 90% amino acid identity with a polypeptide selected from the group consisting of the polypeptides of SEQ ID NO. 1, SEQ ID NO. 2 and SEQ ID NO. 3.

3. The polysaccharide-oxidizing composition according to claim 1, wherein the polysaccharide-oxidizing enzyme is encoded by a nucleic acid having at least 90% nucleotide identity with a nucleic acid selected a from the group consisting of the nucleic acids of SEQ ID NO. 4, SEQ ID NO. 5 and SEQ ID NO. 6.

4. The polysaccharide-oxidizing composition according to claim 1, which further comprises one or more lytic polysaccharide monooxygenases.

5. A polysaccharide-degrading composition comprising the polysaccharide-oxidizing enzyme of claim 1 and one or more polysaccharide-degrading enzymes selected from the group consisting of cellulases, hemicellulases, ligninases, and carbohydrate oxidases.

6. The polysaccharide-degrading composition according to claim 5, wherein the cellulases are selected from the group consisting or exoglucanases, endoglucanases, cellobiohydrolases, cellulose phosphorylases, pectinases, pectate lyases, polygalacturonase, pectin esterases, cellobiose dehydrogenases, beta mannanases, arabinofuranosidases, feruoyl esterases, arabinofuranosidases, fructofuranosidases, alpha galactosidases, beta galactosidases, alpha amylases, acetylxylan esterases, chitin deacetylases, chitinases, and beta glucosidases.

7. The polysaccharide-degrading composition according to claim 5, which comprises a lytic polysaccharide monooxygenase selected from the group consisting of AA9, AA10, AA11 and AA13.

8. The polysaccharide-degrading composition according to claim 5, wherein the one or more polysaccharide degrading enzymes are comprised in an enzyme preparation containing the one or more polysaccharide degrading enzymes.

9. The polysaccharide-degrading composition according to claim 8, wherein the enzyme preparation comprises one or more enzymes originating from one or more fungus organisms or one or more bacterial organisms.

10. The polysaccharide-degrading composition according to claim 9, wherein the one or more fungus organisms is selected from group consisting of fungi of the genus Achlya Acremonium, Gliocladium, Humicola, Hypocrea, Myceliophthora, Mucor, Neurospora, Penicillium, Pyricularia, Thielavia, Tolypocladium, Trichoderma, Podospora, Pycnoporus, Fusarium, Thermonospora, Hypocrea, Humicola, Penicillium, Myceliophthora and Aspergillus.

11. The polysaccharide-degrading composition according to claim 8 wherein the enzyme preparation comprises an enzyme extract from one or more fungus organisms or from one or more bacterial organisms.

12. The polysaccharide-degrading composition according to claim 5, wherein one or more of the one or more polysaccharide degrading enzymes are recombinant proteins.

13. A yeast cell recombinantly expressing the polysaccharide-oxidizing enzyme of claim 1.

14. A method for oxidizing a polysaccharide comprising a step of contacting one or more polysaccharides with the polysaccharide-oxidizing enzyme of claim 1 or with a composition comprising the polysaccharide-oxidizing enzyme.

15. The method according to claim 14, wherein the one or more poly saccharides are comprised in a poly saccharide-containing biomass.

16. The method according to claim 14, wherein the one or more polysaccharides are contained in a lignocellulosic-containing material.

17. A method for the preparation of a sugar product from a polysaccharide-containing material comprising a step of treating the polysaccharide-containing material in the presence of the polysaccharide degrading composition according to claim 5.

18. The method according to claim 17, which comprises the steps of:
a) providing the polysaccharide-containing material,
b) subjecting the polysaccharide-containing material to hydrolysis in the presence of the polysaccharide-degrading composition according to claim 5, and
c) collecting the sugar product obtained at the end of step b).

19. A method for the preparation of a fermentation product from a polysaccharide-containing material comprising the steps or:
a) providing the polysaccharide-containing material,
b) subjecting the polysaccharide-containing material to hydrolysis in the presence of the polysaccharide-degrading composition according to claim 5, whereby a sugar product is obtained, and
c) fermenting the sugar product, whereby a fermentation product is obtained.

20. The method according to claim 17, wherein the polysaccharide-containing material is a lignocellulosic material.


SEQUENCE LISTING (9 sequences)

SEQ ID NO: 1 - protein, 270 aa, Pycnoporus coccineus
  amino acid sequence of polysaccharide oxidizing enzyme
HAAFWDKSMYGFNVTAQTFPYDNRPQVPLYNMTFDQWWFHGHKDYPPNEGDFFELPAGGEVNSIISCDKGATPFYESSPGGDSGYGSNSPCPGQPMSEYHTTGIDDVKGCCMAIAYKPDVNDVQPDDFVVFSCNSTCVWEMNTKFEIPKLPACPEGGCHCAWFWIHSYDSGAEQIYMNGFKCKVTGDVGTQPLGKPAVPRRCGADPDHGKPDPTPGNCTIGAKTPMYWYQREGNNMFEDTYDAPYYNPLYGFNDGAQNDIFMDGVIASLA

SEQ ID NO: 2 - protein, 269 aa, Pycnoporus coccineus
  amino acid sequence of polysaccharide oxidizing enzyme
HIAFWHNSMYGFNVTEQTFPYDNRPVVPLQYMTFQEWWFHNHLDYPPHPGDFFDFPAGKAATAELACNKGATTWFNSSEGGNIQNGNDPCPGSPPSEYHTTGIDDVKGCAMAIAYESDVRKIKPEDFTVFSVNQTCVWYRFTDFQVPERMPPCPPGGCHCAWFWIHSPDSGGEQIYMNGFQCNITGSTSHVPLAKPKVARRCGADPDHGKPDAVPGNCTYGAKQPLYWLQKEGNNEFDDYIAPPFYNDLYNFKDGAQNDIFVDSYPDGI

SEQ ID NO: 3 - protein, 269 aa, Pycnoporus coccineus
  amino acid of polysaccharise enzyme
HVAAFVKGMYCEGGPDPNNYNPNSNTPVNPLWDLPFEQWWMQADRGCNKAPPPDGASVALPAGGQFTVELAHNQAQTTLSFNGQFAGEWPDGQPHPENWSGPGSPPDCIQDDGAMHTNNQTMAAGTAWAISYNDDISKVTMDNLVVFSVLEHTPWKRIATYDVPKDLPACPAGGCYCAWLWVPNGCGEPNMYMANYRCHVTNTTSTKQLAQAKPPTWCGGDSSKCTKGAKQMIAWNQATGNNVQVPNGASPGYNINMGWAPGAQNDIFA

SEQ ID NO: 4 - DNA, 810 bp, Pycnoporus coccineus
  nucleic sequence of polysaccharide enzyme
cacgccgcgttctgggacaagtccatgtacggcttcaacgtcaccgcacagaccttcccctacgacaaccggccccaggttccgctctacaacatgactttcgaccaatggtggttccacggtcacaaagactacccacccaacgagggcgatttcttcgaactccctgcgggtggagaggtgaacagcatcatctcctgcgataagggtgcgactccgttctacgagtcgtctccgggcggagattcgggctatggcagcaactctccttgccccgggcagcccatgtccgagtaccacacgacgggcattgatgatgtcaagggctgctgcatggccatcgcgtacaagcccgatgtcaacgatgtgcagccagacgacttcgtggtcttctcctgcaactcgacgtgcgtttgggagatgaacaccaagtttgagatcccgaagctccctgcctgccccgaaggtggttgccattgcgcctggttctggattcactcctacgatagcggtgctgagcagatctatatgaatggtttcaagtgcaaagtaaccggcgacgtaggcactcagcctctcggcaagcctgccgtcccgcgcaggtgcggtgccgaccctgatcacggcaagcctgatcctactcccggtaattgtactatcggggccaagacgcccatgtactggtaccagcgggagggtaacaacatgttcgaagacacttatgacgccccgtactataacccactctacggcttcaacgatggggcgcagaacgacattttcatggatggtgtcatcgcgtccctcgca

SEQ ID NO: 5 - DNA, 807 bp, Pycnoporus coccineus
  nucleic sequence of polysaccharide enzyme
cacatcgccttctggcataacagcatgtacgggttcaatgtgacggaacagacgttcccttacgacaaccgccccgtcgtcccgcttcagtacatgaccttccaagaatggtggttccacaaccacctcgactacccgccccacccgggcgacttcttcgacttcccggccggcaaggccgcgacggcggagctcgcgtgcaacaagggcgcgaccacctggttcaactcctccgagggcggcaacatccagaacggcaacgacccgtgcccggggagccccccgagcgagtaccacacgacgggcatcgacgacgtgaagggctgcgcgatggcgatcgcgtacgagtccgacgtcaggaagatcaagcccgaggacttcaccgtcttcagcgtgaaccagacgtgcgtctggtaccgcttcacggacttccaggtccccgagcgcatgccgccgtgccctcctggcggctgtcactgcgcgtggttctggatccactcgcccgatagcggcggtgagcagatctacatgaacggcttccagtgcaacatcaccggctccacgtcccacgtcccgctcgcaaagcccaaagtcgctcgccgctgcggtgcggacccggaccacggcaagcccgacgccgtccccggcaactgcacatacggtgcgaagcagcccctctactggctccagaaggagggcaacaacgagttcgacgactacatcgcgccgccgttctacaacgacctgtacaacttcaaggacggcgcgcagaacgacatcttcgtcgactcgtaccccgacggcatc

SEQ ID NO: 6 - DNA, 861 bp, Pycnoporus coccineus
  nucleic sequence of polysaccharide enzyme
atgttctggacggttcccgttacactggctctcgcctccggggcctctgcccatgttgccgccttcgtgaagggcatgtactgcgagggtggccccgaccctaacaactacaaccccaactccaacactcccgtcaaccctctctgggatcttcctttcgagcagtggtggatgcaggccgaccgtggctgcaacaaggctcctcctcctgacggtgcctccgttgctctgcctgccggcggccagttcactgttgagctcgcccacaaccaggcccagacgaccttatccttcaatggacagttcgctggcgagtggcctgatggccagcctcacccagagaactggagcggccccggtagcccccctgactgcatccaggacgacggtgccatgcataccaacaaccagacaatggccgctggtaccgcttgggccatttcctacaatgacgacatctccaaggtgaccatggacaacctggttgttttctctgtccttgagcacaccccttggaagcgcatcgccacctatgatgtccccaaggatctccctgcttgccctgctggcggctgctactgcgcctggctttgggttcccaacgggtgcggcgagcccaacatgtacatggccaactacaggtgccatgtcaccaacaccacctcgaccaagcagcttgctcaagccaagccccccacctggtgtggcggtgactcttccaagtgcaccaagggcgctaagcagatgattgcttggaaccaggccacaggcaacaatgtccaagttcccaatggcgcttcccccggctacaacatcaacatgggctgggctcccggtgctcagaacgacatcttcgcgtag

SEQ ID NO: 7 - protein, 185 aa, Pycnoporus coccineus
  amino acid sequence of polysaccharide oxidizing enzyme
HIAFWHNSMYGFNVTEQTFPYDNRPVVPLQYMTFQEWWFHNHLDYPPHPGDFFDFPAGKAATAELACNKGATTWFNSSEGGNIQNGNDPCPGSPPSEYHTTGIDDVKGCAMAIAYESDVRKIKPEDFTVFSVNQTCVWYRFTDFQVPERMPPCPPGGCHCAWFWIHSPDSGGEQIYMNGFQCNIT

SEQ ID NO: 8 - protein, 17 aa, Artificial Sequence
  Synthetic signal peptide
MFWTVPVTLALASGASA

SEQ ID NO: 9 - protein, 286 aa, Pycnoporus coccineus
  amino acid sequence of polysaccharide oxidizing enzyme
MFWTVPVTLALASGASAHVAAFVKGMYCEGGPDPNNYNPNSNTPVNPLWDLPFEQWWMQADRGCNKAPPPDGASVALPAGGQFTVELAHNQAQTTLSFNGQFAGEWPDGQPHPENWSGPGSPPDCIQDDGAMHTNNQTMAAGTAWAISYNDDISKVTMDNLVVFSVLEHTPWKRIATYDVPKDLPACPAGGCYCAWLWVPNGCGEPNMYMANYRCHVTNTTSTKQLAQAKPPTWCGGDSSKCTKGAKQMIAWNQATGNNVQVPNGASPGYNINMGWAPGAQNDIFA